(19) United States
(12) Patent Application Publication
(10) Pub. No.: US 2010/0048545 A1
(43) Pub. Date: Feb. 25, 2010

(54) Compounds and Compositions for Use in the Prevention and Treatment of Disorders of Fat Metabolism and Obesity

(75) Inventors: Lucie Jette, Montreal (CA); Patricia Mcnicol, Vancover (CA); Manjinder Gill, Mississauga (CA); André Marette, Saint-Nicolas (CA)

Correspondence Address: CLARK & ELBING LLP, 101 FEDERAL STREET, BOSTON, MA, 02110, US

(73) Assignee: INNODIA INC., LAVAL, QC (CA)

(21) Appl. No.: 12/293,957

(22) PCT Filed: Mar. 22, 2007
(86) PCT No.: PCT/CA2007/000471

§ 371 Date: Aug. 7, 2009

Related U.S. Application Data
(60) Provisional application No. 60/785,174, filed on Mar. 22, 2006.
(60) Provisional application No. 60/836,648, filed on Aug. 10, 2006.

(30) Foreign Application Priority Data
Mar. 22, 2006 (US) 11387534

(51) Int. Cl. A61K 31/535; A61K 31/195; A61K 31/341; A61K 31/40; A61K 31/445; C07C 229/00; C07D 207/00; C07D 211/78; C07D 221/02; C07D 265/04; C07D 305/12; A61P 3/00; A61P 3/04

(52) U.S. Cl. 514/228.8; 514/561; 514/472; 514/423; 514/354; 562/567; 562/507; 548/532; 546/326; 546/112; 544/88; 549/323

(57) ABSTRACT

The invention relates to 4-hydroxyisoleucine, isomers, analogs, lactones, salts, and prodrugs thereof, to processes for their preparation, and to pharmaceutical compositions comprising the same. More particularly, the invention relates to the use of those compounds in the prevention and treatment of disorders of fat metabolism and related syndromes. The invention further relates to the use of those compounds in the prevention and treatment of obesity and related syndromes including, but not limited to, the cosmetic treatment of a mammal in order to effect a cosmetically beneficial loss of body weight, and more particularly loss of body fat.

### RELATED APPLICATIONS

This application claims the benefit of priority of U.S. Provisional Application 60/785,174 filed Mar. 22, 2006, U.S. Provisional Application 60/836,648 filed on Aug. 10, 2006 and U.S. patent application Ser. No. 11/387,534 filed Mar. 22, 2006, the disclosure of which are incorporated herein by reference in their entirety.

### BACKGROUND OF THE INVENTION

a) Field of the Invention

The invention relates to the use of 4-hydroxyisoleucine, isomers, analogs, lactones, salts and prodrugs thereof, in the prevention and treatment of disorders of fat metabolism. The invention further relates to the use of 4-hydroxyisoleucine, isomers, analogs, lactones, salts and prodrugs thereof, in the prevention and treatment of obesity and related syndromes including, but not limited to, the cosmetic treatment of a mammal in order to effect a cosmetically beneficial loss of body weight, and more particularly loss of body fat.

b) Brief Description of the Related Art

Proper regulation of the metabolism, distribution, storage, and blood levels of fat and lipids is critical to the functioning of major organs and tissues including, for example, the heart, liver, peripheral vasculature, musculature, and nervous system. Disorders of fat metabolism are highly prevalent, and are due to a diversity of causes including genetics (e.g., hereditary hypercholesterolemia, Fabry's disease, Gaucher disease, and Niemann-Pick disease), behavior (e.g., obesity and sedentary life style), and side effects of drugs (e.g., anti-HIV protease inhibitors), and can be components of syndromes associated with other disorders (e.g., disorders of carbohydrate metabolism, such as diabetes and Metabolic Syndrome). In addition to treating the symptoms of disorders of fat metabolism themselves, treating such disorders is also critical in many cases to avoid conditions that such disorders can lead to including, for example, heart disease, peripheral vascular disease (including stroke), liver disease, and obesity.

Fenugreek (Trigonella foenum-graecum) is a legume grown in the Middle East and Asia, which has been used as a medicinal plant for centuries to heal ailments ranging from indigestion to baldness (Madar and Stark, British Journal of Nutrition (2002), 88, Suppl. 3, S287-S292). 4-Hydroxy-3-methylpentanoic acid (4-hydroxyisoleucine or 4-OH) is an unusual substance, which represents about 0.6% of the content of the seeds of fenugreek. It has been demonstrated that the (2S,3R,4S) isomer of 4-hydroxyisoleucine possesses insulinotropic and insulin sensitizing activities (Broca et al., Am. J. Physiol. 277:E617-E623, 1999; Broca et al., Eur. J. Pharmacol. 390:339-345, 2000; Broca et al., Am. J. Physiol. Endocrinol. Metab. 287:E463-E471, 2004; PCT publication Nos. WO 97/32577 and WO 01/15689). It has also been shown that 4-hydroxyisoleucine has antidyslipidemic activities (Narender et al., Biorganic & Medicinal Chemistry Letters, 2006, 16:293-296). Numerous chemical analogs of 4-hydroxyisoleucine have been synthesized (see PCT application PCT/IB2006/001666 filed Feb. 17, 2006 (WO 2006/120574A1; originally designated PCT/US2006/005763, filed on Feb. 17, 2006) and these analogs have been suggested to be effective for the treatment of disorders of carbohydrate metabolism, including diabetes mellitus (type 1 and type 2 diabetes), pre-diabetes, and Metabolic Syndrome. However, none of the above-mentioned studies have ever shown or suggested that 4-hydroxyisoleucine, isomers, or analogs thereof could be useful to address the problem of disorders of lipid metabolism, as noted above.

In summary, notwithstanding the growing body of evidence on the positive activities of 4-hydroxyisoleucine, isomers, and analogs thereof for the treatment of diabetes, it has not been demonstrated that 4-hydroxyisoleucine, isomers, or analogs thereof could be useful for the prevention and/or treatment of disorders of fat metabolism and related syndromes.

In view of the above, there is an important need for new medicinal products to address the urgency created in the medical field by the prevalence of disorders of fat metabolism. More particularly, there is a need for alternative and improved methods, compounds, and compositions for preventing and treating disorders of fat metabolism and related syndromes.

There is also a need for pharmaceutical compositions and therapeutic methods of preventing the onset or progression of excessive weight gain leading to obesity, of reducing body weight and/or body fat in overweight and/or obese people, and of decreasing appetite and/or food intake.

The present invention provides such compounds and methods for their use. Accordingly, the present invention fulfils the above-mentioned needs and also other needs as will be apparent to those skilled in the art upon reading the following specification.

### SUMMARY OF THE INVENTION

The invention provides methods of regulating fat metabolism in a mammal. The invention further provides methods of preventing and/or treating obesity and related syndromes. The invention further provides methods for the cosmetic treatment of a mammal in order to effect a cosmetically beneficial loss of body weight, and more particularly loss of body fat. The methods of the invention involve administering to the mammal a compound selected from the group consisting of: isomers of 4-hydroxyisoleucine, analogs of 4-hydroxyisoleucine, and pharmaceutically acceptable lactones, salts, or prodrugs of said isomers and analogs. The mammal may be afflicted with, for example, a disease or condition selected from the group consisting of a disorder of lipid metabolism, lipodystrophy, hypercholesterolemia, atherosclerosis, and non-alcoholic fatty liver disease (for example, non-alcoholic steatohepatitis). The methods of the invention can result in, for example, one or more of the following effects in said mammal: reducing caloric intake/food consumption and/or apetite, reducing body fat, producing cosmetically beneficial changes, increasing energy expenditure, increasing oxygen consumption, stimulation of lipolysis by adipocytes, modulating or increasing expression of genes related to lipid metabolism (e.g., FABP4/aP2, HSL, ATGL, FatB1, and CPT-1), reducing intestinal lipid absorption, modulation of AMP kinase. The methods of the invention are also useful for lowering the plasma lipid levels (e.g. triglycerides, cholesterol) and reducing cardiac risk.

The invention also provides compounds and pharmaceutical compositions (comprising a pharmaceutical carrier) for preventing and/or treating obesity and related syndromes. The invention also provides compounds and pharmaceutical compositions for preventing and/or treating disorders of fat metabolism. The compounds of the present inventions may be used for reducing cholesterol and/or triglycerides in an obese or non-obese mammal. The compounds of the present invention may therefore be used for avoiding weight gain or for loosing weight.

In one aspect of the invention, the compound is an isomer of 4-hydroxyisoleucine or a pharmaceutically acceptable lactone, salt, metabolite, solvate, and/or prodrug thereof.

As an example, the compound can be the following isomer of 4-hydroxyisoleucine:

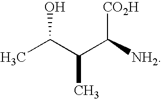

In other examples, the compound can be one of the following isomers:

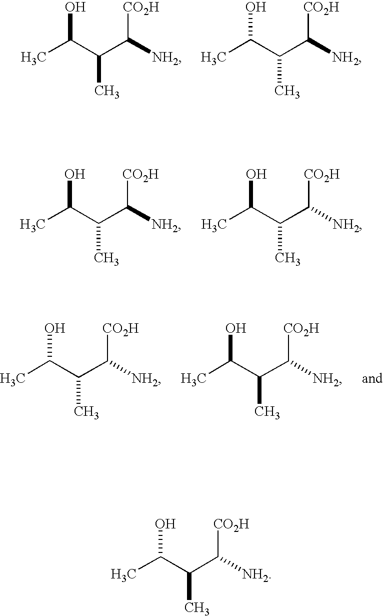

In further examples, the compound can be one of the following lactones of 4-hydroxyisoleucine:

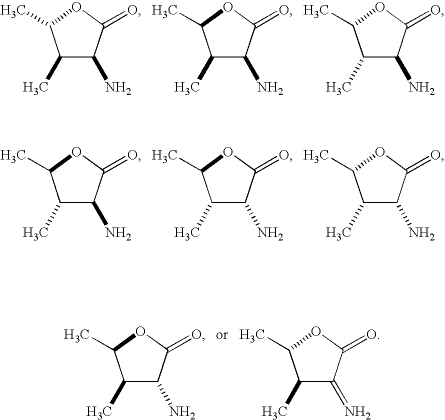

In another aspect of the invention, the compound is an analog of 4-hydroxyisoleucine or a pharmaceutically acceptable lactone, salt, metabolite, solvate, and/or prodrug thereof.

In one example of this aspect of the invention, the compound is an analog within Formula (I):

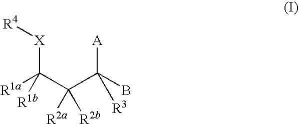

where
A is CO₂R^{A1}, C(O)SR^{A1}, C(S)SR^{A1}, C(O)NR^{A2}R^{A3}, C(S)NR^{A2}R^{A3}, C(O)R^{A4}, SO₃H, S(O)₂NR^{A2}R^{A3}, C(O)R^{A5}, C(OR^{A1})R^{A9}R^{A10}, C(SR^{A1})R^{A9}R^{A10}, C(═NR^{A1})R^{A5}, O═P(OH)₂

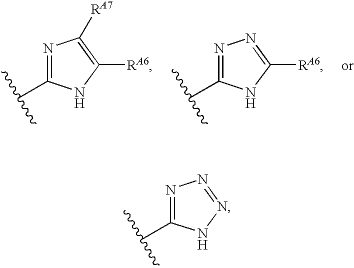

wherein
R^{A1} is hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms,
each of R^{A2} and R^{A3} is, independently, selected from the group consisting of (a) hydrogen, (b) substituted or unsubstituted C₁₋₆ alkyl, (c) substituted or unsubstituted C₃₋₈ cycloalkyl, (d) substituted or unsubstituted alkcycloalkyl, where the cycloalkyl group is of three to eight carbon atoms and the alkylene group is of one to four carbon atoms, (e) substituted or unsubstituted C₆ or C₁₀ aryl, and (f) substituted or unsubstituted C₇₋₁₆ alkaryl, where the alkylene group is of one to six carbon atoms, or R^{A2} taken together with R^{A3} and N forms a substituted or unsubstituted 5- or 6-membered ring, optionally containing O or NR^{A8}, wherein R^{A8} is hydrogen or C₁₋₆ alkyl,
R^{A4} is hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms,
R^{A5} is a peptide chain of 1-4 natural or unnatural amino acids, where the peptide is linked via its terminal amine group to C(O),
each of R^{A6} and R^{A7} is, independently, hydrogen, substituted or unsubstituted C₁₋₆ alkyl, C₁₋₄ perfluoroalkyl, substituted or unsubstituted C₁₋₆ alkoxy, amino, C₁₋₆ alkylamino, C₂₋₁₂ dialkylamino, N-protected amino, halo, or nitro, and
each of R^{A9} and R^{A10} is, independently, selected from the group consisting of (a) hydrogen, (b) substituted or unsubstituted C₁₋₆ alkyl, (c) substituted or unsubstituted C₃₋₈ cycloalkyl, (d) substituted or unsubstituted alkcycloalkyl, where the cycloalkyl group is of three to eight carbon atoms and the alkylene group is of one to four carbon atoms, (e) substituted or unsubstituted C₆ or C₁₀ aryl, and (f) substituted or unsubstituted C₇₋₁₆ alkaryl, where the alkylene group is of one to six carbon atoms, or R^{A9} taken together with R^{A10} and their parent carbon atom forms a substituted or unsubstituted 5- or 6-membered ring, optionally containing O or NR^{A8}, wherein R^{A8} is hydrogen or C₁₋₆ alkyl;
B is NR^{B1}R^{B2}, where
(i) each of R^{B1} and R^{B2} is, independently selected from the group consisting of (a) hydrogen, (b) an N-protecting group, (c) substituted or unsubstituted C₁₋₆ alkyl, (d) substituted or unsubstituted C₂₋₆ alkenyl, (e) substituted or unsubstituted C₂₋₆ alkynyl, (f) substituted or unsubstituted C₃₋₈ cycloalkyl, (g) substituted or unsubstituted alkcycloalkyl, where the cycloalkyl group is of three to eight carbon atoms, and the alkylene group is of one to ten carbon atoms, (h) substituted or unsubstituted C₆ or C₁₀ aryl, (i) substituted or unsubstituted C₇₋₁₆ alkaryl, where the alkylene group is of one to six carbon atoms, (j) substituted or unsubstituted C₁₋₉ heterocyclyl, (k) substituted or unsubstituted C₂₋₁₅ alkheterocyclyl, where the alkylene group is of one to six carbon atoms, (l) (CH₂)ₙC(O)R^{B3}, wherein n is 0, 1, 2, or 3 where R^{B3} is selected from the group consisting of hydrogen, substituted or unsubstituted C₁₋₆ alkyl, substituted or unsubstituted C₆ or C₁₀ aryl, substituted or unsubstituted C₇₋₁₆ alkaryl, where the alkylene group is of one to six carbon atoms, substituted or unsubstituted C₁₋₉ heterocyclyl, or substituted or unsubstituted C₂₋₁₅ alkheterocyclyl, where the alkylene group is of one to six carbon atoms, (m) (CH₂)ₙ CO₂R^{B4}, wherein n is 0, 1, 2 or 3, where R^{B4} is selected from the group consisting of hydrogen, substituted or unsubstituted C₁₋₆ alkyl, substituted or unsubstituted C₆ or C₁₀ aryl, substituted or unsubstituted C₇₋₁₆ alkaryl, where the alkylene group is of one to six carbon atoms, substituted or unsubstituted C₁₋₉ heterocyclyl, or substituted or unsubstituted C₂₋₁₅ alkheterocyclyl, where the alkylene group is of one to six carbon atoms, (n) C(O)NR^{B5}R^{B6}, where each of R^{B5} and R^{B6} is, independently, selected from the group consisting of hydrogen, substituted or unsubstituted C₁₋₆ alkyl, substituted or unsubstituted C₆ or C₁₀ aryl, substituted or unsubstituted C₇₋₁₆ alkaryl, where the alkylene group is of one to six carbon atoms, substituted or unsubstituted C₁₋₉ heterocyclyl, and substituted or unsubstituted C₂₋₁₅ alkheterocyclyl, where the alkylene group is of one to six carbon atoms, or R^{B5} taken together with R^{B6} and N forms a substituted or unsubstituted 5- or 6-membered ring, optionally containing a non-vicinal O, S, or NR′, where R′ is H or C₁₋₆ alkyl, (o) S(O)₂R^{B7}, where R^{B7} is selected from the group consisting of hydrogen, substituted or unsubstituted C₁₋₆ alkyl, substituted or unsubstituted C₆ or C₁₀ aryl, substituted or unsubstituted C₇₋₁₆ alkaryl, where the alkylene group is of one to six carbon atoms, substituted or unsubstituted C₁₋₉ heterocyclyl, or substituted or unsubstituted C₂₋₁₅ alkheterocyclyl, where the alkylene group is of one to six carbon atoms, and (p) a peptide chain of 1-4 natural or unnatural alpha-amino acid residues, where the peptide is linked via its terminal carboxy group to N, with the proviso that no two groups are bound to the nitrogen atom through a carbonyl group or a sulfonyl group, or
(ii) R^{B1} taken together with R^{B2} and N forms a substituted or unsubstituted 5- or 6-membered ring, optionally containing O or NR^{B8}, wherein R^{B8} is hydrogen or C₁₋₆ alkyl, or
(iii) a 5- to 8-membered ring is formed when R^{B1} taken together with R^{1a} is a substituted or unsubstituted C₁₋₄ alkylene, or
(iv) a [2.2.1] or [2.2.2] bicyclic ring system is formed when R^{B1} taken together with R^{1a} is a substituted or unsubstituted C₂ alkylene and R^{B1} taken together with R^{2a} is a substituted or unsubstituted C₁₋₂ alkylene, or
(v) a 4- to 8-membered ring is formed when R^{B1} taken together with R³ is a substituted or unsubstituted C₂₋₆ alkylene, or
(vi) a 6- to 8-membered ring is formed when R^{B1} taken together with R⁴ is a substituted or unsubstituted C₁₋₃ alkylene, or
(vii) R^{B1} taken together with A and the parent carbon of A and B forms the following ring:

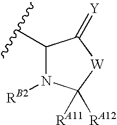

where each of Y and W is, independently, O, S, NR^{B8}, or CR^{A9}R¹⁰; each of R^{A9} and R^{A10} is as previously defined and each of R^{A11} and R^{A12} is, independently, selected from the group consisting of (a) hydrogen, (b) substituted or unsubstituted C₁₋₆ alkyl, (c) substituted or unsubstituted C₃₋₈ cycloalkyl, (d) substituted or unsubstituted alkcycloalkyl, where the cycloalkyl group is of three to eight carbon atoms and the alkylene group is of one to four carbon atoms, (e) substituted or unsubstituted C₆ or C₁₀ aryl, and (f) substituted or unsubstituted C₇₋₁₆ alkaryl, where the alkylene group is of one to six carbon atoms, or R^{A9} taken together with R^{A10} and their parent carbon atom forms a substituted or unsubstituted 5- or 6-membered ring, optionally containing O or NR^{A8}, wherein R^{A8} is hydrogen or C₁₋₆ alkyl;
X is (i) absent (ii) hydrogen, (iii) a substituted or unsubstituted C₁₋₆, (iv) substituted or unsubstituted C₃₋₈ cycloalkyl, (v) substituted or unsubstituted alkcycloalkyl, where the cycloalkyl group is of three to eight carbon atoms and the alkylene group is of one to four carbon atoms (vi) substituted or unsubstituted C₆ or C₁₀ aryl, (vii) substituted or unsubstituted C₇₋₁₆ alkaryl, where the alkylene group is of one to four carbon atoms, (viii) SO₃H; (ix) O, (x) S, or (xi) NR^{X1}, where R^{X1} is selected from the group consisting of (a) hydrogen, (b) an N-protecting group, (c) substituted or unsubstituted C₁₋₆ alkyl, (d) substituted or unsubstituted C₂₋₆ alkenyl, (e) substituted or unsubstituted C₂₋₆ alkynyl, (f) substituted or unsubstituted C₃₋₈ cycloalkyl, (g) substituted or unsubstituted alkcycloalkyl, where the cycloalkyl group is of three to eight carbon atoms, and the alkylene group is of one to ten carbon atoms, (h) substituted or unsubstituted C₆ or C₁₀ aryl, (i) substituted or unsubstituted C₇₋₁₆ alkaryl, where the alkylene group is of one to six carbon atoms, (j) substituted or unsubstituted C₁₋₉ heterocyclyl, or (k) substituted or unsubstituted C₂₋₁₅ alkheterocyclyl, where the alkylene group is of one to six carbon atoms;
each of R^{1a} and R^{1b} is, independently, (a) hydrogen, (b) substituted or unsubstituted C₁₋₆ alkyl, (c) substituted or unsubstituted C₃₋₈ cycloalkyl, (d) substituted or unsubstituted alkcycloalkyl, where the cycloalkyl group is of three to eight carbon atoms and the alkylene group is of one to four carbon atoms, (e) substituted or unsubstituted C₂₋₆ alkenyl, (f) substituted or unsubstituted C₂₋₆ alkynyl, (g) substituted or unsubstituted C₆ or C₁₀ aryl, (h) substituted or unsubstituted C₇₋₁₆ alkaryl, where the alkylene group is of one to four carbon atoms, (i) substituted or unsubstituted C₁₋₉ heterocyclyl, (j) substituted or unsubstituted C₂₋₁₅ alkheterocyclyl, where the alkylene group is of one to four carbon atoms, or R^{1a} together with R^{2a} and their base carbon atoms form a substituted or unsubstituted C₅₋₁₀ mono or fused ring system, or a 3- to 6-membered ring is formed when R^{1a} together with R⁴ is a substituted or unsubstituted C₁₋₄ alkylene, (k) NR^{B1}R^{B2}, (l) a OR₄ group, or (m) R^{1a} and R^{1b} together are ═O, ═N(C₁₋₆ alkyl), ═CR^{1c}R^{1d}, where each of R^{1c} and R^{1d} is, independently, hydrogen or substituted or unsubstituted C₁₋₆ alkyl, or a substituted or unsubstituted C₂₋₅ alkylene moiety forming a spiro ring;
each of R^{2a} and R^{2b} is, independently, hydrogen, halogen (e.g., F, Cl, Br, I) substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms, or R^{2a} and R^{2b} together are ═O, ═N(C₁₋₆ alkyl), ═CR^{2c}R^{2d}, where each of R^{2c} and R^{2d} is, independently, hydrogen or substituted or unsubstituted C₁₋₆ alkyl, or a substituted or unsubstituted C₂₋₅ alkylene moiety forming a spiro ring, or R^{2a} together with R^{1a} and their base carbon atoms form a substituted or unsubstituted C₅₋₁₀ mono or fused ring system;
R³ is hydrogen, COOR^{A1}, substituted or unsubstituted C₁₋₆ alkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₇₋₁₆ alkaryl, where the alkylene group is of one to four carbon atoms, or substituted or unsubstituted C₂₋₁₅ alkheterocyclyl, where the alkylene group is of one to four carbon atoms; and
R⁴ is absent, hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms, or a 3- to 6-membered ring is formed when R⁴ together with R^{1a} is a substituted or unsubstituted C₁₋₄ alkylene, or a 6- to 8-membered ring is formed when R⁴ taken together with R^{B1} is a substituted or unsubstituted C₁₋₃ alkylene.

In other examples, the compound is an analog within Formula (II):

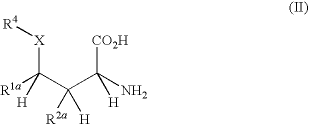

where each of X and R⁴ is as previously defined in reference to Formula (I) and each of R^{1a} and R^{2a} is, independently, substituted or unsubstituted C₁₋₆ alkyl or R^{1a} together with R^{2a} and their base carbon atoms form a substituted or unsubstituted 6 membered ring.

In additional examples, the compound is an analog of Formula (III):

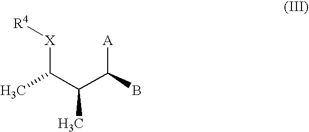

where A is CO₂R^{A1}, C(O)SR^{A1}, C(O)NR^{A2}R^{A3}, or C(O)R^{A5}; and each of R^{A1}, R^{A2}, R^{A3}, R^{A5}, B, X, and R⁴ is as previously defined in reference to Formula (I).

In further examples, the compound is an analog of Formula (IV):

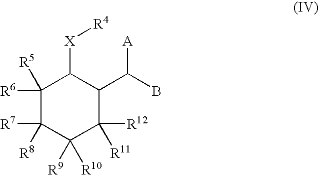

where A is CO₂R^{A1}, C(O)SR^{A1}, C(O)NR^{A2}R^{A3}, or C(O)R^{A5}; each of B, X, and R⁴ is as previously defined in reference to Formula (I); and each of R⁵, R⁶, R⁷, R⁸, R⁹, R¹⁰, R¹¹, and R¹² is, independently, hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms.

Additional compounds of the invention are within the following formulae:

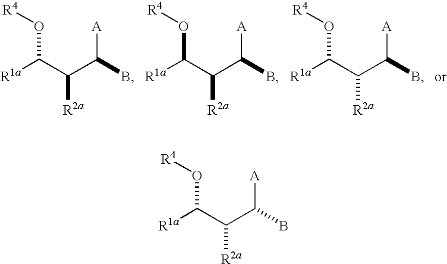

where each of A, B, and R⁴ is as previously defined in reference to Formula (I), and each of R^{1a} and R²¹ is, individually, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms.

In various embodiments of this aspect of the invention, and in reference to the formulae noted above, A is CO₂H, B is NH-p-toluenesulfonyl, R⁴ is H, and each of R^{1a} and R^{2a} is CH₃; A is CO₂H, B is NH₂, R⁴ is H, and each of R^{1a} and R^{2a} is a substituted or unsubstituted C₁₋₆ alkyl; or A is CO₂H, B is NH₂, X is O, and R⁴ is H.

In other examples of this aspect of the invention, the compound is within one of the following formulae:

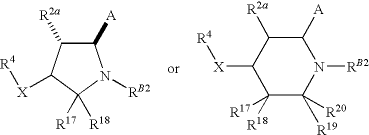

where each of A, X, R^{2a}, R⁴, and R^{B2} is as previously defined in reference to Formula (I), and each of R¹⁷, R¹⁵, R¹⁹, and R²⁰ is hydrogen or substituted or unsubstituted C₁₋₆ alkyl.

In additional examples, the compound is within:

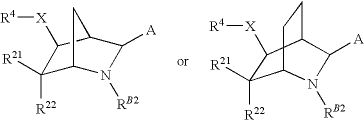

where each of A, X, R⁴, and R^{B2} is as previously defined in reference to Formula (I), and each of R²¹ and R²² is hydrogen or substituted or unsubstituted C₁₋₆ alkyl.

In a further example, the compound is within:

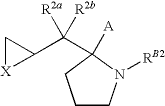

where each of A, X, R^{2a}, R^{2b}, and R^{B2} is as previously defined in reference to Formula (I).

In yet an additional example, the compound is within:

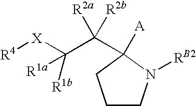

where each of A, X, R^{1a}, R^{1b}, R^{2a}, R^{2b}, R⁴, and R^{B2} is as previously defined in reference to Formula (I).

In additional embodiments, and in reference to the formulae noted above, R^{1a} together with R^{2a} and their base carbon atoms form a substituted or unsubstituted C₅₋₁₀ mono or fused ring system, optionally containing a non-vicinal O, S, or NR′, where R′ is H or C₁₋₆ alkyl.

Further examples of compounds of Formula (I) are as follows:

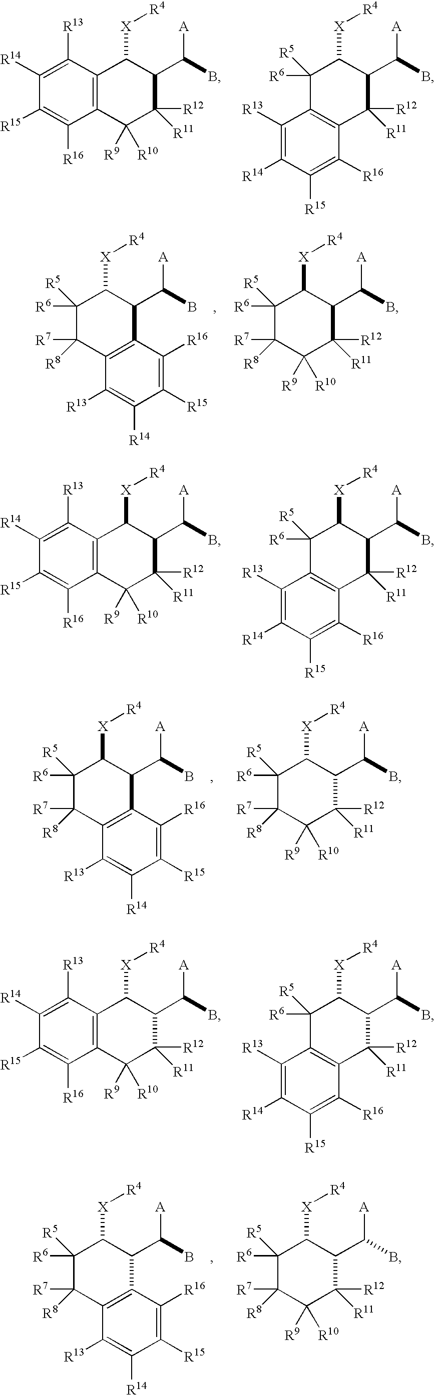

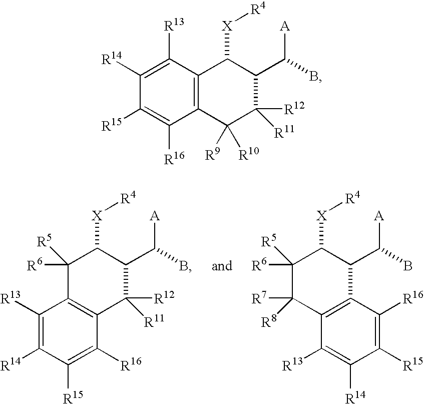

where each of A, B, X, and R⁴ is as defined previously in reference to Formula (I), and each of R⁵, R⁶, R⁷, R⁸, R⁹, R¹⁰, R¹¹, and R¹² is, independently, hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms; and
each of R¹³, R¹⁴, R¹⁵, and R¹⁶ is, independently, hydrogen, substituted or unsubstituted C₁₋₆ alkyl, C₁₋₄ perfluoroalkyl, substituted or unsubstituted C₁₋₆ alkoxy, amino, C₁₋₆ alkylamino, C₂₋₁₂ dialkylamino, N-protected amino, halo, or nitro.

Specific examples of compounds that can be used in the methods of the invention are as follows:

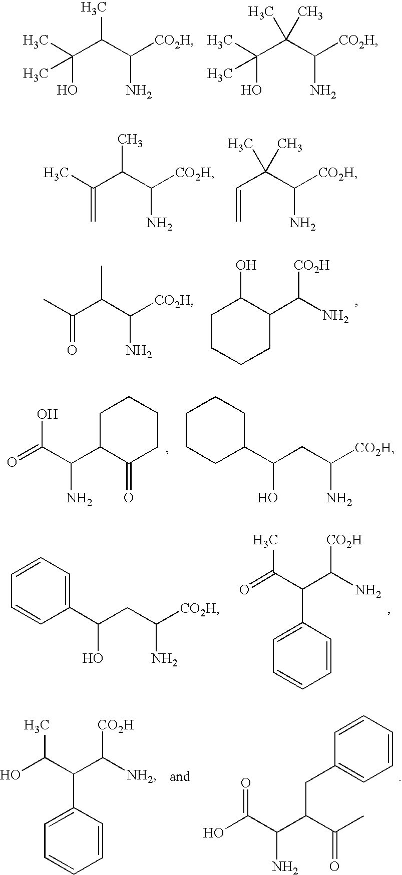

Additional specific examples include the following:

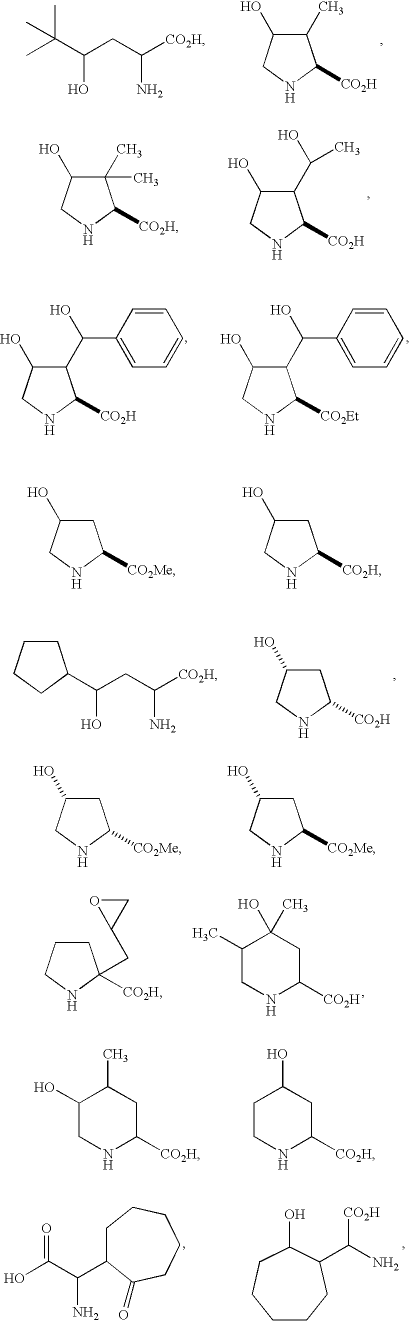

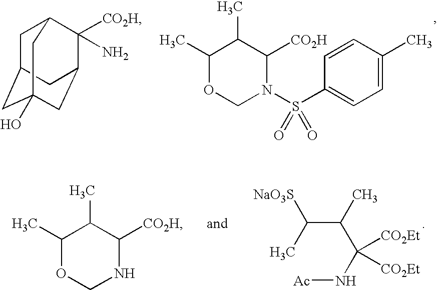

Further examples include those compounds shown in Table 1 hereinafter.

Other examples of compounds that can be used in the methods of the invention are described as follows. The invention also includes these compounds themselves, as compositions of matter (and pharmaceutically acceptable lactones, salts, metabolites, solvates, and/or prodrugs thereof), and in the context of pharmaceutical compositions.

The additional compounds include analogs of Formula (V):

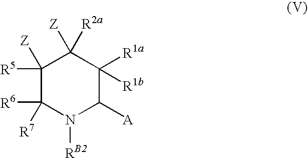

where each of A, R^{1a}, R^{1b}, R^{2a}, R⁴, and R^{B2}, are as defined previously in reference to Formula (I); R⁵, R⁶, and R⁷ are each, independently, hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms; and Z is XR⁴ or NR^{B1}R^{B2}, where X is O, or S, and R^{B1} and R^{B2} are each selected, independently, from the group consisting of (a) hydrogen, (b) an N-protecting group, (c) substituted or unsubstituted C₁₋₆ alkyl, (d) substituted or unsubstituted C₂₋₆ alkenyl, (e) substituted or unsubstituted C₂₋₆ alkynyl, (f) substituted or unsubstituted C₃₋₈ cycloalkyl, (g) substituted or unsubstituted alkcycloalkyl, where the cycloalkyl group is of three to eight carbon atoms, and the alkylene group is of one to ten carbon atoms, (h) substituted or unsubstituted C₆ or C₁₀ aryl, (i) substituted or unsubstituted C₇₋₁₆ alkaryl, where the alkylene group is of one to six carbon atoms, (j) substituted or unsubstituted C₁₋₉ heterocyclyl, (k) substituted or unsubstituted C₂₋₁₅ alkheterocyclyl, where the alkylene group is of one to six carbon atoms, (l) C(O)R^{B3}, where R^{B3} is selected from the group consisting of substituted or unsubstituted C₁₋₆ alkyl, substituted or unsubstituted C₆ or C₁₀ aryl, substituted or unsubstituted C₇₋₁₆ alkaryl, where the alkylene group is of one to six carbon atoms, substituted or unsubstituted C₁₋₉ heterocyclyl, or substituted or unsubstituted C₂₋₁₅ alkheterocyclyl, where the alkylene group is of one to six carbon atoms, (m) CO₂R^{B4}, where R^{B4} is selected from the group consisting of substituted or unsubstituted C₁₋₆ alkyl, substituted or unsubstituted C₆ or C₁₀ aryl, substituted or unsubstituted C₇₋₁₆ alkaryl, where the alkylene group is of one to six carbon atoms, substituted or unsubstituted C₁₋₉ heterocyclyl, or substituted or unsubstituted C₂₋₁₅ alkheterocyclyl, where the alkylene group is of one to six carbon atoms, (n) C(O)NR^{B5}R^{B6} where each of R^{B5} and R^{B6} is, independently, selected from the group consisting of hydrogen, substituted or unsubstituted C₁₋₆ alkyl, substituted or unsubstituted C₆ or C₁₀ aryl, substituted or unsubstituted C₇₋₁₆ alkaryl, where the alkylene group is of one to six carbon atoms, substituted or unsubstituted C₁₋₉ heterocyclyl, and substituted or unsubstituted C₂₋₁₅ alkheterocyclyl, where the alkylene group is of one to six carbon atoms, or R^{B5} taken together with R^{B6} and N forms a substituted or unsubstituted 5- or 6-membered ring, optionally containing a non-vicinal O, S, or NR′, where R′ is H or C₁₋₆ alkyl, (O)S(O)₂R^{B7}, where R^{B7} is selected from the group consisting of substituted or unsubstituted C₁₋₆ alkyl, substituted or unsubstituted C₆ or C₁₀ aryl, substituted or unsubstituted C₇₋₁₆ alkaryl, where the alkylene group is of one to six carbon atoms, substituted or unsubstituted C₁₋₉ heterocyclyl, or substituted or unsubstituted C₂₋₁₅ alkheterocyclyl, where the alkylene group is of one to six carbon atoms, (p) a peptide chain of 1-4 natural or unnatural alpha-amino acid residues, where the peptide is linked via its terminal carboxy group to N; or R^{B1} taken together with R^{B2} and N forms a substituted or unsubstituted 5- or 6-membered ring, optionally containing O or NR^{B8}, wherein R^{B8} is hydrogen or C₁₋₆ alkyl.

Additional compounds are of Formula (V-A):

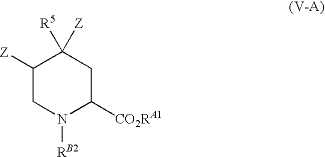

where each of R^{A1}, R^{B2}, and R⁴, are as defined previously in reference to Formula (I); R⁵ is hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms; and Z is XR⁴ or NR^{B1}R^{B2} as defined previously in reference to Formula (V).

As specific examples, the compound can be selected from the group consisting of:

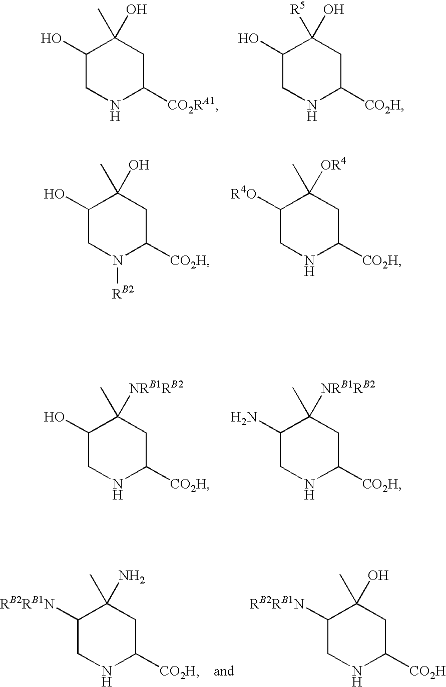

where R^{A1}, R^{B1}, R^{B2}, and R⁴ are as defined previously in reference to Formula (I), and R⁵ is hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms.

Additional compounds are of Formula (VI):

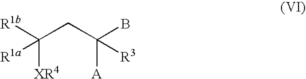

where A, B, X, R^{1a}, R^{1b}, R³, and R⁴ are as defined previously in reference to Formula (I).

In further examples the compound is within one of the following formulae:

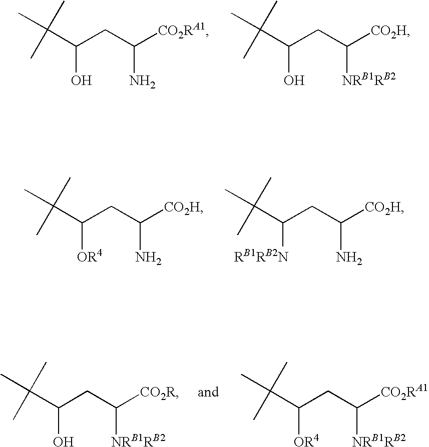

where R^{A1}, R^{B1}, R^{B2}, and R⁴ are as defined previously in reference to Formula (I).

Specific examples compounds within the above-noted formulae that are included in the invention are as follows:

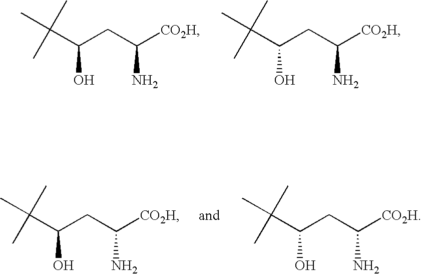

Further specific examples of compounds of the invention are as follows:

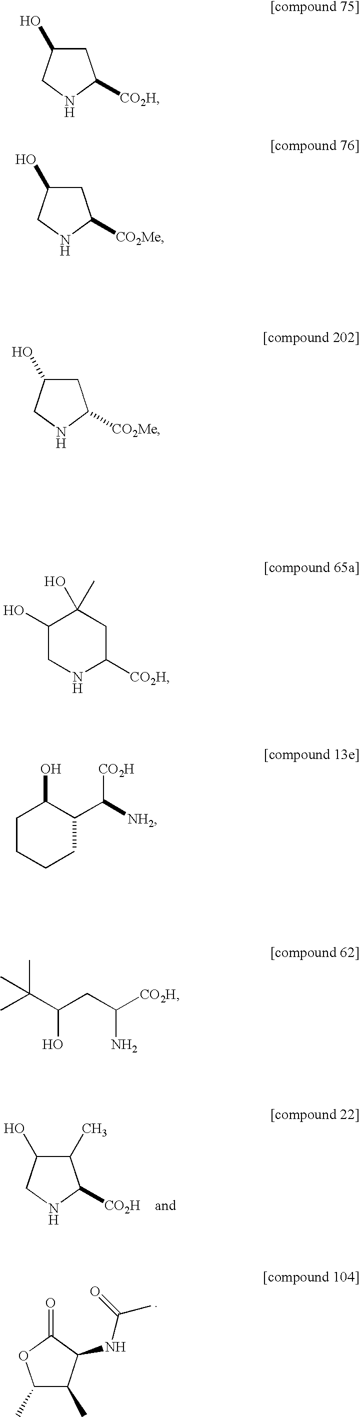

In one example of this aspect of the invention, the compound is an analog within Formula (I′):

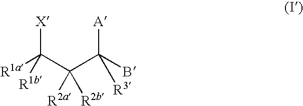

wherein
A′ may be CO₂R^{A1′}, C(O)SR^{A1′}, C(S)SR^{A1′}, C(O)NR^{A2′}R^{A3′}, C(S)NR^{A2′}R^{A3′}, C(O)R^{A4′}, S₃H, S(O)₂NR^{A2′}R^{A3′}, C(O)R^{A5′}, C(OR^{A1′})R^{A9}R^{A10′}, C(SR^{A1′})R^{A9′}R^{10′}, C(═NR^{A1′})R^{A5′}, O═P(OH)₂, or C(═O) and when A′ is C(═O), A′ forms together with X′ a 5 or 6 members ring,

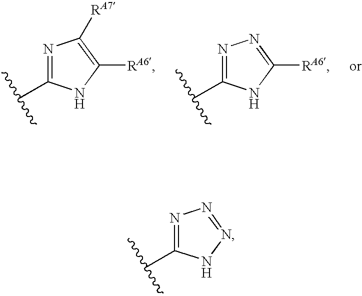

wherein R^{A1′} may be hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms,
each of R^{A2′} and R^{A3′} may, independently, selected from the group consisting of (a) hydrogen, (b) substituted or unsubstituted C₁₋₆ alkyl, (c) substituted or unsubstituted C₃₋₈ cycloalkyl, (d) substituted or unsubstituted alkcycloalkyl, where the cycloalkyl group is of three to eight carbon atoms and the alkylene group is of one to four carbon atoms, (e) substituted or unsubstituted C₆ or C₁₀ aryl, and (f) substituted or unsubstituted C₇₋₁₆ alkaryl, where the alkylene group is of one to six carbon atoms, or R^{A2′} taken together with R^{A3′} and N forms a substituted or unsubstituted 5- or 6-membered ring, optionally containing O or NR^{A8′}, wherein R^{A8′} may be hydrogen or C₁₋₆ alkyl,
R^{A4′} may be hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms,
R^{A5′} may be a peptide chain of 1-4 natural or unnatural amino acids, where the peptide may be linked via its terminal amine group to C(O),
each of R^{A6′} and R^{A7′} may be, independently, hydrogen, substituted or unsubstituted C₁₋₆ alkyl, C₁₋₄ perfluoroalkyl, substituted or unsubstituted C₁₋₆ alkoxy, amino, C₁₋₆ alkylamino, C₂₋₁₂ dialkylamino, N-protected amino, halo, or nitro, and
each of R^{A9′} and R^{A10′} may be, independently, selected from the group consisting of (a) hydrogen, (b) substituted or unsubstituted C₁₋₆ alkyl, (c) substituted or unsubstituted C₃₋₈ cycloalkyl, (d) substituted or unsubstituted alkcycloalkyl, where the cycloalkyl group is of three to eight carbon atoms and the alkylene group is of one to four carbon atoms, (e) substituted or unsubstituted C₆ or C₁₀ aryl, and (f) substituted or unsubstituted C₇₋₁₆ alkaryl, where the alkylene group is of one to six carbon atoms, or R^{A9′} taken together with RA^{10′} and their parent carbon atom forms a substituted or unsubstituted 5- or 6-membered ring, optionally containing O or NR^{A8′}, wherein R^{A8′} is hydrogen or C₁₋₆ alkyl;
B′ may be NR^{B1′}R^{B2′} or NR^{B2′} and when B′ is NR^{B2′}, B′ is connected by the base nitrogen atom to a carbon atom of X′ to form a 5 or 6 membered ring or to the carbon of one of R^{1a′} or R^{1b′}, when one of R^{1a′} or R^{1b′} is OCH₂, wherein each of R^{B1′} and R^{B2′} may be, independently selected from the group consisting of (a) hydrogen, (b) an N-protecting group, (c) substituted or unsubstituted C₁₋₆ alkyl, (d) substituted or unsubstituted C₂₋₆ alkenyl, (e) substituted or unsubstituted C₂₋₆ alkynyl, (f) substituted or unsubstituted C₃₋₈ cycloalkyl, (g) substituted or unsubstituted alkcycloalkyl, where the cycloalkyl group is of three to eight carbon atoms, and the alkylene group is of one to ten carbon atoms, (h) substituted or unsubstituted C₆ or C₁₀ aryl, (i) substituted or unsubstituted C₇₋₁₆ alkaryl, where the alkylene group is of one to six carbon atoms, (j) substituted or unsubstituted C₁₋₉ heterocyclyl, (k) substituted or unsubstituted C₂₋₁₅ alkheterocyclyl, where the alkylene group is of one to six carbon atoms, (l) (CH₂)ₙC(O)R^{B3}, where n may be, for example, 0, 1, 2 or 3, where R^{B3′} may be selected from the group consisting of hydrogen, substituted or unsubstituted C₁₋₆ alkyl, substituted or unsubstituted C₆ or C₁₀ aryl, substituted or unsubstituted C₇₋₁₆ alkaryl, where the alkylene group is of one to six carbon atoms, substituted or unsubstituted C₁₋₉ heterocyclyl, or substituted or unsubstituted C₂₋₁₅ alkheterocyclyl, where the alkylene group may be of one to six carbon atoms, (m) (CH₂)ₙCO₂R^{B4′}, where n may be 0, 1, 2 or 3, where R^{B4′} may be selected from the group consisting of hydrogen, substituted or unsubstituted C₁₋₆ alkyl, substituted or unsubstituted C₆ or C₁₀ aryl, substituted or unsubstituted C₇₋₁₆ alkaryl, where the alkylene group may be of one to six carbon atoms, substituted or unsubstituted C₁₋₉ heterocyclyl, or substituted or unsubstituted C₂₋₁₅ alkheterocyclyl, where the alkylene group may be of one to six carbon atoms, (n) C(O)NR^{B5′}R^{B6′}, where each of R^{B5′} and R^{B6′} may be, independently, selected from the group consisting of hydrogen, substituted or unsubstituted C₁₋₆ alkyl, substituted or unsubstituted C₆ or C₁₀ aryl, substituted or unsubstituted C₇₋₁₆ alkaryl, where the alkylene group may be of one to six carbon atoms, substituted or unsubstituted C₁₋₉ heterocyclyl, and substituted or unsubstituted C₂₋₁₅ alkheterocyclyl, where the alkylene group may be of one to six carbon atoms, or R^{B5′} taken together with R^{B6′} and N forms a substituted or unsubstituted 5- or 6-membered ring, optionally containing a non-vicinal O, S, or NR*, where R* may be H or C₁₋₆ alkyl, (o) S(O)₂R^{B7′}, where R^{B7′} may be selected from the group consisting of hydrogen, substituted or unsubstituted C₁₋₆ alkyl, substituted or unsubstituted C₆ or C₁₀ aryl, substituted or unsubstituted C₇₋₁₆ alkaryl, where the alkylene group may be of one to six carbon atoms, substituted or unsubstituted C₁₋₉ heterocyclyl, or substituted or unsubstituted C₂₋₁₅ alkheterocyclyl, where the alkylene group may be of one to six carbon atoms, and (p) a peptide chain of 1-4 natural or unnatural alpha-amino acid residues, where the peptide may be linked via its terminal carboxy group to N, with the proviso that no two groups are bound to the nitrogen atom through a carbonyl group or a sulfonyl group,
X′ may be (a) hydrogen, (b) substituted or unsubstituted C₁₋₆, substituted or unsubstituted C₃₋₈ cycloalkyl, substituted or unsubstituted alkcycloalkyl, where the cycloalkyl group may be of three to eight carbon atoms and the alkylene group may be of one to four carbon atoms, substituted or unsubstituted C₆ or C₁₀ aryl, substituted or unsubstituted C₇₋₁₆ alkaryl, where the alkylene group may be of one to four carbon atoms, (c) SO₃H group, (d) a OR₄′ group, wherein R₄′ may be hydrogen, substituted or unsubstituted C₁₋₆ alkyl, substituted or unsubstituted C₃₋₈ cycloalkyl, substituted or unsubstituted alkcycloalkyl, where the cycloalkyl group may be of three to eight carbon atoms and the alkylene group may be of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group may be of one to four carbon atoms, substituted or unsubstituted C₁₋₉ heterocyclyl, or substituted or unsubstituted C₂₋₁₅ alkheterocyclyl, where the alkylene group may be of one to four carbon atoms, (e) a C₁₋₂ alkyl linked to the base nitrogen atom of B′ so as to form a 5 or 6 member ring, wherein the C₁₋₂ alkyl may be unsubstituted or substituted with one or more group selected from the group consisting of OR₄′, a C₁₋₆ straight, branched alkyl and NR^{B1′}R^{B2′} or combination thereof (f) a C₃₋₄ alkyl linked to the carbon atom of R^{2a′} or R^{2b′} so as to form a 6 (aromatic or not (one of R^{1a′} or R^{1b′} may be absent when an aromatic ring is formed between one of R^{2a′} or R^{2b′} and X′, also the other R^{2a′} or R^{2b′} may be absent when an aromatic ring is formed between one of R^{2a′} or R^{2b′} and X′)) or 7 member ring, unsubstituted or substituted with one or more group selected from the group consisting of OR₄′, a C₁₋₆ straight or branched alkyl and NR^{B1′}R^{B2′} or combination thereof or (g) oxygen, S, NR^{X1}′ and X′ together with the base carbon atom of A′ forms a 5 or 6 members ring, wherein R^{X1}′ may be selected from the group consisting of (i) hydrogen, (ii) an N-protecting group, (iii) substituted or unsubstituted C₁₋₆ alkyl, (iv) substituted or unsubstituted C₂₋₆ alkenyl, (v) substituted or unsubstituted C₂₋₆ alkynyl, (vi) substituted or unsubstituted C₃₋₈ cycloalkyl, (vii) substituted or unsubstituted alkcycloalkyl, where the cycloalkyl group may be of three to eight carbon atoms, and the alkylene group may be of one to ten carbon atoms, (viii) substituted or unsubstituted C₆ or C₁₀ aryl, (ix) substituted or unsubstituted C₇₋₁₆ alkaryl, where the alkylene group may be of one to six carbon atoms, (x) substituted or unsubstituted C₁₋₉ heterocyclyl, or (xi) substituted or unsubstituted C₂₋₁₅ alkheterocyclyl, where the alkylene group may be of one to six carbon atoms,
each of R^{1a}′ and R^{1b}′ the same or different may be (a) hydrogen, (b) NR^{B1′}R^{B2′}, (c) a OR₄′ group, wherein R₄′ may be (i) hydrogen, (ii) substituted or unsubstituted C₁₋₆ alkyl, (iii) substituted or unsubstituted C₃₋₈ cycloalkyl, (iv) substituted or unsubstituted alkcycloalkyl, where the cycloalkyl group may be of three to eight carbon atoms and the alkylene group may be of one to four carbon atoms, (v) substituted or unsubstituted C₂₋₆ alkenyl, (vi) substituted or unsubstituted C₂₋₆ alkynyl, (vii) substituted or unsubstituted C₆ or C₁₀ aryl, (viii) substituted or unsubstituted C₇₋₁₆ alkaryl, where the alkylene group may be of one to four carbon atoms, (ix) substituted or unsubstituted C₁₋₉ heterocyclyl, or (x) substituted or unsubstituted C₂₋₁₅ alkheterocyclyl, where the alkylene group may be of one to four carbon atoms, (d) substituted or unsubstituted C₁₋₆ alkyl, (e) substituted or unsubstituted C₃₋₈ cycloalkyl, (f) substituted or unsubstituted alkcycloalkyl, where the cycloalkyl group may be of three to eight carbon atoms and the alkylene group may be of one to four carbon atoms, (g) substituted or unsubstituted C₂₋₆ alkenyl, (h) substituted or unsubstituted C₂₋₆ alkynyl, (i) substituted or unsubstituted C₆ or C₁₀ aryl, (j) substituted or unsubstituted C₇₋₁₆ alkaryl, where the alkylene group may be of one to four carbon atoms, (k) substituted or unsubstituted C₁₋₉ heterocyclyl, (l) substituted or unsubstituted C₂₋₁₅ alkheterocyclyl, where the alkylene group may be of one to four carbon atoms, (m) R^{a1′} and R^{1b′} together are ═O, ═N(C₁₋₆ alkyl), or ═CR^{1c}′R^{1d}′, where each of R^{1c}′ and R^{1d}′ is, independently, hydrogen or substituted or unsubstituted C₁₋₆ alkyl, or (n) one of R^{1a}′ or R^{1b}′ may be O—CH₂ and is linked by the CH₂ group with the base nitrogen atom of B′ to form a 6 members ring. In addition, X′ together with R¹a′ and R^{1b}′ may also form a substituted or unsubstituted aryl group such as exemplified by compound 77 of Table 1.

As indicated herein one of R^{1a′} or R^{1b′} may be absent when an aromatic ring is formed between one of R^{2a′} or R^{2b′} and X′. Also the other R^{2a′} or R^{2b′} may also be absent when an aromatic ring is formed between one of R^{2a′} or R^{2b′} and X′.

each of R^{2a}′ and R^{2b}′ the same or different may be hydrogen, F, Cl, Br, I, substituted or unsubstituted C₁₋₆ alkyl group, substituted or unsubstituted C₃₋₈ cycloalkyl, substituted or unsubstituted alkcycloalkyl, where the cycloalkyl group may be of three to eight carbon atoms and the alkylene group may be of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group may be of one to four carbon atoms, substituted or unsubstituted C₁₋₉ heterocyclyl, or substituted or unsubstituted C₂₋₁₅ alkheterocyclyl, where the alkylene group may be of one to four carbon atoms, or R^{2a′} or R^{2b′} may be a C₁₋₂ alkyl linked to X′ to form a 6 (aromatic or not) or 7 members ring, and;
R³′ may be hydrogen, COOR^{A1}′, substituted or unsubstituted C₁₋₆ alkyl, substituted or unsubstituted alkcycloalkyl, where the cycloalkyl group may be of three to eight carbon atoms and the alkylene group may be of one to four carbon atoms, substituted or unsubstituted C₂₋₆ alkenyl, substituted or unsubstituted C₂₋₆ alkynyl, substituted or unsubstituted C₇₋₁₆ alkaryl, where the alkylene group may be of one to four carbon atoms, substituted or unsubstituted C₂₋₁₅ alkheterocyclyl, where the alkylene group may be of one to four carbon atoms or

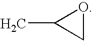

In an exemplary embodiment of the invention the compound of Formula I′ may be those where A′ may be selected from the group consisting of COOR^{A1}′, CONR^{A2}′R^{A3}′, wherein R^{A2′} taken together with R^{A3′} and N forms a substituted or unsubstituted 5- or 6-membered ring, O═P(OH)₂,

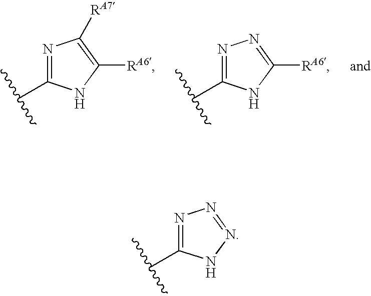

In accordance with the present invention R^{A1} or R^{A1}′ may be more particularly hydrogen or substituted or unsubstituted C₁₋₆ alkyl or even more particularly, hydrogen or an unsubstituted C₁₋₆ alkyl.

In another exemplary embodiment of the invention the compound of Formula I′ may be those where R^{B1}′ and R^{B2}′ is independently selected from the group consisting of hydrogen, N-protecting group, substituted or unsubstituted C₁₋₆ alkyl, substituted or unsubstituted C₂₋₆ alkenyl, substituted or unsubstituted C₂₋₆ alkynyl, substituted or unsubstituted C₃₋₈ cycloalkyl, substituted or unsubstituted alkcycloalkyl, where the cycloalkyl group is of three to eight carbon atoms, and the alkylene group is of one to ten carbon atoms, substituted or unsubstituted C₆ or C₁₀ aryl, substituted or unsubstituted C₇₋₁₆ alkaryl, where the alkylene group is of one to six carbon atoms and substituted or unsubstituted C₁₋₉ heterocyclyl.

In an exemplary embodiment of the invention, B or B′ may more particularly be NR^{B1}′R^{B2}′ where R^{B1}′ and R^{B2}′ may be independently selected from the group consisting of hydrogen, N-protecting group, substituted or unsubstituted C₁₋₆ alkyl, substituted or unsubstituted C₂₋₆ alkenyl, substituted or unsubstituted C₆ aryl, or substituted or unsubstituted C₇₋₁₆ alkaryl, where the alkylene group is of one to six carbon atoms.

In another exemplary embodiment of the invention, B or B′ may more particularly be NR^{B1}′R^{B2}′ where R^{B1}′R^{B2}′ is independently selected from the group consisting of hydrogen, S(O)₂R^{B7}′ wherein R^{B7}′ is selected from the group consisting of unsubstituted or substituted (e.g., NO₂, C₁₋₆ alkyl (methyl)) C₆ aryl, (CH₂)ₙCO₂RB^{3′}, wherein n is 0, 1 or 2 and wherein R^{B3′} is selected from the group consisting of hydrogen, unsubstituted C₁₋₆ alkyl, unsubstituted C₆ aryl.

In accordance with the present invention at least one of R^{B1}′, R^{B2}′ may be p-toluenesulfonyl.

In accordance with the present invention, n may be more specifically 0 or 1.

In a further exemplary embodiment of the invention R^{3′} may be hydrogen, COOR^{A1}′ where R^{A1}′ is hydrogen, substituted or unsubstituted C₁₋₆ alkyl or

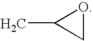

In a more particular embodiment of the invention R³ or R³′ may be hydrogen.

In an additional exemplary embodiment of the invention, R^{1a}′ and R^{1b}′, the same or different may be, for example, (a) hydrogen, (b) NR^{B1′}R^{B2′}, (c) a OR₄′ group, wherein R₄′ may be hydrogen or substituted or unsubstituted C₁₋₆ alkyl, (d) substituted or unsubstituted C₁₋₆ alkyl, (e) substituted or unsubstituted C₆ aryl (f) substituted or unsubstituted C₇₋₁₆ alkaryl where the alkylene group may be of one to four carbon atoms (g) R^{a1′} and R^{1b′} together are ═O, ═N(C₁₋₆ alkyl), or ═CR^{1c}′R^{1d}′, where each of R^{1c}′ and R^{1d}′ may be, independently, hydrogen or substituted or unsubstituted C₁₋₆ alkyl or (h) one of R^{a1}′ and R^{1b}′ may be O—CH₂ and is linked by the CH₂ group with the base nitrogen atom of B′ to form a six membered ring.

In accordance with the present invention R^{1a}′ and R^{1b}′, the same or different may be for example hydrogen or an unsubstituted C₁₋₆ alkyl.

In accordance with the present invention at least one of R^{a1}′ and R^{1b}′ may be NR^{B1′}R^{B2′} and at least one of R^{B1′} or R^{B2′} is hydrogen, an unsubstituted C₁₋₆ alkyl or a N-protecting group.

In an exemplary embodiment of the invention at least one of R^{1a}′ and R^{1b}′ is OR⁴′ where R⁴′ may be more particularly, hydrogen, an unsubstituted C₁₋₆ alkyl, an unsubstituted C₆ aryl, or an unsubstituted C₇₋₁₀ alkaryl where the alkylene part is of one to four carbon atoms.

In another embodiment of the invention, one of R^{1a}′ and R^{1b}′ is O—CH₂ and is linked by the CH₂ group with the base nitrogen atom of B′ to form a six membered ring. The other of R^{1a}′ and R^{1b}′ may be, for example, hydrogen.

In a further embodiment of the invention, R^{2a′} and R^{2b}′ the same or different may be, for example, hydrogen, F, Cl, Br, I, substituted or unsubstituted alkyl group, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms.

In accordance with the present invention, at least one of R^{2a}′ or R^{2b}′ may be more particularly, hydrogen, F, substituted or unsubstituted C₁₋₆ alkyl, a substituted or unsubstituted C₆ or C₁₀ aryl, substituted or unsubstituted C₇₋₁₆ alkaryl where the alkylene group is of one to four carbon atoms.

In an exemplary embodiment of the invention, R^{2a′} or R^{2b′} may be a C₁₋₂ alkyl linked to X′ to form a 6 or 7 membered ring.

In a further embodiment of the invention, X′ may be, for example, hydrogen, substituted or unsubstituted C₁₋₆, substituted or unsubstituted C₃₋₈ cycloalkyl, substituted or unsubstituted alkcycloalkyl, where the cycloalkyl group may be of three to eight carbon atoms and the alkylene group may be of one to four carbon atoms substituted or unsubstituted C₆ or C₁₀ aryl, substituted or unsubstituted C₇₋₁₆ alkaryl, where the alkylene group may be of one to four carbon atoms, a SO₃H group, a OR₄′ group, wherein R₄′ may be hydrogen, substituted or unsubstituted C₁₋₆ alkyl, substituted or unsubstituted C₃₋₈ cycloalkyl, substituted or unsubstituted alkcycloalkyl, where the cycloalkyl group may be of three to eight carbon atoms and the alkylene group may be of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group may be of one to four carbon atoms, substituted or unsubstituted C₁₋₉ heterocyclyl, or substituted or unsubstituted C₂₋₁₅ alkheterocyclyl, where the alkylene group may be of one to four carbon atoms.

In an exemplary embodiment of the invention, X′ may be, for example hydrogen, substituted or unsubstituted C₁₋₆, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₆ or C₁₀ aryl, or substituted or unsubstituted C₇₋₁₆ alkaryl, where the alkylene group is of one to four carbon atoms.

More particularly, X′ may be, for example, hydrogen, substituted or unsubstituted C₁₋₆, substituted or unsubstituted C₃₋₈ cycloalkyl, substituted or unsubstituted alkcycloalkyl, where the cycloalkyl group is of three to eight carbon atoms and the alkylene group is of one to four carbon atoms or a substituted or unsubstituted C₆ aryl.

In another exemplary embodiment, X′ may be a C₁₋₂ alkyl linked to the base nitrogen atom of B′ so as to form a 5 or 6 member ring, wherein the C₁₋₂ alkyl is unsubstituted or substituted with a group selected from the group consisting of OR₄′, a C₁₋₆ straight or branched alkyl and NR^{B1′}R^{B2′}.

In a further embodiment of the invention, X′ may be a C₁₋₂ alkyl linked to the base nitrogen atom of B′ so as to form a 5 or 6 member ring, and the C₁₋₂ alkyl is unsubstituted or substituted with OR₄′, where OR₄′ is for example, hydrogen or a C₁₋₆ alkyl.

In yet a further embodiment the invention, X′ may be a C₁₋₂ alkyl linked to the base nitrogen atom of B′ so as to form a 5 or 6 member ring, and the C₁₋₂ alkyl is unsubstituted or substituted with C₁₋₆ straight or branched alkyl.

In another embodiment the invention, X′ may be a C₁₋₂ alkyl linked to the base nitrogen atom of B′ so as to form a 5 or 6 member ring, and the C₁₋₂ alkyl is unsubstituted or substituted with NR^{B1′}R^{B2′}. In accordance with the present invention at least one of R^{B1′} or R^{B2′} may be for example, hydrogen, a C₁₋₆ alkyl or a N-protecting group.

In an additional embodiment of the invention X′ may be a C₃₋₄ alkyl linked to the carbon atom of R^{2a′} or R^{2b′} so as to form a 6 or 7 member ring, unsubstituted or substituted with a group selected from the group consisting of OR₄′, a C₁₋₆ straight or branched alkyl and NR^{B1′}R^{B2′}.

In yet another embodiment of the invention X′ may be oxygen, S or NR^{X1′} and X′ together with the base carbon atom of A′ forms a 5 or 6 members ring, wherein R^{X1}′ is selected from the group consisting of (i) hydrogen, (ii) an N-protecting group, (iii) substituted or unsubstituted C₁₋₆ alkyl, (iv) substituted or unsubstituted C₂₋₆ alkenyl, (v) substituted or unsubstituted C₂₋₆ alkynyl, (vi) substituted or unsubstituted C₃₋₈ cycloalkyl, (vii) substituted or unsubstituted alkcycloalkyl, where the cycloalkyl group is of three to eight carbon atoms, and the alkylene group is of one to ten carbon atoms, (viii) substituted or unsubstituted C₆ or C₁₀ aryl, (ix) substituted or unsubstituted C₇₋₁₆ alkaryl, where the alkylene group is of one to six carbon atoms, (x) substituted or unsubstituted C₁₋₉ heterocyclyl, or (xi) substituted or unsubstituted C₂₋₁₅ alkheterocyclyl, where the alkylene group is of one to six carbon atoms.

In an exemplary embodiment of the invention, X′ may be, more particularly, oxygen and X′ together with the base carbon atom of A′ may form a 5 or 6 membered ring.

In another exemplary embodiment of the invention, X′ may be, SO₃H or a salt thereof.

Additional compounds which are encompassed by the present invention are those of Formula (II′):

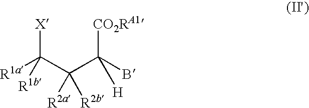

wherein X′, R^{1a}′, R^{1b}′, R^{2a}′, R^{2b}′, R^{A1}′ and B′ are as defined for Formula I′.

In an exemplary embodiment of the invention, B′ may be, for example NR^{B1}′R^{B2}′, wherein R^{B1}′ and R^{B2}′ may be independently selected from the group consisting of hydrogen, N-protecting group, substituted or unsubstituted C₁₋₆ alkyl, substituted or unsubstituted C₂₋₆ alkenyl, substituted or unsubstituted C₂₋₆ alkynyl, substituted or unsubstituted C₃₋₈ cycloalkyl, substituted or unsubstituted alkcycloalkyl, where the cycloalkyl group may be of three to eight carbon atoms, and the alkylene group may be of one to ten carbon atoms, substituted or unsubstituted C₆ or C₁₀ aryl, substituted or unsubstituted C₇₋₁₆ alkaryl, where the alkylene group may be of one to six carbon atoms and substituted or unsubstituted C₁₋₉ heterocyclyl.

In another embodiment of the invention, R₁ₐ′ and R^{1b}′, the same or different may be (a) hydrogen, (b) NR^{B1′}R^{B2′}, (c) a OR₄′ group, wherein R₄′ may be hydrogen or substituted or unsubstituted C₁₋₆ alkyl, (d) substituted or unsubstituted C₁₋₆ alkyl, or (e) R^{1a}′ and R^{1b}′ together are ═O, ═N(C₁₋₆ alkyl), or ═CR^{1c}′R^{1d}′, where each of R^{1c}′ and R_{1d}′ is, independently, hydrogen or substituted or unsubstituted C₁₋₆ alkyl. In accordance with the present invention, R^{A1}′ may be hydrogen or a straight or branched C₁₋₆ alkyl group. More particularly, R^{1a}′ or R^{1b}′ may be O—CH₂ and may be linked by the CH₂ group with the base nitrogen atom of B′.

In yet another embodiment of the invention, R^{2a}′ and R^{2b}′ the same or different may be hydrogen, F, Cl, Br, I, substituted or unsubstituted alkyl group, substituted or unsubstituted C₃₋₈ cycloalkyl, substituted or unsubstituted alkcycloalkyl, where the cycloalkyl group may be of three to eight carbon atoms and the alkylene group may be of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group may be of one to four carbon atoms.

In a further embodiment of the invention X′ may be hydrogen, substituted or unsubstituted C₁₋₆, substituted or unsubstituted C₃₋₈ cycloalkyl, substituted or unsubstituted alkcycloalkyl, where the cycloalkyl group may be of three to eight carbon atoms and the alkylene group may be of one to four carbon atoms, substituted or unsubstituted C₆ or C₁₀ aryl, or substituted or unsubstituted C₇₋₁₆ alkaryl, where the alkylene group may be of one to four carbon atoms.

More particularly X′ may be a C₃₋₄ substituted or unsubstituted alkyl linked to the carbon atom of R^{2a′} so as to form a 6 or 7 membered ring of Formula IIIA′ or IIIB′

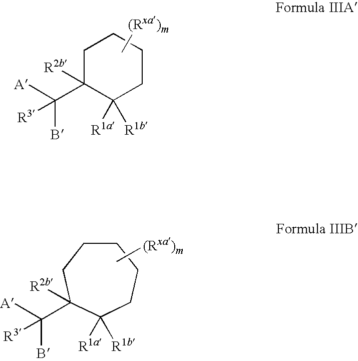

wherein B′ R^{1a}′, R^{1b}′, R^{2b}′ and R³′ is as defined with respect to Formula I′ and wherein R^{xa}′ may be selected, for example, from the group consisting of OR₄′, a C₁₋₆ straight or branched alkyl group and NR^{B1′}R^{B2} and combination thereof.

In accordance with the present invention, m may be from 0 to 8 (for compounds of Formula IIIA′) or more particularly from 0 to 6 or 0 to 4 (including 0, 1, 2, 3 or 4). For compounds of Formula IIIB′, m may be from 0 to 10 or more particularly, from 0 to 8 or 0 to 6 (e.g., 0 to 4, including 0, 1, 2, 3 or 4), More particularly, m may be 0, 1 or 2 (e.g., 0 or 1).

In a further embodiment of the invention, R^{xa}′ may be OR^{4′} and R⁴′ may be, for example, hydrogen or a straight or branched C₁₋₆ alkyl group.

In yet a further embodiment of the invention, R^{xa}′ may be NR^{B1′}R^{B2} and R^{B1′} and R^{B2}′ may be independently (a) hydrogen, (b) a N-protecting group, (c) substituted or unsubstituted C₁₋₆ alkyl, (d) substituted or unsubstituted C₂₋₆ alkenyl, (e) substituted or unsubstituted C₂₋₆ alkynyl, (f) substituted or unsubstituted C₃₋₈ cycloalkyl, (g) substituted or unsubstituted alkcycloalkyl, where the cycloalkyl group may be of three to eight carbon atoms, and the alkylene group may be of one to ten carbon atoms, (h) substituted or unsubstituted C₆ or C₁₀ aryl, or (i) substituted or unsubstituted C₇₋₁₆ alkaryl, where the alkylene group may be of one to six carbon atoms. More particularly and in accordance with the present invention R^{xa}′ may be OR₄′, where R_{4′} is for example, hydrogen, C₁₋₆ alkyl. Also in accordance with the present invention R^{xa}′ may be NR^{B1′}R^{B2} where at least one of R^{B1′} or R^{B2′} is hydrogen, N-protecting group, C₁₋₆ alkyl.

In an exemplary embodiment of the invention, A′ may be COOR^{A1}′. In accordance with the present invention R^{A1}′ may be H or a straight of branched C₁₋₆ alkyl.

In another embodiment of the invention, R^{3′} may be, for example, hydrogen.

In yet another embodiment of the invention B′ may be NR^{B1}′R^{B2}′ and where R^{B1}′ and R^{B2}′ may be independently selected from the group consisting of hydrogen, N-protecting group, substituted or unsubstituted C₁₋₆ alkyl, substituted or unsubstituted C₂₋₆ alkenyl, substituted or unsubstituted C₂₋₆ alkynyl, substituted or unsubstituted C₃₋₈ cycloalkyl, substituted or unsubstituted alkcycloalkyl, where the cycloalkyl group is of three to eight carbon atoms, and the alkylene group is of one to ten carbon atoms, substituted or unsubstituted C₆ or C₁₀ aryl, substituted or unsubstituted C₇₋₁₆ alkaryl, where the alkylene group is of one to six carbon atoms and substituted or unsubstituted C₁₋₉ heterocyclyl.

Also in accordance with the present invention, X′ together with the base carbon atom of A′ forms a 5 or 6 members ring of Formula IVA′ or Formula IVB′ wherein R^{X1}′ is as defined with respect to Formula I′,

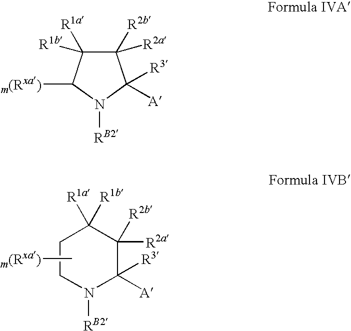

Wherein A′, R^{B2}′, R^{1a}′, R^{1b}′, R^{2a}′, R^{2b}′, R^{3′} are as defined with respect to Formula I′.

More particularly, R^{xa}′ may be selected from the group consisting of hydrogen, OR^{4′}, a C₁₋₆ straight or branched alkyl group and NR^{B1′}R^{B2} and when considering Formula IVB′ combination of OR^{4′}, a C₁₋₆ straight or branched alkyl group and NR^{B1′}R^{B2} thereof.

In accordance with the present invention A′ may be COOR^{A1}′. Also in accordance with the present invention R^{A1}′ may be H or a C₁₋₆ branched or straight alkyl group.

In an exemplary embodiment of the invention R^{B2}′ may be selected from the group consisting of (a) hydrogen, (b) a N-protecting group, (c) substituted or unsubstituted C₁₋₆ alkyl, (d) substituted or unsubstituted C₂₋₆ alkenyl, (e) substituted or unsubstituted C₂₋₆ alkynyl, (f) substituted or unsubstituted C₃₋₈ cycloalkyl, (g) substituted or unsubstituted alkcycloalkyl, where the cycloalkyl group may be of three to eight carbon atoms, and the alkylene group may be of one to ten carbon atoms, (h) substituted or unsubstituted C₆ or C₁₀ aryl, or (i) substituted or unsubstituted C₇₋₁₆ alkaryl, where the alkylene group may be of one to six carbon atoms.

In accordance with the present invention, m may be from 0, 1 or 2 (for compounds of Formula IVA′). For compounds of Formula IVB′, m may be from 0 to 4, including 0, 1, 2, 3 or 4).

In another exemplary embodiment of the invention, R^{1a}′ and R^{1b}′ may be independently, hydrogen, OR⁴′, substituted or unsubstituted C₁₋₆ alkyl, substituted or unsubstituted C₃₋₈ cycloalkyl, substituted or unsubstituted alkcycloalkyl, where the cycloalkyl group may be of three to eight carbon atoms and the alkylene group may be of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group may be of one to four carbon atoms, substituted or unsubstituted C₁₋₉ heterocyclyl, or substituted or unsubstituted C₂₋₁₅ alkheterocyclyl, where the alkylene group may be of one to four carbon atoms. More particularly, R^{1a}′ and R^{1b}′ may independently be, hydrogen, OR⁴′ or a straight or branched C₁₋₆ alkyl group.

In another exemplary embodiment of the invention R³′ may be, for example, hydrogen.

In yet another exemplary embodiment of the invention R^{2a}′ and R^{2b}′ may both be hydrogen.

In a further embodiment of the invention, R^{xa}′ may be OR^{4′} and R⁴′ may be, for example, hydrogen or a straight or branched C₁₋₆ alkyl group.

In yet a further embodiment of the invention, R^{xa}′ may be NR^{B1′}R^{B2} and R^{B1′} and R^{B2′} may be independently (a) hydrogen, (b) a N-protecting group, (c) substituted or unsubstituted C₁₋₆ alkyl, (d) substituted or unsubstituted C₂₋₆ alkenyl, (e) substituted or unsubstituted C₂₋₆ alkynyl, (f) substituted or unsubstituted C₃₋₈ cycloalkyl, (g) substituted or unsubstituted alkcycloalkyl, where the cycloalkyl group may be of three to eight carbon atoms, and the alkylene group may be of one to ten carbon atoms, (h) substituted or unsubstituted C₆ or C₁₀ aryl, or (i) substituted or unsubstituted C₇₋₁₆ alkaryl, where the alkylene group may be of one to six carbon atoms.

The present invention also encompasses compound of Formula (V′):

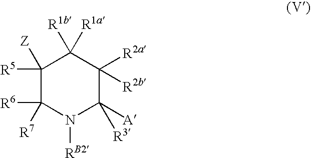

where each of A′, R^{1a′}, R^{1b′}, R^{2a′}, and R^{B2′}, are as defined with respect to Formula I′.

R⁵, R⁶, and R⁷ are each, independently, hydrogen, substituted or unsubstituted C₁₋₆ alkyl, substituted or unsubstituted C₃₋₈ cycloalkyl, substituted or unsubstituted alkcycloalkyl, where the cycloalkyl group may be of three to eight carbon atoms and the alkylene group may be of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group may be of one to four carbon atoms, substituted or unsubstituted C₁₋₉ heterocyclyl, or substituted or unsubstituted C₂₋₁₅ alkheterocyclyl, where the alkylene group may be of one to four carbon atoms; R^{3′} may be hydrogen, and R^{1a} and Z may be independently OR^{4′} or NR^{B1}′R^{B2}′.

The present invention also encompasses compound of Formula (V-A′):

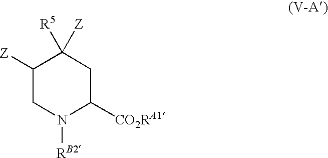

where each of R^{A1′}, R^{B2′} are as defined with respect to Formula I′ and where R⁵ may be, for example hydrogen, substituted or unsubstituted C₁₋₆ alkyl, substituted or unsubstituted C₃₋₈ cycloalkyl, substituted or unsubstituted alkcycloalkyl, where the cycloalkyl group may be of three to eight carbon atoms and the alkylene group may be of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group may be of one to four carbon atoms, substituted or unsubstituted C₁₋₉ heterocyclyl, or substituted or unsubstituted C₂₋₁₅ alkheterocyclyl, where the alkylene group may be of one to four carbon atoms; and Z is OR⁴ or NR^{B1}′R^{B2}′.

Other aspects of the invention relates to a method for reducing body weight and/or body fat in a mammal, the method may comprise modulating expression of one or more genes related to lipid metabolism.

The invention also provides a method for preventing onset or progression of excessive weight gain in a mammal, the method may comprise modulating the expression of one or more genes related to lipid metabolism.

In another aspect the invention provides a method for improving bodily appearance of a mammal, the method comprising modulating expression of one or more genes related to lipid metabolism.

The modulating aspect may comprise or consist in increasing the expression of the one or more genes.

In accordance with the present invention, the one or more genes may be selected from the group consisting of FABP4/aP2, HSL, ATGL, FatB1 and CPT-1. More specifically and in accordance with the present invention the gene may be ATGL.

In accordance with the present invention, the mammal may be a human (e.g., non-obese, overweight or obese). Also in accordance with the present invention, the human may have a Body Mass Index (BMI) of at least 25. Further in accordance with the present invention, the human may have a Body Mass Index (BMI) of at least 30.

In addition to the methods and compounds described above, the invention also includes pharmaceutical kits, as well as pharmaceutical compositions. The compounds in the kits and compositions of the invention are as described above, in reference to methods of the invention.

The pharmaceutical kits can include: (1) a compound selected from the group consisting of: isomers of 4-hydroxyisoleucine, analogs of 4-hydroxyisoleucine, and pharmaceutically acceptable lactones, salts, metabolites, solvates, and/or prodrugs of said isomers and analogs; and (2) instructions for the use of said compound for preventing or treating a disorder of fat metabolism. Optionally, the kits can also include an antiobesity agent (e.g., Orlistat, Rimonabant, Sibutramine, and/or a phentermine) and/or an antidiabetic agent (e.g., Rosiglitazone, Exendin-4, Glyburide, and Metformin), and instructions to said compound and said agent in conjunction with each other.

Further, the invention includes pharmaceutical compositions including: (1) a compound selected from the group consisting of: isomers of 4-hydroxyisoleucine, analogs of 4-hydroxyisoleucine and pharmaceutically acceptable lactones, salts, metabolites, solvates, and/or prodrugs of said isomers and analogs, and (2) an antiobesity agent (e.g., Orlistat, Rimonabant, Sibutramine, and/or a phentermine) and/or an antidiabetic agent (e.g., Rosiglitazone, Exendin-4, Glyburide, and Metformin).

In an example of a pharmaceutical composition of the invention, the composition includes: (1) a compound selected from the group consisting of: isomers of 4-hydroxyisoleucine, analogs of 4-hydroxyisoleucine and pharmaceutically acceptable lactones, salts, metabolites, solvates, and/or prodrugs of the isomers and analogs, and (2) an antiobesity agent (e.g., Orlistat, Rimonabant, Sibutramine, and/or a phentermine) and/or an antidiabetic agent (e.g., Rosiglitazone, Exendin-4, Glyburide, and Metformin).

In the kits and compositions of the invention, the compound and any other pharmaceutical agent (such as any additional antiobesity and/or antidiabetic agents) can be formulated together or separately. Further, additional antiobesity and antidiabetic agents other than those noted above can be used in the invention. Examples of such other agents are provided elsewhere herein.

Another aspect of the invention concerns a nutritional composition in form of a dietary supplement, medical food, complete meal, food additive or beverage comprising a compound selected from the group consisting of: isomers of 4-hydroxyisoleucine, analogs of 4-hydroxyisoleucine and pharmaceutically acceptable lactones, salts, metabolites, solvates, and/or prodrugs of these isomers and analogs.

An advantage of the invention is that it provides new tools for addressing the growing problem and unmet medical need of preventing and treating disorders of fat metabolism. The invention also helps addressing the growing problem and unmet medical need of obesity and related syndromes. The invention also addresses the highly demanded need for cosmetically beneficial effects associated with loss of body weight, and more particularly loss of body fat.

Additional objects, advantages, and features of the present invention will become more apparent upon reading of the following non-restrictive description of preferred embodiments with reference to the accompanying drawings, which are exemplary and should not be interpreted as limiting the scope of the present invention.

### BRIEF DESCRIPTION OF THE DRAWINGS

FIG. 1 is a synthetic scheme showing the synthesis of various analogs of 4-hydroxyisoleucine with SSS, SSR, SRS, and SRR configuration.

FIG. 2 is a synthetic scheme showing the synthesis of compounds 16 to 34.

FIG. 3 is a synthetic scheme showing the synthesis of compounds 35 to 38.

FIG. 4 is a synthetic scheme showing the synthesis of compounds 39 and 40.

FIG. 5 is a synthetic scheme showing the synthesis of compounds 41 to 62.

FIG. 6 is a synthetic scheme showing the synthesis of compounds 63 to 65a.

FIG. 7 is a synthetic scheme showing the synthesis of compounds 66 to 69.

FIG. 8 is a synthetic scheme showing the synthesis of compounds 70 to 76.

FIG. 9 is a synthetic scheme showing the synthesis of compounds 77 and 78.

FIG. 10 is a synthetic scheme showing the synthesis of compounds 79 to 85.

FIG. 11 is a synthetic scheme showing the synthesis of compounds 86a to 102b.

FIG. 12 is a synthetic scheme showing the synthesis of compounds 103 to 123.

FIG. 13 is a synthetic scheme showing the synthesis of compounds 124 to 133.

FIG. 14 is a synthetic scheme showing the synthesis of two diastereoisomers and an analog of (2S,3R,4S)-4-hydroxyisoleucine (compounds 12b and 13b).

FIG. 15A is a synthetic scheme showing the synthesis of each of the eight (8) configurational isomers of 4-hydroxyisoleucine.

FIGS. 15B and 15C are synthetic schemes showing the synthesis of compounds 137 to 147.

FIG. 16A is a line graph showing delta body weight of DIO mice treated with 25, 50, and 100 mg/kg 4-hydroxyisoleucine (4-OH, compound 14a) for 11 weeks (77 days). Delta body weight values are expressed as the body weight of a specific day minus body weight value prior to initiation of treatment. Values represent mean±SEM. N=7-8 mice per group. *p<0.05; **p<0.01; ***p<0.001.

FIG. 16B is a line graph showing food consumption of DIO-mice during and after the 11 weeks (77 days) treatment with 4-OH shown in FIG. 16A. Food consumption was measured per cage daily, and the values are expressed as the food consumption (g) per mouse, per week. Values represent mean±SEM. N=2-3 cages per group. **p<0.01.

FIG. 17A is a graph showing the effect of constant administration of fixed dosage of 4-hydroxyisoleucine (compound 14a) on body weight in diet induced obese (DIO) mice.

FIG. 17B is a graph illustrating the results of the body weight gain over time in animals of FIG. 17A.

FIG. 17C is a bar graph showing the effect of 4-hydroxyisoleucine (compound 14a) on epididymal fat in the diet induced obese (DIO) mice of FIGS. 17A and 17B at the end of treatment.

FIG. 17D is s a bar graph showing the effect of 4-hydroxyisoleucine (compound 14a) on fund consumption in the diet induced obese (DIO) mice of FIGS. 17A and 17B.

FIG. 18A is a line graph showing weekly body weight changes of DIO mice treated with 50 or 100 mg/kg 4-hydroxyisoleucine (4-OH, compound 14a) for 5 weeks (35 days).

FIG. 18B is a bar graph showing food consumption of DIO-mice treated with 50 or 100 mg/kg 4-OH for 5 weeks (35 days). Values represent mean±SEM.

FIG. 18C is a line graph showing weekly body weight changes of DIO mice treated for 5 weeks (35 days) with either 50 mg/kg 4-OH or 1.5 mg/kg Rosiglitazone, alone and in combination.

FIG. 18D is a bar graph showing food consumption of DIO-mice treated with for 5 weeks (35 days) with either 50 mg/kg 4-OH or 1.5 mg/kg Rosiglitazone, alone and in combination. Values represent mean±SEM.

FIG. 19A is a graph showing the body weight of C57BL mice on a normal diet (ND) and either kept on a normal diet, or fed with a high fat diet (HFD), without treatment (control) or with 4-OH treatment (100 mg/kg or 150 mg/kg).

FIG. 19B is a graph showing the body weight gain of the animals of FIG. 19A.

FIG. 19C is a graph showing food consumption as a function of time in animals of FIG. 19A

FIG. 19D is a bar graph showing the epididymal fat weight of the animals of FIG. 19A at the end of treatment.

FIG. 20A is a graph showing the effect of 4-hydroxyisoleucine (compound 14a) on body weight gain in Agouti mice in comparison with vehicle treated control animals.

FIG. 20B is a graph showing the effect of 4-hydroxyisoleucine (compound 14a) on food consumption in animals of FIG. 20A.

FIG. 21A is a line graph showing weekly delta body weight values from pre-treatment value of ob/ob mice treated with 100 mg/kg 4-hydroxyisoleucine (4-OH, compound 14a) for 8 weeks (56 days). Delta body weight values are expressed as the body weight of a specific day minus body weight value prior to initiation of treatment. Values represent mean±SEM. N=7-8 mice/group. *p<0.05; **p<0.01.

FIG. 21B is a line graph showing food consumption of ob/ob-mice during and after the 8 weeks (56 days) treatment with 4-OH shown in FIG. 21A. Food consumption was measured per cage daily and the values are expressed as the food consumption (g) per mouse, per week. Treatment of mice started on the first day of week 1 (Day 1, 6-7 week-old mice). N=7-8 mice/group, 2 cages/group.

FIG. 22A is a bar graph showing the prevention of weight gain by 4-hydroxyisoleucine in normal wistar rats fed a high fat, high sucrose diet (HFHS). All data are expressed as mean±SEM, n=10 rats/group.

FIG. 22B is a bar graph showing the reversal of weight gain by 4-hydroxyisoleucine in obese wistar rats. All data are expressed as mean±SEM, n=10 rats/group.

FIG. 23A is a bar graph showing the relative change of total body fat as measured by DEXA analysis of Wistar rats fed a high fat, high sucrose (HFHS) diet and treated or untreated with 4-hydroxyisoleucine (compound 14a) for 4 weeks (28 days). ** Statistically significant at p≦0.01. All data are expressed as mean±SEM.

FIG. 23B is a bar graph showing weight of epididymal fat pads of Wistar rats fed a high fat, high sucrose (HFHS) diet and treated or untreated with 4-hydroxyisoleucine (compound 14a) for 4 weeks (28 days). * Statistically significant at p≦0.05. *** Statistically significant at p≦0.001. All data are expressed as mean±SEM.

FIG. 23C is a bar graph showing weight of retroperitoneal fat pads of Wistar rats fed a high fat, high sucrose (HFHS) diet and treated or untreated with 4-hydroxyisoleucine (compound 14a) for 4 weeks (28 days). ** Statistically significant at p≦0.01. All data are expressed as mean±SEM.

FIG. 23D is a bar graph showing weight of inguinal fat pads of Wistar rats fed a high fat, high sucrose (HFHS) diet and treated or untreated with 4-hydroxyisoleucine (compound 14a) for 4 weeks (28 days). ** Statistically significant at p≦0.01. All data are expressed as mean±SEM.

FIG. 24 is a bar graph showing mean oxygen consumption during the night phase of Wistar rats fed a high fat, high sucrose (HFHS) diet and treated or untreated with 4-hydroxyisoleucine (compound 14a) for 4 weeks (28 days). * Statistically significant at p≦0.05. ** Statistically significant at p≦0.01. All data are expressed as mean±SEM.

FIG. 25 is a bar graph showing the levels of phosphorylated of ACC from control and 4-OH-treated rats, as measured by Western blot (picture insert). Values represent mean±SEM.

FIG. 26A is a line graph showing total body weight of male rats treated with three different doses of 4-Hydroxyisoleucine for 3 months (91 days). All data are expressed as mean, n=10 rats/group.

FIG. 26B is a line graph showing total body weight of female rats treated with three different doses of 4-hydroxyisoleucine for 3 months (91 days). All data are expressed as mean, n=10 rats/group.

FIG. 26C is a bar graph showing triglyceride levels of rats treated with three different doses of 4-hydroxyisoleucine for 3 months (91 days). Measurements were taken at the end of the treatment period (Day 92). All data are expressed as mean±SEM, n=10 rats/group. * Statistically significant when compared with Control group (0 mg/kg/day) at p≦0.05 (Dunnett's).

FIG. 26D is a bar graph showing total cholesterol levels of rats treated with three different doses of 4-hydroxyisoleucine for 3 months (91 days). Measurements were taken at the end of the treatment period (Day 92). All data are expressed as mean±SEM, n=10 rats/group. * Statistically significant when compared with Control group (0 mg/kg/day) at p≦0.05 (Dunnett's).

FIG. 27A is a graph showing the effect of 4-hydroxyisoleucine on oxygen comsumption during the day/night cycle on Day 21 in the model of prevention of obesity.

FIG. 27B is a graph showing the effect of 4-hydroxyisoleucine on oxygen comsumption during the day/night cycle on Day 21 in the model of reversal of obesity.

FIG. 28A is a graph showing the effect of 4-hydroxyisoleucine on respiratory quotient (RQ) during the light phase of day/night cycle in the model of prevention of obesity.

FIG. 28B is a graph showing the effect of 4-hydroxyisoleucine on respiratory quotient (RQ) during the light phase of day/night cycle in the model of reversal of obesity.

FIG. 29A is a bar graph showing reduction of body weight of DIO mice after 21 days of treatment with 25 or 50 mg/kg Compound 13e.

FIG. 29B is a bar graph showing a reduction of epididymal fat pad of DIO mice after 21 days of treatment with 25 or 50 mg/kg Compound 13e.

FIG. 30A and FIG. 30B are bar graphs showing the effect of selected analogs and isomers according to the invention on the relative change in body weight of mice. The body weight is expressed in grams (g) as delta body weight from pre-treatment. All data are expressed as mean±SEM, n=6 mice/group.

FIG. 31A is a graph showing the body weight gain in C57BL/6 mice fed with a high fat diet and receiving 4-hydroxyisoleucine (compound 14a) or compound 22.

FIG. 31B is a bar graph showing the effect of 4-hydroxyisoleucine (compound 14a) or compound 22 on epididymal fat in animals of FIG. 31A at the end of treatment.

FIGS. 32A, 32B and 32C are bar graphs showing the decrease of accumulation of lipids into 3T3-L1 pre-adipocytes committed to differentiation into mature adipocytes and treated with compound 75 (FIG. 32A), compound 76 (FIG. 32A), and compound 62 (FIG. 32C). All data are expressed as mean±SEM.

FIG. 33 is a bar graph showing energy (food intake consumption) of 4-OH treated and pair-fed rats fed a high fat, high sucrose (HFHS) diet over four weeks. All data are expressed as mean±SEM. N=10 rats/group.

FIG. 34 is a bar graph showing the relative change of total body fat of Wistar rats fed a high fat, high sucrose (HFHS) diet and treated with 4-hydroxyisoleucine (compound 14a) for 4 weeks (28 days). * Statistically significant at p≦0.05. All data are expressed as mean±SEM. N=10 rats/group.

FIG. 35 is a bar graph showing lower triglycerides plasma levels of Wistar rats fed a high fat, high sucrose (HFHS) diet and treated with 4-hydroxyisoleucine (compound 14a) for 4 weeks (28 days), as compared to other groups. * Statistically significant at p≦0.05. All data are expressed as mean±SEM. N=10 rats/group.

FIG. 36A is a bar graph showing the relative release and uptake of free fatty acids by ex vivo cultured white adipocytes collected from Wistar rats fed a high fat, high sucrose (HFHS) diet and treated or untreated with 4-hydroxyisoleucine (compound 14a) for 4 weeks (28 days). ** Statistically significant at p≦0.01. All data are expressed as mean±SEM.

FIG. 36B is a bar graph showing the insulin stimulated release of fatty acids by ex vivo cultured white adipocytes collected from Wistar rats fed a high fat, high sucrose (HFHS) diet and treated or untreated with 4-hydroxyisoleucine (compound 14a) for 4 weeks (28 days). ** Statistically significant at p≦0.01. All data are expressed as mean±SEM.

FIG. 36C is a bar graph showing the insulin stimulated fatty acids (NAFA) uptake by ex vivo cultured white adipocytes collected from Wistar rats fed a high fat, high sucrose (HFHS) diet and treated or untreated with 4-hydroxyisoleucine (compound 14a) for 4 weeks (28 days). All data are expressed as mean±SEM.

FIG. 37A is a graph showing the effect of 4-hydroxyisoleucine (compound 14a) or compound 65a on body weight in the diet-induced obesity model.

FIG. 37B is a bar graph showing the effect of 4-hydroxyisoleucine (compound 14a) or compound 65a on food consumption in the diet-induced obesity model.

### DETAILED DESCRIPTION OF THE INVENTION

The invention relates to the use of 4-hydroxyisoleucine, isomers, analogs, lactones, salts and prodrugs thereof, in the prevention and treatment of disorders of fat metabolism and related syndromes. Examples of such disorders include lipodystrophy, hypercholesterolemia, atherosclerosis, and non-alcoholic fatty liver disease, including non-alcoholic steatohepatitis (NASH). The invention provides therapeutic methods and pharmaceutical compositions for the prevention or treatment of disorders of fat metabolism such as those noted above and others known to those of skill in the art.

The invention also relates to the use of 4-hydroxyisoleucine, isomers, analogs, lactones, salts and prodrugs thereof, in the prevention and treatment of obesity and related syndromes. The invention further relates to methods for the cosmetic treatment of a mammal in order to provide a cosmetically beneficial loss of body weight, and more particularly loss of body fat. The invention provides therapeutic methods and pharmaceutical compositions for such methods. The invention further relates to methods and compositions wherein 4-hydroxyisoleucine, isomers, analogs, lactones, salts and prodrugs thereof, are used for reducing the apetite of a subject, reducing the weight of a subject, lowering plasma lipid level of a subject and/or reducing the cardiac risk of a subject.

In order to provide an even clearer and more consistent understanding of the specification and the claims, including the scope given herein to such terms, the following definitions are provided:

### A) DEFINITIONS

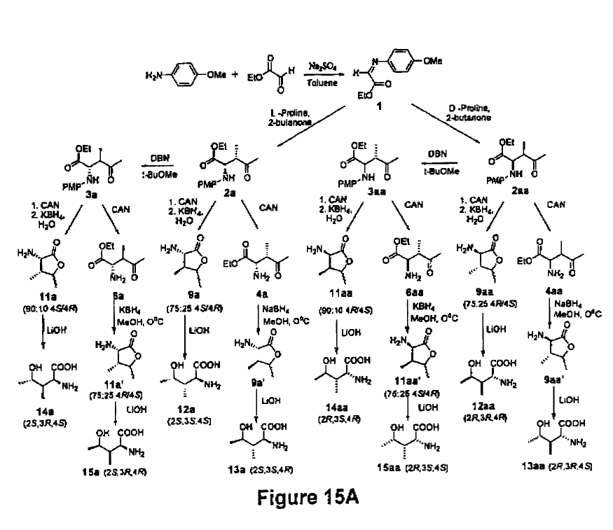

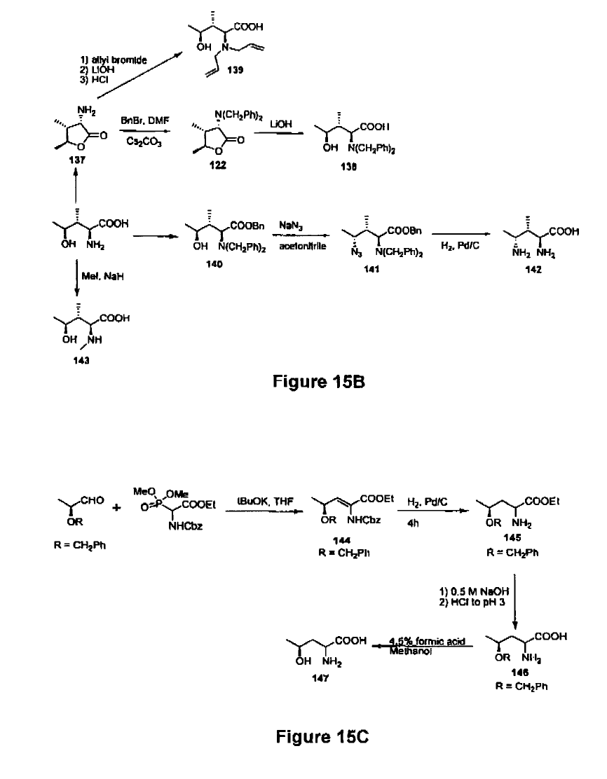

The terms “4-hydroxyisoleucine,” “4-OH,” “isomer(s) of 4-hydroxyisoleucine,” and “configurational isomer(s) of 4-hydroxyisoleucine,” as used herein, generally refer to 4-hydroxy-3-methylpentanoic acid and include all the diastereoisomers and isomers of that compound (See FIG. 15A), and also include pharmaceutically acceptable lactones, salts, crystal forms, metabolites, solvates, esters, and prodrugs thereof.

The terms “administration” and “administering” refer to a method of giving a dosage of a pharmaceutical composition to a mammal, such as a human, where the method is, e.g., oral, subcutaneous, topical, intranasal, intravenous, intraperitoneal, or intramuscular. The preferred method of administration can vary depending on various factors, e.g., the components of the pharmaceutical composition, site of the potential or actual disease, and severity of disease.

The term “alkenyl,” as used herein, represents monovalent straight or branched chain groups of, unless otherwise specified, from 2 to 12 carbons, such as, for example, 2 to 6 carbon atoms or 2 to 4 carbon atoms, containing one or more carbon-carbon double bonds and is exemplified by ethenyl, 1-propenyl, 2-propenyl, 2-methyl-1-propenyl, 1-butenyl, 2-butenyl and the like and may be optionally substituted with one, two, three, or four substituents independently selected from the group consisting of: (1) alkoxy of one to six carbon atoms; (2) alkylsulfinyl of one to six carbon atoms; (3) alkylsulfonyl of one to six carbon atoms; (4) alkynyl of two to six carbon atoms; (5) amino; (6) aryl; (7) arylalkoxy, where the alkylene group is of one to six carbon atoms; (8) azido; (9) cycloalkyl of three to eight carbon atoms; (10) halo; (11) heterocyclyl; (12) (heterocycle)oxy; (13) (heterocycle)oyl; (14) hydroxyl; (15) hydroxyalkyl of one to six carbons; (16) N-protected amino; (17) nitro; (18) oxo or thiooxo; (19) perfluoroalkyl of one to four carbons; (20) perfluoroalkoxyl of one to four carbons; (21) spiroalkyl of three to eight carbon atoms; (22) thioalkoxy of one to six carbon atoms; (23) thiol; (24) OC(O)R^{A}, where R^{A} is selected from the group consisting of (a) substituted or unsubstituted C₁₋₆ alkyl, (b) substituted or unsubstituted C₆ or C₁₀ aryl, (c) substituted or unsubstituted C₇₋₁₆ arylalkyl, where the alkylene group is of one to six carbon atoms, (d) substituted or unsubstituted C₁₋₉ heterocyclyl, and (e) substituted or unsubstituted C₂₋₁₅ heterocyclylalkyl, where the alkylene group is of one to six carbon atoms; (25) C(O)R^{B}, where R^{B} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms; (26) CO₂R^{B}, where R^{B} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms; (27) C(O)NR^{C}R^{D}, where each of R^{C} and R^{D} is, independently, selected from the group consisting of (a) hydrogen, (b) alkyl, (c) aryl, and (d) arylalkyl, where the alkylene group is of one to six carbon atoms; (28) S(O)R^{E}, where R^{E} is selected from the group consisting of (a) alkyl, (b) aryl, (c) arylalkyl, where the alkylene group is of one to six carbon atoms, and (d) hydroxyl; (29) S(O)₂R^{E}, where R^{E} is selected from the group consisting of (a) alkyl, (b) aryl, (c) arylalkyl, where the alkylene group is of one to six carbon atoms, and (d) hydroxyl; (30) S(O)₂NR^{F}R^{G}, where each of RF and R^{G} is, independently, selected from the group consisting of (a) hydrogen, (b) alkyl, (c) aryl, and (d) arylalkyl, where the alkylene group is of one to six carbon atoms; and (31) NR^{H}R^{I}, where each of R^{H} and R^{I} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group is of one to six carbon atoms; (h) cycloalkyl of three to eight carbon atoms, (i) alkcycloalkyl, where the cycloalkyl group is of three to eight carbon atoms, and the alkylene group is of one to ten carbon atoms, (j) alkanoyl of one to six carbon atoms, (k) aryloyl of 6 to 10 carbon atoms, (l) alkylsulfonyl of one to six carbon atoms, and (m) arylsulfonyl of 6 to 10 carbons atoms, with the proviso that no two groups are bound to the nitrogen atom through a carbonyl group or a sulfonyl group.

The terms “alkoxy” and “alkyloxy,” as used interchangeably herein, represent an alkyl group attached to the parent molecular group through an oxygen atom. Exemplary unsubstituted alkoxy groups are of from 1 to 6 carbons.

The term “alkyl” and “alk” as used herein, represent a monovalent group derived from a straight or branched chain saturated hydrocarbon of, unless otherwise specified, from 1 to 6 carbons and is exemplified by methyl, ethyl, n- and iso-propyl, n-, sec-, iso- and tert-butyl, neopentyl, and the like and may be optionally substituted with one, two, three or, in the case of alkyl groups of two carbons or more, four substituents independently selected from the group consisting of: (1) alkoxy of one to six carbon atoms; (2) alkylsulfinyl of one to six carbon atoms; (3) alkylsulfonyl of one to six carbon atoms; (4) alkynyl of two to six carbon atoms; (5) amino; (6) aryl; (7) arylalkoxy, where the alkylene group is of one to six carbon atoms; (8) azido; (9) cycloalkyl of three to eight carbon atoms; (10) halo; (11) heterocyclyl; (12) (heterocycle)oxy; (13) (heterocycle)oyl; (14) hydroxyl; (15) hydroxyalkyl of one to six carbons; (16) N-protected amino; (17) nitro; (18) oxo or thiooxo; (19) perfluoroalkyl of 1 to 4 carbons; (20) perfluoroalkoxyl of 1 to 4 carbons; (21) spiroalkyl of three to eight carbon atoms; (22) thioalkoxy of one to six carbon atoms; (23) thiol; (24) OC(O)R^{A}, where R^{A} is selected from the group consisting of (a) substituted or unsubstituted C₁₋₆ alkyl, (b) substituted or unsubstituted C₆ or C₁₀ aryl, (c) substituted or unsubstituted C₇₋₁₆ arylalkyl, where the alkylene group is of one to six carbon atoms, (d) substituted or unsubstituted C₁₋₉ heterocyclyl, and (e) substituted or unsubstituted C₂₋₁₅ heterocyclylalkyl, where the alkylene group is of one to six carbon atoms; (25) C(O)R^{B}, where R^{B} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms; (26) CO₂R^{B}, where R^{B} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms; (27) C(O)NR^{C}R^{D}, where each of R^{C} and R^{D} is, independently, selected from the group consisting of (a) hydrogen, (b) alkyl, (c) aryl, and (d) arylalkyl, where the alkylene group is of one to six carbon atoms; (28) S(O)R^{E}, where R^{E} is selected from the group consisting of (a) alkyl, (b) aryl, (c) arylalkyl, where the alkylene group is of one to six carbon atoms, and (d) hydroxyl; (29) S(O)₂R^{E}, where R^{E} is selected from the group consisting of (a) alkyl, (b) aryl, (c) arylalkyl, where the alkylene group is of one to six carbon atoms, and (d) hydroxyl; (30) S(O)₂NR^{F}R^{G}, where each of RF and R^{G} is, independently, selected from the group consisting of (a) hydrogen, (b) alkyl, (c) aryl, and (d) arylalkyl, where the alkylene group is of one to six carbon atoms; and (31) NR^{H}R^{I}, where each of R^{H} and R^{I} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group is of one to six carbon atoms; (h) cycloalkyl of three to eight carbon atoms, (i) alkcycloalkyl, where the cycloalkyl group is of three to eight carbon atoms, and the alkylene group is of one to ten carbon atoms, (j) alkanoyl of one to six carbon atoms, (k) aryloyl of six to ten carbon atoms, (l) alkylsulfonyl of one to six carbon atoms, and (m) arylsulfonyl of six to ten carbons atoms, with the proviso that no two groups are bound to the nitrogen atom through a carbonyl group or a sulfonyl group.

The term “alkylene,” as used herein, represents a saturated divalent hydrocarbon group derived from a straight or branched chain saturated hydrocarbon by the removal of two hydrogen atoms, and is exemplified by methylene, ethylene, isopropylene, and the like.

The term “alkylsulfinyl,” as used herein, represents an alkyl group attached to the parent molecular group through an S(O) group. Exemplary unsubstituted alkylsulfinyl groups are of from 1 to 6 carbons.

The term “alkylsulfonyl,” as used herein, represents an alkyl group attached to the parent molecular group through an S(O)₂ group. Exemplary unsubstituted alkylsulfonyl groups are of from 1 to 6 carbons.

The term “arylsulfonyl,” as used herein, represents an aryl group attached to the parent molecular group through an S(O)₂ group.

The term “alkylthio,” as used herein, represents an alkyl group attached to the parent molecular group through a sulfur atom. Exemplary unsubstituted alkylthio groups are of from 1 to 6 carbons.

The term “alkynyl,” as used herein, represents monovalent straight or branched chain groups of from two to six carbon atoms containing a carbon-carbon triple bond and is exemplified by ethynyl, 1-propynyl, and the like, and may be optionally substituted with one, two, three or four substituents independently selected from the group consisting of: (1) alkoxy of one to six carbon atoms; (2) alkylsulfinyl of one to six carbon atoms; (3) alkylsulfonyl of one to six carbon atoms; (4) alkynyl of two to six carbon atoms; (5) amino; (6) aryl; (7) arylalkoxy, where the alkylene group is of one to six carbon atoms; (8) azido; (9) cycloalkyl of three to eight carbon atoms; (10) halo; (11) heterocyclyl; (12) (heterocycle)oxy; (13) (heterocycle)oyl; (14) hydroxyl; (15) hydroxyalkyl of one to six carbons; (16) N-protected amino; (17) nitro; (18) oxo or thiooxo; (19) perfluoroalkyl of one to four carbons; (20) perfluoroalkoxyl of one to four carbons; (21) spiroalkyl of three to eight carbon atoms; (22) thioalkoxy of one to six carbon atoms; (23) thiol; (24) OC(O)R^{A}, where R^{A} is selected from the group consisting of (a) substituted or unsubstituted C₁₋₆ alkyl, (b) substituted or unsubstituted C₆ or C₁₀ aryl, (c) substituted or unsubstituted C₇₋₁₆ arylalkyl, where the alkylene group is of one to six carbon atoms, (d) substituted or unsubstituted C₁₋₉ heterocyclyl, and (e) substituted or unsubstituted C₂₋₁₅ heterocyclylalkyl, where the alkylene group is of one to six carbon atoms; (25) C(O)R^{B}, where R^{B} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms; (26) CO₂R^{B}, where R^{B} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms; (27) C(O)NR^{C}R^{D}, where each of R^{C} and R^{D} is, independently, selected from the group consisting of (a) hydrogen, (b) alkyl, (c) aryl, and (d) arylalkyl, where the alkylene group is of one to six carbon atoms; (28) S(O)R^{E}, where R^{E} is selected from the group consisting of (a) alkyl, (b) aryl, (c) arylalkyl, where the alkylene group is of one to six carbon atoms, and (d) hydroxyl; (29) S(O)₂R^{E}, where R^{E} is selected from the group consisting of (a) alkyl, (b) aryl, (c) arylalkyl, where the alkylene group is of one to six carbon atoms, and (d) hydroxyl; (30) S(O)₂NR^{F}R^{G}, where each of RF and R^{G} is, independently, selected from the group consisting of (a) hydrogen, (b) alkyl, (c) aryl, and (d) arylalkyl, where the alkylene group is of one to six carbon atoms; and (31) NR^{H}R^{I}, where each of R^{H} and R^{I} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group is of one to six carbon atoms; (h) cycloalkyl of three to eight carbon atoms; (i) alkcycloalkyl, where the cycloalkyl group is of three to eight carbon atoms, and the alkylene group is of one to ten carbon atoms; (j) alkanoyl of one to six carbon atoms; (k) aryloyl of six to ten carbon atoms; (l) alkylsulfonyl of one to six carbon atoms; and (m) arylsulfonyl of six to ten carbons atoms, with the proviso that no two groups are bound to the nitrogen atom through a carbonyl group or a sulfonyl group.

The term “alpha-amino acid residue,” as used herein, represents a N(R^{A})C(R^{B})(R^{C})C(O) linkage, where R^{A} is selected from the group consisting of (a) hydrogen, (b) alkyl, (c) aryl, and (d) arylalkyl, as defined herein; and each of R^{B} and R^{C} is, independently, selected from the group consisting of: (a) hydrogen, (b) optionally substituted alkyl, (c) optionally substituted cycloalkyl, (d) optionally substituted aryl, (e) optionally substituted arylalkyl, (f) optionally substituted heterocyclyl, and (g) optionally substituted heterocyclylalkyl, each of which is as defined herein. For natural amino acids, R^{B} is H and R^{C} corresponds to those side chains of natural amino acids found in nature, or their antipodal configurations. Exemplary natural amino acids include alanine, cysteine, aspartic acid, glutamic acid, phenylalanine, glycine, histidine, isoleucine, lysine, leucine, methionine, aspartamine, ornithine, proline, glutamine, arginine, serine, threonine, valine, tryptophan, and tyrosine, each of which, except glycine, as their D- or L-form. As used herein, for the most part, the names of naturally-occurring amino acids and acylamino residues follow the naming conventions suggested by the IUPAC Commission on the Nomenclature of Organic Chemistry and the IUPAC-IUB Commission on Biochemical Nomenclature as set out in Nomenclature of α-Amino Acids (Recommendations, 1974), Biochemistry 14 (2), 1975. The present invention also contemplates non-naturally occurring (i.e., unnatural) amino acid residues in their D- or L-form such as, for example, homophenylalanine, phenylglycine, cyclohexylglycine, cyclohexylalanine, cyclopentyl alanine, cyclobutylalanine, cyclopropylalanine, cyclohexylglycine, norvaline, norleucine, thiazoylalanine (2-, 4- and 5-substituted), pyridylalanine (2-, 3- and 4-isomers), naphthylalanine (1- and 2-isomers), and the like. Stereochemistry is as designated by convention, where a bold bond indicates that the substituent is oriented toward the viewer (away from the page) and a dashed bond indicates that the substituent is oriented away from the viewer (into the page). If no stereochemical designation is made, it is to be assumed that the structure definition includes both stereochemical possibilities.

The term “amino,” as used herein, represents an —NH₂ group.

The term “aminoalkyl” represents an amino group attached to the parent molecular group through an alkyl group.

The terms “analog(s) of 4-hydroxyisoleucine” and “analog(s)s of 4-OH,” as used herein, refer to the compounds of any of Formulae I, II, III, IV, IV-A, IV-B, IV-C, IV-D, V, V-A, VI, as well as Formulae I′, II′, IIIA′, IIB′, IVA, IVB′, V′, V-A′ and/or VI′, as described hereinafter (including the specific compounds shown in Table 1 and the figures), and also include pharmaceutically acceptable lactones, salts, crystal forms, metabolites, solvates, esters, and prodrugs of the compounds of Formulae I, II, III, IV, IV-A, IV-B, IV-C, IV-D, V, V-A, VI as well as Formulae I′, II′, IIIA′, IIB′, IVA., IVB′, V′, V-A′ and/or VI′.

The term “aryl,” as used herein, represents a mono- or bicyclic carbocyclic ring system having one or two aromatic rings and is exemplified by phenyl, naphthyl, 1,2-dihydronaphthyl, 1,2,3,4-tetrahydronaphthyl, fluorenyl, indanyl, indenyl, and the like and may be optionally substituted with one, two, three, four, or five substituents independently selected from the group consisting of: (1) alkanoyl of one to six carbon atoms; (2) alkyl of one to six carbon atoms; (3) alkoxy of one to six carbon atoms; (4) alkoxyalkyl, where the alkyl and alkylene groups are independently of one to six carbon atoms; (5) alkylsulfinyl of one to six carbon atoms; (6) alkylsulfinylalkyl, where the alkyl and alkylene groups are independently of one to six carbon atoms; (7) alkylsulfonyl of one to six carbon atoms; (8) alkylsulfonylalkyl, where the alkyl and alkylene groups are independently of one to six carbon atoms; (9) aryl; (10) arylalkyl, where the alkyl group is of one to six carbon atoms; (11) amino; (12) aminoalkyl of one to six carbon atoms; (13) aryl; (14) arylalkyl, where the alkylene group is of one to six carbon atoms; (15) aryloyl; (16) azido; (17) azidoalkyl of one to six carbon atoms; (18) carboxaldehyde; (19) (carboxaldehyde)alkyl, where the alkylene group is of one to six carbon atoms; (20) cycloalkyl of three to eight carbon atoms; (21) alkcycloalkyl, where the cycloalkyl group is of three to eight carbon atoms and the alkylene group is of one to ten carbon atoms; (22) halo; (23) haloalkyl of one to six carbon atoms; (24) heterocyclyl; (25) (heterocyclyl)oxy; (26) (heterocyclyl)oyl; (27) hydroxy; (28) hydroxyalkyl of one to six carbon atoms; (29) nitro; (30) nitroalkyl of one to six carbon atoms; (31) N-protected amino; (32) N-protected aminoalkyl, where the alkylene group is of one to six carbon atoms; (33) oxo; (34) thioalkoxy of one to six carbon atoms; (35) thioalkoxyalkyl, where the alkyl and alkylene groups are independently of one to six carbon atoms; (36) (CH₂)_{q}CO₂R^{A}, where q is an integer of from zero to four and R^{A} is selected from the group consisting of (a) alkyl, (b) aryl, and (c) arylalkyl, where the alkylene group is of one to six carbon atoms; (37) (CH₂)_{q}C(O)NR^{B}R^{C}, where R^{B} and R^{C} are independently selected from the group consisting of (a) hydrogen, (b) alkyl, (c) aryl, and (d) arylalkyl, where the alkylene group is of one to six carbon atoms; (38) (CH₂)_{q}S(O)₂R^{D}, where R^{D} is selected from the group consisting of (a) alkyl, (b) aryl, and (c) arylalkyl, where the alkylene group is of one to six carbon atoms; (39) (CH₂)_{q}S(O)₂NR^{E}R^{F}, where each of R^{E} and RF is, independently, selected from the group consisting of (a) hydrogen, (b) alkyl, (c) aryl, and (d) arylalkyl, where the alkylene group is of one to six carbon atoms; (40) (CH₂)_{q}NR^{G}R^{H}, where each of R^{G} and R^{H} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group is of one to six carbon atoms; (h) cycloalkyl of three to eight carbon atoms; and (i) alkcycloalkyl, where the cycloalkyl group is of three to eight carbon atoms, and the alkylene group is of one to ten carbon atoms, with the proviso that no two groups are bound to the nitrogen atom through a carbonyl group or a sulfonyl group; (41) oxo; (42) thiol; (43) perfluoroalkyl; (44) perfluoroalkoxy; (45) aryloxy; (46) cycloalkoxy; (47) cycloalkylalkoxy; and (48) arylalkoxy.

The term “alkaryl” represents an aryl group attached to the parent molecular group through an alkyl group. Exemplary unsubstituted arylalkyl groups are of from 7 to 16 carbons.

The term “alkheterocyclyl” represents a heterocyclic group attached to the parent molecular group through an alkyl group. Exemplary unsubstituted alkheterocyclyl groups are of from 2 to 10 carbons.

The term “alkcycloalkyl” represents a cycloalkyl group attached to the parent molecular group through an alkylene group.

The term “alkylsulfinylalkyl” represents an alkylsulfinyl group attached to the parent molecular group through an alkyl group.

The term “alkylsulfonylalkyl” represents an alkylsulfonyl group attached to the parent molecular group through an alkyl group.

The term “aryloxy,” as used herein, represents an aryl group that is attached to the parent molecular group through an oxygen atom. Exemplary unsubstituted aryloxy groups are of 6 or 10 carbons.

The terms “aryloyl” and “aroyl” as used interchangeably herein, represent an aryl group that is attached to the parent molecular group through a carbonyl group. Exemplary unsubstituted aryloxycarbonyl groups are of 7 or 11 carbons.

The term “azido” represents an N₃ group, which can also be represented as N═N═N.

The term “azidoalkyl” represents an azido group attached to the parent molecular group through an alkyl group.

The term “carbonyl,” as used herein, represents a C(O) group, which can also be represented as C═O.

The term “carboxyaldehyde” represents a CHO group.

The term “carboxaldehydealkyl” represents a carboxyaldehyde group attached to the parent molecular group through an alkyl group.

The terms “carboxy” and “carboxyl,” as used interchangeably herein, represent a CO₂H group.

The terms “carboxy protecting group” and “carboxyl protecting group,” as used herein, represent those groups intended to protect a CO₂H group against undesirable reactions during synthetic procedures. Commonly used carboxy-protecting groups are disclosed in Greene, “Protective Groups In Organic Synthesis,” 3^{rd} Edition (John Wiley & Sons, New York, 1999), which is incorporated herein by reference.

The terms “compound(s) of the invention” and “compound(s) according to the invention,” as used herein, refer to both isomer(s) of 4-hydroxyisoleucine and analogs of 4-hydroxyisoleucine as defined hereinabove.

Compounds that have the same molecular formula but differ in the nature or sequence of bonding of their atoms or the arrangement of their atoms in space are termed “isomers.” Isomers in which the connectivity between atoms is the same but which differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn, Ingold, and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

Asymmetric or chiral centers may exist in the compounds of the present invention. Unless indicated otherwise, the description or naming of a particular compound in the specification and claims is intended to include all individual enantiomers and mixtures, racemic or otherwise, thereof. The methods for the determination of stereochemistry and the separation of stereoisomers are well-known in the art (see discussion in Chapter 4 of “Advanced Organic Chemistry,” 4th edition J. March, John Wiley and Sons, New York, 1992). Individual stereoisomers of compounds of the present invention are prepared synthetically from commercially available starting materials that contain asymmetric or chiral centers or by preparation of mixtures of enantiomeric compounds followed by resolution well-known to those of ordinary skill in the art. These methods of resolution are exemplified by (1) attachment of a racemic mixture of enantiomers, designated (+/−), to a chiral auxiliary, separation of the resulting diastereomers by recrystallization or chromatography and liberation of the optically pure product from the auxiliary, or (2) direct separation of the mixture of optical enantiomers on chiral chromatographic columns. Enantiomers are designated herein by the symbols “R” or “S,” depending on the configuration of substituents around the chiral carbon atom, or are drawn by conventional means with a bolded line defining a substituent above the plane of the page in three-dimensional space and a hashed or dashed line defining a substituent beneath the plane of the printed page in three-dimensional space.

As generally understood by those skilled in the art, an optically pure compound is one that is enantiomerically pure. As used herein, the term “optically pure” is intended to mean a composition that comprises at least a sufficient amount of a single enantiomer to yield a composition having the desired pharmacological activity. Preferably, “optically pure” is intended to mean a compound that comprises at least 90% of a single isomer (80% enantiomeric excess, i.e., “e.e.”), preferably at least 95% (90% e.e.), more preferably at least 97.5% (95% e.e.), and most preferably at least 99% (98% e.e.). Preferably, the compounds of the invention are optically pure.

The term “cycloalkyl,” as used herein, represents a monovalent saturated or unsaturated non-aromatic cyclic hydrocarbon group of from three to eight carbons, unless otherwise specified, and is exemplified by cyclopropyl, cyclobutyl, cyclopentyl, cyclohexyl, cycloheptyl, bicyclo[2.2.1.]heptyl and the like. The cycloalkyl groups of this invention can be optionally substituted with (1) alkanoyl of one to six carbon atoms; (2) alkyl of one to six carbon atoms; (3) alkoxy of one to six carbon atoms; (4) alkoxyalkyl, where the alkyl and alkylene groups are independently of one to six carbon atoms; (5) alkylsulfinyl of one to six carbon atoms; (6) alkylsulfinylalkyl, where the alkyl and alkylene groups are independently of one to six carbon atoms; (7) alkylsulfonyl of one to six carbon atoms; (8) alkylsulfonylalkyl, where the alkyl and alkylene groups are independently of one to six carbon atoms; (9) aryl; (10) arylalkyl, where the alkyl group is of one to six carbon atoms; (11) amino; (12) aminoalkyl of one to six carbon atoms; (13) aryl; (14) arylalkyl, where the alkylene group is of one to six carbon atoms; (15) aryloyl; (16) azido; (17) azidoalkyl of one to six carbon atoms; (18) carboxaldehyde; (19) (carboxaldehyde)alkyl, where the alkylene group is of one to six carbon atoms; (20) cycloalkyl of three to eight carbon atoms; (21) alkcycloalkyl, where the cycloalkyl group is of three to eight carbon atoms and the alkylene group is of one to ten carbon atoms; (22) halo; (23) haloalkyl of one to six carbon atoms; (24) heterocyclyl; (25) (heterocyclyl)oxy; (26) (heterocyclyl)oyl; (27) hydroxy; (28) hydroxyalkyl of one to six carbon atoms; (29) nitro; (30) nitroalkyl of one to six carbon atoms; (31) N-protected amino; (32) N-protected aminoalkyl, where the alkylene group is of one to six carbon atoms; (33) oxo; (34) thioalkoxy of one to six carbon atoms; (35) thioalkoxyalkyl, where the alkyl and alkylene groups are independently of one to six carbon atoms; (36) (CH₂)_{q}CO₂R^{A}, where q is an integer of from zero to four and R^{A} is selected from the group consisting of (a) alkyl, (b) aryl, and (c) arylalkyl, where the alkylene group is of one to six carbon atoms; (37) (CH₂)_{q}C(O)NR^{B}R^{C}, where each of R^{B} and R^{C} is, independently, selected from the group consisting of (a) hydrogen, (b) alkyl, (c) aryl, and (d) arylalkyl, where the alkylene group is of one to six carbon atoms; (38) (CH₂)_{q}S(O)₂R^{D}, where R^{D} is selected from the group consisting of (a) alkyl, (b) aryl, and (c) arylalkyl, where the alkylene group is of one to six carbon atoms; (39) (CH₂)_{q}S(O)₂NR^{E}R^{F}, where each of R^{E} and RF is, independently, selected from the group consisting of (a) hydrogen, (b) alkyl, (c) aryl, and (d) arylalkyl, where the alkylene group is of one to six carbon atoms; (40) (CH₂)_{q}NR^{G}R^{H}, where each of R^{G} and R^{H} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group is of one to six carbon atoms; (h) cycloalkyl of three to eight carbon atoms and (i) alkcycloalkyl, where the cycloalkyl group is of three to eight carbon atoms, and the alkylene group is of one to ten carbon atoms, with the proviso that no two groups are bound to the nitrogen atom through a carbonyl group or a sulfonyl group; (41) oxo; (42) thiol; (43) perfluoroalkyl; (44) perfluoroalkoxy; (45) aryloxy; (46) cycloalkoxy; (47) cycloalkylalkoxy; and (48) arylalkoxy.

By “disorder of lipid metabolism” is meant a metabolic disorder in which the subject having the disorder cannot properly metabolize, distribute, and/or store fat. Examples of such disorders include, but are not limited to, lypodystrophy, hypercholesterolemia, and non-alcoholic fatty liver disease (e.g., non-alcoholic steatohepatitis).

By “effective amount” is meant the amount of a compound required to treat or prevent a disorder of fat metabolism or a related syndrome. The effective amount of active compound(s) used to practice the present invention for therapeutic or prophylactic treatment of conditions caused by or contributed to by disorders of fat metabolism varies depending upon the particular disorder, the manner of administration, and the age, body weight, and general health of the subject. Ultimately, the attending physician or veterinarian will decide the appropriate amount and dosage regimen. An effective amount can also be that which provides some amelioration of one or more symptoms of the disorder or decreases the likelihood of incidence of the disorder.

The terms “halogen” and “halo,” as used interchangeably herein, represent F, Cl, Br, and I.

The term “haloalkyl” represents a halo group, as defined herein, attached to the parent molecular group through an alkyl group.

The term “heteroaryl,” as used herein, represents that subset of heterocycles, as defined herein, which are aromatic: i.e., they contain 4n+2 pi electrons within the mono- or multicyclic ring system. Exemplary unsubstituted heteroaryl groups are of from 1 to 9 carbons.

The terms “heterocycle” and “heterocyclyl,” as used interchangeably herein, represent a 5-, 6-, or 7-membered ring, unless otherwise specified, containing one, two, three, or four heteroatoms independently selected from the group consisting of nitrogen, oxygen, and sulfur. The 5-membered ring has zero to two double bonds and the 6- and 7-membered rings have zero to three double bonds. The term “heterocycle” also includes bicyclic, tricyclic, and tetracyclic groups in which any of the above heterocyclic rings is fused to one or two rings independently selected from the group consisting of an aryl ring, a cyclohexane ring, a cyclohexene ring, a cyclopentane ring, a cyclopentene ring, and another monocyclic heterocyclic ring such as indolyl, quinolyl, isoquinolyl, tetrahydroquinolyl, benzofuryl, benzothienyl, and the like. Heterocyclics include pyrrolyl, pyrrolinyl, pyrrolidinyl, pyrazolyl, pyrazolinyl, pyrazolidinyl, imidazolyl, imidazolinyl, imidazolidinyl, pyridyl, piperidinyl, homopiperidinyl, pyrazinyl, piperazinyl, pyrimidinyl, pyridazinyl, oxazolyl, oxazolidinyl, isoxazolyl, isoxazolidiniyl, morpholinyl, thiomorpholinyl, thiazolyl, thiazolidinyl, isothiazolyl, isothiazolidinyl, indolyl, quinolinyl, isoquinolinyl, benzimidazolyl, benzothiazolyl, benzoxazolyl, furyl, thienyl, thiazolidinyl, isothiazolyl, isoindazoyl, triazolyl, tetrazolyl, oxadiazolyl, uricyl, thiadiazolyl, pyrimidyl, tetrahydrofuranyl, dihydrofuranyl, tetrahydrothienyl, dihydrothienyl, dihydroinidolyl, tetrahydroquinolyl, tetrahydroisoquinolyl, pyranyl, dihydropyranyl, dithiazolyl, benzofuranyl, benzothienyl and the like. Heterocyclic groups also include compounds of the formula

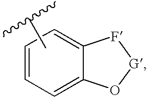

where

F′ is selected from the group consisting of CH₂, CH₂O, and O, and G′ is selected from the group consisting of C(O) and (C(R″)(R′″))ᵥ, where each of R″ and R′″ is, independently, selected from the group consisting of hydrogen or alkyl of one to four carbon atoms, and v is one to three and includes groups such as 1,3-benzodioxolyl, 1,4-benzodioxanyl and the like. Any of the heterocycle groups mentioned herein may be optionally substituted with one, two, three, four, or five substituents independently selected from the group consisting of: (1) alkanoyl of one to six carbon atoms; (2) alkyl of one to six carbon atoms; (3) alkoxy of one to six carbon atoms; (4) alkoxyalkyl, where the alkyl and alkylene groups are independently of one to six carbon atoms; (5) alkylsulfinyl of one to six carbon atoms; (6) alkylsulfinylalkyl, where the alkyl and alkylene groups are independently of one to six carbon atoms; (7) alkylsulfonyl of one to six carbon atoms; (8) alkylsulfonylalkyl, where the alkyl and alkylene groups are independently of one to six carbon atoms; (9) aryl; (10) arylalkyl, where the alkyl group is of one to six carbon atoms; (11) amino; (12) aminoalkyl of one to six carbon atoms; (13) aryl; (14) arylalkyl, where the alkylene group is of one to six carbon atoms; (15) aryloyl; (16) azido; (17) azidoalkyl of one to six carbon atoms; (18) carboxaldehyde; (19) (carboxaldehyde)alkyl, where the alkylene group is of one to six carbon atoms; (20) cycloalkyl of three to eight carbon atoms; (21) alkcycloalkyl, where the cycloalkyl group is of three to eight carbon atoms and the alkylene group is of one to ten carbon atoms; (22) halo; (23) haloalkyl of one to six carbon atoms; (24) heterocycle; (25) (heterocycle)oxy; (26) (heterocycle)oyl; (27) hydroxy; (28) hydroxyalkyl of one to six carbon atoms; (29) nitro; (30) nitroalkyl of one to six carbon atoms; (31) N-protected amino; (32) N-protected aminoalkyl, where the alkylene group is of one to six carbon atoms; (33) oxo; (34) thioalkoxy of one to six carbon atoms; (35) thioalkoxyalkyl, where the alkyl and alkylene groups are independently of one to six carbon atoms; (36) (CH₂)_{q}CO₂R^{A}, where q is an integer of from zero to four and R^{A} is selected from the group consisting of (a) alkyl, (b) aryl, and (c) arylalkyl, where the alkylene group is of one to six carbon atoms; (37) (CH₂)_{q}C(O)NR^{B}R^{C}, where each of R^{B} and R^{C} is, independently, selected from the group consisting of (a) hydrogen, (b) alkyl, (c) aryl, and (d) arylalkyl, where the alkylene group is of one to six carbon atoms; (38) (CH₂)_{q}S(O)₂R^{D}, where R^{D} is selected from the group consisting of (a) alkyl, (b) aryl, and (c) arylalkyl, where the alkylene group is of one to six carbon atoms; (39) (CH₂)_{q}S(O)₂NR^{E}R^{F}, where each of R^{E} and RF is, independently, selected from the group consisting of (a) hydrogen, (b) alkyl, (c) aryl, and (d) arylalkyl, where the alkylene group is of one to six carbon atoms; (40) (CH₂)_{q}NR^{G}R^{H}, where each of R^{G} and R^{H} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group is of one to six carbon atoms; (h) cycloalkyl of three to eight carbon atoms and (i) alkcycloalkyl, where the cycloalkyl group is of three to eight carbon atoms, and the alkylene group is of one to ten carbon atoms, with the proviso that no two groups are bound to the nitrogen atom through a carbonyl group or a sulfonyl group; (41) oxo; (42) thiol; (43) perfluoroalkyl; (44) perfluoroalkoxy; (45) aryloxy; (46) cycloalkoxy; (47) cycloalkylalkoxy; and (48) arylalkoxy.

The terms “heterocyclyloxy” and “(heterocycle)oxy,” as used interchangeably herein, represent a heterocycle group, as defined herein, attached to the parent molecular group through an oxygen atom. Exemplary unsubstituted heterocyclyloxy groups are of from 1 to 9 carbons.

The terms “heterocyclyloyl” and “(heterocycle)oyl,” as used interchangeably herein, represent a heterocycle group, as defined herein, attached to the parent molecular group through a carbonyl group. Exemplary unsubstituted heterocyclyloyl groups are of from 2 to 10 carbons.

The terms “hydroxy” and “hydroxyl,” as used interchangeably herein, represent an —OH group.

The term “hydroxyalkyl,” as used herein, represents an alkyl group, as defined herein, substituted by one to three hydroxy groups, with the proviso that no more than one hydroxy group may be attached to a single carbon atom of the alkyl group and is exemplified by hydroxymethyl, dihydroxypropyl, and the like.

The term “N-protected amino,” as used herein, refers to an amino group, as defined herein, to which is attached an N-protecting or nitrogen-protecting group, as defined herein.

The terms “N-protecting group” and “nitrogen protecting group,” as used herein, represent those groups intended to protect an amino group against undesirable reactions during synthetic procedures. Commonly used N-protecting groups are disclosed in Greene, “Protective Groups In Organic Synthesis,” 3^{rd} Edition (John Wiley & Sons, New York, 1999), which is incorporated herein by reference. N-protecting groups comprise acyl, aroyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L or D, L-amino acids such as alanine, leucine, phenylalanine, and the like; sulfonyl groups such as benzenesulfonyl, p-toluenesulfonyl, and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyl oxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, arylalkyl groups such as benzyl, triphenylmethyl, benzyloxymethyl, and the like and silyl groups such as trimethylsilyl, and the like. Preferred N-protecting groups are formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “nitro,” as used herein, represents an NO₂ group.

The term “nitroalkyl” represents a nitro group attached to the parent molecular group through an alkyl group.

The term “non-vicinal O, S, or NR′” is meant an oxygen, sulfur, or nitrogen heteroatom substituent in a linkage, where the heteroatom substituent does not form a bond to a saturated carbon that is bonded to another heteroatom.

The term “obesity” as used herein, refers to a mammal (e.g., a human) that is or is at risk of becoming overweight, obese, or afflicted with a syndrome associated with being overweight or obese. According to established standards, people are “overweight” when they have a Body Mass Index (BMI) of >25 and they are “obese” when they have a BMI>30.

By “obesity and related syndromes” is meant obesity as defined hereinabove and additional diseases or conditions associated with obesity, including but not limited to eating disorders, depression, type 2 diabetes, dyslipidemia, respiratory complications, sleep apnea, hypertension, gall bladder disease, heart disease (e.g., coronary artery disease), ostheoarthritis, atherosclerosis and certain forms of cancer (e.g., endometrial, breast, prostate, and colon cancers).

The term “oxo” as used herein, represents ═O.

The term “perfluoroalkyl,” as used herein, represents an alkyl group, as defined herein, where each hydrogen radical bound to the alkyl group has been replaced by a fluoride radical. Perfluoroalkyl groups are exemplified by trifluoromethyl, pentafluoroethyl, and the like.

The term “perfluoroalkoxy,” as used herein, represents an alkoxy group, as defined herein, where each hydrogen radical bound to the alkoxy group has been replaced by a fluoride radical.

The term “pharmaceutically acceptable salt,” as use herein, represents those salts which are, within the scope of sound medical judgment, suitable for use in contact with the tissues of humans and animals without undue toxicity, irritation, allergic response, and the like and are commensurate with a reasonable benefit/risk ratio. Pharmaceutically acceptable salts are well known in the art. For example, S. M. Berge et al. describe pharmaceutically acceptable salts in detail in J. Pharmaceutical Sciences 66:1-19, 1977. The salts can be prepared in situ during the final isolation and purification of the compounds of the invention or separately by reacting the free base group with a suitable organic acid. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to, ammonium, tetramethylammonium, tetraethylammonium, methylamine, dimethylamine, trimethylamine, triethylamine, ethylamine, and the like.

The term “pharmaceutically acceptable ester,” as used herein, represents esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic, and alkanedioic acids, in which each alkyl or alkenyl group preferably has not more than 6 carbon atoms. Examples of particular esters include formates, acetates, propionates, butyates, acrylates, and ethylsuccinates.

The term “prodrug,” as used herein, represents compounds that are rapidly transformed in vivo to a parent compound of the above formula, for example, by hydrolysis in blood. A thorough discussion is provided in T. Higuchi and V. Stella, “Pro-drugs as Novel Delivery Systems,” Vol. 14 of the A.C.S. Symposium Series, Edward B. Roche, ed., “Bioreversible Carriers in Drug Design,” American Pharmaceutical Association and Pergamon Press, 1987, and Judkins et al., Synthetic Communications 26(23):4351-4367, 1996, each of which is incorporated herein by reference.

Prodrugs of isomers and analogs according to the invention can be prepared by modifying functional groups in such a way that the modifications may be cleaved in vivo to release the parent isomer or analog. Prodrugs include modified isomers or analogs in which a hydroxy or amino group in any of the isomer or analog is bonded to any group that may be cleaved in vivo to regenerate the free hydroxyl or amino group, respectively. Examples of prodrugs include, but are not limited to esters (e.g., acetate, formate, and benzoate derivatives), and carbamates (e.g., N,N-dimethylaminocarbonyl) of hydroxy functional groups in compounds of Formulae I, II, III, IV, IV-A, IV-B, IV-C, IV-D, V, V-A, VI, as well as Formulae I′, II′, IIIA′, IIB′, IVA., IVB′, V′, V-A′ and/or VI′ and the like.

The term “pharmaceutically acceptable prodrugs,” as used herein, represents those prodrugs of the compounds of the present invention which are, within the scope of sound medical judgment, suitable for use in contact with the tissues of humans and animals without undue toxicity, irritation, allergic response, and the like, commensurate with a reasonable benefit/risk ratio, and effective for their intended use, as well as the zwitterionic forms, where possible, of the compounds of the invention.

A “pharmaceutically acceptable active metabolite” is intended to mean a pharmacologically active product produced through metabolism in the body of a compound according to the invention.

A “pharmaceutically acceptable solvate” is intended to mean a solvate that retains the biological effectiveness and properties of the biologically active components of isomers and analogs according to the invention. Examples of pharmaceutically acceptable solvates include, but are not limited to water, isopropanol, ethanol, methanol, DMSO, ethyl acetate, acetic acid, and ethanolamine.

“Prevention or treatment of a disorder of fat metabolism” is intended to mean any beneficial prophylactic or therapeutic activity related to fat metabolism in a mammal (preferably a human), including but not limited to beneficial effects on any one or more of lipolysis, oxygen consumption, lipid storage, lipid processing, lipid profiles, lipid blood levels, body weight and/or body fat, the onset or progression of excessive weight gain, appetite, levels of food intake, energy expenditure and/or modulation of genes related to fat metabolism.

By “ring system substituent” is meant a substituent attached to an aromatic or non-aromatic ring system. When a ring system is saturated or partially saturated the “ring system substituent” further includes methylene (double bonded carbon), oxo (double bonded oxygen), or thioxo (double bonded sulfur).

The term “spiroalkyl,” as used herein, represents an alkylene diradical, both ends of which are bonded to the same carbon atom of the parent group to form a spirocyclic group.

The term “sulfonyl,” as used herein, represents an S(O)₂ group.

The term “thioalkoxy,” as used herein, represents an alkyl group attached to the parent molecular group through a sulfur atom. Exemplary unsubstituted thioalkoxy groups are of from 1 to 6 carbons.

The term “thioalkoxyalkyl” represents a thioalkoxy group attached to the parent molecular group through an alkyl group.

By the terms “thiocarbonyl” and “thiooxo” is meant a C(S) group, which can also be represented as C═S.

By the terms “thiol” and “sulfhydryl” is meant an SH group.

By the phrase “in conjunction with” is meant the administration of two or more compounds (for example, a compound 1, compound 2, compound 3, etc.) prior to, after, and/or simultaneously with the other. In this context, the phrase “administration of two compounds simultaneously” refers to administration of compounds 1 and 2 within 48 hours (e.g., 24 hours) of each other. In some embodiments, “in conjunction with” includes administration of compounds 1 and 2 sufficiently closely in time for there to be a beneficial effect for the patient, that is greater, over the course of the treatment, than if either of compounds 1 and 2 are administered alone, in the absence of the other, over the same course of treatment. In some embodiments, the beneficial effect is the treatment of diabetes with reduction or prevention of weight-gain.

### B) COMPOUNDS ACCORDING TO THE INVENTION

As will be described in detail hereinafter, the inventors have found that hydroxylated amino acids and more particularly, 4-hydroxyisoleucine, configurational isomers, analogs, lactones, prodrugs, pharmaceutical salts, pharmaceutical esters, metabolites, and solvates thereof have properties indicating that they can be effective (i) in the prevention and/or treatment of disorders of fat metabolism; (ii) in the prevention and/or treatment of obesity and related syndromes; and (iii) for cosmetically beneficial loss of body weight, as described herein.

The invention thus provides methods, compounds, and pharmaceutical compositions for treating a mammal (e.g., a human) that has or is at risk of developing a disorder of fat metabolism. Particular uses of the methods, compounds, and pharmaceutical compositions of the invention include, but are not limited to, the prevention or treatment of disorders including lipodystrophy, hypercholesterolemia, atherosclerosis, and non-alcoholic fatty liver disease, including non-alcoholic steatohepatitis (NASH). Additional uses of the methods, compounds, and pharmaceutical compositions of the invention include, but are not limited to, the prevention and/or treatment of obesity and related syndromes and to the cosmetic treatment of a mammal in order to effect a cosmetically beneficial loss of body weight, and more particularly loss of body fat.

### i) Isomers of 4-Hydroxyisoleucine

According to an embodiment, the compounds for use according to the invention are chosen among any of the configurational isomers of 4-hydroxyisoleucine, and pharmaceutically acceptable lactones, salts, crystal forms, prodrugs, esters, metabolites, or solvates thereof. In certain embodiments, the isomer of 4-hydroxyisoleucine is selected from the group consisting of:

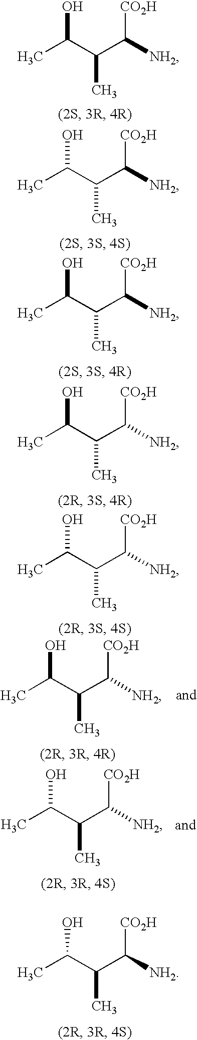

In a preferred embodiment, the isomer of 4-hydroxyisoleucine is the (2S,3R,4S) isomer (compound 14a). In another preferred embodiment, the isomer of 4-hydroxyisoleucine is the (2R,3S,4R) isomer.

Exemplary prodrugs of isomers of 4-hydroxyisoleucine include those compounds in which the carboxylate group and the hydroxyl group are condensed to form one of the following lactones:

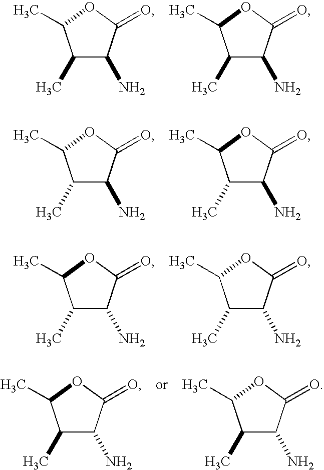

The isomers of 4-hydroxyisoleucine can be prepared by employing techniques available in the art using starting materials that are readily available. For instance, methods for the preparation of (2S,3R,4S)-4-hydroxyisoleucine have been described, see for example U.S. Patent Application Publication No. US 2003/0219880; Rolland-Fulcrand et al., Eur. J. Org. Chem. 873-877, 2004; and Wang et al., Eur. J. Org. Chem. 834-839, 2002. In addition, this compound can be isolated from the seeds of fenugreek (Trigonella foenum-graecum). Methods for making additional configurational isomers of 4-hydroxyisoleucine, or prodrugs thereof, have also been described in PCT application PCT/EP2005/013975 filed Nov. 10, 2005 (published as WO 2006/051000 on May 18, 2006) and PCT application PCT/IB2006/001758 filed Feb. 17, 2006 (published as WO 2006/117696A1; originally designated as PCT/US2006/005794, filed on Feb. 17, 2006), which are each incorporated herein by reference. FIG. 15A shows a synthetic scheme for the synthesis of each eight (8) configurational isomers of 4-hydroxyisoleucine.

### ii) Analogs of 4-Hydroxyisoleucine

As is noted above, in addition to 4-hydroxyisoleucine in all isomeric forms, the invention also concerns the use and/or administration of analogs of 4-hydroxyisoleucine (in any isomeric form) for the prevention and/or treatment of disorders of fat metabolism and/or any of their related syndromes.

In one embodiment, the analogs of 4-hydroxyisoleucine according to the present invention are represented by the generalized Formula (I):

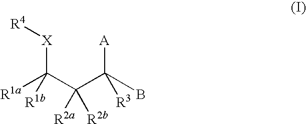

and pharmaceutically acceptable lactones, salts, prodrugs, metabolites, or solvates thereof.

The substituent A in a compound of Formula (I) can be CO₂R^{A1}, C(O)SR^{A1}, C(S)SR^{A1}, C(O)NR^{A2}R^{A3}, C(S)NR^{A2}R^{A3}, C(O)R^{A4}, SO₃H, S(O)₂NR^{A2}R^{A3}, C(O)R^{A5}, C(OR^{A1})R^{A9}A¹⁰, C(SR^{A1})R^{A9}R^{A10}, C(═NR^{A1})R^{A}, O═P(OH)₂,

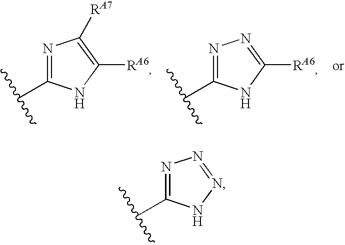

where

R^{A1} is hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms,

each of R^{A2} and R^{A3} is, independently, selected from the group consisting of (a) hydrogen, (b) substituted or unsubstituted C₁₋₆ alkyl, (c) substituted or unsubstituted C₃₋₈ cycloalkyl, (d) substituted or unsubstituted alkcycloalkyl, where the cycloalkyl group is of three to eight carbon atoms and the alkylene group is of one to four carbon atoms, (e) substituted or unsubstituted C₆ or C₁₀ aryl, and (f) substituted or unsubstituted C₇₋₁₆ alkaryl, where the alkylene group is of one to six carbon atoms, or R^{A2} taken together with R^{A3} and N forms a substituted or unsubstituted 5- or 6-membered ring, optionally containing O or NR^{A8}, where R^{A8} is hydrogen or C₁₋₆ alkyl,

R^{A4} is hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms,

R^{A5} is a peptide chain of 1-4 natural or unnatural amino acids, where the peptide is linked via its terminal amine group to C(O),

each of R^{A6} and R^{A7} is, independently, hydrogen, substituted or unsubstituted C₁₋₆ alkyl, C₁₋₄ perfluoroalkyl, substituted or unsubstituted C₁₋₆ alkoxy, amino, C₁₋₆ alkylamino, C₂₋₁₂ dialkylamino, N-protected amino, halo, or nitro, and

each of R^{A9} and R^{A10} is, independently, selected from the group consisting of (a) hydrogen, (b) substituted or unsubstituted C₁₋₆ alkyl, (c) substituted or unsubstituted C₃₋₈ cycloalkyl, (d) substituted or unsubstituted alkcycloalkyl, where the cycloalkyl group is of three to eight carbon atoms and the alkylene group is of one to four carbon atoms, (e) substituted or unsubstituted C₆ or C₁₀ aryl, and (f) substituted or unsubstituted C₇₋₁₆ alkaryl, where the alkylene group is of one to six carbon atoms, or R^{A9} taken together with R^{A10} and their parent carbon atom forms a substituted or unsubstituted 5- or 6-membered ring, optionally containing O or NR^{A8}, wherein R^{A8} is hydrogen or C₁₋₆ alkyl.

The substituent B in a compound of Formula (I) can be NR^{B1}R^{B2}, where each of R^{B1} and R^{B2} is, independently selected from the group consisting of (a) hydrogen, (b) an N-protecting group, (c) substituted or unsubstituted C₁₋₆ alkyl, (d) substituted or unsubstituted C₂₋₆ alkenyl, (e) substituted or unsubstituted C₂₋₆ alkynyl, (f) substituted or unsubstituted C₃₋₈ cycloalkyl, (g) substituted or unsubstituted alkcycloalkyl, where the cycloalkyl group is of three to eight carbon atoms, and the alkylene group is of one to ten carbon atoms, (h) substituted or unsubstituted C₆ or C₁₀ aryl, (i) substituted or unsubstituted C₇₋₁₆ alkaryl, where the alkylene group is of one to six carbon atoms, (j) substituted or unsubstituted C₁₋₉ heterocyclyl, (k) substituted or unsubstituted C₂₋₁₅ alkheterocyclyl, where the alkylene group is of one to six carbon atoms, (l) (CH₂)ₙC(O)R^{B3}, wherein n is 0, 1, 2 or 3, where R^{B3} is selected from the group consisting of hydrogen, substituted or unsubstituted C₁₋₆ alkyl, substituted or unsubstituted C₆ or C₁₀ aryl, substituted or unsubstituted C₇₋₁₆ alkaryl, where the alkylene group is of one to six carbon atoms, substituted or unsubstituted C₁₋₉ heterocyclyl, or substituted or unsubstituted C₂₋₁₅ alkheterocyclyl, where the alkylene group is of one to six carbon atoms, (m) (CH₂)ₙCO₂R^{B4}, wherein n is 0, 1, 2, or 3, where R^{B4} is selected from the group consisting of hydrogen, substituted or unsubstituted C₁₋₆ alkyl, substituted or unsubstituted C₆ or C₁₀ aryl, substituted or unsubstituted C₇₋₁₆ alkaryl, where the alkylene group is of one to six carbon atoms, substituted or unsubstituted C₁₋₉ heterocyclyl, or substituted or unsubstituted C₂₋₁₅ alkheterocyclyl, where the alkylene group is of one to six carbon atoms, (n) C(O)NR^{B5}R^{B6}, where each of R^{B5} and R^{B6} is, independently, selected from the group consisting of hydrogen, substituted or unsubstituted C₁₋₆ alkyl, substituted or unsubstituted C₆ or C₁₀ aryl, substituted or unsubstituted C₇₋₁₆ alkaryl, where the alkylene group is of one to six carbon atoms, substituted or unsubstituted C₁₋₉ heterocyclyl, and substituted or unsubstituted C₂₋₁₅ alkheterocyclyl, where the alkylene group is of one to six carbon atoms, or R^{B5} taken together with R^{B6} and N forms a substituted or unsubstituted 5- or 6-membered ring, optionally containing a non-vicinal O, S, or NR′, where R′ is H or C₁₋₆ alkyl, (O)S(O)₂R^{B7}, where R^{B7} is selected from the group consisting of hydrogen, substituted or unsubstituted C₁₋₆ alkyl, substituted or unsubstituted C₆ or C₁₀ aryl, substituted or unsubstituted C₇₋₁₆ alkaryl, where the alkylene group is of one to six carbon atoms, substituted or unsubstituted C₁₋₉ heterocyclyl, or substituted or unsubstituted C₂₋₁₅ alkheterocyclyl, where the alkylene group is of one to six carbon atoms, and (p) a peptide chain of 1-4 natural or unnatural alpha-amino acid residues, where the peptide is linked via its terminal carboxy group to N, with the proviso that no two groups are bound to the nitrogen atom through a carbonyl group or a sulfonyl group. Alternatively, R^{B1} can form ring systems when combined with other substituents of Formula I. In one ring system, R^{B1} taken together with R^{B2} and N forms a substituted or unsubstituted 5- or 6-membered ring, optionally containing O or NR^{B8}, wherein R^{B5} is hydrogen or C₁₋₆ alkyl. Alternatively, a 5- to 8-membered ring is formed when R^{B1} taken together with R^{1a} is a substituted or unsubstituted C₁₋₄ alkyl or a [2.2.1] or [2.2.2] bicyclic ring system is formed when R^{B1} taken together with R^{1a} is a substituted or unsubstituted C₂ alkylene and R^{B1} taken together with R^{2a} is a substituted or unsubstituted C₁₋₂ alkylene. Alternatively, a 4- to 8-membered ring is formed when R^{B1} taken together with R³ is a substituted or unsubstituted C₂₋₆ alkyl. A 6- to 8-membered ring can be formed when R^{B1} taken together with R⁴ is a substituted or unsubstituted C₁₋₃ alkyl. Yet another ring is formed when R^{B1} taken together with A and the parent carbon of A and B form the following ring:

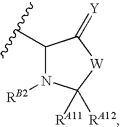

where each of Y and W is, independently, O, S, NR^{B8}, or CR^{A9}R^{A10}, where each of R^{A9} and R^{A10} is as previously defined and each of R^{A11} and R^{A12} is, independently, selected from the group consisting of (a) hydrogen, (b) substituted or unsubstituted C₁₋₆ alkyl, (c) substituted or unsubstituted C₃₋₈ cycloalkyl, (d) substituted or unsubstituted alkcycloalkyl, where the cycloalkyl group is of three to eight carbon atoms and the alkylene group is of one to four carbon atoms, (e) substituted or unsubstituted C6 or C₁₀ aryl, and (f) substituted or unsubstituted C₇₋₁₆ alkaryl, where the alkylene group is of one to six carbon atoms, or R^{A9} taken together with R^{A10} and their parent carbon atom forms a substituted or unsubstituted 5- or 6-membered ring, optionally containing O or NR^{A8}, wherein R^{A8} is hydrogen or C₁₋₆ alkyl. In one embodiment, the B′ substituent does not form rings with R^{1a}, R^{1b}, or R⁴.

The substituent X in a compound of Formula (I) may be either (i) absent (ii) hydrogen, (iii) a substituted or unsubstituted C₁₋₆, (iv) substituted or unsubstituted C₃₋₈ cycloalkyl, (v) substituted or unsubstituted alkcycloalkyl, where the cycloalkyl group is of three to eight carbon atoms and the alkylene group is of one to four carbon atoms, (vi) substituted or unsubstituted C₆ or C₁₀ aryl, (vii) substituted or unsubstituted C₇₋₁₆ alkaryl, where the alkylene group is of one to four carbon atoms, (viii) SO₃H; (ix) O, (x) S, or (xi) NR^{X1}, where R^{X1} is selected from the group consisting of (a) hydrogen, (b) an N-protecting group, (c) substituted or unsubstituted C₁₋₆ alkyl, (d) substituted or unsubstituted C₂₋₆ alkenyl, (e) substituted or unsubstituted C₂₋₆ alkynyl, (f) substituted or unsubstituted C₃₋₈ cycloalkyl, (g) substituted or unsubstituted alkcycloalkyl, where the cycloalkyl group is of three to eight carbon atoms, and the alkylene group is of one to ten carbon atoms, (h) substituted or unsubstituted C6 or C₁₀ aryl, (i) substituted or unsubstituted C₇₋₁₆ alkaryl, where the alkylene group is of one to six carbon atoms, (j) substituted or unsubstituted C₁₋₉ heterocyclyl, or (k) substituted or unsubstituted C₂₋₁₅ alkheterocyclyl, where the alkylene group is of one to six carbon atoms.

For a compound of Formula (I), each of the R^{1a} and R^{1b} substituents is, independently, (a) hydrogen, (b) substituted or unsubstituted C₁₋₆ alkyl, (c) substituted or unsubstituted C₃₋₈ cycloalkyl, (d) substituted or unsubstituted alkcycloalkyl, where the cycloalkyl group is of three to eight carbon atoms and the alkylene group is of one to four carbon atoms, (e) substituted or unsubstituted C₂₋₆ alkenyl, (f) substituted or unsubstituted C₂₋₆ alkynyl, (g) substituted or unsubstituted C₆ or C₁₀ aryl, (h) substituted or unsubstituted C₇₋₁₆ alkaryl, where the alkylene group is of one to four carbon atoms, (i) substituted or unsubstituted C₁₋₉ heterocyclyl, (j) substituted or unsubstituted C₂₋₁₅ alkheterocyclyl, where the alkylene group is of one to four carbon atoms, or R^{1a} together with R^{2a} and their base carbon atoms form a substituted or unsubstituted C₅₋₁₀ mono or fused ring system, or a 3- to 6-membered ring is formed when R^{1a} together with R⁴ is a substituted or unsubstituted C₁₋₄ alkylene, (k) NR^{B1}R^{B2}, (l) a OR₄ group, or (m) R^{1a} and R^{1b} together are ═O, ═N(C₁₋₆ alkyl), ═CR^{1c}R^{1d}, where each of R_{1c} and R^{1d} is, independently, hydrogen or substituted or unsubstituted C₁₋₆ alkyl, or a substituted or unsubstituted C₂₋₅ alkylene moiety forming a spiro ring.

For a compound of Formula (I), each of the R^{2a} and R^{2b} is, independently, hydrogen, halogen (e.g., F, Cl, Br, I), substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms, or R^{2a} and R^{2b} together are ═O, ═N(C₁₋₆ alkyl), ═CR^{2a}R^{2d}, where each of R^{2c} and R^{2d} is, independently, hydrogen or substituted or unsubstituted C₁₋₆ alkyl, or a substituted or unsubstituted C₂₋₅ alkylene moiety forming a spiro ring, or R^{2a} together with R^{1a} and their base carbon atoms form a substituted or unsubstituted C₅₋₁₀ mono or fused ring system.

The substituent R³ in a compound of Formula (I) may be hydrogen, COOR^{A1}, substituted or unsubstituted C₁₋₆ alkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₇₋₁₆ alkaryl, where the alkylene group is of one to four carbon atoms, or substituted or unsubstituted C₂₋₁₅ alkheterocyclyl, where the alkylene group is of one to four carbon atoms. Alternatively, a 4- to 8-membered ring can be formed when R³ taken together with R^{B1} is a substituted or unsubstituted C₂₋₆ alkylene.

The substituent R⁴ in a compound of Formula (I) is either absent, hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms, or a 3- to 6-membered ring is formed when R⁴ together with R^{1a} is a substituted or unsubstituted C₁₋₄ alkylene, or a 6- to 8-membered ring is formed when R⁴ taken together with R^{B1} is a substituted or unsubstituted C₁₋₃ alkylene.

In certain embodiments, the analogs of the present invention are represented by generalized Formula (I) and the attendant definitions, wherein A is CO₂H, B is NH-p-toluenesulfonyl, R⁴ is H, and each of R^{1a} and R^{2a} is CH₃.

In certain embodiments, the analogs of the present invention are represented by generalized Formula (I) and the attendant definitions, wherein A is CO₂H, B is NH₂, R⁴ is H, and each of R^{1a} and R^{2a} is a substituted or unsubstituted C₁₋₆ alkyl.

In certain embodiments, the analogs of the present invention are represented by generalized Formula (I) and the attendant definitions, wherein R^{1a} together with R^{2a} and their base carbon atoms form a substituted or unsubstituted C₅₋₁₀ mono or fused ring system, optionally containing a non-vicinal O, S, or NR′, where R′ is H or C₁₋₆ alkyl.

In certain embodiments, the analogs of the present invention are represented by the generalized Formula (II), or a pharmaceutically acceptable lactone, salt, metabolite, solvate, and/or prodrug thereof:

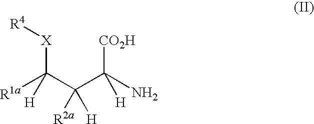

where each of R^{1a} and R^{2a} is, independently, substituted or unsubstituted C₁₋₆ alkyl or R^{1a} together with R^{2a} and their base carbon atoms form a substituted or unsubstituted C₆ alicyclic ring system. In certain embodiments, the analogs of the present invention are represented by generalized Formula (II) and the attendant definitions, wherein R^{1a} represents an ethyl group, R^{2a} represents a methyl group, X represents O, and R⁴ represents an hydrogen atom. Some examples of this embodiment include compounds identified as having ID Nos 13b, 12b, 218, 219, 220, 221, 222, and 223 in Table 1 hereinafter.

In another embodiment, the analogs of 4-hydroxyisoleucine according to the present invention are represented by Formula I′, II′, IIIA′, IIB′, IVA., IVB′, V′, V-A′ and/or VI′, as described herein.

In certain embodiments, the analogs of the present invention are represented by generalized Formula (II) and the attendant definitions, wherein X represents O, R⁴ represents an hydrogen atom, and R^{1a} and R^{2a} join to form a six or seven membered ring structure. Some examples of this embodiment include compounds identified as having ID Nos 12e, 13e, 14e, 15e, 213, 214, 215, 216, 217, 12f, 13f, 14f, 15f, 231, 232, 233, 234, and 235 in Table 1 hereinafter.

In certain embodiments, the analogs of the present invention are represented by generalized Formula (II) and the attendant definitions, wherein R^{1a} represents a methyl group, R^{2a} represents a benzyl group, X represents O, and R⁴ represents an hydrogen atom. Some examples of this embodiment include compounds identified as having ID Nos 12d, 13d, 14d, 15d, 238, 239, 240, and 241 in Table 1 hereinafter.

Yet, in some embodiments, the analogs of the present invention are represented by generalized Formula (I) and the attendant definitions, wherein R^{1a}, R^{1b} and R^{2a} represent methyl groups, X represents O, and R⁴ represents a hydrogen atom. Some examples of this embodiment include compounds identified as having ID Nos 207, 101a, 101b, 208, 209, and 210 in Table 1 hereinafter. Desirable compounds of this embodiment have the 2S,3R configuration.

In certain embodiments, the analogs of the present invention are represented by generalized Formula (III), or a pharmaceutically acceptable lactone, salt, metabolite, solvate, and/or prodrug thereof:

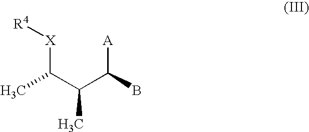

where each of B, X, and R⁴ is as defined elsewhere herein (see Formula I, above) and A is CO₂R^{A1}, C(O)SR^{A1}, C(O)NR^{A2}R^{A3}, or C(O)R^{A}.

In certain embodiments, the analogs of the present invention are represented by generalized Formula (IV), or a pharmaceutically acceptable lactone, salt, metabolite, solvate, and/or prodrug thereof:

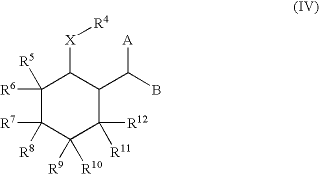

where each of B, X, and R⁴ is as defined elsewhere herein (see Formula I, above), A is CO₂R^{A1}, C(O)SR^{A1}, C(O)NR^{A2}R^{A3}, or C(O)R^{A5}, and R⁵, R⁶, R⁷, R⁸, R⁹, R¹⁰, R¹¹, and R¹² are, independently, hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms. Desirable compounds of this embodiment have the SSR-configuration.

In certain embodiments, the compounds of the present invention are represented by the following generalized formulae, or a pharmaceutically acceptable lactone, salt, solvate, and/or prodrug thereof:

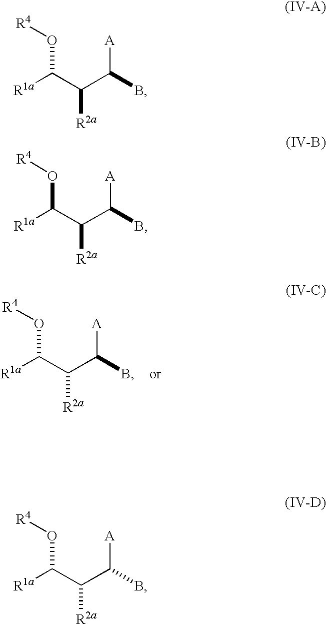

where each of R^{1a} and R^{2a} is, individually, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms.

In one preferred example of this embodiment, A is CO₂H, B is NH₂, R⁴ is H, and each of R^{1a} and R^{2a} is a substituted or unsubstituted C₁₋₆ alkyl. In another example, preferable analogs of 4-OH include those compounds where R^{1a} together with R^{2a} and their base carbon atoms form a substituted or unsubstituted C₅₋₁₀ mono or fused ring system, such as, for example, a compound selected from the group consisting of:

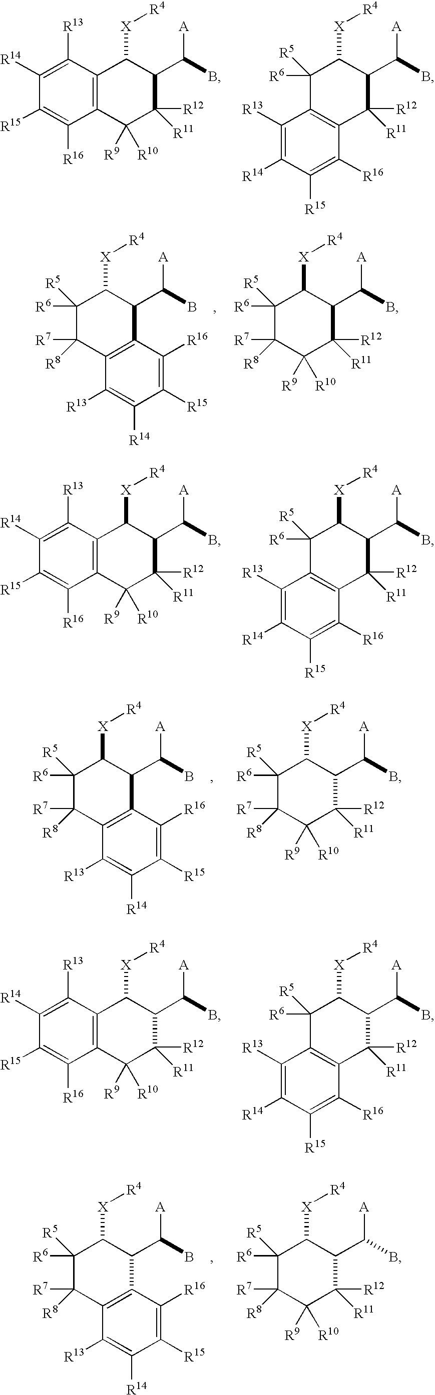

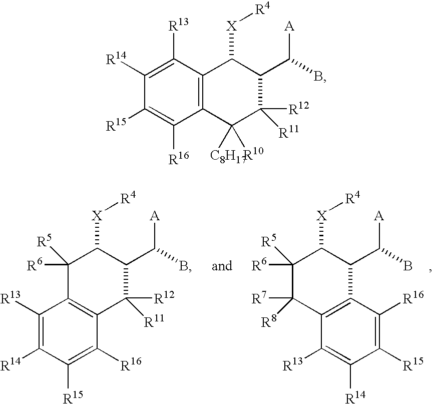

where each of R⁵, R⁶, R⁷, R⁸, R⁹, R¹⁰, R¹¹, and R¹² is, independently, hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms; and each of R¹³, R¹⁴, R¹⁵, and R¹⁶ is, independently, hydrogen, substituted or unsubstituted C₁₋₆ alkyl, C₁₋₄ perfluoroalkyl, substituted or unsubstituted C₁₋₆ alkoxy, amino, C₁₋₆ alkylamino, C₂₋₁₂ dialkylamino, N-protected amino, halo, or nitro. Most preferable compounds in this series are those in which A is CO₂H and B is NH₂.

In another embodiment, the compound of Formula (I) is

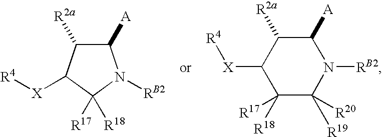

where each of R¹⁷, R¹⁸, R¹⁹, and R²⁰ is hydrogen or substituted or unsubstituted C₁₋₆ alkyl.

In another embodiment, the compound of Formula (I) is

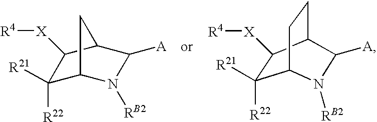

where each of R²¹ and R²² is hydrogen or substituted or unsubstituted C₁₋₆ alkyl.

In yet another embodiment, the compound of Formula (I) is

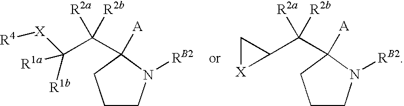

Other examples of compounds of Formula (I) include a compound selected from the group of compounds identified as having ID Nos 22, 26, 33, 34, 75, 76, 205, 206, 65, 59, 60, 61, 62, 200, 201, 202, 38, 99, 99a, 99b, 100, 100a, 100b, 207, 101a, 101b, 12c, 13c, 14c, 226, 230, 253, and 254 in Table 1 hereinafter.

Additional examples of compounds of Formula (I) include compounds selected from the group of compounds identified as having ID Nos 204, 102a, 102b, 211, 5a, 82, 203, 5c, 7c, and 225 in Table 1 hereinafter.

In certain embodiments, the analogs of the present invention are represented by generalized Formula (V), or a pharmaceutically acceptable lactone, salt, metabolite, solvate, and/or prodrug thereof:

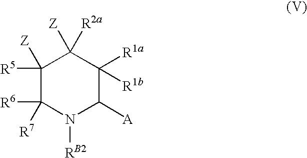

where each of A, R^{1a}, R^{1b}, R², R⁴, and R^{B2} are defined as described above in reference to Formula I; where R⁵, R⁶, and R⁷ are each, independently, hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms; and where Z=XR⁴ or NR^{B1}R^{B2} are as defined as described above in reference to Formula V.

In certain embodiments, the analogs of the present invention are represented by generalized Formula (V-A):

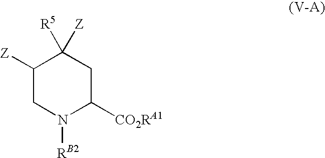

or a pharmaceutically acceptable lactone, salt, metabolite, solvate, and/or prodrug thereof, where each of R^{A1}, R^{B2}, and R⁴, are as defined previously with respect to Formula I; where R⁵ is hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms; and where Z=XR⁴ or NR^{B1}R^{B2} are as defined as described above in reference to Formula V.

Examples of a compound of Formula (V) include a compound selected from the group of compounds identified as having ID Nos 256-263 in Table 1 hereinafter.

In certain embodiments, the analogs of the present invention are represented by generalized Formula (VI), or a pharmaceutically acceptable lactone, salt, metabolite, solvate and/or prodrug thereof:

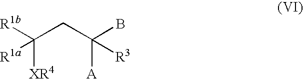

where A, B, X, R^{1a}, R^{1b}, R³, and R⁴ are as defined previously in reference to Formula I.

Examples of a compound of Formula (VI) include a compound selected from the group of compounds identified as having ID Nos 264-269 in Table 1 hereinafter and set forth below:

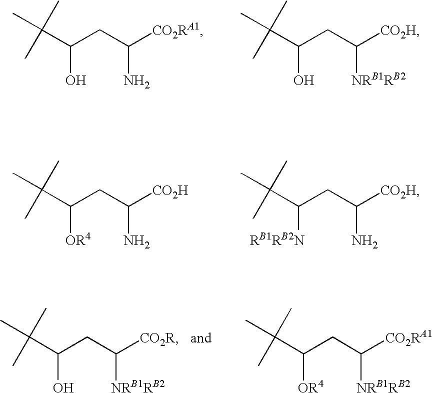

wherein R^{A1}, R^{B1}, R^{B2}, and R⁴ are as defined previously in reference to Formula I.

Specific examples of four preferred compounds of the invention, in isomeric forms SS, SR, RS, and RR, respectively, are as follows and are also present as compounds 270-273 in Table 1.

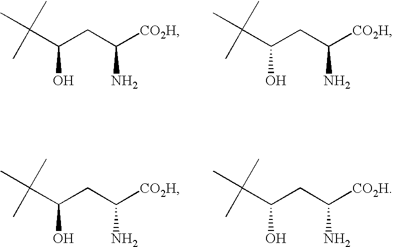

Further examples of preferred compounds of the invention are as follows:

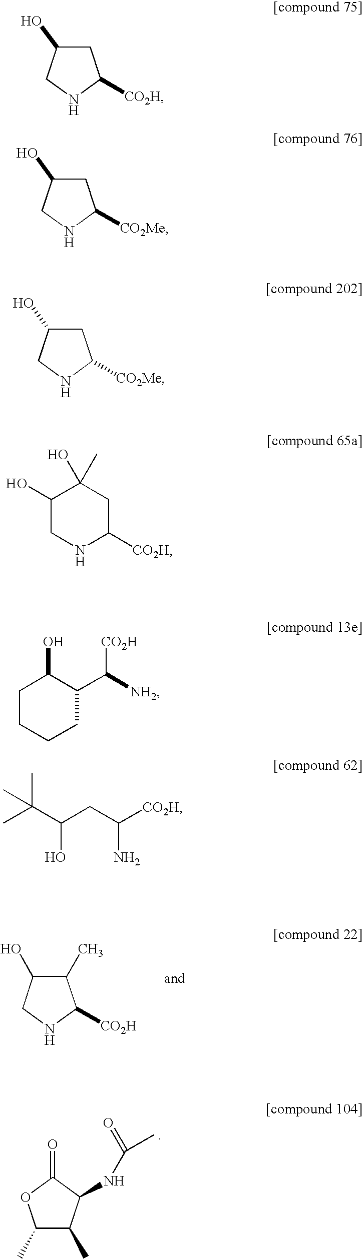

The invention also encompasses salts, solvates, crystal forms, active metabolites, and prodrugs of the compounds of Formulae (I), (II), (III), (IV), (IV-A), (IV-B), (IV-C), (IV-D), (V), (V-A), and (VI). Specific examples of prodrugs include, but are not limited to compounds of Formulae (I), (II), (III), (IV), (IV-A), (IV-B), (IV-C), (IV-D), (V), (V-A), and (VI) in which a suitable functionality, such as, but not exclusively, a hydroxy, amino, or sulfhydryl group in these Formulae is properly derivatized with a biologically or chemically labile molecular moiety that may be cleaved in vivo to regenerate a compound of the respective Formula.

In other embodiments, the compound(s) of the invention are selected from the group consisting of the compounds listed hereinafter in Table 1. It should be noted that in Table 1 hereinafter and throughout the present document when an atom is shown without hydrogen(s), but hydrogens are required or chemically necessary to form a stable compound, hydrogens should be inferred to be part of the compound.

TABLE 1
Structures of Exemplary Compounds

| Cpd # | Structure |
|---|---|
| 5a | 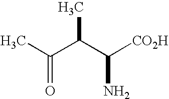 |
| 5b | 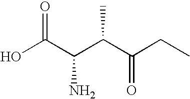 |
| 5c | 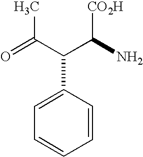 |
| 5d | 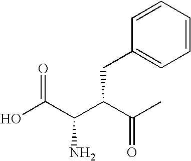 |
| 5e | 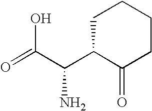 |
| 5f | 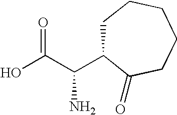 |
| 7b | 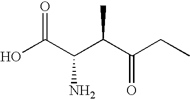 |
| 7c | 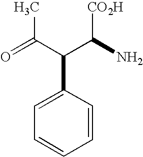 |
| 7d | 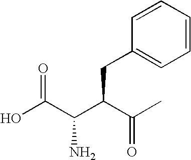 |
| 7e | 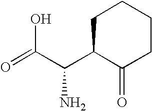 |
| 7f | 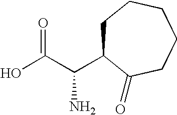 |
| 12b | 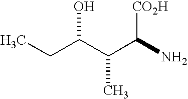 |
| 12a | 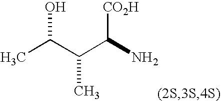 |
| 12aa | 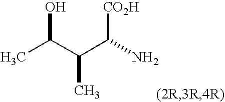 |
| 13a | 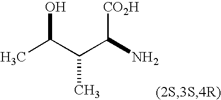 |
| 13aa | 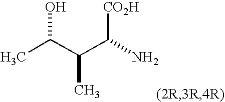 |
| 14a | 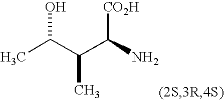 |
| 14aa | 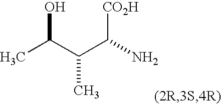 |
| 15a | 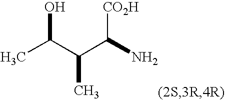 |
| 15aa | 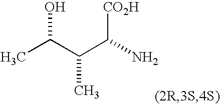 |
| 12c | 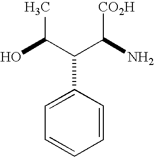 |
| 12d | 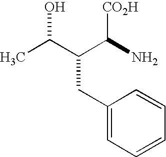 |
| 12e | 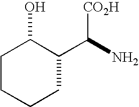 |
| 12f | 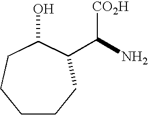 |
| 13b | 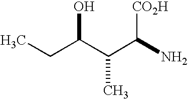 |
| 13c | 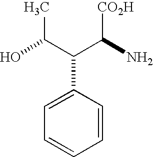 |
| 13d | 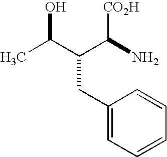 |
| 13e | 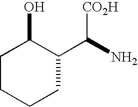 |
| 13f | 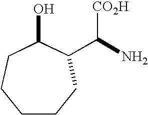 |
| 14a | 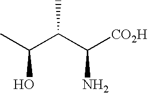 |
| 14c | 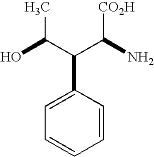 |
| 14d | 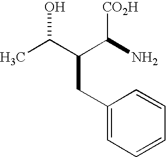 |
| 14e | 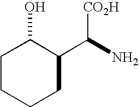 |
| 14f | 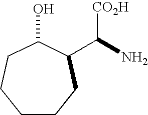 |
| 15b | 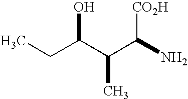 |
| 15c | 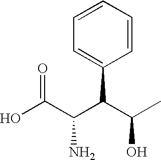 |
| 15d | 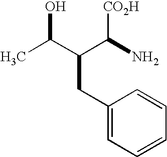 |
| 15e | 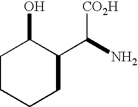 |
| 15f | 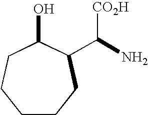 |
| 22 | 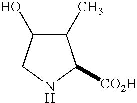 |
| 26 | 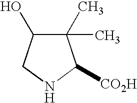 |
| 33 | 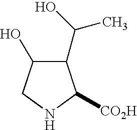 |
| 34 | 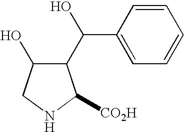 |
| 38 | 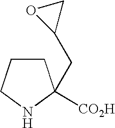 |
| 40 | 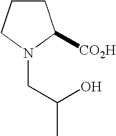 |
| 59 | 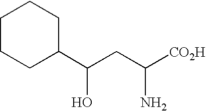 |
| 60 | 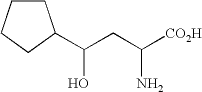 |
| 61 | 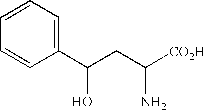 |
| 62 | 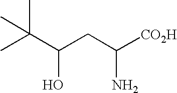 |
| 65 | 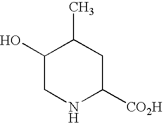 |
| 65a | 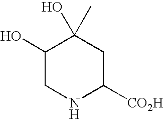 |
| 67 | 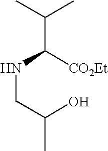 |
| 75 | 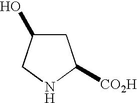 |
| 76 | 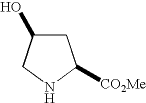 |
| 77 | 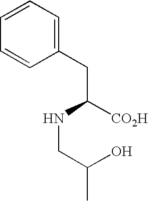 |
| 82 | 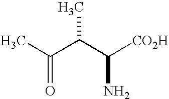 |
| 99 | 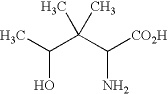 |
| 99a | 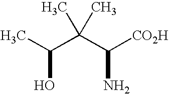 |
| 99b | 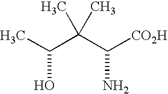 |
| 100 | 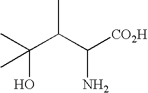 |
| 100a | 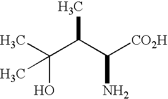 |
| 100b | 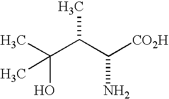 |
| 101a | 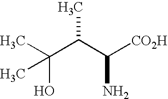 |
| 101b | 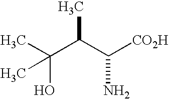 |
| 102a | 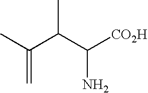 |
| 102b | 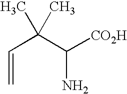 |
| 104 | 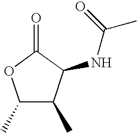 |
| 105 | 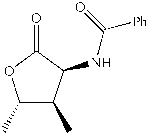 |
| 107a | 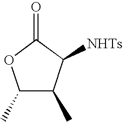 |
| 107b | 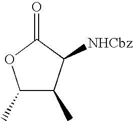 |
| 108a | 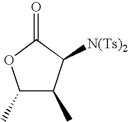 |
| 108b | 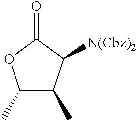 |
| 109 | 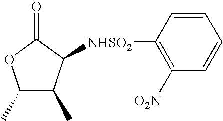 |
| 110 | 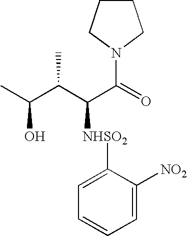 |
| 111a | 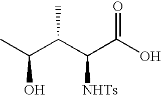 |
| 111b | 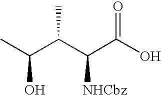 |
| 112a | 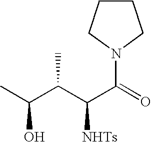 |
| 112b | 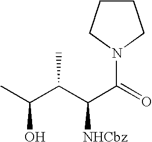 |
| 113a | 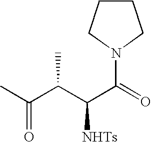 |
| 113b | 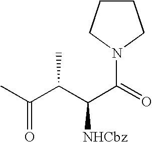 |
| 116 | 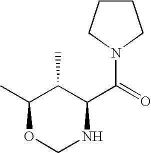 |
| 117 | 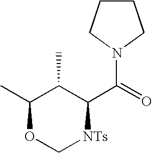 |
| 118 | 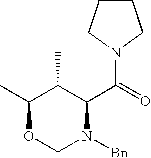 |
| 119 | 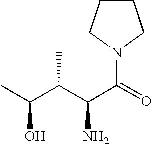 |
| 120 | 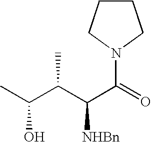 |
| 121a | 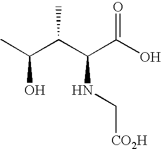 |
| 121b | 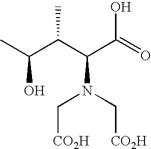 |
| 122 | 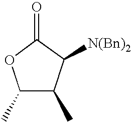 |
| 123 | 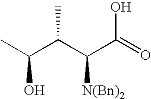 |
| 128 | 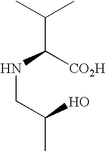 |
| 133 | 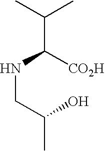 |
| 138 | 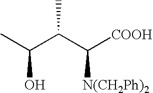 |
| 139 | 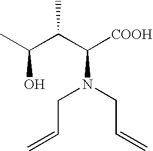 |
| 142 | 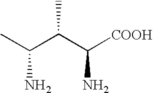 |
| 143 | 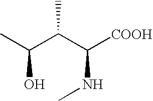 |
| 146 | 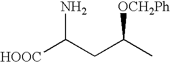 |
| 147 | 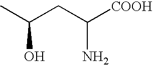 |
| 200 | 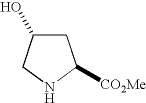 |
| 201 | 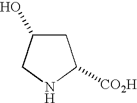 |
| 202 | 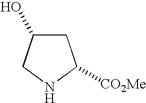 |
| 203 | 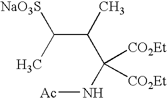 |
| 204 | 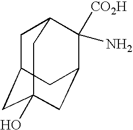 |
| 205 | 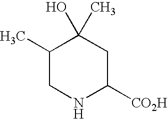 |
| 206 | 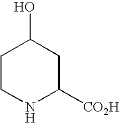 |
| 207 | 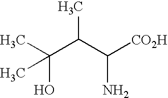 |
| 208 | 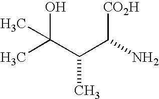 |
| 209 | 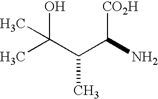 |
| 210 | 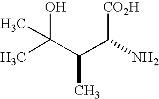 |
| 211 | 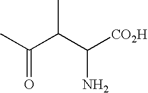 |
| 212 | 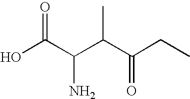 |
| 213 | 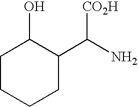 |
| 214 | 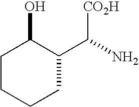 |
| 215 | 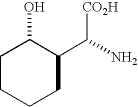 |
| 216 | 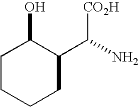 |
| 217 | 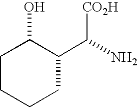 |
| 218 | 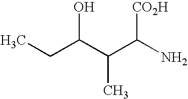 |
| 219 | 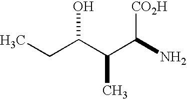 |
| 220 | 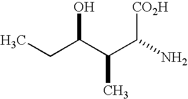 |
| 221 | 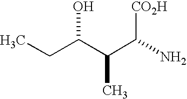 |
| 222 | 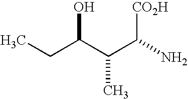 |
| 223 | 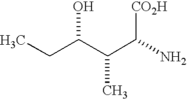 |
| 224 | 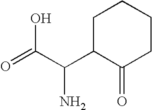 |
| 225 | 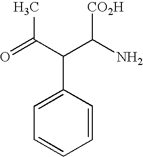 |
| 226 | 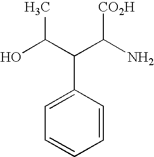 |
| 229 | 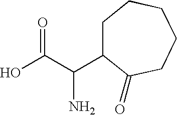 |
| 230 | 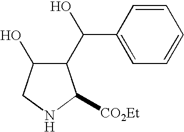 |
| 231 | 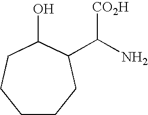 |
| 232 | 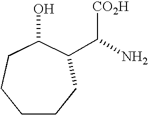 |
| 233 | 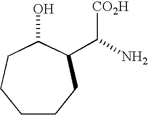 |
| 234 | 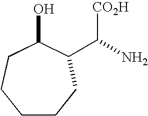 |
| 235 | 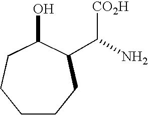 |
| 236 | 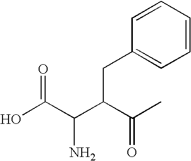 |
| 238 | 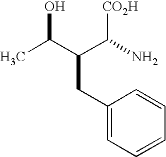 |
| 239 | 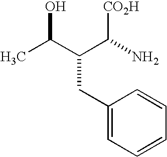 |
| 240 | 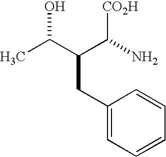 |
| 241 | 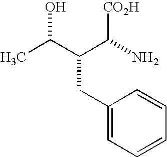 |
| 242 | 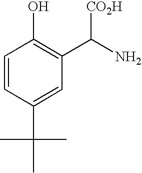 |
| 243 | 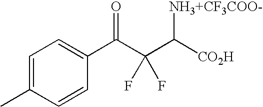 |
| 244 | 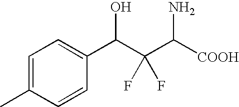 |
| 245 | 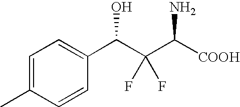 |
| 246 | 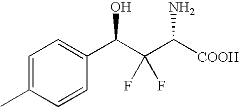 |
| 247 | 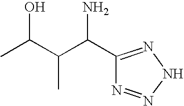 |
| 248 | 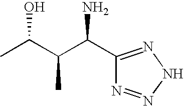 |
| 249 | 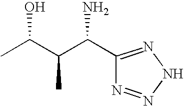 |
| 250 | 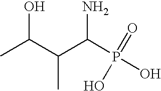 |
| 251 | 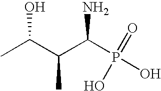 |
| 252 | 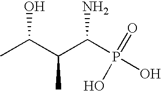 |
| 253 | 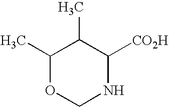 |
| 254 | 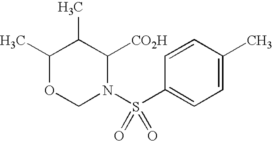 |
| 255 | 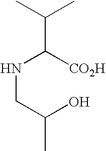 |
| 256 | 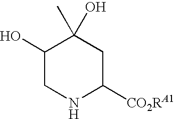 |
| 257 | 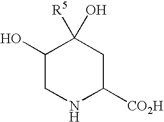 |
| 258 | 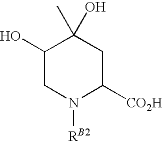 |
| 259 | 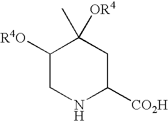 |
| 260 | 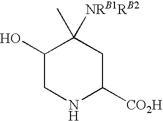 |
| 261 | 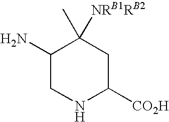 |
| 262 | 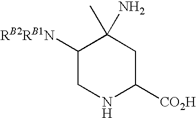 |
| 263 | 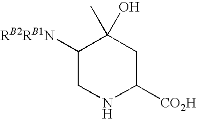 |
| 264 | 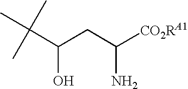 |
| 265 | 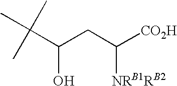 |
| 266 | 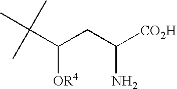 |
| 267 | 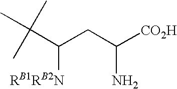 |
| 268 | 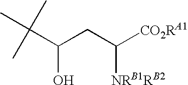 |
| 269 | 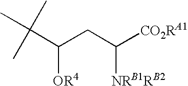 |
| 270 | 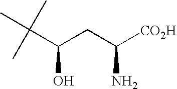 |
| 271 | 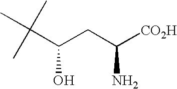 |
| 272 | 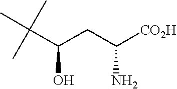 |
| 273 | 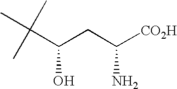 |

The compounds and compositions (see hereinafter) of the invention may be prepared by employing the techniques available in the art using starting materials that are readily available. For instance, PCT application PCT/IB2006/001666 (published as WO 2006/120574A1; originally designated PCT/US2006/005763) and U.S. patent application Ser. No. 11,356,848, both filed Feb. 17, 2006 and incorporated herein by reference, describe compounds of Formulae I, II, III, IV, IV-A, IV-B, IV-C, and/or IV-D.

An additional aspect of the invention concerns new methods for the synthesis of analogs according to the invention. Certain novel and exemplary methods of preparing the inventive compounds are described in the Exemplification section. Such methods are within the scope of this invention.

### D) PHARMACEUTICAL COMPOSITIONS AND THERAPEUTIC APPLICATIONS

Without wishing to be bound by theory, the inventors have observed that compounds according to the invention can be used for the prevention and treatment of disorders of fat metabolism and related syndromes and also for the prevention and treatment of obesity and related syndromes.

Therefore, according to one aspect, the present invention pertains to therapeutic methods, compounds, and pharmaceutical compositions for the prevention or treatment of disorders of fat metabolism, including but not limited to lipodystrophy, hypercholesterolemia, atherosclerosis, and non-alcoholic fatty liver disease, including non-alcoholic steatohepatitis (NASH).

According to another aspect, the present invention pertains to therapeutic methods, compounds, and pharmaceutical compositions for the prevention or treatment of obesity and related syndromes, including but not limited to preventing the onset or progression of excessive weight gain, reducing body weight and/or body fat, and decreasing appetite, and/or food intake. In one embodiment, the methods, compounds, and pharmaceutical compositions of the invention modulate (increase and/or decrease) expression of genes related to fat metabolism. In another embodiment, the methods, compounds, and pharmaceutical compositions of the invention reduce adipogenesis. In another embodiment, the methods, compounds, and pharmaceutical compositions of the invention reduce fat synthesis. In another embodiment, the methods, compounds, and pharmaceutical compositions of the invention increase lipolylis. In another embodiment they increased oxidation.

According to a further aspect, the present invention pertains to therapeutic and cosmetic methods, compounds, and pharmaceutical and cosmetical compositions for the cosmetic treatment of a mammal in order to effect a cosmetically beneficial loss of body weight, and more particularly loss of body fat.

Lipodystrophy is a disorder of adipose tissue that is characterized by a selective loss of body fat. Patients afflicted with this condition have a tendency to develop insulin resistance, type II diabetes, hypertriglyceridemia, and fatty liver. Lipodystrophy occurs in different forms, which can be genetic or acquired. Examples of genetic lipodystrophy include congenital generalized lipodystrophy, which is also known as Berardinelli-Seip syndrome, as well as familial partial lipodystrophy (e.g., the Dunnigan type, the Köbberling type, and the mandibuloacral dysplasia type). Acquired forms of lipodystrophy include acquired generalized lipodystrophy (the Lawrence syndrome), acquired partial lipodystrophy (the Barraquer-Simons syndrome), and lipodystrophy induced by protease inhibitors used to treat HIV infection. The compounds, compositions, and methods of the invention can be used in the prevention and treatment of all of these (and other) types of lipodystrophy.

Hypercholesterolemia is high blood cholesterol, and can be sporadic or familial (due, e.g., to a mutation in the LDL receptor ligand-defective apolipoprotein B-100 (APOB); and autosomal dominant hypercholesterolemia 3 (HCHOLA3) which is caused by mutation in the PCSK9 gene). Hypercholesterolemia is a type of hyperlipidemia, and is associated with increased risks of arteriosclerosis, including coronary artery disease with heart attacks occurring at an unusually young age.

Atherosclerosis is a process of progressive thickening and hardening of the walls of medium-sized and large arteries, as a result of the accumulation of fat deposits on their inner lining. Risk factors for atherosclerosis include high levels of HDL, hypertension, smoking, diabetes, and genetic history. Atherosclerosis is responsible for much coronary artery disease (angina and heart attacks) and many strokes.

Non-alcoholic steatohepatitis (NASH) is characterized by fatty inflammation of the liver in people who do not abuse alcohol, and tends to occur especially in overweight women with diabetes. It is typically a chronic condition that causes no symptoms or very mild symptoms, but can sometimes cause progressive scarring and cirrhosis of the liver.

The invention provides several advantages. For example, individuals diagnosed as having disorders of fat metabolism are at risk of developing serious conditions such as heart disease (e.g., coronary artery disease), stroke, hypertension, type 2 diabetes mellitus, dyslipidemia, respiratory complications, sleep apnea, osteoarthritis, gall bladder disease, depression, and certain forms of cancer (e.g., endometrial, breast, prostate, and colon cancers). Thus, use of the methods, compositions, and compounds of the invention decrease the risk of developing such conditions. Similarly, overweight or obese individuals are at risk of developing serious conditions such as depression, type 2 diabetes, dyslipidemia, respiratory complications, sleep apnea, hypertension, gall bladder disease, heart disease (e.g., coronary artery disease), ostheoarthritis, and certain forms of cancer (e.g., endometrial, breast, prostate, and colon cancers). In being effective at decreasing body weight and/or appetite, the methods of the present invention can decrease the risk of overweight and obese patients developing these conditions. In addition, it is well established that even a 5-10% reduction in body weight can be helpful in improving the health of overweight and obese individuals, and the methods of the invention can be used to achieve such a reduction. Furthermore, many overweight and/or obese individuals are generally desirous of improving their physical appearance by losing body weight, and more particularly by losing body fat.

According to preferred embodiments of the invention, the mammal is a human subject in need of treatment by the methods, compounds, and/or composition of the invention, and is selected for treatment based on this need. A human in need of treatment according to the invention can be identified by those of skill in the art, using methods appropriate for the pertinent condition. A human in need of treatment, especially when referring to obesity is art-recognized and includes individuals that are or are at risk of becoming overweight (Body Mass Index (BMI)>25) or obese (BMI>30) or who are afflicted with a syndrome associated with being overweight or obese. A human in need of treatment may also have or take medicine for the prevention or treatment of disorders of carbohydrate or lipid metabolism, including diabetes mellitus (type 1 and type 2 diabetes), pre-diabetes, and Metabolic Syndrome. Humans in need of treatment may also be at risk of such diseases or disorders, and would be expected, based on diagnosis, e.g., medical diagnosis, to benefit from treatment (e.g., curing, healing, preventing, alleviating, relieving, altering, remedying, ameliorating, improving, or affecting the disease or disorder, the symptom of the disease or disorder, or the risk of the disease or disorder).

Therefore, a related aspect of the invention concerns the use of a compound according to the invention as an active ingredient in a pharmaceutical composition for treatment or prevention purposes. As used herein, “treating” or “treatment” is intended to mean at least the mitigation of a disease or condition associated with disorders of fat metabolism and related syndromes, and/or associated with obesity and related syndromes in a mammal, such as a human, that is alleviated by taking one or more compound(s) according to the invention, and includes curing, healing, inhibiting (e.g., arresting or reducing the development of the disease or its clinical symptoms), relieving from, improving and/or alleviating, in whole or in part, the disease condition (e.g., causing regression of the disease or its clinical symptoms).

As used herein, “prophylaxis,” “prevent,” or “prevention” is intended to mean at least the reduction of likelihood of a disease or condition associated with disorders of fat metabolism and related syndromes, and/or associated with obesity and related syndromes. Fat metabolism disorder predisposing factors identified or proposed in the scientific literature include, among others, (i) a genetic predisposition to having the disease condition but not yet diagnosed as having it, (ii) having a sedentary life style, (iii) nutrition, (iv) concurrent or prior therapy, and/or (v) a genetic mutation. Obesity predisposing factors identified or proposed in the scientific literature include, among others, (i) a genetic predisposition to having the disease condition but not yet diagnosed as having it, (ii) having a disregulation of fat metabolism, (iii) having a sedentary life style, (iv) nutrition, and/or (v) a genetic mutation (e.g., leptin receptor).

The subject may be a female human or a male human, and it may be a child, a teenager, or an adult.

According to a specific aspect, the invention features a method for reducing body weight and/or body fat in a mammal that includes administering to the mammal a compound according to the invention, and/or a composition comprising the same. In a preferred embodiment the mammal is a human that is overweight or obese.

According to another aspect, the invention features a method for treating a mammal, such as a human, that is overweight or obese, which includes administering to the mammal a compound according to the invention, and/or a composition comprising the same.

According to another aspect, the invention features a method of preventing the onset or progression of excessive weight gain in mammals, preferably humans, that includes administering to the mammal a compound according to the invention, and/or a composition comprising the same. In a related aspect, the method, compounds and/or composition according to the invention are used for preventing the onset or progression of weight gain associated with administration of antidiabetic agent that stimulates weight gain.

According to another aspect, the invention features a method of modulating (increasing or decreasing) expression of genes related to the regulation of lipolysis, adipogenesis and/or satiety, including but not limited to FABP4/aP2, HSL, ATGL, FatB1, CPT-1, AMP kinase, cAMP, leptin, adiponectin, AMP kinase, mTOR, PI3 kinase, MSH, NPY, POMC, noradrenaline, dopamine, serotonine (5-HT), MCH, orexin, POMC, CART, AgRP, the method comprising administering to the mammal a compound according to the invention, and/or a composition comprising the same. In one embodiment, expression of AMP kinase is activated in the preriferal tissues. In another embodiment, expression of AMP kinase is inhibited in the hypothalamus.

According to a specific aspect, the invention features a method for treating a mammal, such as a human, that is (1) overweight or obese, and (2) diabetic or taking an antidiabetic agent, the method including the administration of a compound according to the invention, and/or a composition comprising the same, in an amount sufficient to decrease the mammal's circulating glucose level.

According to certain embodiments, the compounds, compositions, and methods of the invention are administered at a therapeutically effective dosage sufficient to reduce the body weight and/or body fat of a treated subject, from about at least 1, 2, 3, 4, 5, 10, 15, 20 25, 30, 35, 40, 45, 50, 75, percent or more, when compared to original levels prior to treatment. Typically, the compounds or compositions of the invention are given until body weight and/or body fat are back to normal, for instance a BMI <30. Due to the nature of the disorders and conditions targeted by the compounds of the invention, it is possible that for certain subjects, chronic or lifetime administration may be required. In preferred embodiments, compounds and pharmaceutical compositions according to the invention are administered once to thrice per day.

Typically, the compounds or compositions of the invention are given until the indicators of the disorders of fat metabolism are back to normal. Due to the nature of the disorders and conditions targeted by the compounds of the invention, it is possible that for certain subjects, chronic or lifetime administration may be required. In preferred embodiments, compounds and pharmaceutical compositions according to the invention are administered once to thrice per day.

Therefore, the present invention provides pharmaceutical compositions comprising a therapeutically effective amount of 4-hydroxyisoleucine, isomers, analogs, lactones, salts, and prodrugs thereof as described herein in combination with a pharmaceutically acceptable carrier or excipient. Suitable carriers or excipients include, but are not limited to saline, buffered saline, dextrose, water, glycerol, ethanol, and combinations thereof. The pharmaceutical compositions may be administered in any effective, convenient manner including, for instance, administration by topical, parenteral, oral, anal, intravaginal, intravenous, intraperitoneal, intramuscular, intraocular, subcutaneous, intranasal, intrabronchial, or intradermal routes among others.

Acceptable methods of preparing suitable pharmaceutical forms of the pharmaceutical compositions are known to those skilled in the art. For example, pharmaceutical preparations may be prepared following conventional techniques of the pharmaceutical chemist involving steps such as mixing, granulating, and compressing when necessary for tablet forms, or mixing, filling, and dissolving the ingredients as appropriate, to give the desired products for various routes of administration.

Toxicity and therapeutic efficacy of the compound(s) according to the invention can be evaluated by standard pharmaceutical procedures in cell cultures or experimental animals. The therapeutic efficacy of the compound(s) according to the invention can be evaluated in an animal model system that may be predictive of efficacy in human diseases. Parameters that can be measured in such animals include, but are not limited to, energy expenditure, oxygen consumption, caloric intake/food consumption, intestinal lipid adsorption, weight, etc. Animal models for evaluating efficacy in glucose uptake include animal models for diabetes and other relevant animal models in which glucose infusion rates can be measured. Animal models for evaluating insulinotropic efficacy include animal models for diabetes or other relevant animal models in which secretion of insulin can be measured. Alternatively, the biological and/or physiological activity of a compound according to the invention can be evaluated in vitro, by examining the ability of the compound in adipocytes to stimulate lipolysis, to increase the expression of genes related to lipid metabolism (e.g., FABP4/aP2, HSL, ATGL, FatB1 and CPT-1 and more particularly ATGL) and/or to modulate AMP kinase levels or activity. While agents that exhibit toxic side effects may be used, care should be taken to design a delivery system that targets such agents to the site of affected tissue in order to minimize potential damage to unaffected cells and, thereby, reduce side effects.

A wide range of drugs can be used with the compounds, compositions, and methods of the present invention. Such drugs may be selected from antiobesity agents, weight-control drugs, appetite reducers, antidiabetic agents, antihypertensive agents, anti-inflammatory agents, antidepressant, etc. Examples of anti-obesity agents that can be used with the compounds according to the invention include Xenical™ (Roche), Meridia™ (Abbott), Acomplia™ (Sanofi-Aventis), Pramlintide (Amylin), and sympathomimetic phentermine. A non-limitative list of potentially useful antiobesity agents is set forth in Table 2, provided hereinafter.

TABLE 2
Known and Emerging Anti-obesity agents

| Name | | |
|---|---|---|
| (Trade name) | Company | Drug description |
| Phentermine* (lonamin ®, | Generic drug | Sympathomimetic appetite suppressant |
| Adipex-P ®, and generics) | | |
| Benzphetamine (Didrex ®) | Pharmacia/Pfizer | Sympathomimetic appetite suppressant |
| Phendimetrazine | Generic drug | Sympathomimetic appetite suppressant |
| Orlistat | Roche | Lipase inhibitor |
| (Xenical ®, Zenical ®) | | |
| Sibutramine | Abbott | Norepinephrine reuptake inhibitor, |
| (Meridia ®, Reductase ®, | | Monoamine uptake inhibitor, Serotonin |
| Reductil ®, Reductyl ™) | | reuptake inhibitor |
| 856464 | GlaxoSmithKline | Melanin concentrating hormone |
| | | antagonist |
| 869682 (KGT-1251) | GlaxoSmithKline/ | SGLT2 antagonist |
| | Kissei | |
| Rimonabant | Sanofi-Aventis | Cannabinoid 1 (CB1) receptor |
| (Acomplia ®) | | antagonist |
| SR 147778 | Sanofi-Aventis | CB1 antagonist |
| AVE1625 | Sanofi-Aventis | CB1 antagonist |
| APD 356 | Arena | Serotonin 2C receptor agonists |
| | Pharmaceuticals | |
| AOD 9604 | Metabolic | Peptide variant of hGH |
| | Pharmaceuticals | |
| P 57 | Phytopharm/ | Apetite suppressant derived from |
| | Unilever | cactus |
| ATL 962 (Celistat ®) | Alizyme/Takeda | Lipase inhibitor |
| c-2624, c-5093, c-2735 | Merck | Not disclosed |
| CP-946,598 | Pfizer | CB1 receptor antagonist |
| BAX 74-4113 | Pfizer/Bayer | DGAT-1 inhibitor |
| SLV-319 | Solvay Pharm./ | CB1 receptor antagonist |
| | Bristol-Myers Squibb | |
| TM30338 | 7TM Pharma | Peptide targeting PYY3-36 receptor |
| AMG 076 | Amgen | Apetite Supressant (antagonist of the |
| | | MCHR1 receptor) |
| AC162352 | Amylin | Apetite Supressant (PYY3-36 gut |
| | | hormone) |
| Oleoyl estrone | Manhattan | Apetite supressant |
| | Pharmaceuticals | |
| GT389-255 | Peptimmune | Lipase inhibitor |
| S-2367 | Shionogi and Co. | Not disclosed |

Typical dosages of a few examples of these antiobesity drugs are provided in Table 3.

TABLE 3
Typical dosages of common antiobesity drugs.

| Drug substance | Dosage and/or administration |
|---|---|
| Ciglitazone | 0.1 mg/tablet |
| Phentermine | 15-37.5 mg/day |
| Benzphetamine | 25-50 mg - 1 to 3 times/day |
| Phendimetrazine | 17.5-35 mg - 2-3 times/day |
| Orlistat | 120 mg/tablet - 3 tablets/day |
| Sibutramine | 10-15 mg/day |

A non-limitative list of useful weight-control drugs that can be used in combination with a compound of the invention includes, but is not limited to, amphetamines, fenfluramine, phenylpropanolamine, or mazindol.

A non-limitative list of useful antidiabetic agents that can be used in combination with a compound of the invention includes, but is not limited to, insulin, biguanides, such as, for example metformin (Glucophage®, Bristol-Myers Squibb Company, U.S.; Stagid®, Lipha Santé, Europe); sulfonylurea drugs, such as, for example, gliclazide (Diamicron®), glibenclamide, glipizide (Glucotrol® and Glucotrol XL®, Pfizer), glimepiride (Amaryl®, Aventis), chlorpropamide (e.g., Diabinese®, Pfizer), tolbutamide, and glyburide (e.g., Micronase®, Glynase®, and Diabeta®); glinides, such as, for example, repaglinide (Prandin® or NovoNorm®; Novo Nordisk), ormitiglinide, nateglinide (Starlix®), senaglinide, and BTS-67582; insulin sensitizing agents, such as, for example, glitazones, a thiazolidinedione, such as rosiglitazone maleate (Avandia®, Glaxo Smith Kline), pioglitazone (Actos®, Eli Lilly, Takeda), troglitazone, ciglitazone, isaglitazone, darglitazone, englitazone, CS-011/CI-1037, T 174, GI 262570, YM-440, MCC-555, JTT-501, AR-H039242, KRP-297, GW-409544, CRE-16336, AR-H049020, LY510929, MBX 102, CLX-0940, GW-501516, and the compounds described in WO 97/41097 (DRF-2344), WO 97/41119, WO 97/41120, WO 98/45292, WO 99/19313 (NN622/DRF-2725), WO 00/23415, WO 00/23416, WO 00/23417, WO 00/23425, WO 00/23445, WO 00/23451, WO 00/41121, WO 00/50414, WO 00/63153, WO 00/63189, WO 00/63190, WO 00/63191, WO 00/63192, WO 00/63193, WO 00/63196, and WO 00/63209; glucagon-like peptide 1 (GLP-1) receptor agonists, such as, for example, Exendin-4 (1-39) (Ex-4), Byetta™ (Amylin Pharmaceuticals Inc.), CJC-1134-PC (Conjuchem Inc.), N,N-2211 (Scios Inc.), and those GLP-1 agonists described in WO 98/08871 and WO 00/42026; agents that slow down carbohydrate absorption, such as, for example, α-glucosidase inhibitors (e.g., acarbose, miglitol, voglibose, and emiglitate); agents that inhibit gastric emptying, such as, for example, glucagon-like peptide 1, cholescystokinin, amylin, and pramlintide; glucagon antagonists, such as, for example, quinoxaline derivatives (e.g., 2-styryl-3-[3-(dimethylamino)propylmethylamino]-6,7-dichloroquinoxaline; Collins et al., Bioorganic and Medicinal Chemistry Letters 2(9):915-918, 1992), skyrin and skyrin analogs (e.g., those described in WO 94/14426), 1-phenyl pyrazole derivatives (e.g., those described in U.S. Pat. No. 4,359,474), substituted disilacyclohexanes (e.g., those described in U.S. Pat. No. 4,374,130), substituted pyridines and biphenyls (e.g., those described in WO 98/04528), substituted pyridyl pyrroles (e.g., those described in U.S. Pat. No. 5,776,954), 2,4-diaryl-5-pyridylimidazoles (e.g., those described in WO 98/21957, WO 98/22108, WO 98/22109, and U.S. Pat. No. 5,880,139), 2,5-substituted aryl pyrroles (e.g., those described in WO 97/16442 and U.S. Pat. No. 5,837,719), substituted pyrimidinone, pyridone, and pyrimidine compounds (e.g., those described in WO 98/24780, WO 98/24782, WO 99/24404, and WO 99/32448), 2-(benzimidazol-2-ylthio)-1-(3,4-dihydroxyphenyl)-1-ethanones (see Madsen et al., J. Med. Chem. 41:5151-5157, 1998), alkylidene hydrazides (e.g., those described in WO 99/01423 and WO 00/39088), and other compounds, such as those described in WO 00/69810, WO 02/00612, WO 02/40444, WO 02/40445, and WO 02/40446; and glucokinase activators, such as, for example, those described in WO 00/58293, WO 01/44216, WO 01/83465, WO 01/83478, WO 01/85706, and WO 01/85707.

Other examples of antidiabetic agents that can be used in combination with one or more compounds according to the invention include, but is not limited to, imidazolines (e.g., efaroxan, idazoxan, phentolamine, and 1-phenyl-2-(imidazolin-2-yl)benzimidazole); glycogen phosphorylase inhibitors (see, e.g., WO 97/09040); oxadiazolidinediones, dipeptidyl peptidase-IV (DPP-IV) inhibitors, protein tyrosine phosphatase (PTPase) inhibitors, inhibitors of hepatic enzymes involved in stimulation of gluconeogenesis and/or glycogenolysis, glucose uptake modulators, glycogen synthase kinase-3 (GSK-3) inhibitors, compounds that modify lipid metabolism (e.g., antihyperlipidemic agents and antilipidemic agents), peroxisome proliferator-activated receptor (PPAR) agonists or antagonists in general, retinoid X receptor (RXR) agonists (e.g., ALRT-268, LG-1268, and LG-1069), and antihyperlipidemic agents or antilipidemic agents (e.g., cholestyramine, colestipol, clofibrate, gemfibrozil, lovastatin, pravastatin, simvastatin, probucol, and dextrothyroxine).

Examples of antihypertensive agents that can be used with the compound(s) of the invention include, but is not limited to, β-blockers (e.g., alprenolol, atenolol, timolol, pindolol, propranolol, and metoprolol), angiotensin converting enzyme (ACE) inhibitors (e.g., benazepril, captopril, enalapril, fosinopril, lisinopril, quinapril, and ramipril), calcium channel blockers (e.g., nifedipine, felodipine, nicardipine, isradipine, nimodipine, diltiazem, and verapamil), and α-blockers (e.g., doxazosin, urapidil, prazosin, and terazosin).

Examples of anti-inflammatory agents that can be used with the compound(s) of the invention include, but is not limited to, anti-histamines, and anti-TNFα.

Examples of antidepressants that can be used with the compound(s) of the invention include, but is not limited to, Bupropion (Quomem®, Wellbutrin XL®, Zyban®), and radafaxine (GlaxoSmithKline). The pharmaceutical agents described herein, when used in combination, can be administered separately (e.g., as two pills administered at or about the same time), which may be convenient in the case of drugs that are already commercially available in individual forms. Alternatively, for drug combinations that can be taken at the same time, by the same route (e.g., orally), the drugs can be conveniently formulated to be within the same delivery vehicle (e.g., a tablet, capsule, or other pill).

Accordingly, another aspect of the invention relates to a pharmaceutical kit or pharmaceutical composition that includes any of the compounds or compositions according to the invention as described herein, or any combination thereof, and an antiobesity agent and/or an antidiabetic agent. The pharmaceutical kit or composition can include compound(s) or composition(s) according to the invention and an antiobesity agent and/or an antidiabetic agent that are formulated into a single composition, such as, for example, a tablet or a capsule.

In another embodiment, pharmaceutical kit could include compound(s) or composition(s) according to the invention and an antiobesity agent and/or an antidiabetic agent formulated separately (e.g., one tablet, pill, or capsule for each compound) with instructions regarding for instance the order, the interval, and/or the frequency of administration in order to achieve a desired effect (e.g., positive impact on an indicator of the pertinent disorder of fat metabolism, e.g., lipolysis, oxygen consumption, energy expenditure, modulating expression of genes relating to fat metabolism, intestinal lipid adsorption, modulation of AMP kinase, caloric intake/food consumption, decrease of appetite, reduction of body weight and/or body fat).

Thus, in addition to the therapeutic methods described above, the invention also includes kits or pharmaceutical packs that can be used in carrying out the methods. Such kits can include the compound(s) or composition(s) according to the invention with instructions to use the drug in the methods described herein, optionally in combination with one or more of the additional drugs described herein.

One or more of the drugs described herein can be administered in a single dose or multiple doses. When multiple doses are administered, the doses may be separated from one another by, for example, several hours, one day, or one week. It is to be understood that, for any particular subject, specific dosage regimes should be adjusted over time according to the individual need and the professional judgment of the person administering or supervising the administration of the compositions. For example, treatment may be modified or ceased upon achieving a desired level of improvement of the disorder of fat metabolism, or when reaching a desired body weight or desired amount of total body fat.

Another related aspect of the invention relates to methods for the prevention and treatment of disorders of fat metabolism and related syndromes, which include administering to a patient one or more compound(s) or composition(s) according to the invention as described herein, in combination with one or more antiobesity agents. The combination of agents can be administered at or about the same time as one another or at different times (5 min, 15 min, 30 min, 1 h, 2 h, 4 h, 12 h, 24 h, or 48 h apart). The combinations of the invention provide several advantages. For example, because the drug combinations described herein can be used to obtain an improved (e.g., additive or synergistic) effect, it is possible to consider administering less of each drug, leading to a decrease in the overall exposure of patients to the drugs, as well as any untoward side effects of any of the drugs. In addition, greater control of the disease may be achieved, because the drugs can combat the disease through different mechanisms. The compounds, compositions, and methods according to the invention as described herein can also be used in combination with other approaches to treatment of the disorder of fat metabolism, and/or be used in combination with other approaches to weight loss and management, including approaches involving changes in diet or physical activity, as well as surgical procedures.

### Administration

With respect to the therapeutic methods of the invention, it is not intended that the administration of compounds to a mammal be limited to a particular mode of administration, dosage, or frequency of dosing; the present invention includes all modes of administration, including oral, intraperitoneal, intramuscular, intravenous, intra-articular, intralesional, subcutaneous, by inhalation, or any other route sufficient to provide a dose adequate to prevent or treat a disorder of fat metabolism and/or related syndromes. One or more compounds may be administered to the mammal in a single dose or multiple doses. When multiple doses are administered, the doses may be separated from one another by, for example, several hours, one day, or one week. It is to be understood that, for any particular subject, specific dosage regimes should be adjusted over time according to the individual need and the professional judgment of the person administering or supervising the administration of the compositions. Exemplary mammals that can be treated using the compound(s), compositions, and methods of the invention include humans, primates, such as monkeys, animals of veterinary interest (e.g., cows, pigs, sheep, goats, buffaloes, and horses), and domestic pets (e.g., dogs and cats). The compound(s) and compositions of the invention can also be administered to laboratory animals such as rodents (e.g., mice, rats, gerbils, hamsters, guinea pigs, and rabbits) for treatment purposes and/or for experimental purposes (e.g., studying the compounds' mechanism(s) of action, screening, and testing efficacy of the compound(s), structural design, etc.).

For clinical applications in therapy or in prophylaxis, analogs or compositions of the present invention can generally be administered, e.g., orally, subcutaneously, parenterally, intravenously, intramuscularly, colonically, nasally, intraperitoneally, rectally, by inhalation, or buccally. Compositions containing at least one compound according to the invention that is suitable for use in human or veterinary medicine can be presented in forms permitting administration by a suitable route. These compositions can be prepared according to customary methods, using one or more pharmaceutically acceptable carriers or excipients. The carriers can comprise, among other things, diluents, sterile aqueous media, and various non-toxic organic solvents. Acceptable carriers or diluents for therapeutic use are well known in the pharmaceutical field, and are described, for example, in Remington: The Science and Practice of Pharmacy (20th ed.), ed. A. R. Gennaro, Lippincott Williams & Wilkins, 2000, Philadelphia, and Encyclopedia of Pharmaceutical Technology, eds. J. Swarbrick and J. C. Boylan, 1988-1999, Marcel Dekker, New York. The compositions can be presented in the form of tablets, pills, granules, powders, aqueous solutions or suspensions, injectable solutions, elixirs, or syrups, and the compositions can optionally contain one or more agents chosen from the group comprising sweeteners, flavorings, colorings, and stabilizers in order to obtain pharmaceutically acceptable preparations.

The choice of vehicle and the content of active substance in the vehicle are generally determined in accordance with the solubility and chemical properties of the product, the particular mode of administration, and the provisions to be observed in pharmaceutical practice. For example, excipients such as sodium citrate, calcium carbonate, and dicalcium phosphate and disintegrating agents such as starch, alginic acids, and certain complex silicates combined with lubricants (e.g., magnesium stearate, sodium lauryl sulfate, and talc) can be used for preparing tablets. To prepare a capsule, it is advantageous to use high molecular weight polyethylene glycols. When aqueous suspensions are used, they can contain emulsifying agents that facilitate suspension. Diluents such as ethanol, polyethylene glycol, propylene glycol, glycerol, chloroform, or mixtures thereof can also be used. In addition, low calorie sweeteners, such as, for example, isomalt, sorbitol, xylitol, can be used in a formulation of the invention.

For parenteral administration, emulsions, suspensions, or solutions of the compositions of the invention in vegetable oil (e.g., sesame oil, groundnut oil, or olive oil), aqueous-organic solutions (e.g. water and propylene glycol), injectable organic esters (e.g. ethyl oleate), or sterile aqueous solutions of the pharmaceutically acceptable salts can be used. The solutions of the salts of the compositions of the invention are especially useful for administration by intramuscular or subcutaneous injection. Aqueous solutions that include solutions of the salts in pure distilled water can be used for intravenous administration with the proviso that (i) their pH is adjusted suitably, (ii) they are appropriately buffered and rendered isotonic with a sufficient quantity of sodium chloride, and (iii) they are sterilized by heating, irradiation, or microfiltration. Suitable compositions containing the compounds of the invention can be dissolved or suspended in a suitable carrier for use in a nebulizer or a suspension or solution aerosol, or can be absorbed or adsorbed onto a suitable solid carrier for use in a dry powder inhaler. Solid compositions for rectal administration include suppositories formulated in accordance with known methods.

A dose of the pharmaceutical composition contains at least a therapeutically effective amount of a compound according to the invention and is preferably made up of one or more pharmaceutical dosage units. The selected dose can be administered to a human subject in need of treatment. A “therapeutically effective amount” is intended to mean that amount of analog(s) of the invention that confers a therapeutic effect on the subject treated. The therapeutic effect can be objective (i.e., measurable by some test or marker (e.g., weight loss) or subjective (i.e., the subject gives an indication of or feels an effect).

It is understood that the amount that will correspond to a “therapeutically effective amount” and the appropriate doses and concentrations of the agent(s) in the formulations (i.e., compound(s) of the invention alone and/or in combination with other drug(s)) will vary, depending on a number of factors, including the dosages of the agents to be administered, the route of administration, the nature of the agent(s), the frequency and mode of administration, the therapy desired, the form in which the agent(s) are administered, the potency of the agent(s), the sex, age, weight, and general condition of the subject to be treated, the nature and severity of the condition treated, any concomitant diseases to be treated, the possibility of co-usage with other agents for treating a disease, and other factors. Nevertheless the therapeutically effective amount can be readily determined by one of skill in the art.

For administration to mammals, and particularly humans, it is expected that in the treatment of an adult dosages from about 0.1 mg to about 50 mg (e.g., about 5 mg to about 100 mg, about 1 mg to about 50 mg, or about 5 mg to about 25 mg) of each active compound per kg body weight per day can be used. A typical oral dosage can be, for example, in the range of from about 50 mg to about 5 g per day (e.g., about 100 mg to about 4 g, 250 mg to 3 g, or 500 mg to 2 g), administered in one or more dosages, such as 1 to 3 dosages. Dosages can be increased or decreased as needed, as can readily be determined by those of skill in the art. For example, the amount of a particular agent can be decreased when used in combination with another agent, if determined to be appropriate. In addition, reference can be made to standard amounts and approaches that are used to administer the agents mentioned herein. The physician in any event will determine the actual dosage that will be most suitable for an individual.

As for dosing, it is understood that duration of a treatment using any of the compounds or compositions of the invention will vary depending on several factors, such as those listed herein before for dosing. Nevertheless, appropriate duration of administration can be readily determined by one of skill in the art. According to certain embodiments, the compounds of the invention are administered on a daily, weekly, or continuous basis.

The compounds and compositions of the invention are conceived to be effective primarily in the prevention and treatment of disorders of fat metabolism and related syndromes, and also in the prevention and treatment of obesity and related syndromes. As is noted elsewhere herein, a non-limiting list of examples of fat metabolism related disorders includes lipodystrophy, hypercholesterolemia, atherosclerosis, and nonalcoholic steatohepatitis because they may influence fat distribution.

### E) NUTRITIONAL COMPOSITIONS

Alternatively, the compounds and composition of the invention may be administered in form of a nutritional composition, e.g. in form of a dietary supplement, or medical food, e.g. in form of a complete meal, as part of a meal, or a food additive, or beverage, e.g. in form of a powder for dissolution. The powder may be combined with a liquid, e.g. water, or other liquid, such as milk or fruit juice, to obtain a ready-to-consume composition, e.g. ready-to-drink composition or instant drink. Alternatively, the beverage may be a soft drink, juice, milk-shake, yogurt drink, smoothie or soy-based drink. The nutritional compositions may be in form of a bar, or dispersed in foods of any sort, such as baked products, cereal bars, dairy bars, snack-foods, soups, breakfast cereals, muesli, candies, tabs, cookies, biscuits, crackers, such as a rice crackers, and dairy products.

Suitable product formats according to the present invention include solution, ready-for-consumption composition, e.g. ready-to-drink compositions, instant drink, liquid comestibles, like soft drinks, juice, sports drinks, milk drinks, milk-shakes, yogurt drinks or soup. In a further embodiment of the invention, the nutritional compositions of the present invention may be manufactured and sold in the form of a concentrate, a powder, or granules, e.g. effervescent granules, which are diluted with water or other liquid, such as milk or fruit juice, to yield a ready-for-consumption composition, e.g. ready-to-drink compositions or instant drink.

Optionally, the compositions according to the invention may be nutritionally complete, i.e. may include vitamins, minerals, trace elements as well as additional nitrogen, carbohydrate and additional fatty acid sources so that they may be used as the sole source of nutrition supplying essentially all the required daily amounts of vitamins, minerals, carbohydrates, fatty acids, proteins and the like. Accordingly, the nutritional compositions of the invention may be provided in the form of a nutritionally balanced complete meal, e.g. suited for oral or tube feeding. Preferably the nutritional compositions of the invention are for oral administration.

The compound(s) according to the invention can be present in the nutritional composition according to the present invention in an amount of about 0.0001% to about 0.001% by weight, or from about 0.001% to about 0.01% by weight, or from about 0.01% to about 0.1% by weight, or from about 0.1% to about 1% by weight, or from about 1% to about 5% by weight.

It may be desirable to provide the nutritional compositions of the invention in the form of a low calorie meal replacement or other nutritional product. Suitably, a single serving of a low calorie meal replacement will have a caloric value of less than about 1000 kcal (4.2 MJ), and preferably between about 200 kcal (0.8 MJ) and about 500 kcal (2.1 MJ). Suitable low calorie nutritional product may include any nutritional product described hereinabove.

Conventional additives may be included in the nutritional compositions of the invention, including any of those selected from preservatives, chelating agents, osmotic agents, buffers or agents for pH adjustment, effervescing agents, sweeteners, e.g. artificial sweeteners, flavoring agents, coloring agents, taste masking agents, acidulants, emulsifiers, stabilizers, thickening agents, suspending agents, dispersing or wetting agents, antioxidants, acidulants, texturizers, antifoam agents, and the like. For example the pharmaceutical or nutritional compositions of the invention may contain curcumin, chlorogenic acid or cinnamon.

According to the invention, the nutritional compositions of the invention may comprise natural botanical materials such as Fenugreek.

In addition to the foregoing, the present invention also provides a process for the production of a composition, e.g. nutritional or pharmaceutical formulation, as hereinbefore defined, which process comprises bringing the individual components thereof into intimate admixture and, when required compounding the obtained composition in a food or beverage product, for example ready-made drink, or in unit dosage form, for example filling said composition into a sachet.

Dependent on the form of application of the nutritional compositions of the invention, i.e. as complete meal, part of a meal, food additive, drink, sachet, tablet or capsule, the compositions of the invention may be taken once daily to e.g. five times daily. Preferably the unit doses are taken five or three times, e.g. with the main meals, e.g. without restriction to time of day. Preferably, the unit doses are taken together with, or shortly before, e.g. 15 minutes before, the main meals, e.g. in the morning, at noon, and in the evening.

### EXAMPLES

The invention is based, in part, on the experimental examples set forth as Examples 1 to 8 below. These examples are given to enable those skilled in the art to more closely understand and to practice the present invention and should not be construed as specifically limiting its scope.

The Examples set forth herein below provide exemplary syntheses of certain representative compounds of the invention. Also provided are exemplary methods for assaying the compounds of the invention for their impact on fat metabolism and related parameters. These examples are given to enable those skilled in the art to more closely understand and to practice the present invention and are not intended to either define or limit its scope.

### Example 1

### General Procedure for the Preparation of Isomers and Analogs of 4-hydroxyisoleucine

### A) General Experimental Procedures

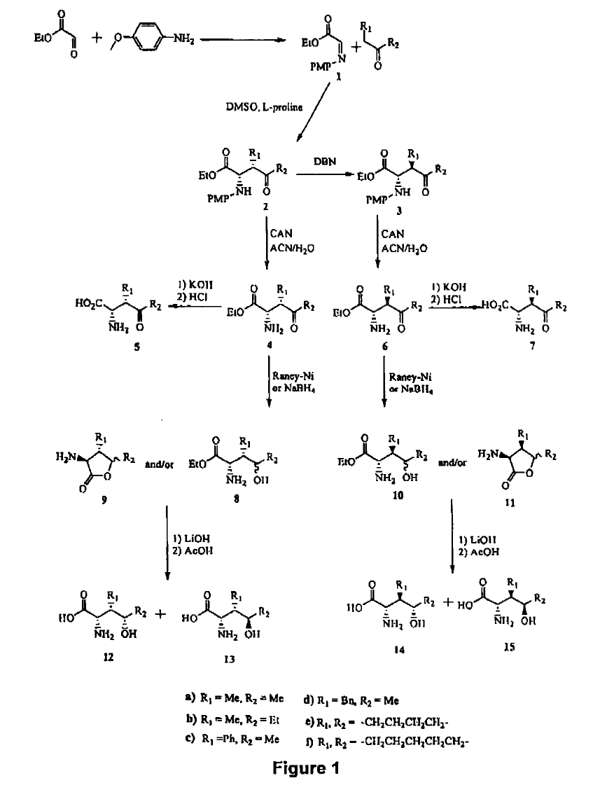

Reference is made to FIG. 15 showing a synthetic scheme for the synthesis of eight different configurational isomers of 4-hydroxyisoleucine, and reference is made to FIGS. 1 to 14 showing synthetic schemes for the synthesis of exemplary linear and cyclic analogs of 4-hydroxyisoleucine.

FIG. 15 shows a synthetic scheme for the synthesis of eight different configurational isomers (SRS, SRR, SSS, SSR, RSR, RSS, RRR, and RRS) of 4-hydroxyisoleucine. Imine intermediate 1 was prepared from p-anisidine and ethyl glyoxalate (Cordova et al., J. Am. Chem. Soc. 124:1842-43, 2002). The reaction of imine 1 with 2-butanone in the presence of L-proline as a catalyst followed by silica gel chromatography yielded 2S,3S isomer 2a. Epimerization at C-3 was achieved with 1,5-diazabicyclo[4.3.0]non-5-ene (DBN) to yield 2S,3R isomer 3a. The (2S,3R,4S); (2S,3R,4R); (2S,3S,4S); and (2S,3S,4R) isomers of 4-hydroxyisoleucine are obtained from either 2a or 3a as follows:

Deprotection of amine moiety of 3a (removal of p-methoxyphenyl group) with ceric ammonium nitrate (CAN) and subsequent reduction with KBH₄ in water and concomitant cyclization provided lactone 11a, which upon base hydrolysis with lithium hydroxide and recrystallization from absolute ethanol gave pure (2S,3R,4S)-4-hydroxyisoleucine 14a. Alternatively, deprotection of the amine moiety of 3a with CAN was followed by isolation of amine intermediate 6a, which was subsequently reduced with potassium borohydride in methanol to give the lactone intermediate 11a′, which upon base hydrolysis with lithium hydroxide and recrystallization from ethanol gave (2S,3R,4R) 4-hydroxyisoleucine (compound 15a). Further purification of compound 15a was carried out using preparative HPLC.

Similar reactions starting from compound 2a, using sodium borohydride instead of potassium borohydride for preparation of lactone 9a′ from aminoketone 4a lead to the isolation of (2S,3S,4S) 4-hydroxyisoleucine (compound 12a) and (2S,3S,4R) 4-hydroxyisoleucine (compound 13a).

When compound 1 was reacted with 2-butanone in the presence of a catalytic amount of D-proline, compound 2aa, which is the enantiomer of compound 2a, was formed. As above, epimerization of the C-3 of compound 2aa was achieved with 1,5-diazabicyclo[4.3.0]non-5-ene (DBN) to yield 2R,3S isomer 3aa. By reaction sequences identical to those used for the preparation of compounds 14a, 15a, 12a, and 13a, the (2R,3S,4R); (2R,3S,4S); (2R,3R,4R); and (2R,3R,4S) isomers (compounds 14aa, 15aa, 12aa, and 13aa, respectively) were obtained from compounds 2aa and 3aa.

FIG. 1 shows synthesis of various analogs of 4-hydroxyisoleucine with SSS, SSR, SRS, and SRR configurations. Imine intermediate 1 was prepared from p-anisidine and ethyl glyoxalate (Cordova et al., J. Am. Chem. Soc. 124:1842-43, 2002). The reaction of imine 1 with a suitable ketone in the presence of L-Proline as a catalyst yielded 2S,3S isomer (2). Epimerization at C-3 was achieved with a base, e.g., 1,5-diazabicyclo[4.3.0]non-5-ene (DBN) to yield 2S,3R isomer (3). The (2S,3S,4S), (2S,3S,4R), (2S,3R,4S), and (2S,3R,4R) analogs of 4-hydroxyisoleucine were obtained from 2 or 3, respectively, as follows.

Deprotection of amine moiety of 2 (removal of p-methoxyphenyl group) with ceric ammonium nitrate (CAN) to yield 4 and subsequent hydrolysis led to (2S,3S)-4-keto analogs (5). Similarly, deprotection of 3 yielded 6, which upon base hydrolysis gave (2S,3R)-4-keto analogs (7). The reduction of 4 and 6 with NaBH₄ or Raney nickel or as a single step deprotection/reduction of 2 and 3 generated a diastereomeric mixture of a lactone (9 and 11) and an open chain intermediate (8 and 10), respectively. The hydrolysis of a mixture of 8 and 9, followed by purification, gave (2S,3S,4S) and (2S,3S,4R) analogs, 12 and 13, respectively. Similarly, (2S,3R,4S) and (2S,3R,4R) analogs, i.e., 14 and 15, were obtained from the hydrolysis of a mixture of compounds 10 and 11.

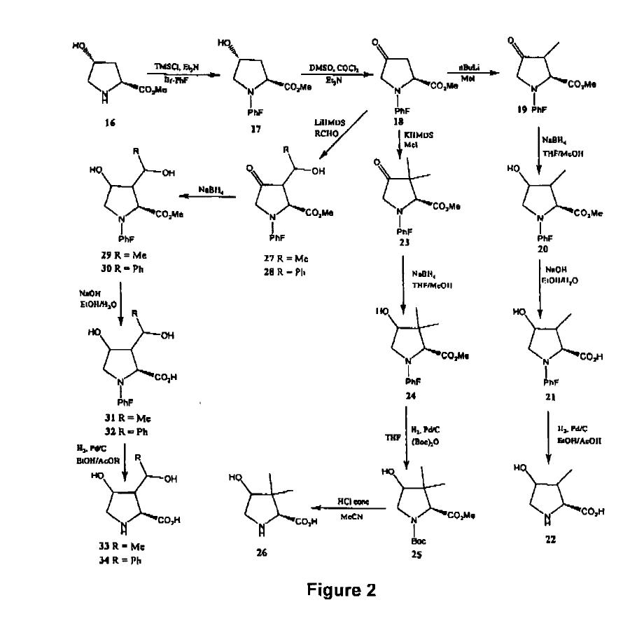

3-substitued 4-hydroxyproline based analogs were synthesized as depicted in FIG. 2. 4-Hydroxyproline methyl ester (16) reaction with chlorotrimethylsilane, triethylamine, followed by reaction with bromo-phenylfluorene/Pb(NO₃)₂ gave the protected intermediate (17). Swern oxidation of 17 with oxalylchloride and DMSO led to the key intermediate PhF-4-oxoproline methyl ester (18). Alkylation at C-3 of this intermediate gave various 3-substituted analogs. Mono-alkylation of 18 was achieved using n-Buthyllithium as a base to give compound 19, while di-alkylation was performed using KHMDS as a base gave compound 23. The reduction of alkylated oxoproline intermediates (19 and 23) gave the hydroxyl intermediates, 20 and 24, respectively. The base hydrolysis of 20 gave the acid (21), which upon catalytic hydrogenolysis afforded the desired 3-methyl analog (22). The corresponding dimethyl intermediate (24) underwent catalytic hydrogenolysis and in-situ protection with Boc anhydride to yield the Boc intermediate (25), which upon deprotection and acid hydrolysis afforded the desired 3-dimethyl analog (26). The alkylation of the key intermediate PhF-4-oxoproline methyl ester (18) with aldehydes was followed by the reaction sequence described above for the synthesis of compound 22, i.e., reduction, base hydrolysis, and a catalytic hydrogenation, led to 3-substituted analogs 33 and 34.

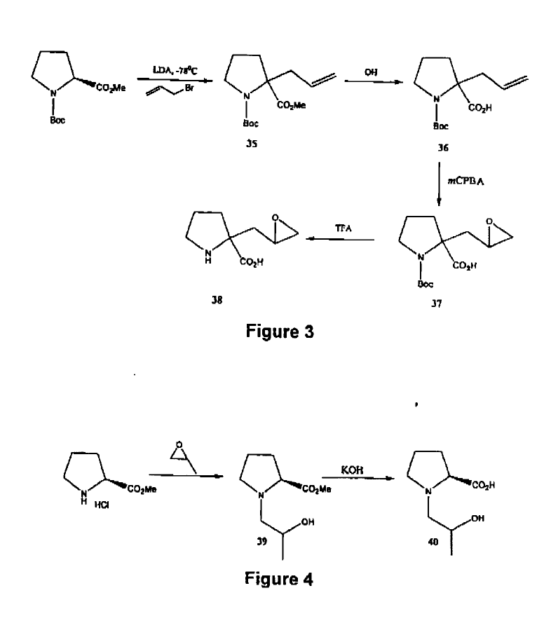

Boc-proline methyl ester was alkylated using allylbromide and LDA to give N-Boc-α-allylproline methyl ester (35), as shown in FIG. 3, which was subsequently converted to the free carboxylic acid (36) via basic hydrolysis. N-Boc-α-allylproline was then reacted with m-chloroperbenzoic acid to yield the epoxy-derivative (37). The removal of Boc-protecting group with TFA, followed by several lyophilizations to remove excess TFA, yielded the desired α-oxiranylmethyl-proline analog (38).

The route to synthesis of compound 40 is shown in FIG. 4. Propylene oxide was used to neutralize the L-proline HCl salt. Exothermic reaction of propylene oxide with the acid salt led to further reaction of the epoxide with the amine moiety to form N-hydroxypropyl substituted amino acid (39). The base hydrolysis of compound 39 gave the desired acid (40).

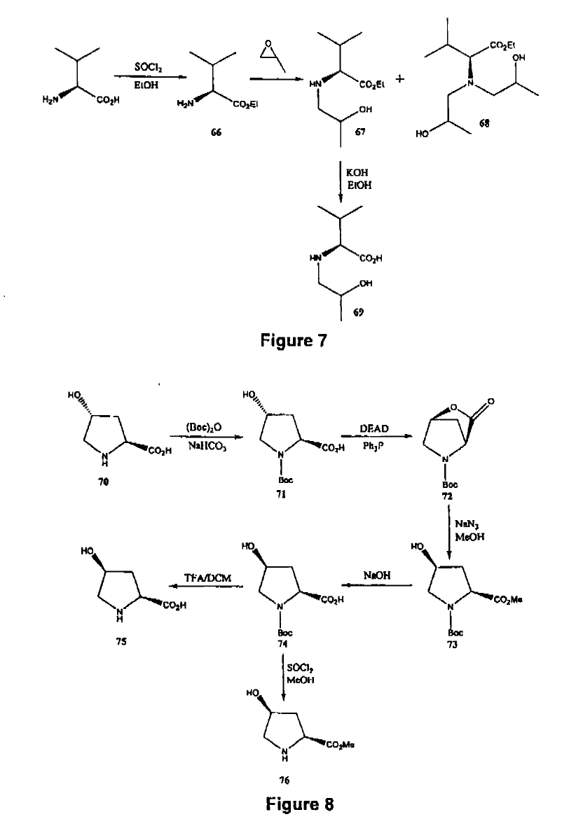

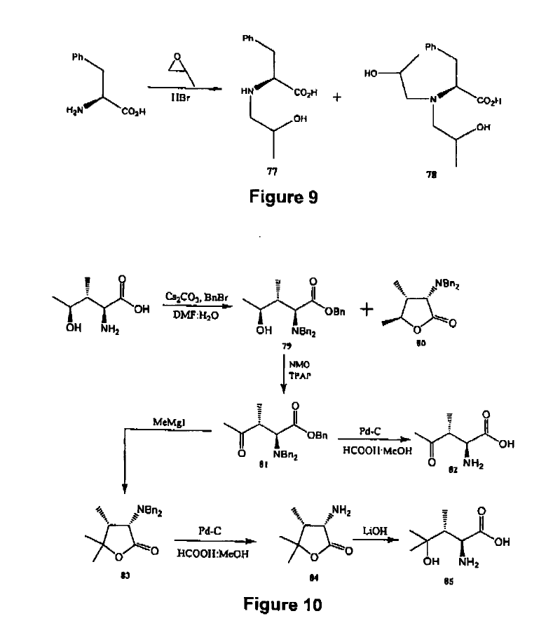

Similar reactivity of L-valine ethyl ester (66), synthesized from L-valine by reaction with thionyl chloride in ethanol, with propylene oxide led to the mono substituted amino acid (67) and also the di-substituted amino acid (68) (FIG. 7). The desired N-(2-hydroxypropyl)-L-valine (69) was isolated after base hydrolysis of mono substituted amino acid (67) (FIG. 7). Similar chemistry, shown in FIG. 9, depicts the one step synthesis of N-(2-hydroxypropyl)-L-phenylalanine (77). In this case L-phenylalanine was used as such, i.e., the acid moiety was not protected as an ester as in the case of valine compound 69. The disubstituted compound (78) was also observed as a by-product.

The analogs shown in FIG. 5 were prepared starting either from the corresponding acid or the ketone. For example, cyclohexyl acid was transformed into a hydroxamate (41) from the reaction with TBTU and N-methyl O-methylhydroxylamine. The hydroxamate (41) was then converted into the ketone (43) by reaction with methyllithium. The reaction of this cyclohexyl methyl ketone (43) with diethyloxalate gave 4-cyclohexyl-2-hydroxy-4-oxo-but-2-enoic acid ethyl ester (47). The reaction of compound 47 with hydroxylamine led to an oxazole intermediate (51). The base hydrolysis of 51 gave the acid (55) which, upon hydrogenolysis with Raney nickel, gave the desired analog, 2-amino-4-cyclohexyl-4-hydroxy-butyric acid (59). The chemistry described above was repeated with the corresponding acid and the ketone to obtain analogs such as 2-amino-4-cyclopentyl-4-hydroxy-butyric acid (60), 2-amino-4-hydroxy-4-phenyl-butyric acid (61), and 2-amino-4-hydroxy-5,5-dimethyl-hexanoic acid (62).

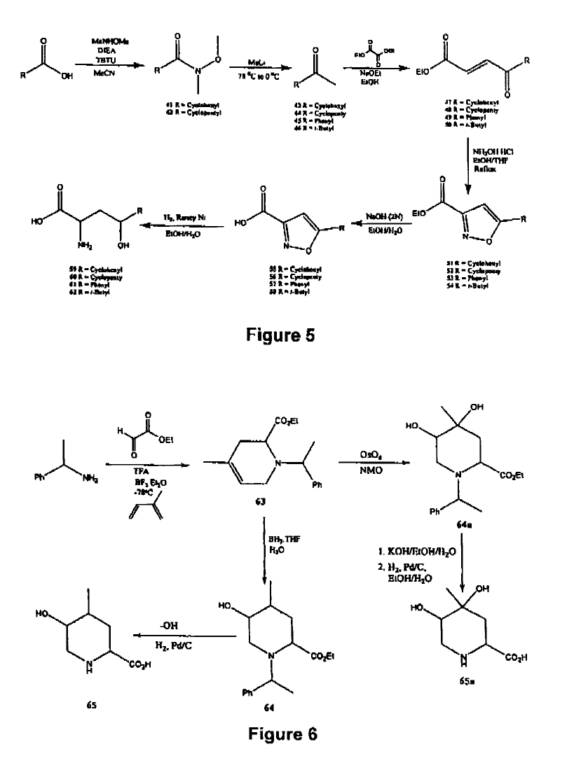

Dipipecolic intermediate (63) was prepared from the condensation reaction of α-methyl benzylamine with ethylglyoxylate (FIG. 6). Hydroboration with BH₃.THF gave the protected form of 5-hydroxy-4-methyl-2-piperidine carboxylic acid (64). The hydrolysis and catalytic hydrogenolysis led to the isolation of 5-hydroxy-4-methyl-2-piperidine carboxylic acid (65).

The chirality of Boc-protected trans-4-hydroxyproline (71) was inverted to compound 72 using Mitsunobu reaction conditions (Silverman et al., Org. Lett. 3: 2481-2484, 2001; and Org. Lett. 3: 2477, 2001) (FIG. 8). The hydrolysis of compound 72 to compound 73 to compound 74 and removal of Boc with TFA/DCM of intermediate 74 gave the desired compound 75. The methyl ester derivative of compound 75, i.e., compound 76, was prepared from compound 74 by reacting with thionyl chloride in methanol.

The protection of the amino acid moiety of (2S,3R,4S)-4-hydroxyisoleucine was achieved in one step using Cs₂CO₃ as base, and BnBr in DMF/water mixture in good overall yield (FIG. 10). The reaction mixture contained mainly open chain compound (79), and some amount of the corresponding lactone (80). The oxidation the of open chain intermediate (79), followed by hydrogenolysis, gave the desire 4-keto analog (82) in a good yield. Grinyard addition of methyl magnesium iodide to the protected keto intermediate (81) gave dibenzyl lactone (83) in moderate yield. The deprotection using formic acid and Pd—C catalyst reaction conditions or hydrogenolysis gave the lactone (84) in good yield. Finally, the hydrolysis of lactone with LiOH afforded the desired (2S,3R) analog 85 in an isolated yield of 90% (FIG. 10).

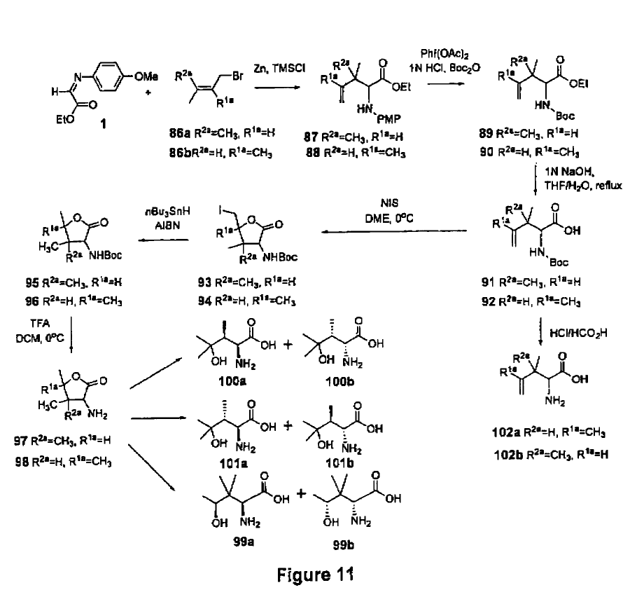

The analogs described in FIG. 11 were synthesized starting from a reaction of imine (1) either with 1-bromo-3-methylbut-2-ene or 1-bromo-2-methylbut-2-ene to give the condensation products 87 and 88, respectively. The removal of the PMP group was accomplished with iodosobenzene diacetate, followed by in-situ protection of amino groups with Boc anhydride to yield compounds 89 and 90, respectively. The hydrolysis of the ester moiety, followed by reaction with N-iodosuccinimide in DME, led to the iodolactone (compounds 93 and 94). nBuSnH and AIBN were to used to remove the iodo functional group, and subsequent removal of Boc group with TFA in dichloromethane gave the key lactone intermediate (compounds 97 and 98, respectively). The hydrolysis of compound 97 under basic conditions led to the isolation of an enantiomeric mixture (SS and RR isomers) of compounds 99a and 99b. Similarly, base hydrolysis of compound 98 led to the isolation of compounds 100a and 100b (again, an enantiomeric mixture of SS and RR isomers), and compounds 101a and 101b (an enantiomeric mixture of SR and RS isomers). Compounds 102a and 102b were obtained from compounds 92 and 91, respectively, by removal of the Boc group under acidic conditions.

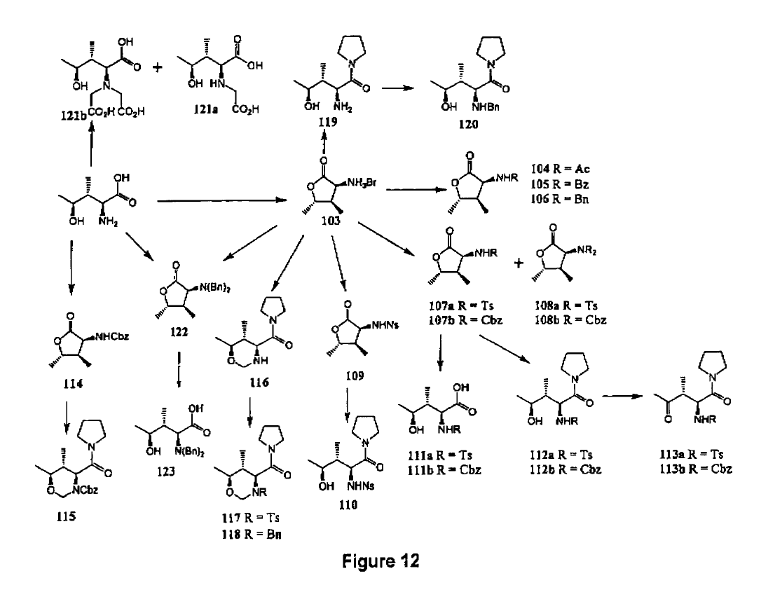

The compounds shown in FIG. 12 were either obtained starting from (2S,3R,4S)-4-hydroxyisoleucine or its lactone form (103). The direct derivatization of the lactone (103) led to N-Ac (104), N-Bz (105), and N-Bn (106) derivatives. N-tosylate (107a) and N,N-ditosylate (108a) derivatives were isolated from a reaction mixture involving reaction of the lactone (103) with p-toluenesulfonyl chloride in dichloromethane in the presence of triethylamine. The base hydrolysis of mono tosylated lactone (107a) gave the N-Ts derivative (11a) of (2S,3R,4S)-4-hydroxyisoleucine and, similarly, reaction of compound 107a with pyrrolidine in dichloromethane led to the amide analog (112a). The oxidation of amide (112a) with PCC gave the corresponding 4-keto derivative (113a). The reaction of o-nitrobenzenesulfonyl chloride with lactone (103) led to the N-Ns derivative (109), which upon further reaction with pyrrolidine in dichloromethane in the presence of triethylamine gave, the corresponding N-Ns amide analog (110).

Surprisingly, the reaction of the lactone (103) with pyrrolidine in dichloromethane gave a compound that showed extra methylene signals in ¹H NMR. It turned out to be a compound in which N and O are bridged with a —CH₂— group, i.e., amide (116). It seems reasonable to conclude that the source of —CH₂— group is solvent, in this case, i.e., dichloromethane reacts with the intermediate. It also seems reasonable to propose that the opening of lactone to form an amide intermediate with pyrollidine was followed by the reaction of dichloromethane with N and O of the intermediate to afford compound 116. The bridged amide (116) was tosylated and benzylated to give the corresponding derivatives 117 and 118. The reaction of (2S,3R,4S)-4-hydroxyisoleucine with CbzCl gave the Cbz-lactone (114) in almost quantitative yield, which further, upon reaction with pyrrolidine, gave the substituted amide (115). The purification of a reaction mixture from the reaction of (2S,3R,4S)-4-hydroxyisoleucine with bromo ethyl acetate in TBME/water mixture, led to the isolation of monosubstituted diacid (121a) and disubstituted triacid (121b). N,N-dibenzyl derivative (123) of (2S,3R,4S)-4-hydroxyisoleucine was obtained from the hydrolysis of the corresponding lactone (122), which in turn was prepared from (2S,3R,4S)-4-hydroxyisoleucine in two steps.

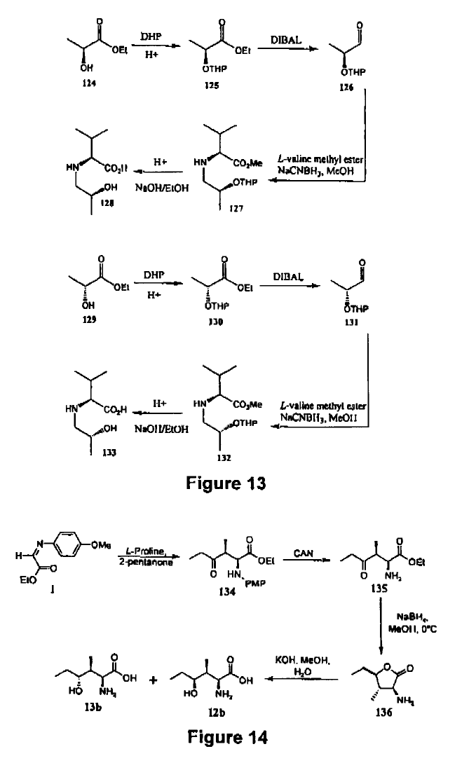

FIG. 13 depicts an enantioselecive synthesis of SS (128) and SR (133) derivatives. A diastereomeric mixture of these two compounds (compound 69) was synthesized using a different method and is given in FIG. 7. (S)-Lactic acid ethyl ester (124) reacted with DHP to give THP protected intermediate (124), which was reduced with DIBAL to give the aldehyde (126). The key transformation, reductive amination, of the aldehyde (126) with L-valine methyl ester hydrochloride and sodium cyanoborohydride gave the protected compound (127). The base hydrolysis to ester moiety, to an acid, and removal of THP group with acid gave the desired SS-isomer (128) in an excellent overall yield. The above reaction sequence was repeated with (R)-lactic acid ethyl ester to obtain the SR-isomer (133), again in an excellent isolated yield.

FIG. 14 depicts the synthesis of two diastereoisomers and an analog of (2S,3R,4S)-4-hydroxyisoleucine (12b and 13b). Mannich condensation of imine (1) with 2-pentanone in the presence of L-proline gave the desired SS-keto intermediate (134). PMP groups were removed with ceric ammonium nitrate, followed by sodium borohydride reaction in methanol to give a lactone (136), as a mixture of two diastereoisomers. The base hydrolysis of the lactone and purification afforded the SSS-isomer (12b) and also the SSR-isomer (13b).

FIGS. 15B and 15C depict the synthesis of compounds 137 through 147. Compound 143 was obtained form the reaction of (2S,3R,4S)-4-Hydroxyisoleucine with methyl iodide and sodium hydride as a base. The compound 142 was synthesized in three steps from (2S,3R,4S)-4-Hydroxyisoleucine: protection of amino acid moiety as benzyl derivative (140), followed by inversion at C-4 with excess sodium azide to yield compound 141, and single step reduction of azide and deprotection of amino acid moiety under hydrogenolysis conditions. N,N-dibenzylated compound (138) was synthesized from (2S,3R,4S)-4-Hydroxyisoleucine via lactone intermediate (137). Base assisted dibenzylation of latone (137) gave the corresponding lactone (122), which upon base hydrolysis led to compound 138. Similarly, base assisted reaction of lactone (137) with allyl bromide gave N,N-diallyl lactone which was hydrolyzed in crude form with LiOH to yield N-substitued derivative (139).

The Wittig type condensation of the aldehyde derived from the corresponding lactone, with glycine derivative lead to the alkene intermediate 144. The reduction of 144 under hydrogenation conditions, followed by base hydrolysis led to compound 146. The removal of O-benzyl group of compound 146 with formic acid in methanol gave the fully unprotected compound 147.

### B) Detailed Experimental Procedures

Detailed reaction conditions used in the preparation of compounds 1 through 136 are as follows.

### Synthesis of Compound 1

To a stirred solution of p-anisidine (50 g, 406 mmol) in toluene (400 mL) in a 1 liter round bottomed flask was added sodium sulfate (200 g, ˜2.5 eq). Ethyl glyoxalate (82 mL, 50% in toluene, 406 mmol) was added slowly to the above-described reaction mixture, and the mixture was stirred for 30 min. After this time, the sodium sulfate was filtered off using celite, and toluene was removed under reduced pressure. Compound 1 (80 g, 95%) was isolated after drying and used as is for the next reaction.

### General Procedure for Asymmetric Condensation of Ketones with Imine (1)

Imine 1 (1 eq) was added dropwise to a mixture of ketone (22 eq) and L-proline (0.35 eq) in dry DMSO (40 mL) at room temperature under nitrogen, and the mixture was stirred at room temperature for 2 h. The reaction mixture was diluted with phosphate buffer (pH 7.4), followed by extraction with ethyl acetate (3×200 mL). The organic phases were combined, dried over MgSO₄, and concentrated under reduced pressure. The desired compound (2) was isolated after purification by silica gel column chromatography. In few cases, excess ketone was removed under reduced pressure or by silica gel column chromatography.

### General Procedure for the Preparation of Isomers of 4-hydroxyisoleucine.

Detailed reaction conditions used in the preparation of compounds 2a through 15a and 2aa through 15aa are as follows. ¹H and ¹³C NMR spectra are of D₂O solutions, and chemical shifts are reported in ppm using methanol (δ 3.34 for ¹H and δ 49.50 for ¹³C) as the internal standard.

### Synthesis of Compound 2a

A mixture of 2-butanone (800 mL, 22 eq) and L-proline (15.8 g, 0.35 eq) in dry DMF (600 mL) was stirred at room temperature under nitrogen. To this reaction mixture was slowly added a solution of compound 1 in dry DMF (200 mL) and Et₃N (22.4 mL, 0.40 eq). After stirring the reaction mixture at room temperature for 8 h, L-proline was filtered off, excess 2-butanone was removed under reduced pressure, and DMF was removed in vacuo at 50° C. The crude amine (compound 2a) was purified by column chromatography (SiO₂, 85:15 hexanes/EtOAc).

### Synthesis of Compound 3a

Compound 2a was dissolved in t-BuOMe (15 mL) and to the stirred reaction mixture was added 1,5-diazabicyclo[4.3.0]non-5-ene (DBN) (1 mL, ˜0.04 eq). The reaction mixture was stirred under nitrogen for 2 h. A solid cake was obtained after overnight evaporation of the solvent at room temperature, which upon recrystallization from hot ethanol gave compound 3a (48 g, 43% yield).

### Synthesis of (2S,3R,4S)-4-Hydroxyisoleucine (compound 14a)

To a solution of compound 3a (11.6 g, 40 mmol) in CH₃CN (20 mL) was added a solution of ammonium cerium (IV) nitrate (CAN) (65.6 g, 3 eq) in water (120 mL) with stirring at 0° C. The color gradually changed from blue to green upon addition of CAN. The reaction mixture was stirred for 2.5 h, and the progress of the reaction followed by TLC analysis. After completion, the reaction mixture was extracted with EtOAc (4×150 mL) and the aqueous phase used for the next step.

The aqueous phase was neutralised to pH 7 with saturated Na₂CO₃, and cooled to −15° C. and stirred. After cooling for 30 min, KBH₄ (3.2 g, 60 mmol, 1.5 eq) was added to the reaction mixture. The reaction was allowed to warm to 0° C. for about 45 min and followed by TLC. The reaction mixture was then made basic with 2 N Na₂CO₃ to a pH of 8-9 and extracted with CH₂Cl₂ (5×400 mL). The organic phase was washed with water, dried over Na₂SO₄ and evaporated under reduced pressure to obtain a 90:10 mixture of lactones (compound 11a (2S,3R,4S) to compound 11a′ (2S,3R,4R); 3.73 g, 62.6%).

To a solution of the 90:10 lactone mixture in water (96 mL, 0.3 M) was added LiOH (1.1 g, 43.3 mmol, 1.5 eq), and the mixture was stirred at room temperature for 2 h. After the reaction was complete, it was acidified by careful addition of AcOH (43.3 mmol, 2.4 mL). The reaction mixture was concentrated under reduced pressure and last traces of water were removed by repeated addition and removal of ethanol. The crude product was crystallised from absolute EtOH to give 1.56 g of 98% pure (2S,3R,4S) 4-hydroxyisoleucine (compound 14a). Further purification by preparative HPLC gave compound 14a as white shiny powder: mp 215-222 (subl.); [α]_{D}^{H2O} +30.7 (c, 1); ¹H NMR (200 MHz) δ 3.90 (m, 1H), 3.84 (m, 1H), 1.91 (m, 1H), 1.23 (d, J=5.6 Hz, 3H) 0.95 (d, J=6.6 Hz, 3H); ¹³C NMR (75 MHz) δ 174.32, 70.46, 57.54, 41.90, 21.30, 12.70.

### Synthesis of (2S,3R,4R)-4-Hydroxyisoleucine (compound 15a)

To a solution of compound 3a (11.6 g, 40 mmol) in CH₃CN (20 mL) was added a solution of ceric ammonium nitrate (CAN) (65.6 g, 3 eq) in water (120 mL) with stirring at 0° C. The color gradually changed from blue to green upon addition of CAN. The reaction mixture was stirred for 45 min, and the progress of the reaction followed by TLC. After completion, the reaction mixture was extracted with EtOAc (4×150 mL) and the aqueous phase was carefully neutralised with saturated Na₂CO₃ solution to slightly basic pH (˜8). The aqueous phase was extracted with CH₂Cl₂ (4×150 mL) and organic extracts were combined, washed with brine, dried over anhydrous Na₂SO₄ and concentrated under reduced pressure to yield 5.52 g (79.7%) of compound 6a as a brownish oil.

To a solution of compound 6a in methanol (15 mL), cooled to 0° C., was quickly added KBH₄ (2.58 g, 47.8 mmol). The reaction mixture was stirred at 0° C. for 45 min and then gradually warmed to room temperature. The solvent was removed in vacuo, and the mixture was diluted with water. The aqueous phase was extracted with CH₂Cl₂ (4×150 mL). The organic phase was washed with brine, dried over anhydrous Na₂SO₄ and evaporated in vacuum to give a 75:25 mixture of compound 11a′ (2S,3R,4R) to compound 11a (2S,3R,4S) (2.9 g, 70.2%).

The solution of compound 11a′/compound 11a mixture in water (100 mL) was treated with LiOH (805 mg, 33.7 mmol) and stirred at room temperature for 1 h before carefully acidifying with AcOH (1.91 mL, 33.72 mmol). After concentrating under reduced pressure, the traces of water were removed by repeated addition and removal of absolute ethanol. A crude greyish solid was obtained from a cold solution of 90% ethanol. Further recrystallization from 90% ethanol yielded 1.4 g of 75:25 diastereomeric ratio of compound 15a to compound 14a. Repeated crystallisations improved the purity of compound 15a to 90%, and further purification using preparative HPLC gave pure (2S,3R,4R) 4-hydroxyisoleucine (compound 15a) as a white shiny material: mp 202-204° C. (subl.); [α]_{D}^{H2O} −21.6 (c, 0.5); ¹H-NMR (300 MHz) δ 4.05 (m, 1H), 3.80 (d, J=4.2 Hz, 1H), 2.13 (m, 1H) 1.20 (d, J=6.3 Hz, 3H), 1.05 (d, J=7.2 Hz, 3H); ¹³C NMR (75 MHz) δ 174.49, 69.13, 59.97, 39.12, 20.71, 9.38.

### Synthesis of (2S,3S,4S)-4-Hydroxyisoleucine (compound 12a)

Compound 2a (5.6 g, 20 mmol) was dissolved in acetonitrile (10 mL), and to this was added a solution of ceric ammonium nitrate (CAN) (33 g, 60 mmol) in water (60 mL) with stirring at 0° C. The reaction mixture color gradually changed from blue to green upon addition of CAN. The reaction mixture was stirred for 45 min and extracted with ethyl acetate (4×150 mL). The aqueous phase was neutralized with saturated Na₂CO₃ and pH was carefully adjusted to 7. After cooling the reaction mixture to −15° C. for 90 min, KBH₄ (1.6 g, 30 mmol, 1.5 eq) was added. The reaction was allowed to warm up to 0° C. for about 45 min and then treated with 2 N Na₂CO₃ to a pH of 8-9, followed by extraction with CH₂Cl₂ (5×400 mL). The organic phase was washed with water, dried over anhydrous Na₂SO₄ and evaporated under reduced pressure to obtain 1.42 g of a 75:25 mixture of lactones (compound 9a (2S,3S,4S) to compound 9a′ (2S,3S,4R)).

To the mixture of lactones in water (35 mL) was added LiOH (395 mg, 16.5 mmol, 1.5 eq) and the mixture was stirred at room temperature for 2 h. After this time, the reaction mixture was carefully acidified with AcOH (16.5 mmol, 0.9 mL). The solvent was removed under vacuum, and repeated addition and removal of absolute ethanol led to complete removal of water. The crude material obtained was dissolved in 90% EtOH and left overnight. The separated white solid was filtered and washed several times with EtOH, and recrystallized from 90% EtOH to obtain white crystals of (2S,3S,4S)-4-hydroxyisoleucine (compound 12a, 500 mg). Further purification using preparative HPLC led to pure shiny material: mp 253-255° C.; [α]_{D}^{H2O} +28 (c, 0.25); ¹H NMR (300 MHz) δ 4.11 (m, 1H), 3.87 (d, J=2.7 Hz, 1H), 2.21 (m, 1H), 1.23 (d, J=6.3 Hz, 3H), 0.92 (d, J=7.5 Hz, 3H); ¹³C NMR (75 MHz) δ 174.64, 71.39, 60.39, 38.97, 21.11, 6.19.

### Synthesis of (2S,3S,4R)-4-Hydroxyisoleucine (compound 13a)

To a solution of compound 2a (11.6 g, 40 mmol) in acetonitrile (20 mL) was added a solution of ammonium cerium (IV) nitrate (CAN) (65.6 g, 120 mmol) in water (120 mL) with stirring at 0° C. The reaction mixture color gradually changed from blue to green upon addition of CAN. The reaction mixture was stirred for 45 min and extracted with ethyl acetate (4×150 mL). The aqueous phase was carefully neutralised with saturated Na₂CO₃ solution to a pH of 8, followed by extraction with CH₂Cl₂ (4×150 mL). The combined organic extracts were washed with brine, dried over anhydrous Na₂SO₄ and concentrated under reduced pressure to yield 4 g of compound 4a as brown oil.

To a solution of 4a in MeOH (15 mL) at 0° C. was quickly added NaBH₄ (962 mg, 1.1 eq, 25.43 mmol). The reaction mixture was vigorously stirred at 0° C. for 45 min and gradually warmed to room temperature. The solvent was removed under reduced pressure, the residue diluted with water, and the aqueous phase extracted with CH₂Cl₂ (4×150 mL). The combined organic phases were washed with brine, dried over anhydrous Na₂SO₄ and evaporated in vacuum to give 2 g of a mixture of compound 9a′ (2S,3S,4R) and compound 9a (2S,3S,4S).

The mixture was dissolved in water (40 mL) and LiOH (556.9 mg, 18.6 mmol) was added. The reaction mixture was stirred at room temperature for 1 h and carefully acidified with AcOH (1.31 mL). The solvent was removed under vacuum. The crude product was dissolved in a minimum amount of water and the compound was loaded on a column packed with dowex 50w×8 (H⁺) resin (50 g). The column was first eluted with water 4×50 mL and then fractions were collected by eluting with 2 M NH₄OH. The isolated product was dissolved in 90% EtOH and left standing over night. The separated solid (250 mg) was filtered, washed with cold EtOH, and recrystallised from 90% EtOH to obtain a mixture of diastereoisomers.

This diastereoisomer mixture of compounds 12a and 13a was purified by preparative HPLC to produce (2S,3S,4R) 4-Hydroxyisoleucine (compound 13a) as a white shiny powder: mp 173-175° C.; [α]_{D}^{H2O} +6.0 (c, 0.25); ¹H NMR (300 MHz) δ 4.02 (d, J=3 Hz, 1H), 3.81 (m, 1H), 2.12 (m, 1H) 1.28 (d, J=6.6 Hz, 3H), 0.97 (d, J=7.2 Hz, 3H); ¹³C NMR (75 MHz) δ 174.93, 70.18, 56.34, 40.46, 21.24, 12.15.

### Syntheses of (2R,3S,4R)-4-Hydroxyisoleucine (compound 14aa), (2R,3S,4S)-4-Hydroxyisoleucine (compound 15aa), (2R,3R,4R)-4-Hydroxyisoleucine (compound 12aa), and (2R,3R,4S)-4-Hydroxyisoleucine (compound 13aa)

The procedures used in the syntheses of compounds 14aa, 15aa, 12aa, and 13aa were identical to those used for compounds 14a, 15a, 12a, and 13a, except that compound 1 was reacted with 2-butanone in the presence of D-proline to produce compound 2aa (the antipode of compound 2a). The physical and NMR data of compounds 14aa, 15aa, 12aa, and 13aa are as follows:

### (2R,3S,4R)-4-Hydroxyisoleucine (compound 14aa)

mp 217-225° C. (subl.); [α]_{D}^{H2O} −31 (c, 1); ¹H NMR (200 MHz) δ53.89 (m, 1H), 3.84 (m, 1H), 1.90 (m, 1H) 1.23 (d, J=6.4 Hz, 3H), 0.95 (d, J=7 Hz, 3H); ¹³C NMR (50 MHz) δ 174.36, 70.43, 57.51, 41.91, 21.30, 12.6.

### (2R,3S,4S)-4-Hydroxyisoleucine (compound 15aa)

mp 200-204° C. (subl.); [α]_{D}^{H2O} +22 (c, 0.5); ¹H NMR (200 MHz) δ 4.04 (m, 1H), 3.80 (m, 1H), 2.12 (m, 1H), 1.19 (d, J=6.2 Hz, 3H) 1.05 (d, J=7.2 Hz, 3H); ¹³C NMR (50 MHz) δ 174.55, 69.12, 59.97, 39.12, 20.73, 9.40.

### (2R,3R,4R)-4-Hydroxyisoleucine (compound 12aa)

mp 250-254° C.; [α]_{D}^{H2O} −30 (c, 0.25); ¹H-NMR (200 MHz) δ 4.10 (m, 1H), 3.87 (d, J=2.6 Hz 1H), 2.23 (m, 1H) 1.23 (d, J=6.6 Hz, 3H), 0.92 (d, J=7.2 Hz, 3H); ¹³C NMR (50 MHz) δ 174.64, 71.29, 60.35, 38.96, 21.12, 6.22.

### (2R,3R,4S)-4-Hydroxyisoleucine (compound 13aa)

mp 173° C.; [α]_{D}^{H2O} −5.6 (c, 0.25); ¹H NMR (300 MHz) δ 4.01 (d, J=2.7 Hz, 1H), 3.80 (m, 1H), 2.11 (m, 1H) 1.27 (d, J=6.3 Hz, 3H), 0.97 (d, J=7.2 Hz, 3H); ¹³C NMR (75 MHz) δ 174.96, 70.18, 56.35, 40.44, 21.23, 12.10.

### General Procedures for the Synthesis of Exemplary Linear and Cyclic Analogs of 4-hydroxyisoleucine:

### General Procedure for Isomerization of the Mannich Condensation Product (2)

To a solution of (2S,3S) isomer (2) in a minimum amount of the solvent was added 0.4 equivalent of DBN (1,4-diazabicyclo[4.3.0]non-5-ene), and the mixture was stirred at room temperature over night in an open flask. The solvent was evaporated by blowing a stream of argon over the reaction mixture. The crude mixture was redissolved in a minimum amount of solvent and the above procedure was repeated several times until the ratio of the two diastereoisomers remained unchanged. The solvent was evaporated under reduced pressure, and the residue was purified using high resolution silica gel chromatography to obtain mainly (2S,3R) diastereoisomer.

The following compounds were prepared using the general procedures as described above.

### Synthesis of (2S,3S)-ethyl 2-(4-methoxyphenyl amino)-3-methyl-4-oxo-hexanoate (2b)

2b: yellow oil (72%). ¹H NMR (CDCl₃, 300 MHz): δ 1.04 (t, ³J (H₈, H₇)=7.2 Hz, 3H, H₈), 1.21 (t, ³J (H₁, H₂)=7.2 Hz, 3H, H₁), 1.24 (d, ³J (H₉, H₅)=7.2 Hz, 3H, Hg), 2.55 (q, ³J (H₇, H₈)=7.2 Hz 2H, H₇), 3.03 (m, 1H, H₅), 3.73 (s, 3H, H₁₇), 3.90 (brs, 1H, H₁₀), 4.15 (q, ³J (H₂, H₁)=7.2 Hz, 1H, H₂), 4.30 (m, 1H, H₄); 6.63-6.66 (d, ³J (H₁₂, H₁₃)=9.1 Hz 12H, H₁₂, H₁₆), 6.75-6.78 (d, ³J (H₁₂, H₁₃)=9.1 Hz, 2H, H₁₃, H₁₅). ¹³C NMR (CDCl₃, 75 MHz): δ 7.53 (C₈), 12.51 (C₉), 14.08 (C₁), 34.32 (C₇), 48.37 (C₅), 55.59 (C₁₇), 59.65 (C₄), 61.43 (C₂), 114.71, 115.61 (C₁₂, C₁₃, C₁₅, C₁₆), 140.76 (C₁₁), 152.96 (C₁₄), 172.85 (C₃), 211.81 (C₆). MS m/z: 294 (M+1), 316 (M+23).

### Synthesis of (2S,3R)-ethyl 2-(4-methoxyphenyl amino)-3-methyl-4-oxo-hexanoate (3b)

3b: yellow oil (60%). ¹H NMR (CDCl₃, 300 MHz): δ 1.06 (t, ³J (H₈, H₇)=7.2 Hz, 3H, H₈), 1.22 (m, 6H, H₁, H₉), 2.55 (q, ³J (H₇, H₈)=7.2 Hz 2H, H₇), 3.03 (m, 1H, H₅), 3.73 (s, 3H, H₁₇), 3.90 (brs, 1H, H₁₀), 4.15 (q, ³J (H₂, H₁)=7.2 Hz, 1H, H₂), 4.26 (m, 1H, H₄), 6.63-6.66 (d, ³J (H₁₂, H₁₃)=9.1 Hz, 2H, H₁₂, H₁₆), 6.75-6.78 (d, ³J (H₁₂, H₁₃)=9.1 Hz, 2H, H₁₃, H₁₅). ¹³C NMR (CDCl₃, 75 MHz): δ 7.46 (C₈), 13.22 (C₉), 14.08 (C₁), 34.94 (C₇), 48.29 (C₅), 55.59 (C₁₇), 60.69 (C₄), 61.07 (C₂), 114.71, 115.77 (C₁₂, C₁₃, C₁₅, C₁₆), 140.70 (C₁₁), 153.03 (C₁₄), 172.68 (C₃), 212.10 (C₆). MS m/z: 294 (M+1), 316 (M+23).

### Synthesis of (S)-ethyl 2-(4-methoxyphenylamino)-2-((S)-2-oxo-cyclohexyl)-acetate (2e)

2e: brown oil (85%). ¹H NMR (CDCl₃, 200 MHz): δ 1.21 (t, ³J (H₁, H₂)=7.2 Hz, 3H, H₁), 1.65-2.49 (m, 8H, H₇, H₈, H₉, H₁₀), 2.81 (m, 1H, H₅), 3.74 (s, 3H, H₁₈), 3.87 (brs, 1H, H₁), 4.14 (q, ³J (H₂, H₁)=7.2 Hz, 1H, H₂), 4.23 (d, ³J (H₄, H₅)=5.3 Hz, 1H, H₄), 6.70-6.73 (d, ³J (H₁₃, H₁₄)=9.2 Hz, 2H, H₁₃, H₁₇), 6.75-6.78 (d, ³J (H₁₂, H₁₃)=9.2 Hz, 2H, H₁₄, H₁₆). ¹³C NMR (CDCl₃, 75 MHz): δ 14.08 (C₁), 24.71 (C₈), 26.81 (C₉), 29.54 (C₁₀), 41.78 (C₇), 53.50 (C₅), 55.64 (C₁₈), 58.05 (C₄), 61.08 (C₂); 114.70, 116.01 (C₁₃, C₁₄, C₁₆, C₁₇), 141.08 (C₁₂), 152.99 (C₁₅), 173.40 (C₃), 210.02 (C₆). MS (IC) m/z: 306 (M+1).

### Synthesis of (S)-ethyl 2-(4-methoxyphenylamino)-2-((R)-2-oxo-cyclohexyl)-acetate (3e)

3e: orange oil (60%, 98% purity). ¹H NMR (CDCl₃, 300 MHz): δ 1.22 (t, ³J (H₁, H₂)=7.2 Hz, 3H, H₁), 1.65-2.49 (m, 8H, H₇, H₈, H₉, H₁₀), 3.11 (m, 1H, H₅), 3.74 (s, 3H, H₁₈), 3.99 (d, ³J (H₄, H₅)=3.7 Hz, 1H, H₄), 4.15 (q, ³J (H₂, H₁)=7.2 Hz, 1H, H₂), 4.24 (brs, 1H, H₁₁), 6.62-6.65 (d, ³J (H₁₃, H₁₄)=8.7 Hz, 2H, H₁₃, H₁₇), 6.75-6.78 (d, ³J (H₁₂, H₁₃)=8.7 Hz, 2H, H₁₄, H₁₆). ¹³C NMR (CDCl₃, 75 MHz): δ 14.04 (C₁), 24.47 (C₈), 26.77 (C₉), 30.45 (C₁₀), 41.73 (C₇), 53.51 (C₅), 55.61 (C₁₈), 58.99 (C₄), 61.09 (C₂), 114.67, 115.53 (C₁₃, C₁₄, C₁₆, C₁₇), 142.09 (C₁₂), 152.69 (C₁₅), 172.97 (C₃), 210.87 (C₆). MS (IC) m/z: 306 (M+1).

### Synthesis of (S)-ethyl 2-(4-methoxyphenylamino)-2-((S)-2-oxo-cycloheptyl)-acetate (2f)

2f: recrystallized from ethyl acetate, yellow solid (65%). ¹H NMR (CDCl₃, 200 MHz): 1.20 (t, ³J (H₁, H₂)=7.1 Hz, 3H, H₁), 1.31-2.02 (m, 8H, H₈, H₉, H₁₀, H₁₁), 2.52 (m, 2H, H₇), 2.92 (m, 1H, H₅), 3.73 (s, 3H, H₁₉), 3.92 (brs, 1H, H₁₂), 4.13 (q, ³J (H₂, H₁)=7.1 Hz, 1H, H₂), 4.26 (d, ³J (H₄, H₅)=5.9 Hz, 1H, H₄), 6.64-6.68 (d, ³J (H₁₄, H₁₅)=9 Hz, 2H, H₁₄, H₁₈), 6.73-6.78 (d, ³J (H₁₄, H₁₅)=9 Hz, 2H, H₁₅, H₁₇). ¹³C NMR (CDCl₃, 75 MHz): δ 14.11 (C₁), 24.71, 27.12, 29.22, 29.80 (C₈, C₉, C₁₀, C₁₁), 43.86 (C₇), 55.16 (C₅), 55.64 (C₁₉), 60.62 (C₄), 61.17 (C₂), 114.72, 115.99 (C₁₄, C₁₅, C₁₇, C₁₈), 140.93 (C₁₃), 153.05 (C₁₆), 173.14 (C₃), 214.34 (C₆). MS (E) m/z: 342 (M+23).

### Synthesis of (S)-ethyl 2-(4-methoxyPhenylamino)-2-((R)-2-oxo-cycloheptyl)-acetate (3f)

3f: yellow oil (99% purity). ¹H NMR (CDCl₃, 300 MHz): δ 1.23 (t, ³J (H₁, H₂)=7.2 Hz, 3H, H₁), 1.32-2.03 (m, 8H, H₈, H₉, H₁₀, H₁₁), 2.54 (m, 2H, H₇), 3.03 (m, 1H, H₅), 3.73 (s, 3H, H₁₉), 4.16 (q, ³J (H₂, H₁)=7.2 Hz, 1H, H₂), 4.29 (brs, 1H, H₁₂), 4.31 (d, ³J (H₄, H₅)=4.7 Hz, 1H, H₄), 6.66-6.69 (d, ³J (H₁₄, H₁₅)=9.1 Hz, 2H, H₁₄, H₁₈), 6.76-6.80 (d, ³J (H₁₄, H₁₅)=9.1 Hz, 2H, H₁₅, H₁₇). ¹³C NMR (CDCl₃ 75 MHz): δ 14.09 (C₁), 24.15, 27.11, 28.94, 29.82 (C₈, C₉, C₁₀, C₁₁), 43.80 (C₇), 54.29 (C₅), 55.62 (C₁₉), 60.60 (C₄), 61.21 (C₂), 114.79, 115.15 (C₁₄, C₁₅, C₁₇, C₁₈), 140.92 (C₁₃), 152.66 (C₁₆), 172.50 (C₃), 214.09 (C₆). MS (E) m/z: 342 (M+23).

### Synthesis of (2S,3S)-ethyl 2-(4-methoxyphenyl amino)-4-methyl-3-phenylpentanoate (2c)

2c: recrystallization from hexane ether, yellow solid (75%). ¹H NMR (CDCl₃, 200 MHz): δ 1.25 (t, ³J (H₁, H₂)=7.1 Hz, 3H, H₁), 2.15 (s, 3H, H₇), 3.51 (brs, 1H, H₁₄), 3.74 (s, 3H, H₂₁), 4.19 (q, ³J (H₂, H₁)=7.1 Hz, 1H, H₂), 4.25 (d, ³J (H₄, H₅)=8.5 Hz, 1H, H₄), 4.64 (d, ³J (H₅, H₄)=8.5 Hz, 1H, H₅), 6.58-6.62 (d, ³J (H₁₆, H₁₇)=9 Hz, 2H, H₁₆, H₂₀), 6.70-6.74 (d. ³J (H₁₆, H₁₇)=9 Hz, 2H, H₁₇, H₁₉), 7.24-7.37 (m, 5H, Hg, H₁₀, H₁₁, H₁₂, H₁₃). ¹³C (CDCl₃, 75 MHz): δ 14.09 (C₁), 29.19 (C₇), 55.60 (C₂₁), 59.78 (C₅) 61.29 (C₂), 61.53 (C₄), 114.49, 116.12 (C₁₆, C₁₇, C₁₉, C₂₀), 128.12 (C₁₁), 129.04. 129.19 (C₉, C₁₀, C₁₂, C₁₃), 134.34 (C₈), 140.61 (C₁₅), 153.01 (C₁₈), 173.22 (C₃), 206.09 (C₆). MS (E) m/z: 364 (M+23).

### Synthesis of (2S,3R)-ethyl 2-(4-methoxyphenyl amino)-4-methyl-3-phenylpentanoate (3c)

3c: yellow oil (90% purity). ¹H NMR (CDCl₃, 300 MHz): δ 0.88 (t, ³J (H₁, H₂)=7.1 Hz, 3H, H₁), 2.17 (s, 3H, H₇), 3.74 (s, 3H, H₂₁), 3.78 (brs, 1H, H₁₄), 3.84 (q, ³J (H₂, H₁)=7.1 Hz, 1H, H₂), 4.11 (d, ³J (H₄, H₅)=8.7 Hz, 1H, H₄), 4.55 (d, ³J (H₅, H₄)=8.7 Hz, 1H, H₅), 6.65-6.68 (d, ³J (H₁₆, H₁₇)=9 Hz, 2H, H₁₆, H₂₀), 6.72-6.75 (d, ³J (H₁₆, H₁₇)=9 Hz, 2H, H₁₇, H₁₉), 7.32 (brs, 5H, Hg, H₁₀, H₁₁, H₁₂, H₁₃). ¹³C NMR (CDCl₃, 75 MHz): δ 13.31 (C₁), 29.53 (C₇), 55.11 (C₂₁), 60.40 (C₂) 61.07, 61.77 (C₄, C₅), 114.30, 116.19 (C₁₆, C₁₇, C₁₉, C₂₀), 127.77 (C₁₁), 128.63, 128.92 (C₉, C₁₀, C₁₂, C₁₃), 133.82 (C₈), 140.70 (C₁₅), 152.96 (C₁₈), 172.54 (C₃), 205.21 (C₆). MS (E) m/z: 364 (M+23).

### Synthesis of (2S,3S)-ethyl 3-benzyl-2-(4-methoxyphenyl amino)-4-oxopentanoate (2d)

2d: yellow solid (60%). ¹H NMR (CDCl₃, 300 MHz): δ 1.26 (t, ³J (H₁, H₂)=7.1 Hz, 3H, H₁), 2.04 (s, 3H, H₇), 3.09 (m, 2H, H₈), 3.34 (m, 1H, H₅), 3.75 (s, 3H, H₂₂), 4.08 (brs, 1H, H₁₅), 4.18 (q, ³J (H₂, H₁)=7.1 Hz, 1H, H₂), 4.19 (m, 1H, H₄), 6.49-6.52 (d, ³J (H₁₇, H₁₈)=9 Hz, 2H, H₁₇, H₂₁), 6.73-6.76 (d, ³J (H₁₇, H₁₈)=9 Hz, 2H, H₁₈, H₂₀), 7.24-7.37 (m, 5H, H₉, H₁₀, H₁₁, H₁₂, H₁₃). ¹³C (CDCl₃, 75 MHz): δ 14.14 (C₁), 30.98 (C₇), 34.67 (C₈), 55.68 (C₂₂), 57.02 (C₅), 58.41 (C₄), 61.52 (C₂), 114.81, 115.32 (C₁₇, C₁₈, C₂₀, C₂₁), 126.69 (C₁₂), 128.64, 129.05 (C₁₀, C₁₁, C₁₃, C₁₄), 138.66 (C₉), 140.35 (C₁₆), 152.93 (C₂₂), 172.52 (C₃), 209.36 (C₆). MS (E) m/z: 356 (M+1), 378 (M+23).

### Synthesis of (2S,3R)-ethyl 3-benzyl-2-(4-methoxyphenyl amino)-4-oxopentanoate (3d)

3d: yellow oil (99% purity). ¹H NMR (CDCl₃, 300 MHz): δ 1.20 (t, ³J (H₁, H₂)=7.2 Hz, 3H, H₁), 2.08 (s, 3H, H₇), 2.98 (m, 2H, H₈), 3.43 (m, 1H, H₅), 3.74 (s, 3H, H₂₂), 4.13 (m, 3H, H₂, H₄), 4.45 (brs, 1H, H₁₅), 6.58-6.61 (d, ³J (H₁₇, H₁₈)=8.8 Hz, 2H, H₁₇, H₂₁), 6.76-6.79 (d, ³J (H₁₇, H₁₈)=8.8 Hz, 2H, H₁₈, H₂₀), 7.17-7.30 (m, 5H, Hg, H₁₀, H₁₁, H₁₂, H₁₃). ¹³C NMR (CDCl₃, 75 MHz): δ 13.93 (C₁), 31.01 (C₇), 34.53 (C₈), 55.33 (C₂₂), 55.67 (C₅), 58.79 (C₄), 60.99 (C₂), 114.48, 115.47 (C₁₇, C₁₈, C₂₀, C₂₁), 126.49 (C₁₂), 128.46, 128.79 (C₁₀, C₁₁, C₁₃, C₁₄), 138.02 (C₉), 140.70 (C₁₆), 152.73 (C₂₂), 172.75 (C₃), 209.77 (C₆). MS (E) m/z: 356 (M+1), 378 (M+23).

### General Procedure for Deprotection of p-methoxypheny (PMP) Group of γ-oxo-α-(4-methoxyphenyl amino) Esters with Ceric Ammonium Nitrate (CAN)

To a solution of γ-oxo-α-(4-methoxyphenyl amino) ester (10 mmol) in CH₃CN (6 mL) at 0° C., was added a solution of ceric ammonium nitrate (CAN, 3 eq) in water (60 mL) with added quickly but dropwise with stirring. The reaction mixture was stirred for 45 min at 0° C. CH₂Cl₂ (60 mL) was added to the reaction mixture, and the phases were separated. The organic phase was washed with 0.1 N aqueous HCl (60 mL). The aqueous phases were combined and extracted with CH₂Cl₂ (3×130 mL), basified with a solution of Na₂CO₃ (2N) to pH 7, and extracted again with CH₂Cl₂ (3×150 mL). The combined organic phases were dried over MgSO₄ and concentrated under reduced pressure to obtain γ-oxo-α-aminoesters. The following compounds were prepared using the general procedures described above.

### Synthesis of (2S,3R)-ethyl 2-amino-3-methyl-4-oxopentanoate (6a)

6a: clear oil (88%). ¹H NMR (CDCl₃, 300 MHz): δ 1.16 (d, ³J (H₈, H₅)=7.5 Hz, 3H, H₈), 1.24 (t, ³J (H₁, H₂)=7.2 Hz, 3H, H₁), 1.70 (brs, 1H, Hg), 2.17 (s, 3H, H₇), 2.92 (m, 1H, H₅), 3.53 (d, ³J (H₄, H₅)=6.4 Hz, 1H, H₄), 4.16 (q, ³J (H₂, H₁)=7.2 Hz, 2H, H₂). ¹³C NMR (CDCl₃, 75 MHz): δ 13.25 (C₈), 14.00 (C₁), 28.73 (C₇), 50.18 (C₅), 56.72 (C₄), 60.89 (C₂), 174.26 (C₃), 210.06 (C₆). MS (IC) m/z: 174 (M+1).

### Synthesis of (2S,3S)-ethyl 2-amino-3-methyl-4-oxopentanoate (4a)

4a: clear oil (88%). ¹H NMR (CDCl₃, 300 MHz): δ 1.11 (d, ³J (H8, H₅)=7.1 Hz, 3H, H₈), 1.25 (t, ³J (H₁, H₂)=7.2 Hz, 3H, H₁), 1.70 (brs, 1H, Hg), 2.20 (s, 3H, H₇), 2.92 (m, 1H, H₅), 3.86 (d, ³J (H₄, H₅)=4.9 Hz, 1H, H₄), 4.16 (q, ³J (H₂, H₁)=7.2 Hz, 2H, H₂). ¹³C (CDCl₃, 50 MHz): δ 10.82 (C₈), 14.07 (C₁), 28.24 (C₇), 49.64 (C₅), 55.26 (C₄), 61.16 (C₂), 174.18 (C₃), 209.80 (C₆). MS (IC) m/z: 174 (M+1).

### Synthesis of (2S,3S)-ethyl 2-amino-3-methyl-4-oxohexanoate (4b)

4b: clear oil (84%). ¹H NMR (CDCl₃, 300 MHz): δ 1.04 (t, ³J (H₈, H₇)=7.2 Hz, 3H, H₈), 1.11 (d, ³J (Hg, H₅)=7.2 Hz, 3H, Hg), 1.25 (t, ³J (H₁, H₂)=7.2 Hz, 3H, H₁), 2.52 (q, ³J (H₇, H₈)=7.2 Hz, 2H, H₇), 2.91 (m, 1H, H₅), 3.84 (d, ³J (H₄, H₅)=5.0 Hz, 1H, H₄), 4.16 (q, ³J (H₂, H₁)=7.2 Hz, 1H, H₂). ¹³C NMR (CDCl₃, 75 MHz): δ 7.58 (C₈), 11.23 (C₉), 14.09 (C₁), 34.03 (C₇), 48.74 (C₅), 55.45 (C₄), 61.10 (C₂), 174.15 (C₃), 212.44 (C₆). MS (IC) m/z: 188 (M+1).

### Synthesis of (2S,3R)-ethyl 2-amino-3-methyl-4-oxohexanoate (6b)

6b: clear oil (84%). ¹H NMR (CDCl₃, 300 MHz): δ 1.02 (t, ³J (H8, H₇)=7.2 Hz, 3H, H₈), 1.14 (d, ³J (Hg, H₅)=7.2 Hz, 3H, Hg), 1.24 (t, ³J (H₁, H₂)=7.2 Hz, 3H, H₁), 2.50 (q, ³J (H₇, H₈)=7.2 Hz, 2H, H₇), 2.91 (m, 1H, H₅), 3.53 (d, ³J (H₄, H₅)=6.5 Hz, 1H, H₄), 4.16 (q, ³J (H₂, H₁)=7.2 Hz, 1H, H₂). ¹³C NMR (CDCl₃ 75 MHz): δ 7.46 (C₈), 13.69 (C₉), 14.09 (C₁), 34.98 (C₇), 49.22 (C₅), 57.04 (C₄), 60.94 (C₂), 174.48 (C₃), 212.89 (C₆). MS (IC) m/z: 188 (M+1).

### Synthesis of (S)-ethyl 2-amino-2-((S)-2-oxocyclohexyl)acetate (4e)

4e: clear oil (80%). ¹H NMR (CDCl₃, 300 MHz): δ 1.26 (t, ³J (H₁, H₂)=7.2 Hz, 3H, H₁), 1.62-2.09 (m, 6H, H₈, H₉, H₁₀), 2.25-2.45 (m, 2H, H₇), 2.78 (m, 1H, H₅), 3.93 (d, ³J (H₄, H₅)=3.8 Hz, 1H, H₄), 4.17 (q, ³J (H₂, H₁)=7.2 Hz, 1H, H₂). ¹³C NMR (CDCl₃ 75 MHz): δ 14.14 (C₁), 24.68, 26.94, 27.68 (C₈, C₉, C₁₀), 41.94 (C₇), 53.44, 53.91 (C₄, C₅), 60.96 (C₂), 174.40 (C₃), 210.90 (C₆).

### Synthesis of (S)-ethyl 2-amino-2-((R)-2-oxocyclohexyl)acetate (6e)

6e: a clear oil (80%). ¹H NMR (CDCl₃, 300 MHz): δ 1.26 (t, ³J (H₁, H₂)=7.2 Hz, 3H, H₁), 1.62-2.09 (m, 6H, H₈, H₉, H₁₀), 2.25-2.45 (m, 2H, H₇), 2.98 (m, 1H, H₅), 3.35 (d, ³J (H₄, H₅)=4.7 Hz, 1H, H₄), 4.17 (q, ³J (H₂, H₁)=7.2 Hz, 1H, H₂). ¹³C NMR (CDCl₃ 75 MHz): δ 14.14 (C₁), 24.87, 27.11, 30.76 (C₈, C₉, C₁₀), 41.94 (C₇), 53.70, 55.33 (C₄, C₅), 60.96 (C₂), 174.40 (C₃), 211.20 (C₆).

### Synthesis of (S)-ethyl 2-amino-2-((S)-2-oxocycloheptyl)acetate (4f)

4f: clear oil (80%). ¹H NMR (CDCl₃, 300 MHz): δ 1.26 (t, ³J (H₁, H₂)=7.2 Hz, 3H, H₁), 1.31-2.02 (m, 8H, H₈, H₉, H₁₀, H₁₁), 2.52 (m, 2H, H₇), 2.92 (m, 1H, H₅), 3.83 (d, ³J (H₄, H₅)=4.7 Hz, 1H, H₄), 4.18 (q, ³J (H₂, H₁)=7.2 Hz, 1H, H₂). ¹³C NMR (CDCl₃, 75 MHz): δ 14.15 (C₁), 23.92, 26.55, 29.57, 29.87 (C₈, C₉, C₁₀, C₁₁), 43.87 (C₇), 55.24, 56.08 (C₄, C₅), 61.03 (C₂), 174.58 (C₃), 214.71 (C₆).

### Synthesis of (S)-ethyl 2-amino-2-((R)-2-oxocyclohexptyl)acetate (6f)

6f: clear oil (80%). ¹H NMR (CDCl₃, 300 MHz): δ 1.28 (t, ³J (H₁, H₂)=7.2 Hz, 3H, H₁), 1.31-2.02 (m, 8H, H₈, H₉, H₁₀, H₁₁), 2.52 (m, 2H, H₇), 3.07 (m, 1H, H₅), 3.56 (d, ³J (H₄, H₅)=4.9 Hz, 1H, H₄), 4.18 (q, ³J (H₂, H₁)=7.2 Hz, 1H, H₂). ¹³C NMR (CDCl₃ 50 MHz): δ 13.95 (C₁), 23.67, 28.19, 29.23, 29.45 (C₈, C₉, C₁₀, C₁₁), 43.73 (C₇), 54.87, 57.20 (C₄, C₅), 60.78 (C₂), 174.23 (C₃), 214.33 (C₆).

### Synthesis of (2S,3S)-ethyl 2-amino-4-oxo-3-phenypentanoate (4c)

4c: clear oil (65%). ¹H NMR (CDCl₃, 200 MHz): δ 1.24 (t, ³J (H₁, H₂)=7.1 Hz, 3H, H₁), 1.47 (brs, 2H, H₁₄), 2.06 (s, 3H, H₇), 4.12 (m, 4H, H₂, H₅, H₄), 7.20-7.33 (m, 5H, Hg, H₁₀, H₁₁, H₁₂, H₁₃). ¹³C NMR (CDCl₃, 50 MHz): δ 13.85 (C₁), 29.03 (C₇), 55.79 (C₄), 60.92 (C₂), 62.20 (C₅), 127.86 (C₁₁), 128.85, 129.02 (C₉, C₁₀, C₁₂, C₁₃), 134.27 (C₈), 173.34 (C₃), 206.69 (C₆).

### Synthesis of (2S,3R)-ethyl 2-amino-4-oxo-3-phenypentanoate (6c)

6c: clear oil (65%). ¹H NMR (CDCl₃, 300 MHz): δ 0.91 (t, ³J (H₁, H₂)=7.1 Hz, 3H, H₁), 1.63 (brs, 2H, H₁₄), 2.08 (s, 3H, H₇), 3.93 (m, 4H, H₂, H₅, H₄), 7.18-7.31 (m, 5H, Hg H₁₀, H₁₁, H₁₂, H₁₃). ¹³C NMR (CDCl₃, 75 MHz): δ 13.56 (C₁), 29.79 (C₇), 57.18 (C₄), 60.50 (C₂), 63.54 (C₅), 127.77 (C₁₁), 128.66, 128.91 (C₉, C₁₀, C₁₂, C₁₃), 134.73 (C₈), 173.73 (C₃), 206.59 (C₆).

### Synthesis of (2S,3S)-ethyl 2-amino-3-benzyl-4-oxopentanoate (4d)

4d: clear oil (50%). ¹H NMR (CDCl₃, 300 MHz): δ 1.26 (t, ³J (H₁, H₂)=7.2 Hz, 3H, H₁), 2.02 (s, 3H, H₇), 2.96 (m, 2H, H₈), 3.27 (m, 1H, H₅), 3.79 (d, ³J (H₄, H₅)=5.3 Hz, 1H, H₄), 4.13 (m, 1H, H₂), 7.14-7.31 (m, 5H, H₁₀, H₁₁, H₁₂, H₁₃, H₁₄). ¹³C NMR (CDCl₃, 75 MHz): δ 14.12 (C₁), 30.61 (C₇), 33.41 (C₈), 55.04 (C₅), 57.41 (C₄), 61.35 (C₂), 126.46 (C₁₂), 128.51, 128.97 (C₁₀, C₁₁, C₁₃, C₁₄), 138.95 (C₉), 173.83 (C₃), 209.71 (C₆).

### Synthesis of (2S,3R)-ethyl 2-amino-3-benzyl-4-oxopentanoate (6d)

6d: clear oil (50%). ¹H NMR (CDCl₃, 300 MHz): 1.27 (t, ³J (H₁, H₂)=7.2 Hz, 3H, H₁), 2.04 (s, 3H, H₇), 2.96 (m, 2H, H₈), 3.27 (m, 1H, H₅), 3.44 (d, ³J (H₄, H₅) 5.9 Hz, 1H, H₄), 4.17 (m, 1H, H₂), 7.17-7.33 (m, 5H, H₁₀, H₁₁, H₁₂, H₁₃, H₁₄). ¹³C NMR (CDCl₃ 75 MHz): δ 14.10 (C₁), 31.18 (C₇), 34.73 (C₈), 55.40 (C₅), 56.55 (C₄), 61.09 (C₂), 126.52 (C₁₂), 128.56, 128.84 (C₁₀, C₁₁, C₁₃, C₁₄), 138.62 (C₉), 174.78 (C₃), 210.43 (C₆).

### General Procedure for the Hydrolysis of γ-oxo-α-aminoesters

To a solution of γ-oxo-α-aminoester in H₂O/MeOH (0.35 M) was added, dropwise, 2N aqueous KOH solution (1.1 equivalents), and the reaction mixture was stirred at room temperature for 24 h. An aqueous solution of 2 N HCl acid was added to adjust the pH to 6. The solvents were evaporated under reduced pressure and the crude product was purified by silica gel column chromatography. The following compounds were prepared using the general procedures described above.

### Synthesis of (2S,3S)-2-amino-3-methyl-4-oxopentanoic acid (5a)

5a: an oil (50%). ¹H NMR (D₂O, 300 MHz): δ 1.26 (d, ³J (H₆, H₃)=7.5 Hz, 3H, H₆), 2.33 (s, 3H, H₅), 3.36 (m, 1H, H₃), 4.10 (d, ³J (H₂, H₃)=3.7 Hz, 1H, H₂). ¹³C NMR (D₂O, 50 MHz): δ 10.85 (C₆), 28.15 (C₅), 46.61 (C₃), 55.17 (C₂), 173.48 (C₁), 214.76 (C₄).

### Synthesis of (2S,3R)-2-amino-3-methyl-4-oxopentanoic acid (7a)

7a: an oil (56%). ¹H NMR (D₂O, 300 MHz): δ 1.31 (d, ³J (H₆, H₃)=7.5 Hz, 3H, H₆), 2.30 (s, 3H, H₅), 3.36 (m, 1H, H₃), 3.95 (d, ³J (H₂, H₃)=5.1 Hz, 1H, H₂). ¹³C NMR (D₂O, 50 MHz): δ 12.48 (C₆), 28.38 (C₅), 46.76 (C₃), 56.39 (C₂), 173.32 (C₁), 214.54 (C₄).

### Synthesis of (2S,3S)-2-amino-3-methyl-4-hexanoic acid (5b)

5b: an orange oil (80%). ¹H NMR (D₂O, 200 MHz): δ 1.02 (t, ³J (H₆, H₅)=6.9 Hz, 3H, H₆), 1.21 (d, ³J (H₇, H₃)=7.5 Hz, 3H, H₇), 2.67 (m, 2H, H₅), 3.35 (m, 1H, H₃), 4.04 (d. ³J (H₂, H₃)=4.1 Hz. 1H, H₂). ¹³C NMR (D₂O. 50 MHz): δ 7.30 (C₆), 11.20 (C₇), 34.56 (C₅), 45.64 (C₃), 56.72 (C₂), 173.53 (C₁), 217.49 (C₄).

### Synthesis of (2S,3R)-2-amino-3-methyl-4-hexanoic acid (7b)

7b: orange oil (80%). ¹H NMR (D₂O. 200 MHz): δ 1.02 (m, 3H, H₆), 1.29 (d, ³J (H₇, H₃)=7.5 Hz, 3H, H₇), 2.67 (m, 2H, H₅), 3.35 (m, 1H, H₃), 3.89 (d, ³J (H₂, H₃)=4.7 Hz, 1H, H₂), ¹³C NMR (D₂O, 50 MHz): δ 7.30 (C₆), 12.99 (C₇), 34.75 (C₅), 45.64 (C₃), 55.50 (C₂), 173.32 (C₁), 217.70 (C₄).

### Synthesis of (S)-2-amino-2-((S)-2-cyclohexyl)acetic acid (5e)

5e: yellow oil (63%). ¹H NMR (D₂O, 300 MHz): δ 1.72 (m, 4H, H₆, H₇), 1.89-2.17 (m, 4H, H₅, H8), 2.54 (m, 1H, H₃), 3.25 (m, 1H, H₃), 4.17 (d, ³J (H₂, H₃)=2.2 Hz, 1H, H₂), ¹³C NMR (D₂O, 50 MHz): δ 24.54 (C₆), 27.10 (C₇), 27.87 (C₈), 41.74 (C₅), 50.75 (C₂), 53.66 (C₃), 173.66 (C₁), 215.30 (C₄).

### Synthesis of (S)-2-amino-2-((R)-2-cyclohexyl)acetic acid (7e)

7e: oil (63%). ¹H NMR (D₂O, 300 MHz): δ 1.72 (m, 4H, H₆, H₇), 1.89-2.17 (m, 4H, H₅, H₈), 2.54 (m, 1H, H₃), 3.25 (m, 1H, H₃), 3.74 (d, ³J (H₂, H₃)=4.9 Hz, 1H, H₂). ¹³C NMR (D₂O, 50 MHz): δ 24.76 (C₆), 27.44 (C₇), 31.34 (C₈), 42.06 (C₅), 50.75 (C₂), 55.14 (C₃), 173.66 (C₁), 215.54 (C₄).

### Synthesis of (S)-2-amino-2-((S)-2-cycloheptyl)acetic acid (5f)

5f: clear oil (70%). ¹H NMR (D₂O, 300 MHz): δ 1.31-2.01 (m, 8H, H₆, H₇, H8, Hg), 2.45-2.77 (m, 2H, H₅), 3.43 (m, 1H, H₃), 4.05 (d, ³J (H₂, H₃)=2.6 Hz, 1H, H₂). ¹³C NMR (D₂O, 75 MHz): δ 23.22, 25.97, 29.29, 29.71 (C₆, C₇, C₈, C₉); 43.48 (C₅), 51.64 (C₃), 55.96 (C₂), 173.73 (C₁), 219.05 (C₄).

### Synthesis of (S)-2-amino-2-((R)-2-cycloheptyl)acetic acid (7f)

7f: clear oil (70%). ¹H NMR (D₂O, 300 MHz): δ 1.31-2.01 (m, 8H, H₆, H₇, H₈, H₉), 2.45-2.77 (m, 2H, H₅), 3.43 (m, 1H, H₃), 3.87 (d, ³J (H₂, H₃)=4.1 Hz, 1H, H₂). ¹³C NMR (D₂O, 75 MHz): δ 23.22, 27.91, 28.93, 29.26 (C₆, C₇, C₈, C₉), 43.79 (C₅), 51.39 (C₃), 57.39 (C₂), 173.53 (C₁), 219.52 (C₄).

### Synthesis of (2S,3S)-2-amino-4-oxo-3-phenylpentanoic acid (5c)

5c: clear oil (60%). ¹H NMR (D₂O, 300 MHz): δ 2.20 (s, 3H, H₅), 4.08 (d, ³J (H₂, H₃)=6.8 Hz, 1H, H₂), 4.59 (d, ³J (H₃, H₂)=6.8 Hz, 1H, H₃), 7.28-7.49 (m, 5H, H₇, H₈, Hg, H₁₀, H₁₁). ¹³C NMR (D₂O, 75 MHz): δ 29.12 (C₅), 57.28 (C₂), 58.55 (C₃), 128.68 (C₉), 129.73, 130.05 (C₇, C₈, C₁₀, C₁₁), 133.44 (C₆), 173.43 (C₁), 211.17 (C₄).

### Synthesis of (2S,3R)-2-amino-4-oxo-3-phenylpentanoic acid (7c)

7c: clear oil (60%). ¹H NMR (D₂O, 300 MHz): δ 2.23 (s, 3H, H₅), 4.37 (d, ³J (H₂, H₃)=6.1 Hz, 1H, H₂), 4.57 (d, ³J (H₃, H₂)=6.1 Hz, 1H, H₃), 7.28-7.49 (m, 5H, H₇, H₈, Hg, H₁₀, H₁₁). ¹³C NMR (D₂O, 75 MHz): δ 29.13 (C₅), 56.01 (C₂), 58.94 (C₃), 129.20 (C₉), 129.50, 130.13 (C₇, C₈, C₁₀, C₁₁), 132.03 (C₆), 173.43 (C₁), 211.17 (C₄).

### Synthesis of (2S,3S)-2-amino-3-benzyl-4-oxopentanoic acid (5d)

5d: clear oil (70%). ¹H NMR (D₂O, 300 MHz): δ 2.01 (s, 3H, H₅), 2.96 (m, 2H, H₆), 3.61 (m, 1H, H₃), 4.01 (m, 1H, H₂), 7.29-7.46 (m, 5H, H8, H₉, H₁₀, H₁₁, H₁₂). ¹³C NMR (D₂O, 75 MHz): δ 31.10 (C₅), 33.69 (C₆), 54.10 (C₃), 55.59 (C₂), 127.40 (C₁₀), 129.32, 129.43 (C₈, C₉, C₁₁, C₁₂), 138.07 (C₇), 173.82 (C₁), 214.92 (C₄).

### Synthesis of (2S,3R)-2-amino-3-benzyl-4-oxopentanoic acid (7d)

7d: clear oil (70%). ¹H NMR (D₂O, 300 MHz): δ 2.10 (s, 3H, H₅), 2.92-3.20 (m, 2H, H₆), 3.76 (m, 1H, H₃), 3.81 (m, 1H, H₂), 7.29-7.46 (m, 5H, H₈, H₉, H₁₀, H₁₁, H₁₂). ¹³C NMR (D₂O, 75 MHz): δ 30.97 (C₅), 34.35 (C₆), 53.77 (C₃), 55.59 (C₂), 127.54 (C₁₀), 129.22, 129.32 (C₈, C₉, C₁, C₁₂), 137.91 (C₇), 173.37 (C₁), 215.26 (C₄).

### General Methods for the Reduction of γ-oxo-α-amino-esters

### General One-Step Process Involving Deprotection-Reduction of γ-oxo-α-amino-esters:

To a solution of γ-oxo-α-amino-esters (10 mmol) in MeCN (6 mL) was added a solution of CAN (3 equivalents) in water (60 mL) quickly but dropwise, while keeping the temperature of the reaction mixture at 0° C. The reaction mixture was stirred at 0° C. for 45 min. Dichloromethane (60 mL) was added to the reaction mixture and the phases were separated. The organic phase was washed with an HCl aqueous solution (0.1 N, 60 mL), and aqueous phases were combined and washed twice with dichloromethane. The aqueous phase was basified with an aqueous solution of Na₂CO₃ (2 N) to pH 7, and cooled to 0° C. To the above-described solution was added NaBH₄ (1.5 equivalents) and the mixture was stirred at 0° C. for 90 min. The reaction mixture was extracted with dichloromethane (3×200 mL). The organic phases were combined, dried over MgSO₄, and concentrated under reduced pressure. The crude products containing amino lactones or γ-hydroxy-α-amino-esters were purified by silica gel column chromatogaphy to obtain the pure compounds.

### General Procedure for Reduction of γ-oxo-α-amino-esters with Sodium Borohydride:

To a solution of γ-oxo-α-amino-esters (10 mmol) in MeCN (6 mL) was added NaBH₄ (1.2 equivalents) and the reaction mixture was stirred for 90 min. Water (40 mL) was added to neutralize the excess hydride, followed by addition of dichloromethane (40 mL). After separating the phases, the aqueous phase was extracted with dichloromethane (2×50 mL). The organic phases were combined, dried over MgSO₄, and concentrated under reduced pressure. The crude γ-hydroxy-α-amino-esters were purified by silica gel column chromatography to obtain pure products.

### General Procedure for Reduction of γ-oxo-α-amino-esters with Sodium Borohydride and CeC/₃.7H₂O:

To a solution of γ-oxo-α-amino-esters (10 mmol) in MeOH (30 mL) at 0° C. was added CeCl₃.7H₂O (0.4 equivalent). The reaction mixture was stirred for 5 min at 0° C., followed by addition of NaBH₄ (1.2 equivalent), and stirring for 90 min. Water (40 mL) was added to neutralize the excess hydride, followed by addition of dichloromethane (40 mL). After separating the phases, the aqueous phase was extracted with dichloromethane (2×50 mL). The organic phases were combined, dried over MgSO₄, and concentrated under reduced pressure. The crude γ-hydroxy-α-amino-esters were purified by silica gel column chromatography to obtain pure products.

### General Procedure for Reduction of γ-oxo-α-amino-esters with Raney Nickel:

To a solution of γ-oxo-α-amino-esters (10 mmol) in MeOH (30 mL) at room temperature, many spatulas of commercially available Raney Nickel were added to obtain a grey-black solution, and the reaction mixture was stirred vigorously. The reaction mixture was cooled to 0° C. and purged with hydrogen gas. The reaction mixture was stirred under hydrogen atmosphere (1 atm) at room temperature for 24 h. The crude reaction mixture was filtered through celite, followed by purification of the complex reaction mixture, containing amino lactones and/or γ-hydroxy-α-amino-esters, by silica gel column chromatography to obtain pure products.

The following compounds were prepared using the general procedures described above.

### Synthesis of Compound 8b

8b: Following a one step deprotection-reduction sequence, a diastereomeric mixture was obtained, 56%, as a clear oil. ¹H NMR (CDCl₃, 300 MHz): δ 0.77 (d, ³J (H₆, H₅)=7.2 Hz, 3H, H₆), 0.91 (t, ³J (Hg, H8)=7.2 Hz, 3H, Hg), 1.25 (t, ³J (H₁, H₂)=7.2 Hz, 3H, H₁), 1.31-1.59 (m, 1H, H₇), 1.99 (m, 1H, H₅), 3.62 (d, ³J (H₄, H₅)=2.8 Hz, 1H, H₄), 3.78 (m, 1H, H₇), 4.16 (q, ³J (H₂, H₁)=7.2 Hz, 2H, H₂).

### Synthesis of Compound 9b

9b: Following either a one step deprotection-reduction sequence or reduction of unprotected ethyl esters, a diastereomeric mixture was obtained, 40%, as a clear oil. ¹H NMR (CDCl₃, 300 MHz): δ 1.07 (t, ³J (H₈, H₇)=7.5 Hz, 3H, H₈), 1.23 (d, ³J (H₅, H₄)=5.3 Hz, 3H, H₅), 1.63 (m, 1H, H₄), 1.85 (m, 1H, H₇), 3.24 (d, ³J (H₂, H₄)=11.3 Hz, 1H, H₂), 3.91 (m, 1H, H₆).

¹H NMR (CDCl₃, 300 MHz): δ 1.06 (t, ³J (H₈, H₇)=7.2 Hz, 3H, H₈), 1.17 (d, ³J (H₅, H₄)=6.8 Hz, 3H, H₅), 1.43-1.67 (m, 1H, H₇), 2.34 (m, 1H, H₄), 3.26 (d, ³J (H₂, H₄)=10.5 Hz, 1H, H₂), 4.41 (m, 1H, H₆), MS (IC) m/z: 144 (M+1).

### Synthesis of Compound 8e

8e: Following either a one step deprotection-reduction sequence or reduction of unprotected ethyl esters with Raney Nickel, a diastereomeric mixture was obtained, 56%, as a clear oil. ¹H NMR (CDCl₃, 200 MHz): δ 1.23 (t, ³J (H₁, H₂)=7.1 Hz, 3H, H₁), 1.15-1.98 (m, 9H, H₅, H₇, H₈, H₉, H₁₀), 3.15 (brs, 3H, H₁₁, H₁₂), 3.46 (m, 1H, H₆), 3.61 (d, ³J (H₄₁, H₅)=2.7 Hz, 1H, H₄₁), 3.91 (d, ³J (H₄₂, H₅)=2.9 Hz, 1H, H₄₂), 4.14 (q, ³J (H₂, H₁)=7.1 Hz, 2H, H₂). ¹³C₁ NMR (CDCl₃, 50 MHz): δ 14.11 (C₁), 19.17, 25.33, 25.61 (C₈, C₉, C₁₀), 33.01 (C₇), 42.33 (C₅), 58.69 (C₄), 61.09 (C₂), 70.77 (C₆), 174.47 (C₃), ¹³C₂ NMR (CDCl₃, 50 MHz) δ 14.11 (C₁), 24.65, 25.07, 25.33 (C₈, C₉, C₁₀), 35.57 (C₇), 47.83 (C₅), 54.51 (C₄), 60.84 (C₂), 70.22 (C₆), 175.10 (C₃).

### Synthesis of compound (3S,3aS,8aS)-3-amino-octahydrocyclohepta[b]furan-2-one (9f-SSS)

9f (SSS): Following a one step deprotection-reduction sequence compound was obtained, 68%, as a clear oil. ¹H NMR (CDCl₃, 300 MHz): δ 1.12-2.37 (m, 10H, H₄, H₅, H₆, H₇, H8), 2.40 (m, 1H, H₃), 3.30 (d, ³J (H₂, H₃)=10.9 Hz, 1H, H₂), 4.51 (m, 1H, Hg). ¹³C NMR (CDCl₃, 75 MHz): δ 25.59, 25.70, 29.59, 30.67, 30.73 (C₄, C₅, C₆, C₇, C₈), 46.47 (C₃), 56.22 (C₂), 82.61 (C₉), 178.30 (C₁).

### Synthesis of compound (3S,3aS,8aR)-3-amino-octahydrocyclohepta[b]furan-2-one (9f-SSR)

9f (SSR): Following Raney Nickel reduction of amino ester intermediate, 55%, a clear oil was obtained. ¹H NMR (CDCl₃, 300 MHz): δ 1.10-2.25 (m, 11H, H₃, H₄, H₅, H₆, H₇, H₈), 3.23 (d, ³J (H₂, H₃)=11.5 Hz, 1H, H₂), 4.02 (m, 1H, Hg). ¹³C NMR (CDCl₃, 75 MHz): δ 24.24, 25.28, 27.11, 28.47, 32.78 (C₄, C₅, C₆, C₇, C₈), 50.42 (C₃), 58.23 (C₂), 82.04 (C₉), 178.04 (C₁).

### Synthesis of compound (2S,3S,4S)-3-amino-5-methyl-4-phenyl-dihydrofuran-2(3H)-one (9c-SSS)

9c (SSS): Obtained either from a one step deprotection-reduction step or from reduction of amino ester with NaBH₄ or NaBH₄/CeCl₃.7H₂O, 37%, as a clear oil. ¹H NMR (CDCl₃, 200 MHz): δ 0.99 (d, ³J (H₅, H₄)=6.6 Hz, 3H, H₅), 1.57 (brs, 2H, H₁₂), 3.62 (dd, ³J (H₃, H₂)=11.7 Hz, ³J (H₃, H₄)=8.1 Hz, 1H, H₃), 4.09 (d, ³J (H₂, H₃)=11.7 Hz, 1H, H₂), 4.86 (quint, ³J (H₄, H₅)=³J (H₄, H₃)=7.1 Hz, 1H, H₄), 7.21-7.37 (m, 5H, H₇, H₈, H₉, H₁₀, H₁₁), ¹³C NMR (CDCl₃, 50 MHz): δ 16.88 (C₅), 52.07, 52.60 (C₂, C₃), 77.10 (C₄), 127.76, 128.96 (C₇, C₈, C₉, C₁₀, C₁₁), 135.11 (C₆), 177.66 (C₁).

### Synthesis of compound (2S,3S,4R)-3-amino-5-methyl-4-phenyl-dihydrofuran-2(3H)-one (9c-SSR)

9c (SSR): Obtained from a reduction of amino ester with Raney Nickel, 37%, as a clear oil. ¹H NMR (CDCl₃, 300 MHz): δ 1.41 (d, ³J (H₅, H₄)=6.0 Hz, 3H, H₅), 1.76 (brs, 2H, H₁₂), 2.93 (t, ³J (H₃, H₂)=³J (H₃, H₄)=11.1 Hz, 1H, H₃), 3.94 (d, ³J (H₂, H₃)=12.1 Hz, 1H, H₂), 4.53 (m, 1H, H₄), 7.27-7.41 (m, 5H, H₇, H8, H₉, H₁₀, H₁₁). ¹³C NMR (CDCl₃, 75 MHz): δ 18.48 (C₅), 58.63, 59.11 (C₂, C₃), 78.79 (C₄), 127.56, 129.08 (C₇, C₈, C₁₀, C₁₁), 127.68 (C₉), 135.80 (C₆), 176.60 (C₁).

### Synthesis of Compound 9d

9d: Obtained from a one step deprotection-reduction sequence, 1:1 diastereomeric mixture, 68%, as a clear oil. ¹H₁NMR (CDCl₃, 300 MHz): δ 1.25 (d, ³J (H₁₂, H₁₁)=6.0 Hz, 3H, H₁₂), 2.14 (m, 1H, H₃), 2.74-3.11 (m, 2H, H₄), 3.45 (d, ³J (H₂, H₃)=11.3 Hz, 1H, H₂), 4.20 (m, 1H, H₁₁), 7.20-7.37 (m, 5H, H₆, H₇, H₈, H₉, H₁₀). ¹³C, NMR (CDCl₃, 75 MHz): δ 19.17 (C₁₂), 35.98 (C₄), 53.34 (C₃), 56.42 (C₂), 78.01 (C₁₁), 126.64 (C₈), 128.58, 128.85 (C₆, C₇, C₉, C₁₀), 138.05 (C₅), 177.32 (C₁₋). 1H₂ NMR (CDCl₃, 300 MHz): δ 1.33 (d, ³J (H₁₂, H₁₁)=6.8 Hz, 3H, H₁₂), 2.72 (m, 1H, H₃), 2.74-3.11 (m, 2H, H₄), 3.52 (d, ³J (H₂, H₃)=10.9 Hz, 1H, H₂), 4.66 (m, 1H, H₁₁), 7.20-7.37 (m, 5H, H₆, H₇, H₈, Hg, H₁₀). ¹³C₂ NMR (CDCl₃, 75 MHz): δ 15.92 (C₁₂), 33.88 (C₄), 47.89 (C₃), 53.91 (C₂), 76.12 (C₁₁), 126.44 (C₈), 128.21, 128.58 (C₆, C₇, C₉, C₁₀), 137.51 (C₅), 177.76 (C₁).

### Synthesis of Compound 11b

11b: Obtained from a one step deprotection-reduction sequence or reduction of the amino ethyl ester, a diastereomeric mixture, 40%, as a clear oil. ¹H₁ NMR (CDCl₃, 300 MHz): δ 1.03 (m, 6H, H₈, H₅), 1.51-1.75 (m, 2H, H₇, H₄), 3.73 (d, ³J (H₂, H₄)=7.8 Hz, 1H, H₂), 3.86 (m, 1H, H₆). ¹H₂NMR (CDCl₃, 300 MHz): δ 0.90 (d, ³J (H₅, H₄)=7.2 Hz, 3H, H₅), 1.04 (t, ³J (H₈, H₇)=7.5 Hz, 3H, H₈), 1.56-1.84 (m, 1H, H₇), 2.57 (m, 1H, H₄), 3.83 (d, ³J (H₂, H₄)=6.9 Hz, 1H, H₂), 4.26 (m, 1H, H₆). ¹³C₂ NMR (CDCl₃, 50 MHz): δ 6.45 (C₈), 9.84 (C₅), 23.08 (C₇), 38.15 (C₄), 56.14 (C₂), 81.73 (C₆), 178.45 (C₁). MS (IC) m/z: 144 (M+1).

### Synthesis of (S)-ethyl 2-amino-2-((1R,2S)-2-hydroxycyclohexyl)acetate (8e-SSR)

8e (SSR): Obtained from a one step deprotection-reduction sequence, 62%, as a clear oil. ¹H NMR (CDCl₃, 300 MHz): δ 1.24 (t, ³J (H₁, H₂)=7.2 Hz, 3H, H₁), 1.00-1.91 (m, 9H, H₅, H₇, H₈, H₉, H₁₀), 3.49 (m, 5H, H₁₁, H₁₂, H₆, H₄), 4.13 (q, ³J (H₂, H₁)=7.2 Hz, 2H, H₂). ¹³C NMR (CDCl₃, 75 MHz): δ 14.07 (C₁), 24.09, 25.28, 27.78 (C₈, C₉, C₁₀), 34.94 (C₇), 46.96 (C₅), 60.37 (C₄), 60.70 (C₂), 75.19 (C₆), 174.65 (C₃).

### Synthesis of Compound 11f

11f: A diastereomeric mixture of amino lactones was obtained either from a one step deprotection-reduction sequence or reduction of the corresponding amino ester with Raney Nickel, 72%, obtained as a clear oil. ¹H₁ NMR (CDCl₃, 200 MHz): δ 1.18-2.55 (m, 11H, H₃, H₄, H₅, H₆, H₇, H₈), 3.82 (d, ³J (H₂, H₃)=8.1 Hz, 1H, H₂), 4.61 (m, 1H, Hg). ¹³C₁ NMR (CDCl₃, 50 MHz): δ 20.63, 21.38, 28.40, 30.45, 31.15 (C₄, C₅, C₆, C₇, C₈), 45.51 (C₃), 54.68 (C₂), 80.28 (C₉), 178.44 (C₁). 1H₂ NMR (CDCl₃, 200 MHz): δ 1.18-2.57 (m, 11H, H₄, H₅, H₆, H₇, H₈, H₃), 3.61 (d, ³J (H₂, H₃)=6.8 Hz, 1H, H₂), 4.44 (m, 1H, Hg). ¹³C₂ NMR (CDCl₃, 50 MHz): δ 22.90, 24.30, 25.42, 26.71, 33.10 (C₄, C5, C₆, C₇, C₈), 46.00 (C₃), 54.68 (C₂), 83.80 (C₉), 177.94 (C₁).

### Synthesis of (2S,3R,4R)-ethyl 2-amino-4-hydroxy-3-phenylpentanoate (10c-SRR)

10c (SRR): Obtained from a one step deprotection-reduction sequence, 60%, as a clear oil. ¹H NMR (CDCl₃, 200 MHz): δ 1.02 (t, ³J (H₁, H₂)=7.1 Hz, 3H, H₁), 1.09 (d, ³J (H₇, H₆)=6.4 Hz, 3H, H₇), 2.59 (brs, 3H, H₁₄, H₁₅), 2.93 (dd, ³J (H₅, H₆)=3.2 Hz, ³J (H₅, H₄)=8.1 Hz, 1H, H₅), 3.98 (q, ³J (H₂, H₁)=7.1 Hz, 2H, H₂), 4.00 (d, ³J (H₄, H₅)=8.1 Hz 1H, H₄), 4.34 (m, 1H, H₆), 7.06-7.33 (m, 5H, H₉, H₁₀, H₁₁, H₁₂, H₁₃). ¹³C NMR (CDCl₃, 50 MHz): δ 13.70 (C₁), 20.40 (C₇), 54.40 (C₅), 57.14 (C₄), 60.65 (C₂), 68.05 (C₆), 126.89 (C₁₁), 128.05, 129.56 (C₉, C₁₀, C₁₂, C₁₃), 138.24 (C₈), 174.38 (C₃).

### Synthesis of (2S,3R,4S)-ethyl 2-amino-4-hydroxy-3-phenylpentanoate (10c-SRS)

10c (SRS): Obtained from reduction of amino ester with NaBH₄ or NaBH₄/CeCl₃.7H₂O as a clear oil. ¹H NMR (CDCl₃, 200 MHz) δ 0.82 (t, ³J (H₁, H₂)=7.2 Hz, 3H, H₁), 0.91 (d, ³J (H₇, H₆)=6.2 Hz, 3H, H₇), 2.71 (brs, 4H, H₁₄, H₁₅, H₅), 3.76 (m, 1H, H₅), 3.86 (d, ³J (H₄, H₅)=10.0 Hz 1H, H₄), 3.98 (q, ³J (H₂, H₁)=7.1 Hz, 2H, H₂), 7.06-7.33 (m, 5H, Hg, H₁₀, H₁, H₁₂, H₁₃).

### Synthesis of (2S,3R,4S)-ethyl 2-amino-4-hydroxy-3-phenylpentanoate (11c-SRR)

11c (SRR): Obtained from reduction of amino ester with NaBH₄ or with Raney nickel, 37%, as a clear oil. ¹H NMR (CDCl₃, 300 MHz) δ: 1.16 (d, ³J (H₅, H₄)=6.5 Hz, 3H, H₅), 3.69 (m, 1H, H₃), 4.09 (d, ³J (H₂, H₃)=8.1 Hz, 1H, H₂), 4.84 (m, 1H, H₄), 7.08-7.39 (m, 5H, H₇, H₈, H₉, H₁₀, H₁₁). ¹³C NMR (CDCl₃, 75 MHz): δ 16.22 (C₅), 51.99, 56.00 (C₂, C₃), 76.75 (C₄), 127.87 (C₉), 128.85, 129.07 (C₇, C₈, C₁₀, C₁₁), 133.20 (C₆), 178.94 (C₁).

### Synthesis of Comnound 11d

11d: SSR isomer was obtained as a major product either from a one step deprotection-reduction sequence or from reduction of the corresponding amino ester with sodium borohydride, 60%, as a clear oil. The SSS isomer was obtained as a major product from the reduction of the corresponding amino ester with NaBH₄ or NaBH₄/CeCl₃, 75%, as a clear oil. ¹H₁ NMR (CDCl₃, 300 MHz): δ 1.26 (m, 3H, H₁₂), 2.24 (brs, 2H, H₁₃), 2.39-3.11 (m, 3H, H₄, H₃), 3.85 (d, ³J (H₂, H₃)=6.5 Hz, 1H, H₂), 4.14 (m, 1H, H₁₁), 7.19-7.33 (m, 5H, H₆, H₇, H₈, H₉, H₁₀). ¹³C (CDCl₃ 75 MHz): δ 20.34 (C₁₂), 30.65 (C₄), 46.82 (C₃), 55.08 (C₂), 68.22 (C₁₁), 126.11 (C₈), 128.66 (C₆, C₇, C₉, C₁₀), 139.74 (C₅), 174.21 (C₁). ¹H₂NMR (CDCl₃, 300 MHz): δ 1.26 (m, 3H, H₁₂), 2.24 (brs, 2H, H₁₃), 2.39-3.11 (m, 3H, H₄, H₃), 3.89 (d, ³J (H₂, H₃)=7.2 Hz, 1H, H₂), 4.42 (m, 1H, H₁₁), 7.19-7.33 (m, 5H, H₆, H₇, H₈, H₉, H₁₀). ¹³C₂ NMR (CDCl₃, 75 MHz): δ 19.80 (C₁₂), 32.00 (C₄), 47.40 (C₃), 52.56 (C₂), 78.07 (C₁₁), 126.51 (C₈), 128.66 (C₆, C₇, C₉, C₁₀), 138.46 (C₅), 178.02 (C₁).

### General Procedure for Hydrolysis of Aminolactones and/or γ-hydroxy-α-amino esters

To a solution of amino lactones and/or γ-hydroxy-α-aminoesters in H₂O/MeOH (0.35 M) was added 1.2 equivalents of LiOH. The reaction mixture was stirred at room temperature for 24 h, followed by additon of 1.2 equivalents of acetic acid. The solvent was removed under reduced pressure, and the crude product was purified by recrystallization and/or using Dowex.

The following compounds were prepared using the general procedures as described above.

### Synthesis of (2S,3S,4S)-2-amino-4-hydroxy-3-methylhexanoic acid (12b)

12b: 75% as a white solid. ¹H NMR (D₂O, 300 MHz): δ 0.90 (d, ³J (H₇, H₃)=7.1 Hz, 3H, H₇), 0.93 (t, ³J (H₆, H₅)=7.2 Hz, 3H, H₆), 1.56 (m, 2H, H₅), 2.35 (m, 1H, H₃), 3.84 (m, 1H, H₄), 3.88 (d, ³J (H₂, H₃)=2.65 Hz, 1H, H₂). ¹³C NMR (D₂O, 75 MHz): δ 5.77 (C₆), 9.86 (C₇), 27.76 (C₅), 36.74 (C₃), 60.48 (C₂), 77.05 (C₄), 174.51 (C₁). MS (EI) m/z: 132.0675 (M−C₂H₅); 150° C.

### Synthesis of (2S,3S,4R)-2-amino-4-hydroxy-3-methylhexanoic acid (13b)

13b: 75% as a white solid. ¹H NMR (D₂O, 300 MHz): δ 0.96 (t, ³J (H₆, H₅)=7.2 Hz, 3H, H₆), 0.99 (d, ³J (H₇, H₃)=7.1 Hz, 3H, H₇), 1.50-1.67 (m, 2H, H₅, H_{5′}), 2.23 (m, 1H, H₃), 3.56 (m, 1H, H₄), 3.99 (d, ³J (H₂, H₃)=3.01 Hz, 1H, H₂). ¹³C NMR (D₂O, 75 MHz): δ 9.52 (C₆), 11.78 (C₇), 27.48 (C₅), 38.02 (C₃), 56.11 (C₂), 75.38 (C₄), 174.77 (C₁). MS (EI) m/z: 116.1068 (M-CO₂H); 165° C.

### Synthesis of (S)-2-amino-2-((1S,2S)-2-hydroxycyclohexyl)acetic acid (12e)

12e: 60% as a white solid. ¹H NMR (D₂O, 300 MHz): δ 1.24-2.01 (m, 8H, H₅, H₆, H₇, H₈), 2.13 (m, 1H, H₃), 3.84 (d, ³J (H₂, H₃)=3.0 Hz, 1H, H₂), 4.22 (m, 1H, H₄). ¹³C NMR (D₂O, 75 MHz) δ: 19.07, 20.20, 25.27 (C₆, C₇, C₈), 33.27 (C₅), 41.11 (C₃), 59.86 (C₂), 70.69 (C₄), 174.44 (C₁). MS (EI) m/z: 128.1070 (M—CO₂H); 175° C.

### Synthesis of (S)-2-amino-2-((1S,2R)-2-hydroxycyclohexyl)acetic acid (13e)

13e: 60% as a white solid. ¹H NMR (D₂O, 300 MHz): δ 1.19-1.40 (m, 4H), 1.62-1.80 (m, 3H), 1.85-2.05 (m, 2H), 3.46 (m, 1H, H₄), 3.98 (d, ³J (H₂, H₃)=2.8 Hz, 1H, H₂). ¹³C (D₂O, 75 MHz): δ (ppm): 24.41, 25.24, 26.44 (C₆, C₇, C₈), 35.49 (C₅, 45.50 (C₃), 56.68 (C₂), 70.94 (C₄), 174.27 (C₁). MS (EI) m/z: 128.1083 (M-CO₂H), 170° C. MS (EI) m/z: 174 (M+H)⁺.

### Synthesis of (S)-2-amino-2-((1S,2S)-2-hydroxycycloheptyl)acetic acid (12f)

12f: 68% as a white solid. ¹H NMR (D₂O, 300 MHz): δ 1.34-1.98 (m, 10H, H₅, H₆, H₇, H₈, H₉), 2.32 (m, 1H, H₃), 3.88 (d, ³J (H₂, H₃)=2.2 Hz, 1H, H₂), 4.26 (m, 1H, H₄). ¹³C NMR (D₂O, 75 MHz): δ 20.89, 21.17, 27.63, 28.63 (C₆, C₇, C₈, C₉), 36.26 (C₇), 43.56 (C₃), 60.67 (C₂), 74.35 (C₄), 174.63 (C₁). MS (EI) m/z: 142.1237 (M-CO₂H); 185° C.

### Synthesis of (S)-2-amino-2-((1S,2R)-2-hydroxycycloheptyl)acetic acid (13f)

13f: 68% as a white solid. ¹H NMR (D₂O, 300 MHz): δ 1.39-1.92 (m, 10H, H₅, H₆, H₇, H₈, H₉), 2.10 (m, 1H, H₃), 3.70 (m, 1H, H₄), 3.99 (d, ³J (H₂, H₃)=2.5 Hz, 1H, H₂). ¹³C NMR (D₂O, 75 MHz): δ 21.43, 25.45, 27.25, 27.69 (C₆, C₇, C₈, C₉), 36.50 (C₅), 47.48 (C₃), 58.31 (C₂), 73.03 (C₄), 174.64 (C₁). MS (EI) m/z: 142.1222 (M-CO₂H); 170° C.

### Synthesis of (2S,3S,4S)-2-amino-4-hydroxy-3-phenylpentanoic acid (12c)

12c: 37% as a white solid. ¹H (D₂O, 300 MHz): δ 1.13 (d, ³J (H₅, H₄)=6.4 Hz, 1H, H₅), 3.20 (dd, ³J (H₃, H₄)=4.9 Hz, ³J (H₃, H₂)=6.5 Hz, 1H, H₃), 4.16 (d, ³J (H₂, H₃)=6.5 Hz, 1H, H₂), 4.43 (m, 1H, H₄), 7.3-7.45 (m, 5H, H₇, H₈, H₉, H₁₀, H₁₁); ¹³C NMR (D₂O, 50 MHz) δ 21.04 (C₅), 52.48 (C₃), 58.54 (C₂), 68.33 (C₄), 128.60 (C₉), 129.35, 130.36 (C₇, C8, C₁₀, C₁₁), 134.89 (C₆), 173.73 (C₁). MS (EI) m/z: 191.0934 (M−H₂O); 125° C.

### Synthesis of (2S,3S,4R)-2-amino-4-hydroxy-3-phenylpentanoic acid (13c)

13c: 37% as a white solid. ¹H NMR (D₂O, 300 MHz): δ 1.19 (d, ³J (H₅, H₄)=6.1 Hz, 3H, H₅), 3.30 (dd, ³J (H₃, H₄)=8.3 Hz, ³J (H₃, H₂)=4.2 Hz, 1H, H₃), 4.27 (d, ³J (H₂, H₃)=4.2 Hz, 1H, H₂), 4.35 (m, 1H, H₄), 7.29-7.45 (m, 5H, H₇, H₈, H₉, H₁₀, H₁₁). ¹³C NMR (D₂O, 75 MHz): δ 21.40 (C₅), 52.92 (C₃), 56.27 (C₂), 67.39 (C₄), 128.50 (C₉), 129.44 (C₇, C₈, C₁₀, C₁₁), 136.14 (C₆), 173.92 (C₁). MS (EI) m/z: 191.0932 (M−H₂O); 160° C.

### Synthesis of a mixture of (2S,3S,4S)-2-amino-3-benzyl-3-hydroxypentanoic acid (12d) and (2S,3S,4R)-2-amino-3-benzyl-3-hydroxypentanoic acid (13d)

12d and 13d: 60:40 mixture of diastereoisomers, 63% as a white solid. ¹H₁NMR (D₂O, 300 MHz): δ 1.24 (d, ³J (H₅, H₄)=6.4 Hz, 3H, H₅), 2.29 (m, 1H, H₃), 2.76 (m, 2H, H₆), 3.95 (m, 1H, H₄), 4.08 (d, ³J (H₂, H₃)=1.5 Hz, 1H, H₂), 7.28-7.42 (m, 5H, H8, H₉, H_{10, 11}, H₁₂). ¹³C₁ NMR (D₂O, 75 MHz): δ 21.17 (C₅), 32.46 (C₆), 46.72 (C₃), 54.95 (C₂), 67.03 (C₄), 126.99 (C₁₀), 129.12, 129.64 (C₈, C₉, C₁₁, C₁₂), 139.64 (C₇), 174.33 (C₁). ¹H₂NMR (D₂O, 300 MHz): δ 1.16 (d, ³J (H₅, H₄)=6.8 Hz, 3H, H₅), 2.61 (m, 1H, H₃), 2.66-2.97 (m, 2H, H₆), 3.90 (d, ³J (H₂, H₃)=1.9 Hz, 1H, H₂), 4.16 (m, 1H, H₄), 7.31-7.40 (m, 5H, H₈, Hg, H₁₀, H₁₁, H₁₂). ¹³C₂ NMR (D₂O, 75 MHz): δ 21.05 (C₅), 29.69 (C₆), 46.22 (C₃), 59.06 (C₂), 70.98 (C₄), 126.99 (C₁₀), 129.02, 129.34 (C₈, C₉, C11, C₁₂), 140.74 (C₇), 173.85 (C₁). MS (EI) m/z: 205.1124 (M−H₂O), 170° C. MS (EI) m/z: 223.1206 (M), 160° C.

### Synthesis of (2S,3R,4S)-2-amino-4-hydroxy-3-methylhexanoic acid (14b)

14b: 75% as a white solid. ¹H NMR (D₂O, 300 MHz): δ 0.96 (m, 6H, H₆, H₇), 1.60 (m, 2H, H₅), 2.01 (m, 1H, H₃), 3.60 (m, 1H, H₄), 3.90 (d, ³J (H₂, H₃)=4.1 Hz, 1H, H₂). ¹³C NMR (D₂O, 75 MHz): δ 9.30 (C₆), 12.59 (C₇), 27.51 (C₅), 39.61 (C₃), 57.27 (C₂), 75.35 (C₄), 174.20 (C₁). MS (EI) m/z: 132.0661 (M−C₂H₅), 140° C.

### Synthesis of (2S,3R,4R)-2-amino-4-hydroxy-3-methylhexanoic acid (15b)

15b: 75% as a white solid. ¹H NMR (D₂O, 300 MHz): δ 0.89 (t, ³J (H₆, H₅)=7.1 Hz, 3H, H₆), 1.06 (d, ³J (H₇, H₃)=7.3 Hz, 3H, H₇), 1.51 (m, 2H, H₅), 2.25 (m, 1H, H₃), 3.73 (m, 1H, H₄), 3.82 (d, ³J (H₂, H₃)=3.2 Hz, 1H, H₂). ¹³C NMR (D₂O, 75 MHz): δ 9.04 (C₆), 9.86 (C₇), 27.60 (C₅), 36.64 (C₃), 60.23 (C₂), 74.37 (C₄), 174.27 (C₁). MS (EI) m/z: 116.1079 (M-CO₂H), 115° C.

### Synthesis of (S)-2-amino-2-((1R,2S)-2-hydroxycyclohexyl)acetic acid (14e)

14e: 60% as a white solid. ¹H NMR (D₂O, 300 MHz): δ 1.05-2.05 (m, 9H, H₅, H₆, H₇, H₈, H₃), 3.65 (m, 1H, H₄), 3.87 (d, ³J (H₂, H₃)=4.9 Hz, 1H, H₂). ¹³C NMR (D₂O, 75 MHz): δ 24.36, 24.98, 26.84 (C₆, C₇, C₈), 35.42 (C₅), 45.88 (C₃), 57.65 (C₂), 72.55 (C₄), 173.97 (C₁); MS (EI) m/z: 128.1070 (M-CO₂H), 165° C.

### Synthesis of (S)-2-amino-2-((1R,2R)-2-hydroxycyclohexyl)acetic acid (15e)

15e: 60% as a white solid. ¹H NMR (D₂O, 300 MHz): δ 1.26-2.11 (m, 9H, H₃, H₅, H₆, H₇, H₈), 3.76 (d, ³J (H₂, H₃)=4.4 Hz, 1H, H₂), 4.12 (m, 1H, H₄). ¹³C NMR (D₂O, 75 MHz): δ 19.36, 23.78, 25.4 (C₆, C₇, C₈), 33.07 (C₅), 40.96 (C₃), 59.35 (C₂), 68.32 (C₄), 174.44 (C₁). MS (EI) m/z: 128.1083 (M-CO₂H); 120° C.

### Synthesis of (S)-2-amino-2-((1R,2S)-2-hydroxycycloheptyl)acetic acid (14f)

14f: 68% as a white solid. ¹H NMR (D₂O, 300 MHz): δ 1.32-1.81 (m, 10H, H₅, H₈, H₇, H₈, H₉), 2.19 (m, 1H, H₃), 3.82 (d, ³J (H₂, H₃)=3.7 Hz, 1H, H₂), 4.16 (m, 1H, H₄). ¹³C NMR (D₂O, 75 MHz): δ 21.12, 24.36, 26.94, 27.86 (C₆, C₇, C₈, C₉), 35.98 (C₅), 43.45 (C₃), 60.92 (C₂), 71.54 (C₄), 174.79 (C₁). MS (EI) m/z: 142.1236 (M-CO₂H), 165° C.

### Synthesis of (S)-2-amino-2-((1R,2R)-2-hydroxycycloheptyl)acetic acid (15f)

15f: 68% as a white solid. ¹H NMR (D₂O, 300 MHz): δ 1.32-1.89 (m, 11H, H₃, H₅, H₆, H₇, H₈, H₉), 3.90 (d, ³J (H₂, H₃)=3.4 Hz, 1H, H₂), 4.05 (m, 1H, H₄). ¹³C NMR (D₂O, 75 MHz): δ 21.89, 24.89, 27.07, 28.27 (C₆, C₇, C₈, C₉), 36.02 (C₅), 48.65 (C₃), 57.68 (C₂), 73.43 (C₄), 174.14 (C₁). MS (EI) m/z: 169.1105 (M−H₂O), 160° C.

### Synthesis of (2S,3R,4R)-2-amino-4-hydroxy-3-phenylpentanoic acid (15c)

15c: 37% as a white solid. ¹H NMR (D₂O, 300 MHz): δ 1.31 (d, ³J (H₅, H₄)=6.2 Hz, 3H, H₅), 3.08 (m, 1H, H₃), 4.14 (d, ³J (H₂, H₃)=5.0 Hz, 1H, H₂), 4.53 (m, 1H, H₄), 7.37-7.42 (m, 5H, H₇, H₈, H₉, H₁₀, H₁). ¹³C NMR (MeOD, 50 MHz): δ 22.13 (C₅), 52.60 (C₃), 60.98 (C₂), 69.71 (C₄), 128.59 (C₉), 129.64, 131.47 (C₇, C₈, C₁₀, C₁₁), 138.01 (C₆), 173.26 (C₁). MS (EI) m/z: 191.0952 (M−H₂O), 180° C.

### Synthesis of (2S,3R,4S)-2-amino-3-benzyl-3-hydroxypentanoic acid (14d)

14d: 63% as a white solid. ¹H NMR (D₂O, 300 MHz): δ 1.31 (d, ³J (H₅, H₄)=6.4 Hz, 3H, H₅), 2.46 (m, 1H, H₃), 2.66-3.14 (m, 2H, H₆), 3.65 (d, ³J (H₂, H₃)=3 Hz, 1H, H₂), 4.12 (m, 1H, H₄), 7.33-7.43 (m, 5H, H₈, H₉, H₁₀, H₁₁, H₁₂). ¹³C NMR (D₂O, 75 MHz): δ 20.79 (C₅), 30.03 (C₆), 45.77 (C₃), 56.95 (C₂), 68.17 (C₄), 127.16 (C₁₀), 129.39 (C₈, C₉, C₁₁, C₁₂), 139.43 (C₇), 174.38 (C₁). MS (EI) m/z: 223.1206 (M), 225° C.

### Synthesis of (2S,3R,4R)-2-amino-3-benzyl-3-hydroxypentanoic acid (15d)

15d: 63% as a white solid. ¹H NMR (D₂O, 300 MHz): δ 1.26 (d, ³J (H₅, H₄)=6.5 Hz, 3H, H₅), 2.45 (m, 1H, H₃), 2.83 (m, 2H, H₆), 3.86 (d, ³J (H₂, H₃)=2.2 Hz, 1H, H₂), 3.91 (m, 1H, H₄), 7.32-7.44 (m, 5H, H₅, Hg, H₁₀, H₁₁, H₁₂). ¹³C NMR (D₂O, 75 MHz): δ 21.49 (C₅), 34.81 (C₆), 46.87 (C₃), 55.19 (C₂), 67.99 (C₄), 127.14 (C₁₀), 129.25, 129.57 (C₈, C₉, C₁₁, C₁₂), 139.43 (C₇), 174.44 (C₁). MS (EI) m/z: 205.1099 (M−H₂O), 180° C.

### Synthesis of Compound 17

A solution of 4-hydroxyproline methyl ester hydrochloride (16) (10.0 g, 55.3 mmol) and chlorotrimethylsilane (15.0 g, 138.1 mmol) in dichloromethane (200 mL) was stirred at 0° C. To this solution was added triethylamine (19.6 g, 193.4 mmol). The solution was then heated to reflux for 1 h. The mixture was cooled to 0° C., and a solution of methanol (3.3 mL) in dichloromethane (16.5 mL) was added. The reaction mixture was stirred at room temperature for 1 h. To the resulting mixture were added PhF—Br (17.7 g, 55.3 mmol), triethylamine (5.59 g, 55.3 mmol), and Pb(NO₃)₂ (16.5 g, 49.8 mmol). The mixture was stirred at room temperature under nitrogen for 12 h. The mixture was filtered and solvent was evaporated. The residue was redissolved in a solution of citric acid (23 g) in methanol (230 mL). The mixture was stirred at room temperature for 1 h. Solvent was evaporated, and the residue was redissolved in ethyl acetate (300 mL), and washed with water (200 mL) and brine. The organic layer was dried with magnesium sulfate and evaporated to obtain crude compound N-PhF-4-hydroxyproline methyl ester (17) (20 g, 94%) with 60% purity. It was used as such without further purification.

### Synthesis of Compound 18

A solution of oxalyl chloride (1.98 g, 15.6 mmol) in dry dichloromethane (45 mL) was stirred at −60° C. under nitrogen. To this solution was added DMSO (2.0 mL, 27.9 mmol) dropwise over a period of 5 min. The mixture was stirred for 15 min at the same temperature. Then, a solution of N-PhF-4-hydroxyproline methyl ester (17) (4.30 g, 11.15 mmol) in dichloromethane (45 mL) was added dropwise using an addition funnel over a period of 10 min. The reaction mixture was stirred at −60° C. for 45 min. Then, triethylamine (5.97 g, 59.0 mmol) was added to the mixture, and the temperature was allowed to reach 0° C. The reaction mixture was poured into an extraction funnel and was washed with water (50 mL). The organic layer was dried with magnesium sulfate and evaporated. The crude product was purified by silica gel chromatography to obtain pure N-PhF-4-oxoproline methyl ester (18) (2.3 g, 54%).

### Synthesis of Compound 19

A solution of N-PhF-4-oxoproline methyl ester (18) (3.00 g, 7.82 mmol) in THF (30 mL) and HMPA (3 mL) was stirred at −55° C. under nitrogen. To this solution was added a 2.5 M solution of butyllithium in hexane (3.30 mL, 8.22 mmol). The mixture was stirred at −55° C. for 1 h. Then iodomethane (1.46 mL, 23.46 mmol) was added and the reaction mixture was allowed to reach −10° C. The mixture was stirred at this temperature for 30 min. It was then cooled to −50° C. and a 10% solution of H₃PO₄ (10 mL) was added. The mixture was extracted with ether (2×50 mL). The combined organic phase was washed with brine and dried over magnesium sulfate. The solvent was removed under reduced pressure and the crude product was purified by silica gel chromatography to obtain pure N-PhF-3-methyl-4-oxoproline methyl ester (19) (1.0 g; 30%). 19: ¹H NMR (500 MHz, CDCl₃): δ 7.71 (m, 2H), 7.50 (m, 2H), 7.41-7.37 (m, 4H), 7.28-7.23 (m, 5H), 3.75 (d, 1H); 3.35 (d, 1H), 3.27 (d, 1H), 3.11 (s, 3H), 2.53 (m, 1H), 1.05 (d, 3H).

### Synthesis of Compound 23

A solution of N-PhF-4-oxoproline methyl ester (18) 34 g, 2.17 mmol) in THF (50 mL) and HMPA (15 mL) was stirred at −78° C. under nitrogen. To this solution was added a 0.5 M solution of KHMDS in toluene (17.4 mL, 8.70 mmol). The mixture was stirred at −78° C. for 1 h. Then iodomethane (1.35 mL, 21.7 mmol) was added and the reaction mixture was stirred for 12 h. To this mixture was added a 10% aqueous solution of KH₂PO₄. The mixture was extracted with ethyl acetate (2×25 mL). The organic extracts were collected, washed with brine, dried with sodium sulfate, and concentrated under reduced pressure. The crude compound was dissolved in hexane:ethyl acetate (3:1) and filtered on silica gel to obtain pure N-PhF-3,3-dimethyl-4-oxoproline methyl ester (23) (0.63 g. 70%). 23: ¹H NMR (500 MHz, CDCl₃): δ 7.74 (d, 1H), 7.67 (d, 1H), 7.43-7.25 (m, 11H), 3.97 (d, 1H), 3.75 (d, 1H), 3.43 (s, 1H), 2.95 (s, 3H), 1.37 (s, 3H), 0.84 (s, 3H).

### Synthesis of Compound 27

A solution of N-PhF-4-oxoproline methyl ester (18) (1.30 g, 3.39 mmol) in THF (10 mL) and HMPA (15 mL) was stirred at −78° C. under nitrogen. To this solution was added a 1.0 M solution of LiHMDS in THF (8.80 mL, 8.80 mmol). The mixture was stirred at −78° C. for 1 h. Acetaldehyde (1.75 eq) was added, and the reaction mixture was allowed to reach−55° C. After stirring for 3 h, 10% aqueous solution of H₃PO₄ (5 mL) was added. The mixture was extracted with ether (2×25 mL). The organic extracts were collected, washed with brine, dried with sodium sulfate, and concentrated under reduced pressure. The crude compound was purified by silica gel chromatography to afford pure N-PhF-3-(2-hydroxy-ethyl)-4-oxoproline methyl ester (27). ¹H NMR was in accord with the structure.

### Synthesis of Compound 28

A solution of N-PhF-4-oxoproline methyl ester (18) (1.30 g, 3.39 mmol) in THF (10 mL) and HMPA (15 mL) was stirred at −78° C. under nitrogen. To this solution was added a 1.0 M solution of LiHMDS in THF (8.80 mL, 8.80 mmol). The mixture was stirred at −78° C. for 1 h. Then benzaldehyde (600 μL, 5.93 mmol, 1.75 eq.) was added and the reaction mixture was allowed to reach −55° C. After stirring for 3 h, a 10% aqueous solution of H₃PO₄ (5 mL) was added. The mixture was extracted with ether (2×25 mL). The organic extracts were collected, washed with brine, dried with sodium sulfate, and concentrated under reduced pressure. The crude compound was purified by silica gel chromatography to afford pure N-PhF-3-hydroxyphenylmethyl-4-oxoproline methyl ester (28) (0.98 g, 60%). ¹H NMR was in accord with the structure.

### Synthesis of Compound 20

A solution of N-PhF-3-methyl-4-oxoproline methyl ester (19) (1.00 g, 2.52 mmol) in THF/methanol (1:1) (20 mL) was stirred at −78° C. To this solution was added a solution of sodium borohydride (0.238 g, 6.29 mmol) in methanol (5 mL). The mixture was stirred for 5 days and reaction was still not complete. The mixture was allowed to reach −10° C. and was stirred for 2 h. LC-MS analysis showed the presence of two compounds of the same molecular weight, but with different retention times, i.e., two diastereoisomers. The reaction mixture was cooled at −70° C. and a 10% aqueous H₃PO₄ solution (10 mL) was added. After concentrating the mixture under reduced pressure, the resulting mixture was extracted with ethyl acetate (2×25 mL). The organic extracts were collected, washed with brine, dried with sodium sulfate, and concentrated. The crude compound was purified by silica gel chromatography to afford pure N-PhF-3-methyl-4-hydroxy-proline methyl ester (20) (0.485 g; 49%). 20: ¹H NMR (500 MHz, CDCl₃): δ 7.74 (d, 1H), 7.67 (d, 1H), 7.43-7.25 (m, 11H), 3.97 (d, 1H), 3.75 (d, 1H), 3.43 (s, 1H), 2.95 (s, 3H), 1.37 (s, 3H), 0.84 (s, 3H).

### Synthesis of Compound 24

A solution of N-PhF-3,3-dimethyl-4-oxoproline methyl ester (23) (0.860 g, 2.09 mmol) in THF/methanol (1:1) (12 mL) was stirred at −78° C. To this solution was added sodium borohydride (0.158 g, 4.18 mmol). The mixture was allowed to reach −10° C. and was stirred for 3 h, and then cooled at −70° C. and a 10% aqueous H₃PO₄ solution (10 mL) was added. After concentrating the reaction mixture under reduced pressure, the resulting mixture was extracted with ethyl acetate (2×25 mL). The organic extracts were collected, washed with brine, dried with sodium sulfate, and concentrated. The crude compound was purified by silica gel chromatography to afford pure N-PhF-3,3-dimethyl-4-hydroxyproline methyl ester (24) (600 mg, 69%). 24: ¹H NMR (500 MHz, CDCl₃): δ 7.75 (d, 1H), 7.60 (m, 3H), 7.54 (d, 1H), 7.44 (t, 1H), 7.30-7.21 (m, 6H), 7.08 (t, 1H), 4.14 (t, 1H), 3.58 (t, 1H), 3.33 (s, 3H), 2.95 (t, 1H), 2.69 (s, 1H), 0.79 (s, 3H), 0.50 (s, 3H).

### Synthesis of Compound 29

A solution of N-PhF-3-hydroxyphenylmethyl-4-oxoproline methyl ester (27) in THF/methanol (1:1) (20 mL) was stirred at −78° C. To this solution was added sodium borohydride (2.5 eq), and the mixture was stirred for 12 h before allowing the temperature to reach −10° C. 10% aqueous H₃PO₄ solution (10 mL) was added, and the mixture was concentrated under reduced pressure. The resulting mixture was extracted with ethyl acetate (2×25 mL). The organic extracts were collected, washed with brine, dried with sodium sulfate, and concentrated. The crude compound was purified by silica gel chromatography to afford N-PhF-3-(2-hydroxy-ethyl)-4-hydroxy-proline methyl ester (29) as an oil (1.3 g). The product was used for further reaction without any purification.

### Synthesis of Compound 30

A solution of N-PhF-3-hydroxyphenylmethyl-4-oxoproline methyl ester (28) (0.980 g, 1.97 mmol) in THF/methanol (1:1) (20 mL) was stirred at −78° C. To this solution was added sodium borohydride (0.187 g, 4.92 mmol). The mixture was stirred for 12 h and then was allowed to reach −10° C. LC-MS analysis showed a complete reaction. Therefore a 10% aqueous H₃PO₄ solution (10 mL) was added. The reaction mixture was concentrated under reduced pressure, and the resulting mixture was extracted with ethyl acetate (2×25 mL). The organic extracts were collected, washed with brine, dried with sodium sulfate, and concentrated to obtain pure N-PhF-3-hydroxyphenylmethyl-4-hydroxy-proline methyl ester (30) as an oil (1.3 g, with 85% purity). The product was used as such for next reaction without any further purification.

### Synthesis of Compound 21

A solution of N-PhF-3-methyl-4-hydroxyproline methyl ester (20) (0.485 g, 1.21 mmol) in ethanol (7 mL) was stirred at room temperature. To this solution was added a 4 N NaOH (6 mL, 24.3 mmol) solution and the mixture was heated to reflux for 5 days. The reaction mixture was neutralized with a 10% aqueous solution of KH₂PO₄ after LC-MS analysis showed no sign of the presence of the starting material. The mixture was extracted with ethyl acetate (2×25 mL). The organic extracts were collected, washed with brine, dried with sodium sulfate, and concentrated under reduced pressure. The crude product was purified by trituration with ethyl acetate/hexane, to afford N-PhF-3-methyl-4-hydroxyproline (21) (0.290 g; 62%) with a HPLC purity of 95%.

### Synthesis of Compound 25

A solution of N-PhF-3,3-dimethyl-4-hydroxyproline methyl ester (24) (0.595 g, 1.44 mmol) in THF (40 mL) was stirred in a Parr reactor at room temperature. To this solution was added (Boc)₂O (0.690 g, 3.17 mmol) and 10% palladium on carbon (200 mg). The reactor was sealed and hydrogen was added (75 psi). The mixture was stirred at room temperature for 12 h. After the reaction was complete, the mixture was filtered and evaporated. The crude compound was triturated with hexane and dried to afford Boc intermediate (25).

### Synthesis of Compound 26

The BOC intermediate (25) (0.163 g, 0.597 mmol) was dissolved in dioxane (3 mL) and concentrated HCl (3 mL) was added. The mixture was stirred at 60° C. for 4 days. At this stage, LC-MS showed completion of the reaction. The white precipitates formed during the reaction were filtered off, the filtrate was concentrated under reduced pressure, and water was removed using a freeze-dryer to afford compound 26.

### Synthesis of Compound 31

A solution of 860 mg N-PhF-3-(2-hydroxy-ethyl)-4-hydroxyproline methyl ester (29) (2 mmol) in ethanol (10 mL) was stirred at room temperature. To this solution was added a 2 N aqueous solution of NaOH (1.5 ml, 3.00 mmol) and the mixture was stirred at room temperature for 5 h. More NaOH pellets (0.100 g, 2.50 mmol) were added. The reaction mixture was stirred at room temperature for another 24 h. As HPLC revealed 25% conversion, 2 N aqueous solution of KOH (1.0 mL, 2.0 mmol) was added, and the mixture was stirred for 6 days. The reaction mixture was concentrated under reduced pressure, and the residue was redissolved in ethyl acetate (25 mL). The mixture was washed with HCl (0.5N). The organic layer was washed with brine, dried with sodium sulfate, and concentrated. The crude compound was purified by silica gel chromatography to afford pure N-PhF-3-(2-hydroxy-ethyl)-4-hydroxyproline (31) (400 mg, 48%).

### Synthesis of Compound 32

To a solution of N-PhF-3-hydroxyphenylmethyl-4-hydroxyproline methyl ester (30) (0.968 g, 1.97 mmol) in ethanol (10 mL), at room temperature, was added 2 N aqueous solution of NaOH (1.5 ml, 3 mmol) and the mixture was stirred for 5 h. As little progress was observed by HPLC, more NaOH(s) (0.100 g, 2.50 mmol) was added and the reaction mixture was stirred at room temperature for another 24 h. At this stage, 25% hydrolysis was observed (HPLC). Therefore, a 2 N aqueous solution of KOH (1.0 mL, 2.0 mmol) was added and the mixture was stirred for 6 more days. The reaction mixture was concentrated under reduced pressure and the residue was dissolved in ethyl acetate (25 mL). The mixture was washed with HCl (0.5 N), followed by washing of the organic layer with brine and drying with sodium sulfate. The reaction mixture was concentrated and the crude product was purified by silica gel chromatography to afford pure N-PhF-3-hydroxyphenylmethyl-4-hydroxyproline (32) (400 mg, 43%).

### Synthesis of Compound 22

A solution of N-PhF-3-methyl-4-hydroxyproline (21) (0.290 g, 0.752 mmol) in ethanol (45 mL) and acetic acid (5 mL) was stirred in a Parr reactor at room temperature. To this solution was added 10% palladium on carbon (0.400 g). The reactor was sealed and hydrogen was added (100 Psi). The mixture was stirred for 2 h. After completion, the catalyst was filtered off and solvent was removed under reduced pressure. Water was added (20 mL) to the reaction mixture, and the mixture was washed with ether (2×25 mL). Water/acetic acid was removed using 3 lyophilization procedures to obtain compound 22.

### Synthesis of Compound 33

A solution of N-PhF-3-hydroxyethyl-4-hydroxyproline (31) (0.300 g, 0.722 mmol) in ethanol (45 mL) and acetic acid (5 mL) was stirred in a Parr reactor at room temperature. To this solution was added 10% palladium on carbon (0.100 g). The reactor was sealed and hydrogen was added (100 Psi). The mixture was stirred for 1 h. After completion, the mixture was filtered and concentrated under reduced pressure. Water was added (20 mL) to the reaction mixture and the mixture was washed with ether (2×25 mL). Water/acetic acid mixture was removed using lyophilization cycles to afford compound 33.

### Synthesis of Compound 34

A solution of N-PhF-3-hydroxyphenylmethyl-4-hydroxyproline (32) (0.420 g, 0.880 mmol) in ethanol (45 mL) and acetic acid (5 mL) was stirred in a Parr reactor at room temperature. To this solution was added 10% palladium on carbon (0.100 g). The reactor was sealed and hydrogen was added (100 Psi). The mixture was stirred for 1 h. After completion, the mixture was filtered and concentrated under reduced pressure. Water was added (20 mL) to the reaction mixture and the mixture was washed with ether (2×25 mL). Water/acetic acid mixture was removed by lyophilization cycles to afford compound 34.

### Synthesis of Compound 35

Boc-proline methyl ester (10 g, 43.67 mmol) was dissolved in anhydrous tetrahydrofuran (100 mL). The solution was cooled to −78° C. To the cooled solution was added 2 M LDA solution (52.4 mmol, 26.2 mL). The enolization reaction was stirred for 45 min at −78° C., followed by addition of 1.2 equivalents of allyl bromide. The alkylation was allowed to proceed overnight at −78° C. The reaction mixture was then allowed to warm to −20° C. The reaction was finally quenched by adding saturated ammonium chloride solution (100 mL) followed by addition of ethyl acetate (100 mL), and the two layers were separated. The organic layer was washed with brine, dried over magnesium sulfate, and concentrated under reduced pressure to give a yellow oil. The crude product was purified by silica gel column chromatography to obtain pure 35 (6 g).

### Synthesis of Compound 36

To a solution of compound 35 in ethanol (30 mL) was added 2 equivalents of 4 N KOH aqueous solution, and the mixture was stirred for 48 h. The reaction mixture was concentrated under reduced pressure, followed by addition of water (50 mL). The basic solution was acidified using HCl 2 N to adjust the pH to 3. This was followed by the extraction of the reaction mixture with ethyl acetate (100 mL). The concentration of the organic phase and subsequent recrystallization from ethyl acetate/hexane mixture gave pure Boc-α-allylproline (36) (2.5 g).

### Synthesis of Boc-α-oxiranylmethylproline (37)

Boc-α-allylproline (36) (2 g) was dissolved in methylene chloride (40 mL) and THF (10 mL). m-Chloroperbenzoic acid (2 g) was added and the reaction was stirred for 24 h. The crude reaction mixture was concentrated and extracted with EtOAc/saturated bicarbonate solution. The crude epoxidized allylproline was purified by silica gel column chromatography to afford pure Boc-α-oxiranylmethylproline (37) (1.1 g).

### Synthesis of α-oxiranylmethyl-proline (38)

The above-obtained Boc-α-oxiranylmethylproline (37) was dissolved in methylene chloride (5 mL), to this was added trifluoroacetic acid (5 mL), and the reaction mixture was stirred overnight. The reaction mixture was concentrated under reduced pressure, followed by addition of methylene chloride and concentration of the mixture again. This was repeated three times, followed by addition of water (30 mL) and freeze-drying, twice, to yield pure α-oxiranylmethyl-proline (38) (680 mg). 38: MS: M+H⁺=172.

### Synthesis of Compound 39

To a solution of L-proline methylester hydrochloride (5 g, 30 mmol) in water (20 mL) was added an excess of propylene oxide (20 mL). An exothermic reaction was observed, and the mixture was stirred overnight. After concentrating the reaction mixture under reduced pressure, the crude product was purified by reverse-phase chromatography to give compound 39 (2.3 g, 42%). 39: MS: M+H⁺=188.

### Synthesis of Compound 40

The above-described methyl ester (39) was hydrolyzed in ethanol with 2 equivalents of 2 N aqueous KOH and stirred for 48 h. The reaction mixture was neutralized using HCl 0.5 N, before freeze-drying. The so-obtained crude product was purified by reverse-phase chromatography to obtain 40 (1.15 g, 52%) as a clear oil. 40: MS: M+H⁺=174.

### Synthesis of cyclohexanecarboxylic acid methoxy-methyl-amide (41)

A solution of cyclohexylcarboxylic acid (6.30 g, 49.1 mmol) in acetonitrile (30 mL) was stirred at room temperature. To this solution was added N,N-diisopropylethylamine (DIEA) (12.7 g, 98.3 mmol) and TBTU (16.6 g, 51.6 mmol). The mixture was stirred for 10 min. Then, a solution of N,O-dimethylhydroxylamine hydrochloride (5.75 g, 59.0 mmol) and DIEA (6.35 g, 49.1 mmol) in acetonitrile (30 mL) was added. The mixture was stirred at room temperature for 24 h. The reaction mixture was concentrated under reduced pressure, and the crude mixture was redissolved in ethyl acetate (250 mL) and washed with 0.5 N NaOH (2×100 mL), 0.5 N HCl (2×100 mL), and brine. The organic layer was dried with magnesium sulfate and concentrated. The resulting oil was redissolved in hexane/ethyl acetate (3:1) and filtered through silica gel. The mixture was concentrated to afford compound 41 (7.4 g, 88%). 41: ¹H NMR (500 MHz, CDCl₃): δ ¹H NMR (CDCl₃): 3.68 (s, 3H), 3.16 (s, 3H), 2.67 (m, 1H), 1.81-1.23 (m, 10H).

### Synthesis of cyclopentanecarboxylic acid methoxy-methyl-amide (42)

To a stirred solution of cyclopentylcarboxylic acid (6.00 g, 52.6 mmol) in acetonitrile (30 mL), at room temperature, was added DIEA (13.6 g, 105.1 mmol) and TBTU (17.7 g, 55.2 mmol), and the mixture was stirred for 10 min. Then, a solution of N,O-dimethylhydroxylamine hydrochloride (6.15 g, 63.1 mmol) and DIEA (6.79 g, 52.6 mmol) in acetonitrile (30 mL) was added. The reaction mixture was stirred at room temperature for 24 h. The reaction mixture was concentrated under reduced pressure, and the crude product was redissolved in ethyl acetate (250 mL) and washed with 0.5 N NaOH (2×100 mL), 0.5 N HCl (2×100 mL), and brine. The organic phase was dried with magnesium sulfate and concentrated. The resulting oil was redissolved in hexane/ethyl acetate (3:1) and filtered through silica gel. After removal of solvent, pure cyclopentanecarboxylic acid methoxy-methyl-amide (42) (8 g, 97%) was obtained.

### Synthesis of 1-cyclohexyl-ethanone (43)

A solution of cyclohexanecarboxylic acid methoxy-methyl-amide (41) (4.1 g, 23.9 mmol) in dry THF (45 mL) was stirred at −78° C. under nitrogen. To this solution was added a 1.6 M solution of methyllithium in THF (15 mL, 23.9 mmol). The reaction mixture was allowed to warm to 0° C., and the mixture was stirred for additional 1 h. A 0.5 M solution of HCl (40 mL) was added and the mixture was extracted with ethyl acetate (2×50 mL). The organic extracts were combined, dried with magnesium sulfate, and concentrated under reduced pressure to afford 1-cyclohexyl-ethanone (43) (2.83 g, 94%) as a colorless oil. 43: ¹H NMR (500 MHz, CDCl₃): δ 2.33 (m, 1H), 2.13 (s, 3H), 1.88-1.66 (m, 5H), 1.37-1.16 (m, 5H).

### Synthesis of 1-cyclopentyl-ethanone (44)

A solution of cyclopentanecarboxylic acid methoxy-methyl-amide (42) (6.20 g, 39.44 mmol) in dry THF (60 mL) was stirred at −78° C. under nitrogen. To this solution was added a 1.6 M solution of methyllithium in THF (24.6 mL, 39.44 mmol). The temperature of the reaction mixture was allowed to reach 0° C., and the mixture was stirred for 1 h. A 0.5 M solution of HCl (20 mL) was added and the mixture was extracted with ethyl acetate (2×50 mL). The organic extracts were combined, dried with magnesium sulfate, and evaporated to obtain 1-cyclopentyl-ethanone (44) (3.40 g, 77%) as a colorless oil. 44: ¹H NMR (500 MHz, CDCl₃): δ 2.86 (m, 1H), 2.16 (s, 3H), 1.84-1.57 (m, 8H).

### Synthesis of 4-cyclohexyl-2-hydroxy-4-oxo-but-2-enoic acid ethyl ester (47)

A solution of sodium ethoxide was prepared by dissolving sodium (1.00 g, 43.7 mmol) in dry ethanol (100 mL). To this solution, was added cyclohexylmethylketone (43) (4.60 g, 36.4 mmol) and diethyl oxalate (5.33 g, 36.4 mmol). The mixture was stirred for 2 h at room temperature. After removal of the solvent, water (25 mL) and ice (14 g) were added. The mixture was treated with concentrated HCl (7 mL) and then extracted with ethyl acetate (2×100 mL). The organic extracts were combined, washed with brine, and dried with sodium sulfate. The crude product obtained after concentrating the reaction mixture under reduced pressure was redissolved in hexane/ethyl acetate (3:1) and filtered through a plug of silica gel. The removal of solvent, afforded 4-cyclohexyl-2-hydroxy-4-oxo-but-2-enoic acid ethyl ester (47) (5.2 g, 63%) as an orange oil. 47: ¹H NMR (500 MHz, CDCl₃): δ 6.39 (s, 1H), 4.35 (q, 2H), 2.37 (m, 1H), 1.91-1.69 (m, 5H), 1.42-1.24 (m, 8H).

### Synthesis of 4-cyclopentyl-2-hydroxy-4-oxo-but-2-enoic acid ethyl ester (48)

A solution of sodium ethoxide was prepared by dissolving sodium (0.84 g, 36.4 mmol) in dry ethanol (80 mL). To this solution was added cyclopentylmethylketone (44) (3.40 g, 30.3 mmol) and diethyl oxalate (4.43 g, 30.3 mmol). The mixture was stirred for 12 h at room temperature. After removal of the solvent, water (15 mL) and ice (10 g) were added. The mixture was treated with concentrated HCl (5 mL) and then extracted with ethyl acetate (2×50 mL). The organic extracts were combined, washed with brine, and dried with sodium sulfate. After removal of the solvent, the crude product was redissolved in hexane/ethyl acetate (3:1) and filtered through silica gel. The removal of solvent gave 4-cyclopentyl-2-hydroxy-4-oxo-but-2-enoic acid ethyl ester (48) (3.7 g, 58%) as an orange oil. 48: ¹H NMR (500 MHz, CDCl₃): δ 6.39 (s, 1H), 4.35 (q, 2H), 2.89 (m, 1H), 1.82-1.64 (m, 8H), 1.36 (t, 3H).

### Synthesis of 2-hydroxy-4-oxo-4-phenyl-but-2-enoic acid ethyl ester (49)

A solution of sodium ethoxide was prepared by dissolving sodium (4.59 g, 200 mmol) in dry ethanol (450 mL). To this solution was added acetophenone (45) (20.0 g, 166.4 mmol) and diethyl oxalate (24.3 g, 166.4 mmol). The mixture was stirred for 12 h at room temperature. After removal of the solvent, water (80 mL) and ice (60 g) was added. The mixture was treated with concentrated HCl (25 mL), and extracted with ethyl acetate (2×200 mL). The organic extracts were combined, washed with brine, and dried with sodium sulfate. The crude product obtained after removal of the solvent was redissolved in hexane/ethyl acetate (3:1) and filtered through silica gel. After removal of the solvent under reduced pressure, 2-hydroxy-4-oxo-4-phenyl-but-2-enoic acid ethyl ester (49) (22 g, 60%) was obtained as an orange oil. 49: ¹H NMR (500 MHz, CDCl₃): δ 8.00 (d, 2H), 7.61 (t, 1H), 7.51 (t, 2H), 7.08 (s, 1H), 4.40 (q, 2H), 1.42 (t, 3H).

### Synthesis of 2-hydroxy-5,5-dimethyl-4-oxo-hex-2-enoic acid ethyl ester (50)

A solution of sodium ethoxide was prepared by dissolving sodium (2.75 g. 120 mmol) in dry ethanol (250 mL). To this solution was added pinacolone (46) (10.0 g, 99.8 mmol) and diethyl oxalate (14.6 g, 99.8 mmol). The mixture was stirred for 12 h at room temperature. After removal of the solvent, water (50 mL) and ice (25 g) was added. The mixture was treated with concentrated HCl (7 mL) and extracted with ethyl acetate (2×150 mL). The organic extracts were combined, washed with brine, and dried with sodium sulfate. The crude product obtained after removal of the solvent was redissolved in hexane/ethyl acetate (3:1) and filtered through silica gel. After removal of the solvent under reduced pressure, 2-hydroxy-5,5-dimethyl-4-oxo-hex-2-enoic acid ethyl ester (50) was obtained as a colorless oil (22 g, 60%). 50: ¹H NMR (500 MHz, CDCl₃): δ 6.54 (s, 1H), 4.35 (q, 2H), 1.38 (t, 3H), 1.22 (s, 9H).

### Synthesis of 5-cyclohexyl-isoxazole-3-carboxylic acid ethyl ester (51)

A solution of the above-described enone (47) (5.10 g, 22.4 mmol) in anhydrous ethanol/THF (1:1) (60 mL) was stirred at room temperature. To this solution was added hydroxylamine hydrochloride (1.72 g, 24.7 mmol) and the resulting mixture was stirred 12 h under nitrogen. The mixture was then heated to reflux with a soxlet filled with molecular sieves for 2 h. After cooling the reaction mixture, solvent was removed under reduced pressure. Water (100 mL) was added and the mixture was extracted with dichloromethane (2×100 mL). The organic extracts were collected and dried with sodium sulfate. After removal of the solvent, the crude product was purified by silica gel chromatography to afford 5-cyclohexyl-isoxazole-3-carboxylic acid ethyl ester (51) as a colorless oil (2.8 g, 56%). 51: ¹H NMR (500 MHz, CDCl₃): δ 6.37 (s, 1H), 4.42 (q, 2H), 2.83 (m, 1H), 2.06 (m, 2H), 1.81 (m, 2H), 1.75 (m, 1H), 1.48-1.26 (m, 8H).

### Synthesis of 5-cyclopentyl-isoxazole-3-carboxylic acid ethyl ester (52)

A solution of the cyclopentyl-enone (48) (3.70 g, 17.4 mmol) in anhydrous ethanol/THF (1:1) (50 mL) was stirred at room temperature. To this solution was added hydroxylamine hydrochloride (1.33 g, 19.1 mmol) and the resulting mixture was stirred for 12 h under nitrogen. The mixture was then heated to reflux with a soxlet filled with molecular sieves during 2 h. After cooling the reaction mixture, solvent was evaporated under reduced pressure. Water (50 mL) was added and the mixture was extracted with dichloromethane (2×50 mL). The organic extracts were combined, dried with sodium sulfate, and concentrated. The crude product was purified by silica gel chromatography to give 5-cyclopentyl-isoxazole-3-carboxylic acid ethyl ester (52) as a colorless oil (2 g, 55%). 52: ¹H NMR (500 MHz, CDCl₃): δ 6.38 (s, 1H), 4.42 (q, 2H), 3.25 (m, 1H), 2.11 (m, 2H), 1.80-1.69 (m, 6H), 1.41 (t, 3H).

### Synthesis of 5-phenyl-isoxazole-3-carboxylic acid ethyl ester (53)

A solution of the phenyl-enone (49) (5.00 g, 22.7 mmol) in anhydrous ethanol/THF (1:1) (60 mL) was stirred at room temperature. To this solution was added hydroxylamine hydrochloride (1.73 g, 25.0 mmol) and the resulting mixture was stirred for 12 h under nitrogen. The mixture was then heated to reflux with a soxlet filled with molecular sieves during 2 h. The mixture was allowed to cool down and the solvent was evaporated. Water (100 mL) was added and the mixture was extracted with dichloromethane (2×100 mL). The organic extracts were combined, dried with sodium sulfate, and concentrated. The crude product was purified by silica gel chromatography to give 5-phenyl-isoxazole-3-carboxylic acid ethyl ester (53) as a colorless oil (3.89 g, 79%). 53: ¹H NMR (500 MHz, CDCl₃): δ 7.80 (d, 2H), 7.50 (m, 3H), 6.93 (s, 1H), 4.47 (q, 2H), 1.44 (t, 3H).

### Synthesis of 5-tert-butyl-isoxazole-3-carboxylic acid ethyl ester (54)

A solution of tert-butyl-enone (50) (6.00 g, 30.0 mmol) in anhydrous ethanol/THF (1:1) (70 mL) was stirred at room temperature. To this solution was added hydroxylamine hydrochloride (2.29 g, 33.0 mmol) and the resulting mixture was stirred for 12 h under nitrogen. The mixture was then heated to reflux with a soxlet filled with molecular sieves during 2 h. The mixture was allowed to cool down and the solvent was evaporated. Water (100 mL) was added and the mixture was extracted with dichloromethane (2×100 mL). The organic extracts were combined, dried with sodium sulfate, and concentrated. The crude product was purified by silica gel chromatography to give 5-tert-butyl-isoxazole-3-carboxylic acid ethyl ester (54) as a colorless oil (3 g, 51%). 54: ¹H NMR (500 MHz, CDCl₃): δ 6.37 (s. 1H), 4.43 (q, 2H), 1.41 (t, 3H), 1.37 (s, 9H).

### Synthesis of 5-cyclohexyl-isoxazole-3-carboxylic acid (55)

A solution of cyclohexyl isoxazole ethyl ester (51) (2.80 g, 12.5 mmol) in ethanol (30 mL) was stirred at room temperature. To this solution was added a 2 M NaOH solution (9.4 mL, 18.8 mmol). Within a few minutes, precipitates were formed and the reaction mixture became a thick paste. TLC showed that the reaction was complete. To the reaction mixture was added 0.5 M HCl to adjust the pH to 3-4, and then the mixture was extracted with ethyl acetate (2×100 mL). The organic extracts were combined, washed with brine, dried over sodium sulfate, and concentrated to afford 5-cyclohexyl-isoxazole-3-carboxylic acid (55) as white crystals (2.2 g. 90%). 55: ¹H NMR (500 MHz, CDCl₃): δ 9.60 (broad, 1H), 6.44 (s, 1H), 2.86 (m, 1H), 2.08 (m, 2H), 1.83 (m, 2H), 1.74 (m, 1H), 1.50-1.28 (m, 5H).

### Synthesis of 5-cyclopentyl-isoxazole-3-carboxylic acid (56)

A solution of cyclopentyl isoxazole ethyl ester (52) (2.00 g, 9.56 mmol) in ethanol (30 mL) was stirred at room temperature. To this solution was added a 2 M NaOH solution (7.2 mL 14.4 mmol). After 5 min, TLC showed that the reaction was complete. To the reaction mixture was added 0.5 M HCl to adjust the pH to 3-4, followed by extraction with ethyl acetate (2×75 mL). The organic extracts were combined, washed with brine, dried over sodium sulfate, and concentrated to afford 5-cyclopentyl-isoxazole-3-carboxylic acid (56) as white crystals (1.6 g, 92%). 56: ¹H NMR (500 MHz, CDCl₃): δ 9.75 (broad, 1H), 6.45 (s, 1H), 3.26 (m, 1H), 2.13 (m, 2H), 1.80-1.70 (m, 6H).

### Synthesis of 5-phenyl-isoxazole-3-carboxylic acid (57)

A solution of phenyl-substituted isoxazole ethyl ester (53) (1.89 g, 8.70 mmol) in ethanol (30 mL) was stirred at room temperature. To this solution was added a 2 M NaOH solution (6.5 mL, 13.1 mmol). After 5 min, TLC showed that the reaction was complete. To the reaction mixture was added 0.5 M HCl to adjust the pH to 3-4, before extracting with ethyl acetate (2×75 mL). The organic extracts were combined, washed with brine, dried over sodium sulfate, and concentrated to afford 5-phenyl-isoxazole-3-carboxylic acid (57) obtained as a white solid (1.54 g, 94%). 57: ¹H NMR (500 MHz, CDCl₃): δ 9.4 (broad, 1H), 7.83 (d, 2H), 7.51 (m, 3H), 6.99 (s, 1H)

### Synthesis of 5-tert-butyl-isoxazole-3-carboxylic acid (58)

A solution of tert-butyl-substituted isoxazole ethyl ester (54) (2.97 g, 15.1 mmol) in ethanol (30 mL) was stirred at room temperature. To this solution was added a 2 M NaOH solution (11.3 mL, 22.6 mmol). After 5 min, TLC showed a complete reaction. To the reaction mixture was added 0.5 M HCl to adjust the pH to 3-4 before extracting with ethyl acetate (2×75 mL). The organic extracts were combined, washed with brine, dried over sodium sulfate, and concentrated to afford 5-tert-butyl-isoxazole-3-carboxylic acid (58) as a colorless oil (1.54 g; 94%). 58: ¹H NMR (500 MHz, CDCl₃): δ 6.44 (s, 1H), 1.39 (s, 9H).

### Synthesis of 2-amino-4-cyclohexyl-4-hydroxy-butyric acid (59)

A solution of the above-described cyclohexyl-substituted isoxazole carboxylic acid (55) (2.20 g, 11.3 mmol) in ethanol/water (1:1) (80 mL) was stirred in a Parr reactor at room temperature. To this solution was added a suspension of Raney-Ni (2 g) (pre-washed 5 times with ethanol/water (1:1)) in ethanol/water. The reactor was sealed and hydrogen was added (120 psi). The mixture was stirred at room temperature for 3 h. LC-MS analysis revealed that reaction was not complete. The mixture was stirred for another 12 h, and at this stage, LC-MS revealed that the starting material was entirely consumed, yet the major compound was a species with one non-hydrogenated double bond. The mixture was filtered and the catalyst was rinsed with ethanol and water. 10% palladium was added to the filtrate on carbon (0.6 g) and acetic acid (10 mL). The reactor was sealed and hydrogen was added (120 psi). The mixture was stirred for 12 h at room temperature. This was followed by heating of the mixture at 50° C. for 4 days with 180 psi pressure of hydrogen. The mixture was filtered, filtrate was concentrated under reduced pressure, and water was removed by lyophilization. So obtained greenish solid of 2-amino-4-cyclohexyl-4-hydroxy-butyric acid (59) was further purified by reverse-phase chromatography (100% water). The pure fractions were identified by LCMS, collected, and lyophilized. 59: MS: M+H⁺=202.

### Synthesis of 2-amino-4-cyclopentyl-4-hydroxy-butyric acid (60)

The procedure described above for compound 59 was followed to synthesize compound 60 using cyclopentyl-substituted isoxazole carboxylic acid (56) (1.48 g, 8.17 mmol) in ethanol/water (1:1) (60 mL), Raney-Ni (1.5 g), 10% palladium on carbon (0.6 g), acetic acid (10 mL), and heating at 50° C. for 4 days with 180 psi of hydrogen. The purification was carried out using reverse-phase chromatography. The pure fractions were identified by LCMS, collected, and lyophilized. 60: MS: M+H⁺=187.

### Synthesis of 2-amino-4-hydroxy-4-phenyl-butyric acid (61)

The procedure described above for compounds 59 and 60 was followed to synthesize compound 61 using phenyl-substituted isoxazole carboxylic acid (57) (0.800 g, 4.23 mmol) in ethanol/water (1:1) (40 mL), Raney-Ni (1 g), 10% palladium on carbon (0.6 g), acetic acid (10 mL), and heating at 50° C. for 4 days with 180 psi of hydrogen. The purification was carried out using reverse-phase chromatography. The pure fractions were identified by LCMS, collected, and lyophilized.

### Synthesis of 2-amino-4-hydroxy-5,5-dimethyl-hexanoic acid (62)

The procedure described above for compounds 59, 60, and 61 was followed to synthesize 2-amino-4-hydroxy-5,5-dimethyl-hexanoic acid (62) using tert-butyl-substituted isoxazole (58) (2.0 g, 11.8 mmol) in ethanol/water (1:1) (40 mL), Raney-Ni (2 g), 10% palladium on carbon (0.6 g), acetic acid (10 mL), and heating at 50° C. for 4 days with 180 psi of hydrogen. The purification was carried out using reverse-phase chromatography. The pure fractions were identified by LCMS, collected, and lyophilized. 62: MS: M+H⁺=17.

### Synthesis of 1-(1-phenylethyl)-6-ethoxycarbonyl-4-methyl-3,4-didehydropiperidine (63)

α-Methylbenzylamine (20 g) was dissolved in toluene (60 mL) and 50% ethylglyoxalate in toluene (20 mL). The flask was equipped with magenetic stir bar and Dean-Stark™ trap. The solution was refluxed (oil bath at 110° C.) for 90 minutes and cooled to room temperature. The crude reaction mixture was evaporated at 35° C. to yield a dark red oil. To this was added methylene chloride (150 mL), followed by the addition of isoprene (22.5 g). The mixture was cooled to −65° C. using a cryocool, and to this was added, dropwise, a mixture of trifluoroacetic acid (19 g) and BF₃.Et₂O (23.5 g). The temperature of the reaction solution was kept in the range of −65° C. to −55° C., the reaction was stirred at −65° C. for 90 minutes, and was then allowed to warm up to −15° C., followed by the addition of water and sodium bicarbonate to adjust pH of the mixture to 8. The organic layer was separated from the aqueous layer, and subsequently dried over MgSO₄. After evaporation, a red oil was obtained. The oil was filtered over silica gel using 95% hexane/ethylacetate. After evaporation, a yellow oil was obtained, which was crystallized from hexane at −75° C. The solids were filtered and subsequently recrystallized again from cold hexane to afford 1-[(1-phenylethyl]-6-ethoxycarbonyl-4-methyl-3,4-didehydropiperidine (63) as an off-white crystalline solid (8.3 g). 63: MS: M+H⁺: 274.

### Synthesis of 1-(1-phenylethyl)-6-ethoxycarbonyl-4-methyl I-3,4-didehydropiperidine (64)

Ethyl 4,5-dehydro-4-methylpipecolate (63) (2 g, 7.3 mmol) was dissolved in THF (40 ml). The reaction mixture was cooled to −78° C., followed by dropwise addition of a 1 M solution of BH₃.THF (21.9 mL, 21.9 mmol). The mixture was allowed to reach 0° C., and was stirred for 1 h at 0° C. A 3 N aqueous solution of NaOH (7.3 mL, 21.9 mmol) was added dropwise, followed by the addition of 30% H₂O₂ (˜2.5 mL, 21.9 mmol). The mixture was stirred at room temperature for 2 h. Water (20 mL) was added, THF was evaporated under reduced pressure, and the final product was extracted using ethyl acetate. A clear oil was obtained, which was purified by flash-chromatography, and the fractions containing the desired final product were identified using LCMS. 64: MS: M+H⁺: 292. ¹H NMR (500 MHz, CDCl₃): δ 7.4-7.2 (m, 5 Hₐ), 4.2 (t, 3H), 3.96 (m, 1H), 3.4 (m, 1H), 3.18 (m, 1H), 2.69 (m, 1H), 2.0-1.3 (m, 4H), 1.3 (m, 3H), 1.0 (d, 3H).

### Synthesis of 5-hydroxy-4-methyl-2-piperidine carboxylic acid (65)

Compound 64 was subjected to base hydrolysis in ethanol using 2 equivalents of 2 N NaOH overnight. The intermediate obtained from this reaction, N-phenylethyl-protected hydroxy-piperidine carboxylic acid, was hydrogenated (H₂, Pd/C 10%) overnight in ethanol/water. After filtration, the final product was lyophilized, purified by reverse phase chromatography (100% water), and lyophilized to obtain pure 5-hydroxy-4-methyl-2-piperidine carboxylic acid (65). 65: MS: M+H⁺=160.

### Synthesis of Compound 64a

Ethyl 4,5-dehydro-4-methylpipecolate (63) (1 g, 3.65 mmol) was dissolved in acetone/water (10 mL). To the solution was added Osmiumtetroxide (50 mg, 0.183 mmol, 5 mol %) and NMO (430 mg, 1 eq.). An exothermic reaction started immediately. The reaction was stirred overnight. HPLC analysis showed a mixture of two isomers in a ratio of ˜60/40 formed. The reaction mixture was concentrated under reduced pressure, and purified by flash silica gel chromatography to yield 20% of the desired compound (64a). 64a: MS: M+H⁺=308.

### Synthesis of 4-methyl-4,5-dihydroxypipecolic acid (65a)

Base hydrolysis of di-hydroxypipecolate (64a) in KOH/EtOH/water mixture was carried out overnight. The reaction mixture was neutralized to pH 7 using 0.5 N HCl, and the free acid was recovered by extraction from water/ethylacetate. Three extractions with ethyl acetate yielded the acid intermediate (310 mg) as a colorless oil. MS: M+H⁺=280. The removal of phenylethyl moiety was accomplished under hydrogenolysis conditions in ethanol/water, using Pd/C 10% (10 wt %), at a 120 PSI hydrogen pressure. After overnight reaction, the reaction mixture was filtered to remove the catalyst, and ethanol was evaporated. Water (20 mL) was added, and the product was lyophilized, followed by purification using RP-chromatography to yield 4-methyl-4,5-dihydroxypipecolic acid (65a) (125 mg). ¹H NMR of the compound 65a was in accord with the structure assigned and showed the presence of a mixture of isomers.

### Synthesis of N-(2-hydroxypropyl)-L-valine ethyl ester (67)

To a suspension of L-valine (2 g) in ethanol (50 mL) cooled to −10° C., was slowly added thionyl chloride (2 equivalents). The reaction mixture was then refluxed for 4 hours, and then left to stir overnight. After removal of solvents under reduced pressure, ethanol was added and the resultant suspension was concentrated again. The desired final product (66) (quantitative yield) was further dried in a dessicator over NaOH. 66: MS: M+H⁺=146. The above ethyl ester (2 g) was then dissolved in water (10 mL) in a sealed pyrex tube, and to this was added propylene oxide (2 g). The reaction mixture was stirred at 50° C. for 4 h, then cooled, concentrated under reduced pressure, and lyophilized. The crude product was purified by reverse-phase column chromatography to afford N-(2-hydroxypropyl)-L-valine ethyl ester (67) (1.5 g). 67: MS: M+H⁺=204. The disubstituted compound (68) was also isolated from the reaction mixture.

### Synthesis of N-(2-hydroxypropyl)-L-valine (69)

The hydrolysis of N-(2-hydroxypropyl)-L-valine ethyl ester (67) was carried out in ethanol using 2 N aqueous KOH (4 equivalents). The resulting mixture was then heated at 50° C. for 4 days. The mixture was evaporated, and water was added. The reaction product was neutralized to pH 7 using HCl (0.5 N). The mixture was lyophilized, and subsequently purified by reverse-phase column chromatography to give N-(2-hydroxypropyl)-L-valine (69) (1.02 g, 34%). 69: MS: M+H⁺=176.

### Synthesis of N-Boc trans-4-hydroxyproline (71)

trans-4-hydroxyproline (70) (5 g, 38 mmol) was dissolved in dioxane/water (1:1) (50 mL), and to the solution was added NaHCO₃ (80 mmol) and Boc anhydride (30 mmol, 6.5 gram). The reaction was stirred for 4 hours. NaHCO₃ was added to keep the pH above 7. The crude reaction mixture was acidified using 0.5 N HCl. Dioxane was evaporated. Boc-trans-4-hydroxyproline was recovered by extraction using EtOAc/water. The organic phase was dried using MgSO₄ and subsequently evaporated to yield N-Boc-4-hydroxyproline (71) as a clear oil (5.6 g, 82%).

### Synthesis of Compound 72

A solution containing N-Boc-trans-4-hydroxyproline (71) (5 g, 21.6 mmol) and triphenylphosphine (11.8 g, 45 mmol) in anhydrous THF (150 mL) was cooled to 4° C. in an ice bath. To this solution was added DEAD (6.5 mL, 45 mmol). The reaction was allowed to stir at room temperature for 24 hours. The reaction mixture was evaporated to give a yellow oil. The crude product was purified by silica gel column chromatography to give the desired cyclic lactone (72) (2.1 g, 45%).

### Synthesis of Compound 73

The cyclic lactone (72) (2.1 g, 9.8 mmol) was dissolved in dry methanol (100 mL). To the solution was added sodium azide (2.34 g, 36 mmol). The reaction mixture was heated overnight at 45° C. After evaporation of the crude reaction mixture, the obtained oil was purified by silica gel column chromatography to give N-Boc-cis-4-hydroxyproline methyl ester (73) (1.3 g, 54%).

### Synthesis of Compound 74

N-Boc-cis-4-hydroxyproline methyl ester (73) (1.3 g, 5.3 mmol) was dissolved in ethanol (20 mL). To the solution was added 2 N NaOH aqueous solution (5.3 mL, 10.6 mmol). The reaction was completed after 4 h, and was acidified with 10% citric acid. Ethanol was evaporated, and the final product recovered by extraction with ethylacetate/water. The organic layer was dried over sodium sulfate, filtered, and concentrated to yield N-Boc-cis-4-hydroxyproline (74) (960 mg, 78%)

### Synthesis of Compound 75

N-Boc-cis-4-hydroxyproline (74) (500 mg) was dissolved in 30% TFA/methylene chloride (10 mL). The reaction was stirred for 1 h and then concentrated under reduced pressure. Water (50 mL) was added, and cis-4-OH proline TFA salt was recovered by lyophilization to yield a yellowish solid. The yellow solid was treated with ether and acetone. The solid was redissolved three times in 50 mL water and lyophilized to obtain cis-4-hydroxyproline (75) (260 mg) as an off-white solid. 75: MS: M+H⁺=132. ¹H NMR (500 MHz, D₂O): δ 4.6 (m, 1H), 4.23 (m, 1H), 3.5 (m, 1H), 3.39 (m, 1H), 2.53 (m, 1H), 2.29 (m, 1H). The ent-75 (compound 201) can be synthesized following the synthetic route (70→75) using D-N-Boc-cis-4-hydroxyproline.

### Synthesis of cis-4-hydroxyproline methyl ester HCl salt (76)

Boc-cis-4-hydroxyproline (74) (450 mg, 1.95 mmol) was dissolved in methanol (10 mL) and cooled to 0° C. To the above solution, 1.8 equivalents of thionyl chloride was added. The solution was heated to 45° C. for 4 hours, and was then stirred overnight at room temperature. The reaction mixture was then concentrated under reduced pressure. Cis-4-hydroxyproline methyl ester HCl salt started to crystallize out during the evaporation. The crystals were filtered off and washed several times with ether. The crystals were finally dried in a vacuum oven for 24 hours (40° C.) to yield 76 (354 mg, ˜100%). 76: MS: M+H⁺=146. ¹H NMR (500 MHz, D₂O): δ 4.47 (m, 2H), 3.91 (s, 3H, OMe), 3.52 (m, 2H), 2.57-2.47 (m, 2H). The ent-76 (compound 202) can be synthesized following the synthetic route (70→74, 74→76) using D-N-Boc-cis-4-hydroxyproline.

### Synthesis of N-(-hydroxypropyl)-L-phenylalanine (77)

To a suspension of L-phenylalanine (1 g, 6 mmol) in water (20 mL) in a capped pyrex tube, was added propylene oxide (10 mL), followed by addition of 48% HBr (1 mL). The suspension was heated at 80° C. for 15 min, and then at ambient temperature for 18 h. The reaction mixture was filtered, and the crude product was purified by reverse-phase chromatography to yield the desired N-(2-hydroxypropyl)-L-phenylalanine (77). 77: MS: M+H+=224. The disubstituted compound (78) was also isolated from the reaction mixture.

### Synthesis of Compounds 79 and 80

A suspension of (2S,3R,4S)-4-hydroxyisoleucine (496.2 mg, 3.4 mmol) and Cs₂CO₃ (1.1 g, 3.4 mmol) in DMF:H₂O (10:1) was stirred at room temperature for 15 min before heating to 40-45° C., followed by portion-wise addition of benzyl bromide (1.2 mL, 10.2 mmol). The reaction mixture was stirred at 40-45° C. for 48-110 h, and then cooled to room temperature. After the addition of water (20 mL), the product was extracted with ethyl acetate (5×10 mL) and concentrated under vacuum to obtain crude product. The crude product was purified by silica gel column chromatography (ethyl acetate:hexanes, 20:80) to obtain compound 79 (436 mg, 31% yield) as a clear liquid and compound 80 (425 mg, 30% yield) as a clear liquid. 79: ¹H NMR (500 MHz, D₂O): δ 0.66 (d, J=6.40 Hz, 3H), 1.06 (d, J=6.18 Hz, 3H), 2.14 (m, 1H), 3.19 (d, J=13.32 Hz, 2H), 3.37 (m, 2H), 4.10 (d, J=13.16 Hz, 2H), 5.21 (d, J=11.75 Hz, 1H), 5.34 (d, J=12.33 Hz, 1H), 7.23-7.32 (m, 10H), 7.34-7.44 (m, 3), 7.47 (d, J=7.65 Hz, 2H). Compound 80: ¹H NMR (500 MHz, CDCl₃): δ 1.23 (d, J=7.30 Hz, 3H), 1.34 (d, J=5.90 Hz, 3H), 2.10 (m, 1H), 3.58 (d, J=10.14 Hz, 1H), 3.78 (s, 4H), 4.25 (m, 1H), 7.25 (m, 2H), 7.33 (t, J=7.45 Hz, 4H), 7.44 (d, J=7.51 Hz, 4H).

### Synthesis of Compound 81

Compound 79 (218 mg, 0.5 mmol), N-methyl morpholine N-oxide (91.5 mg 0.7 mmol) and powdered 4 Å molecular sieves (266 mg) were placed in a flame dried flask under nitrogen atmosphere, and to this was added a 2:1 mixture of anhydrous acetonitrile and dichloromethane (3 ml). Tetrapropylammonium perruthennate (19.6 mg, 0.02 mmol) was added to the above suspension and the progress of the reaction was followed by TLC. After concentrating the reaction mixture under reduced pressure, the crude product was taken up in dichloromethane and filtered through a pad of silica, and the pad was washed with ethyl acetate. After removal of the solvent on rotary evaporator and drying, compound 81 (213 mg, 98% yield) was obtained as a clear oil. Compound 81: ¹H NMR (500 MHz, CDCl₃): δ 0.95 (d, J=6.59 Hz, 3H), 1.73 (s, 3H), 3.15 (m, 1H), 3.25 (d, J=13.39 Hz, 2H), 3.59 (d, J=11.40 Hz, 2H), 3.94 (d, J=13.55 Hz, 2H), 5.23 (d, J=12.19 Hz, 1H), 5.32 (d, J=12.25 Hz, 1H), 7.19-7.29 (m, 10H), 7.36-7.47 (m, 5H).

### Synthesis of Compound 82

A solution of compound 81 (44.4 mg, 0.1 mmol) in a 96:4 mixture of MeOH:HCOOH (1 mL) was added to a suspension of Pd—C (44.4 mg), again in a 96:4 mixture of MeOH:HCOOH (2.5 mL). The reaction mixture was stirred at room temperature for 30 min before adding more of HCOOH (0.5 mL), and the progress of the reaction was monitored by HPLC. The reaction mixture was filtered through filter paper, and solvent was removed on the rotary evaporator to obtain compound 82 (10 mg, 63% yield) as a white solid. Compound 82: ¹H NMR (500 MHz, D₂O): ¹H NMR (500 MHz, D₂O): δ 1.33 (d, J=7.46 Hz, 3H), 2.30 (s, 3H), 3.39 (m, 1H), 4.03 (d, J=3.94 Hz, 1H).

### Synthesis of Compound 83

To a solution of compound 81 (80 mg, 0.19 mmol) in anhydrous THF (1.6 mL) at 0° C. was added slowly a 3 M solution of MeMgl in THF (0.29 mL, 0.29 mmol). The reaction mixture was stirred for 4 h and then the reaction was quenched with a saturated, aqueous solution of ammonium chloride (3 mL), followed by extraction with ethyl acetate (5×3 mL). The organic phase was concentrated under vacuum to obtain the crude product, and the crude product was purified by silica gel column chromatography (ethyl acetate:hexanes, 10:90) to obtain compound 83 (40 mg, 48% yield). Compound 83: ¹H NMR (500 MHz, CDCl₃): δ 1.16 (d, J=7.50 Hz, 3H), 1.23 (s, 3H), 1.32 (s, 3H), 2.32 (quint, J=7.88 Hz, 1H), 3.82 (d, J=14.26 Hz, 2H), 4.01 (d, J=8.89 Hz, 2H), 4.05 (d, J=14.12 Hz, 2H), 7.25 (dd, J=6.32 Hz, J=8.27 Hz, 2H), 7.33 (t, J=7.45 Hz, 4H), 7.44 (d, J=7.51 Hz, 4H).

### Synthesis of Compound 84

A solution of compound 83 (56 mg, 0.17 mmol) in a 96:4 mixture of MeOH:HCOOH (1 mL) was added to a suspension of Pd/C (56 mg), again in a 96:4 mixture of MeOH:HCOOH (2.5 mL). The reaction mixture was stirred at room temperature for 30 min before adding more HCOOH (0.5 mL), and the progress of the reaction was monitored by HPLC. The reaction mixture was filtered through filter paper, and solvent was removed on the rotary evaporator to obtain compound 84 (8 mg, 73% yield) as a white solid. Compound 84: ¹H NMR (500 MHz, D₂O): δ 1.11 (d, J=7.21 Hz, 3H), 1.51 (s, 3H), 1.57 (s, 3H), 2.89 (quint, J=7.5 Hz, 1H), 4.87 (d, J=7.81 Hz, 1H).

### Synthesis of Compound 85

A solution of compound 84 (25 mg, 0.17 mmol) in ethanol (0.5 mL) was added to an aqueous solution of LiOH (0.5 M, 0.5 mL, 0.24 mmol) and the reaction mixture was stirred at room temperature for 30 min. pH of the reaction mixture was made ˜7 with careful addition of aqueous HCl (0.1 M), and after dilution with more water, the mixture was freeze-dried to obtain compound 85 (25 mg, 90% yield) as a white solid. Compound 85: ¹H NMR (500 MHz, D₂O): δ 1.06 (d, J=7.17 Hz, 3H), 1.29 (s, 3H), 1.42 (s, 3H), 2.03 (quint, J=6.69 Hz, 1H), 3.97 (d, J=5.36 Hz, 1H).

### Synthesis of Compound 87

To a solution of imine 1 (200 mg, 0.97 mmol) in dry DMF (2 mL) under argon at 0° C. was added 1-bromo-3-methylbut-2-ene (86a) (146 μL, 1.26 mmol), followed by addition of Zn (82 mg, 1.26 mmol) and a drop of TMSCl. The reaction mixture was allowed to warm to room temperature over a period of 45 min. After cooling to 0° C., the reaction mixture was neutralized with satd. NH₄Cl, and extracted with diethyl ether (3×50 mL). The organic phase was washed with brine, dried over Na₂SO₄, filtered through a cotton swab, concentrated, and purified by silica gel column chromatography (ethyl acetate/hexanes, 10/90) to obtain compound 87 (2.89 g, 83% yield) as an orange oil. The same procedure produces compound 88 when the starting material is 1-bromo-2-methylbut-2-ene (86b) instead of 1-bromo-3-methylbut-2-ene (86a).

### Synthesis of Compound 89

To a solution of iodosobenzene diacetate (930 mg, 2.8 mmol) in dry MeOH (9.5 mL) under argon was added over a period of 30 min a solution of alkene intermediate 87 (200 mg, 0.61 mmol) in dry MeOH (1.5 mL). After stirring the reaction mixture at room temperature for 30 min, it was neutralized with 1 N HCl (25 mL). The reaction mixture was stirred for another 90 min and extracted with CH₂Cl₂ (2×40 mL), followed by washing of the organic phase with 0.1 M HCl (25 mL). CH₂Cl₂ (20 mL) was added to the combined aqueous acidic phases, and the mixture was basified to pH 8-9 with the addition of solid Na₂CO₃, followed by the addition of di-tert-butyldicarbonate (788 mg, 3.6 mmol). The reaction mixture was stirred for 90 min before decanting the aqueous phase and extracting it with CH₂Cl₂ (2×40 mL). The combined organic phases were dried over Na₂SO₄, filtered through a cotton swab, concentrated, and purified by silica gel column chromatography (ethyl acetate/hexanes, 10/90) to obtain compound 89 (106 mg, 54% yield) as a yellowish orange oil. The same procedure produces compound 90 when the starting material is compound 88 instead of compound 87.

### Synthesis of Compound 91

To a solution of compound 89 (707 mg, 2.6 mmol) in a 1:1 mixture of THF:EtOH (10 mL) was added 1 N NaOH solution (83.2 mL, 83.2 mmol) and the mixture was heated to reflux for 12 h. The reaction mixture was cooled to room temperature, concentrated, and extracted with ethyl acetate (2×50 mL). The organic phase was dried over Na₂SO₄, filtered through a cotton swab, and concentrated to obtain unreacted compound 89. The aqueous phase was acidified to pH 2 with careful addition of 1 N HCl, and extracted with ethyl acetate (3×50 mL). The combined organics were dried over Na₂SO₄, filtered through a cotton swab, and concentrated to obtain compound 91. After repeating the above process on the recovered compound 89, the total yield of compound 91, which is obtained as a white solid, was 445.5 mg (72% yield). The same procedure produces compound 92 when the starting material is compound 90 instead of compound 89.

### Synthesis of Compound 93

To a solution of compound 91 (741 mg, 3 mmol) in dimethoxyethane (30 mL) under argon at −20° C. (ice/MeOH mixture) was added N-iodosuccinimide (1.05 g, 4.6 mmol) in portions. The reaction mixture was stirred at room temperature for 12 h, neutralized with brine, and extracted with diethyl ether (3×50 mL). The combined organics were washed with a satd. aqueous solution of Na₂S₂O₅, dried over Na₂SO₄, filtered through a cotton swab, and concentrated to obtain iodolactone intermediate 93 (1.108 g, 98% yield) as a pinkish solid. The same procedure produces compound 94 when the starting material is compound 92 instead of compound 91.

### Synthesis of Compound 95

To a solution of iodolactone 93 (705 mL, 1.9 mmol) in distilled benzene (5 mL) under argon atmosphere were added tetrabutyltin hydride (824 μL, 3 mmol) and AIBN (recrystallized from MeOH, 43.4 mg, 0.19 mmol). The reaction mixture was heated to reflux for 6 h. CCl₄ (5 mL) was added to the reaction mixture and heating was continued at reflux for another 12 h. The reaction mixture was cooled, concentrated under vacuum, and the crude product was purified by silica gel column chromatography (ethyl acetate/hexanes, 10/90) to obtain compound 95 (406 mg, 88% yield) as a white solid. The same procedure produces compound 96 when the starting material is compound 94 instead of compound 93.

### Synthesis of Compound 97

To a stirred solution of compound 95 (210 mg, 0.87 mmol) in dry CH₂Cl₂ at 0° C. under argon was added trifluoroacetic acid (2.34 mL, 30 mmol) and the mixture was allowed to warm to room temperature over a period of 4 h. After concentrating the reaction mixture, amino lactone intermediate 97 (205 mg, 93% yield) was obtained as a white solid. The same procedure produces compound 98 when the starting material is compound 96 instead of compound 95.

### Synthesis of a racemic mixture of (2S,4S)- and (2R,4R)-2-amino-4-hydroxy-3,3-dimethylpentanoic acid (compounds 99a and 99b)

To a solution of amino lactone 97 (144 mg, 0.56 mmol) in distilled water (1.7 mL) was added LiOH (34 mg, 1.4 mmol). The mixture was stirred at room temperature for 25 min and the pH of the reaction mixture was adjusted to 6-7 by the careful addition of acetic acid. The reaction mixture was then concentrated under vacuum. To remove residual water, the crude product was dissolved in absolute EtOH and concentrated again under vacuum, followed by a repeat of this process for three additional times. The crude product was recrystallized from a minimum amount of EtOH at −20° C. The solid was filtered off and washed with cold EtOH to obtain a racemic mixture of (2S,4S)- and (2R,4R)-2-amino-4-hydroxy-3,3-dimethylpentanioc acid (compounds 99a and 99b) (66 mg, 73% yield) as a white solid. ¹H NMR (200 MHz, D₂O): δ 1.04 (2s, 3H), 1.05 (2s, 3H), 1.22 (d, J=6.34 Hz, 3H), 3.65 (s, 1H), 3.83 (q, J=6.10 Hz, 1H). ¹³C (75 MHz, D₂O): δ 17.30, 20.16, 21.68, 38.47, 62.05, 73.93, 173.60. IR (KBr): 3191, 2973, 2880, 1610, 1492, 1398, 1344, 1105 cm⁻¹. MS (m/z): 162 (M+1), 184 (M+Na), 323 (2M+1).

### Synthesis of racemic mixtures of (2S,3S) and (2R,3R)-2-amino-4-hydroxy-3,4-dimethylpentanoic acid (100a and 100b) and (2S,3R) and (2S,3R)-2-amino-4-hydroxy-3,4-dimethylpentanoic acid (101a and 101b)

The procedure used for the synthesis of compounds 100 (a and b) and 101 (a and b) was identical to those used for compound 99, except that amino lactone 98 was used as the starting material instead of compound 97.

The physical and NMR data of a mixture of compounds 100a and 100b is as follows: ¹H NMR (300 MHz, D₂O): δ 1.01 (d, J=7.17 Hz, 3H), 1.25 (s, 3H), 1.37 (s, 3H), 1.98 (m, 1H), 3.93 (d, J=5.61 Hz, 1H). ¹³C NMR (50 MHz, D₂O): δ 11.32, 25.19, 29.16, 43.59, 57.41, 73.86, 174.57. IR (KBr): 32982, 2924, 2659, 1783, 1629, 1527, 1471, 1393, 1278, 1172, 1134, 1061, 934, 549 cm⁻¹. MS (m/z): 162 (M+1), 184 (M+Na), 323 (2M), 345 (2M+Na).

The physical and NMR data of a mixture of compounds 101a and 101b is as follows: ¹H NMR (200 MHz, D₂O): δ 1.01 (d, J=7.34 Hz, 3H), 1.33 (s, 3H), 1.41 (s, 3H), 2.19 (m, 1H), 4.16 (d, J=5.61 Hz, 1H). ¹³C NMR (50 MHz, D₂O): δ 8.17, 25.07, 28.03, 46.14, 56.52, 73.64, 174.91. IR (KBr): 3400, 3120, 3036, 2975, 1781, 1692, 1620, 1598, 1499, 1393, 1356, 1185, 1148, 1083, 942, 883, 680, 531 cm⁻¹. MS (m/z): 162 (M+1), 184 (M+Na), 323 (2M+1), 345 (2M+Na).

### Synthesis of 2-amino-3,4-dimethylpent-4-enoic acid (Compound 102a)

A solution of compound 92 (450 mg, 1.85 mmol) in a 1:3 mixture of 1 N HCl:HCOOH (2.9 mL) was stirred at 50° C. for 12 h. After cooling the reaction mixture to room temperature, toluene (1 mL) was added and the mixture was concentrated under vacuum to remove HCOOH, and this process was repeated twice more. The crude mixture was freeze-dried for 12 h, diluted with a minimum amount of ethyl acetate (250 μL), and treated with excess propylene oxide (3.5 mL). The reaction mixture was stirred for 6 h at room temperature and filtered. The precipitates were washed with hexanes, and freeze-dried for 12 h to obtain a racemic mixture of diastereoisomers of 2-amino-3,4-dimethylpent-4-enoic acid (compound 102a) (186 mg, 70% yield) as a white solid. ¹H NMR (300 MHz, D₂O): 1.06 (d, J=7.17 Hz, 3H), 1.13 (d, J=7.17 Hz, 3H), 1.71 (s, 3H), 1.81 (s, 3H), 2.64 (m, 1H), 2.83 (m, 1H), 3.55 (d, J=8.64 Hz, 2H), 3.88 (d, J=3.75 Hz, 1H), 4.92 (s, 1H), 4.94 (s, 1H), 5.01 (s, 1H), 5.06 (s, 1H). ¹³C NMR (50 MHz, D₂O): δ 12.17, 16.09, 18.79, 21.04, 40.67, 42.90, 56.52, 57.91, 113.84, 114.94, 144.81, 145.01, 174.26, 174.45. IR (KBr): 3092, 2976, 2672, 2102, 1626, 1589, 1516, 1401, 1327, 1185, 901, 716 cm⁻¹. MS (m/z): 166 (M+Na), 287 (2M). Anal. Calcd. for C₇H₁₃NO₂: C, 58.72; H, 9.15; N, 9.78. Found: C, 58.53; H, 9.02; N, 9.61.

Similarly, 102b was synthesized from compound 91. Compound 102b: ¹H (300 MHz, D₂O): δ 1.06 and 1.13 (2d, J=7.17 Hz, 3H, H₆, H₆), 1.71 and 1.81 (2s, 3H, H₇ et H₇), 2.64 and 2.83 (2m, 1H, H₃ et H₃), 3.55 (d, J=8.64 Hz, 2H, NH₂), 3.88 (d, J=3.75 Hz, 1H, H₂), 4.92, 4.94, 5.01, 5.06 (2×2s, 1H, H₅ et H₅). ¹³C NMR (50 MHz, D₂O): δ 12.17, 16.09, 18.79, 21.04, 40.67, 42. 90, 56.52, 57.91, 113.84, 114.94, 144.81, 145.01, 174.26, 174.45. IR (KBr): 3092, 2976, 2672, 2102, 1626, 1589, 1516, 1401, 1327, 1185, 901, 716 cm⁻¹. MS (m/z): 166 (M+Na), 287 (M+M).

### Synthesis of Compound 103

(2S,3R,4S)-4-hydroxyisoleucine (100 mg, 0.68 mmol) was heated to reflux in aqueous HCl (6 N) or HBr for 6 h. The reaction mixture was cooled to room temperature and neutralized using aqueous NaOH to pH 7. After concentration, the crude product was purified using silica gel chromatography (ethyl acetate:hexanes, 1:4) to give compound 103 (62 mg, 70% yield) as a white solid. ¹H NMR (500 MHz, CDCl₃): δ 1.24 (d, J=7.42 Hz, 3H), 1.52 (d, J=7.10 Hz, 3H), 2.85 (quint, J=7.42 Hz, 1H), 4.71 (m, 2H).

### Synthesis of Compound 104

Compound 103 (100 mg, 0.48 mmol) was dissolved in pyridine (2 mL), followed by addition of acetic anhydride (0.07 ml, 0.718 mmol), and the above mixture was stirred at room temperature overnight. After concentrating, the residue was taken up in water and the pH was adjusted to 3-4 with aqueous HCl (0.1 M). The aqueous phase was extracted with ethyl acetate (4×5 ml) and concentrated. Recrystallization from hexanes/ethyl acetate gave compound 104 (18 mg, 22% yield) as a white solid. Compound 104: ¹H NMR (500 MHz, CDCl₃): δ 4.74 (1H, dd, J=5.57 Hz, J=7.65 Hz), 4.41 (1H, quad, J=6.64 Hz), 2.68 (1H, quint, J=7.42 Hz), 2.08 (3H, s), 1.45 (3H, s), 0.95 (3H, d, J=7.30 Hz).

### Synthesis of Compound 105

Pyridine (0.12 mL, 1.44 mmol) was added to a solution of compound 103 (100 mg, 0.48 mmol) in anhydrous CH₂Cl₂ (2 ml), and the mixture was cooled to 0° C. followed by the addition of benzoyl chloride (0.06 ml, 0.53 mmol). The reaction mixture was stirred at 0° C. for 1 h, overnight at room temperature, and then under refluxed for 5.5 h. More pyridine (0.48 mmol) and benzoyl chloride (0.48 mmol) were added to the cooled mixture, which was left stirring overnight. The reaction mixture was diluted with ethyl acetate (5 mL), washed with 1 N HCl (4×8 mL) until the pH was 3-4. The organic phase was washed with saturated NaHCO₃ (5 mL) to pH 8, followed by water (5 mL). The organic layer was concentrated and the crude product was recrystallized from hexanes/ethyl acetate to give compound 105 (40 mg, 36% yield) as a white solid. Compound 105: ¹H NMR (500 MHz, CDCl₃): δ 7.82 (2H, d, J=8.0 Hz), 7.55 (1H, t, J=7.41 Hz), 7.47 (2H, t, J=7.62 Hz), 4.92 (1H, dd, J=5.29 Hz, J=8.02 Hz), 4.47 (1H, quad, J=6.6 Hz), 2.84 (1H, quint, J=7.34 Hz), 1.51 (3H, d, J=7.05 Hz), 1.02 (3H, d, J=7.36 Hz).

### Synthesis of Compound 106

To a solution of compound 103 (100 mg, 0.48 mmol) and triethylamine (0.067 mL, 0.48 mmole) in anhydrous THF (1.8 mL) at 0° C. was added benzaldehyde (0.07 mL, 0.71 mmol) and sodium triacetoxyborohydride (149 mg, 0.67 mmol) in succession. The reaction mixture was stirred at 0° C. for 3 h and extracted with ethyl acetate (4×5 ml) after the addition of water (10 mL). The organic phases were combined and concentrated under vacuum to obtain crude product. The crude product was purified by silica gel column chromatography (ethyl acetate:hexanes, 1:4) to obtain compound 106 (45 mg, 43% yield) as a white solid. Compound 106: ¹H NMR (500 MHz, CDCl₃): δ 7.3-7.2 (5H, m), 4.0 (3H, m), 3.2 (1H, d, J=Hz), 2.0 (1H, m), 1.4 (3H, d, J=Hz), 1.1 (3H, d, J=Hz).

### Synthesis of Compounds 107a,b and 108a,b

To a solution of compound 103 (1 g, 4.76 mmol) in dichloromethane (15 mL) at 0° C. was added triethylamine (2 mL, 14.3 mmol) and after 15 min, p-toluenesulfonyl chloride (1.36 g, 7.14 mmol). The resulting mixture was slowly warmed to room temperature and then stirred overnight. The reaction mixture was extracted with dichloromethane (5×10 mL) and ethyl acetate (2×10 mL) after addition of water (30 mL). The organic phase was combined, washed with saturated aqueous NaHCO₃ and brine, and concentrated under vacuum to obtain crude product as an orange residue. The crude product was purified by silica gel column chromatography (ethyl acetate:hexanes, range varying from 5:95 to 25:75) to obtain 107a (982 mg, 73% yield) as a white solid and 108a (31 mg, 15% yield) as a white solid. 107a: ¹H NMR (500 MHz, CDCl₃): δ 7.79 (2H, d, J=8.17 Hz), 7.34 (2H, d, J=8.20 Hz), 4.83 (1H, d, J=3.59 Hz), 4.37 (1H, q, J=6.72 Hz), 4.10 (1H, dd, J=3.95 Hz, J=7.53 Hz), 2.54 (1H, quint, J=7.27 Hz), 2.44 (3H, s), 1.37 (3H, d, J=6.95 Hz), 1.08 (3H, d, J=7.40 Hz). 108a: ¹HNMR (500 MHz, CDCl₃): δ 7.98 (2H, d, J=8.14 Hz), 7.32 (4H, dd, J=8.08 Hz), 7.16 (2H, d, J=7.95 Hz), 4.78 (1H, d, J=11.29 Hz), 4.52 (1H, m), 2.47 (3H, s), 2.40 (3H, s), 2.34-2.17 (1H, m), 1.41 (3H, d, J=6.26 Hz), 1.15 (3H, d, J=7.28 Hz). The synthesis of the N-Cbz derivatives 107b and 108b follows the above synthetic route using either Cbz-Cl or Cbz-anhydride as electrophile.

### Synthesis of Compound 109

To a solution of compound 103 (1 g, 4.76 mmol) in dichloromethane (15 mL) at 0° C. was added triethylamine (2 mL, 14.3 mmol) and o-nitrobenzenesulfonyl chloride (1.62 g, 7.14 mmol). The resultant mixture was allowed to warm to room temperature and stirred overnight. Water (30 mL) was added and the mixture was stirred for 1 h. The crude product was extracted with dichloromethane (5×15 mL) and ethyl acetate (15 mL). The organic phase was combined, washed with saturated aqueous NaHCO₃ (30 mL) and brine (70 mL), and concentrated. The crude product was purified by silica gel column chromatography to obtain compound 109 (0.77 g, 65% yield) as a white solid. Compound 109: ¹H NMR (500 MHz, CDCl₃): δ 1.17 (d, J=7.43 Hz, 3H), 1.42 (d, J=6.39 Hz, 3H), 2.57 (quint, J=7.44 Hz, 1H), 4.40 (m, 2H), 5.94 (d, NH, 1H), 7.77 (dd, J=3.36 Hz, J=5.54 Hz, 2H), 7.97 (t, J=4.51 Hz, 1H), 8.15 (dd, J=3.57 Hz, J=5.31 Hz, 1H).

### Synthesis of Compound 110

To a solution of compound 109 (476 mg, 1.51 mmol) in anhydrous dichloromethane (8 mL) at 0° C. was dropwise added pyrrolidine (0.38 mL, 4.54 mmol). The mixture was stirred overnight at 5° C., and then for 2 h at room temperature. To the mixture were added dichloromethane (5 mL) and water (4 mL), and the pH was adjusted to 6-7 by careful addition of HCl (1 N), followed by extraction with CH₂Cl₂ (4×5 mL) and ethyl acetate (5 mL). The organic phases were combined, dried over Na₂SO₄, and concentrated to give compound 110 (290 mg, 60% yield) as a white solid. Compound 110: ¹H NMR (500 MHz, CDCl₃): δ 0.97 (d, =6.83 Hz, 3H), 1.18 (d, =5.95 Hz, 3H), 1.69 (bs, 1H), 1.77-1.94 (m, 4H), 2.92 (m, 1H), 3.21 (m, 1H), 3.49 (m, 1H), 3.84 (m, 1H), 4.29 (d, =4.58 Hz, 1H), 7.68 (m, 2H), 7.91 (m, 1H), 8.00 (m, 1H).

### Synthesis of Compound 111a,b

To a solution of compound 107a (200 mg, 0.71 mmol) in ethanol (2.6 mL) and THF (0.7 mL) was added dropwise an aqueous solution of LiOH (33 mg, 0.78 mmol). The reaction mixture was left stirring at room temperature overnight. The pH was adjusted to 6 with careful addition of aqueous HCl (1 N) before removal of the solvents. The product was dried under reduced pressure to give compound 111a (207 mg, 98% yield) as a white solid. Compound 111a: ¹H NMR (500 MHz, CDCl₃): δ 7.77 (2H, d, J=7.88 Hz), 7.47 (2H, d, J=7.79 Hz), 3.96 (1H, quint, J=5.75 Hz), 3.49 (1H, d, J=7.77 Hz), 2.46 (3H, s), 1.87 (1H, m), 1.03 (3H, d, J=6.21 Hz), 0.84 (3H, d, J=6.77 Hz). The synthesis of N-CBz derivative (111b) follows the above synthetic route.

### Synthesis of Compound 112a,b

Pyrrolidine (0.18 mL, 2.12 mmol) was dropwise added to a 0° C. cooled solution of compound 107a (200 mg, 0.71 mmol) in anhydrous CH₂Cl₂, and the mixture was stirred for 48 h at 5° C. To the mixture were added CH₂Cl₂ (5 mL) and water (3 mL) and pH was adjusted to ˜6 with careful addition of aqueous HCl (1 N). The crude product was extracted with CH₂Cl₂ (5 mL) and EtOAc (3×5 mL), the organic phases were combined, dried over Na₂SO₄, and concentrated. The crude product was purified by silica gel column chromatography to obtain compound 112a (154 mg, 62% yield) as a white solid. Compound 112a: ¹H NMR (500 MHz, CDCl₃): 0.93 (d, J=6.64 Hz, 3H), 1.17 (d, J=5.94 Hz, 3H), 1.58 (m, 1H), 1.70-1.76 (m, 2H), 1.88 (m, 2H), 2.42 (s, 3H), 2.97 (m, 1H), 3.05 (m, 1H), 3.11 (m, 1H), 3.21 (m, 1H), 3.34 (m, 1H), 3.89 (m, 2H), 6.07 (d, J=9.12 Hz, 1H), 7.29 (d, J=7.31 Hz, 2H), 7.73 (d, J=7.59 Hz, 2H). ¹³C-NMR (500 MHz, CDCl₃): δ 14.3, 21.0, 22.4, 24.7, 26.7, 44.5, 46.8, 47.3, 58.2, 68.8, 128.3, 130.3, 137.8, 144.4, 170.9. The synthesis of N-CBz derivative (112b) follows the above synthetic route.

### Synthesis of Compound 113a,b

To a solution of compound 112a (100 mg, 0.28 mmol) in anhydrous CH₂Cl₂ (15 mL) was added PCC (225 mg, 1.17 mmol), and the resultant mixture was stirred overnight at room temperature. The reaction mixture was filtered through a pad of celite, and concentrated. The crude product was purified by silica gel column chromatography to obtain compound 113a (86 mg, 82% yield) as an oil. Compound 113a: ¹H NMR (500 MHz, CDCl₃): δ 1.02 (d, J=6.6 Hz, 3H), 1.6 (m, 1H), 1.73 (m, 1H), 1.83 (m, 1H), 2.19 (s, 3H), 2.41 (s, 3H), 2.86 (m, 1H), 3.02 (m, 1H), 3.21 (m, 1H), 3.32 (m, 1H), 4.16 (t, J=8.79 Hz, 1H), 5.62 (bs, 1H), 7.27 (d, J=11.45 Hz, 2H), 7.69 (d, J=8.07 Hz, 2H). The synthesis of N-CBz derivative (113b) follows the above synthetic route.

### Synthesis of Compound 114

To a mixture of (2S,3R,4S)-4-hydroxyisoleucine (442.7 mg, 3.0 mmol), NaOH (132 mg, 3.3 mmol) in water (11 mL), and t-butanol (6 mL), CbzCl (561 mg, 3.3 mmol) was added dropwise. The resulting reaction mixture was stirred overnight at room temperature. The reaction mixture was acidified to pH 2 by using 1 M HCl. The mixture was extracted with DCM (2×100 mL). The organic phase was dried over Na₂SO₄ and evaporated to provide 114 (790 mg, 99%) as a white solid. 114: ¹H NMR (500 MHz, CDCl₃): δ 1.00 (d, J=7.07 Hz, 3H), 1.44 (d, J=6.31 Hz, 3H), 2.59 (m, 1H), 4.39 (m, 1H), 4.66 (m, 1H), 5.14 (s, 2H), 5.52 (br, 1H), 7.37 (m, 5H).

### Synthesis of Compound 115

Pyrrolidine (0.94 mL, 11.4 mmol) was added dropwise to a solution of compound 114 (1 g, 3.8 mmol) in anhydrous CH₂Cl₂ (10 mL) and the mixture was stirred for 6 h at room temperature. Water (3 mL) was added to the reaction mixture and it was extracted with dichloromethane (4×10 mL) and EtOAc (10 mL). The combined organic phases were washed with aqueous HCl (1 N, 6 mL), dried over sodium sulfate, filtered, and concentrated. The crude product was purified by silica gel column chromatography (ethyl acetate:hexanes:methanol, 1:1:1/8) to obtain compound 115 (694 mg, 55% yield) as a clear liquid. Compound 115: ¹H NMR (500 MHz, CDCl₃): δ 0.97 (d, J=7.0 Hz, 3H), 1.19 (d, J=6.14 Hz, 3H), 1.81-1.91 (m, 2H), 1.92-2.00 (m, 3H), 3.40-3.58 (m, 4H), 3.60-3.73 (m, 2H), 4.51 (dd, 1H) 5.10 (s, 2H), 5.82 (d, 1H), 7.27-7.32 (m, 5H).

### Synthesis of Compound 116

Pyrrolidine (2.36 mL, 26.8 mmol) was dropwise added over a period of 5 min to a solution of compound 103 (1 g, 4.76 mmol) in anhydrous CH₂Cl₂ (10 mL) and the resultant yellowish mixture was stirred for overnight at room temperature. Water (10 mL) was added to the reaction mixture and pH was adjusted to ˜5 with aqueous HCl (1 N, 16 mL). The aqueous phase was extracted with dichloromethane (5×10 mL) and EtOAc (10 mL). The combined organic phases were dried over sodium sulfate, filtered, and concentrated. The crude product was purified by silica gel column chromatography (ethyl acetate:hexanes:methanol, 1:1:1/8) to obtain compound 116 (323 mg, 34% yield) as a white solid. Compound 116: ¹H NMR (500 MHz, CDCl₃): δ 4.60 (1H, d, J=10.43 Hz), 4.28 (1H, d, J=10.31 Hz), 3.69 (1H, m), 3.49 (3H, m), 3.34 (2H, m), 2.26 (1H, bs), 2.00-1.83 (4H, m), 1.74 (1H, m), 1.25 (3H, d, J=7.28 Hz), 0.78 (3H, d, J=6.64 Hz).

### Synthesis of Compound 117

To a solution of compound 116 (100 mg, 0.5 mmol) in anhydrous CH₂Cl₂ (3 mL) at 0° C. was added triethylamine (0.21 mL, 1.5 mmol) and the mixture was stirred for 15 min. p-Toluenesulfonyl chloride (105 mg, 0.55 mmol) was added and the reaction mixture, which was allowed to warm to room temperature and stirred overnight. Water (6 mL) was added and the mixture was stirred for another 30 min. The aqueous phase was extracted with dichloromethane (3×15 ml) and EtOAc (2×5 mL). The combined organic phases were washed with saturated NaHCO₃ (15 mL) and brine (30 mL), dried over sodium sulfate, filtered, and concentrated. The crude product was purified by silica gel column chromatography to obtain compound 117 (129 mg, 71% yield) as a white solid. Compound 117: ¹H NMR (500 MHz, CDCl₃): δ 0.75 (d, J=6.62 Hz, 3H), 1.35 (d, J=6.07 Hz, 3H), 1.80-2.07 (m, 4H), 2.42 (s, 3H), 3.09-3.15 (m, 1H), 3.45-3.55 (m, 3H), 3.75 (m, 1H), 3.84 (m, 1H), 4.70 (d, J=10.86 Hz, 1H), 5.44 (d, J=10.62 Hz, 1H), 7.29 (d, J=7.89 Hz, 2H), 7.84 (d, J=7.84 Hz, 2H).

### Synthesis of Compound 118

To a solution of compound 116 (200 mg, 0.94 mmol) in anhydrous THF (4 mL) was added NaH (47 mg, 1.18 mmol), and the mixture was stirred at room temperature for 30 min. Benzyl bromide (177 mg, 1.04 mmol) was added and the reaction mixture was stirred for 15 h. Water (4 mL) was added and the mixture was stirred for another 30 min. The aqueous phase was extracted with dichloromethane (4×4 mL) and EtOAc (4 mL). The combined organic phases were dried over sodium sulfate, filtered, and concentrated. The crude product was purified by silica gel column chromatography to obtain compound 118 (185 mg) as a white solid. Compound 118: ¹H NMR (500 MHz, CDCl₃): δ 0.81 (d, J=6.31 Hz, 3H), 1.30 (d, J=5.98 Hz, 3H), 1.70-1.82 (m, 1H), 1.86-1.94 (m, 1H), 2.14-2.22 (m, 1H), 3.16-3.21 (m, 1H), 3.26-3.32 (m, 1H), 3.36 (d, J=10.63 Hz, 1H), 3.41-3.46 (m, 2H), 3.73 (d, J=14.24 Hz, 1H), 3.96-3.99 (m, 2H), 4.24 (d, J=10.29 Hz, 1H), 4.44 (d, J=10.24 Hz, 1H), 7.18-7.28 (m, 5H).

### Synthesis of Compound 119

To a solution of compound 103 (1.05 g, 5 mmol) in methanol (20 ml) under nitrogen atmosphere was added pyrrolidine (2.2 mL, 25 mmol), and the reaction mixture was stirred overnight at room temperature. After removal of the solvent, the crude product was purified by silica gel column chromatography (dichloromethane:methanol, 90:10) to provide compound 119 (618 mg, 61% yield) as a white solid. Compound 119: ¹H NMR (500 MHz, CDCl₃): δ 0.90 (d, J=6.98 Hz, 3H), 1.87 (d, J=6.11 Hz, 3H), 1.92 (m, 1H), 1.97 (m, 2H), 2.05 (m, 2H), 3.46 (m, 2H), 3.57 (m, 1H), 3.94 (m, 2H), 4.29 (m, 1H). ¹³C NMR (500 MHz, CDCl₃): δ 14.4, 23.3, 25.0, 26.8, 42.7, 47.4, 48.6, 57.9, 73.2, 169.1.

To a solution of compound 119 (50 mg, 0.25 mmol) and triethylamine (0.1 mL, 0.8 mmol) in dichloromethane (3 ml) under nitrogen atmosphere was added a solution of p-toluenesulfonyl chloride (53 mg, 0.28 mmol) in dichloromethane (0.5 mL), and the resultant reaction mixture was stirred overnight at room temperature. After removal of the solvent, the crude product was purified by silica gel chromatography (dichloromethane:methanol, 80:20) to obtain compound 112 (49 mg, 55% yield) as a pale yellow solid.

### Synthesis of Compound 120

To a solution of compound 119 (50 mg, 0.25 mmol) in dichloromethane (1 mL) at 0° C. under nitrogen atmosphere was added a 1 M solution of LiHMDS in hexanes (0.55 mL, 0.55 mmol). After 15 min at 0° C., the reaction mixture was cooled down to −78° C. and benzyl bromide (213 mg, 1.25 mmol) was added. The reaction mixture was allowed to warm to room temperature and stirred overnight. After completion, the reaction was quenched with methanol, concentrated, and the crude product was purified by silica gel chromatography to give compound 120 (40 mg, 55% yield) as a colorless liquid. Compound 120: ¹H NMR (500 MHz, CDCl₃): δ 0.77 (d, J=6.98 Hz, 3H), 1.19 (d, J=5.86 Hz, 3H), 1.67 (m, 1H), 1.92 (m, 4H), 3.27-3.37 (m, 3H), 3.51-3.61 (m, 3H), 3.70 (m, 1H), 3.80 (d, J=13.01 Hz, 1H), 7.32 (m, 5H).

### Synthesis of Compounds 121a and 121b

In a round bottom flask, (2S,3R,4S)-4-hydroxyisoleucine (295 mg, 2.0 mmol), Cs₂CO₃ (1.3 g, 4 mmol), BnEt₃NBr (227 mg, 1.0 mmol), and BrCH₂COOEt (0.24 mL, 2.2 mmol) were added in sequence into tBuOMe/H₂O (1:1, 20 mL). The resulting mixture was stirred at 40° C. for 48 h. Then, the pH of the mixture was adjusted to 4. The solvent was removed under reduced pressure, and the crude product was purified by HPLC to provide compound 121a (360 mg) as a white solid and 121b (20 mg) in overall 92% after freeze-drying. 121s: ¹H NMR (500 MHz, D₂O): δ 3.88 (m, 1H), 3.81 (d, J=5.77 Hz, 1H), 3.53-3.70 (dd, 2H), 1.96 (m, 1H), 1.29 (d, J=6.32 Hz, 3H), 0.98 (d, J=7.22 Hz, 3H). 121b: ¹H NMR (500 MHz, D₂O): δ 3.76-4.08 (m, 6H), 2.10 (m, 1H), 1.37 (d, J=6.50 Hz, 3H), 1.08 (d, J=7.45 Hz, 3H).

### Synthesis of Compound 123

A solution of dibenzyl lactone (122) (154 mg, 0.5 mmol), obtained from (2S,3R,4S)-4-hydroxyisoleucine, in EtOH (3 mL) was added dropwise into LiOH (0.6 mmol, 0.2 M) solution. The resulting mixture was stirred at room temperature overnight and monitored by TLC. After adjustment of the pH to 6, the solvent was removed under reduced pressure, and the crude product was purified by HPLC to provide pure hydrophobic compound 123 (24.5 mg, 15%). A diastereomeric product accounting for 70% of the product was also recovered during purification. 123: ¹H NMR (500 MHz, CD₃OD): δ7.23-7.40 (m, 10H), 3.82-3.96 (m, 5H), 3.37 (d, J=11.77 Hz, 1H), 2.10 (m, 1H), 1.33 (d, J=6.26 Hz, 3H), 1.00 (d, J=6.73 Hz, 3H).

### Synthesis of Compound 125

To commercially available (S)-lactate methyl ester (124) (590 mg, 5.0 mmol) and p-toluenesulfonic acid (a few crystals) in THF (5 mL) under nitrogen was added DHP (0.42 mL, 5.5 mmol) dropwise at 0° C. The resulting mixture was stirred at room temperature for 3 h. After evaporation of the solvent, the crude product was purified by silica gel column chromatography to afford 125 (0.86 g, 92% yield) as a clear oil.

### Synthesis of Compound 126

To a solution of compound 125 (752.4 mg, 4.0 mmol) in toluene (25 mL) under nitrogen at −78° C., DIBAL (10 mL, 10.0 mmol, 1.0 M in toluene) was added dropwise. The resulting mixture was stirred at −78° C. for 2.5 h, followed by quenching with the addition of CH₃OH (3 mL). After 5 min, concentrated potassium sodium tartrate solution (25 mL) was added and the resulting mixture was warmed up to room temperature for 15 min. The mixture was extracted with ethyl acetate (3×100 mL). After removal of solvent under reduced pressure, 126 (620 mg, 98% yield) as a pleasant smelling oil was obtained.

### Synthesis of Compound 127

The above-obtained oil (126) was dissolved in methanol (25 mL) at 0° C. with (iPr)₂NEt (0.70 mL, 4.0 mmol), valine methyl ester hydrochloride (670 mg, 4.0 mmol), and sodium cyanoborohydride (4.0 mL, 4.0 mmol, 1.0 M in THF). The reaction mixture was stirred at room temperature overnight. After evaporation, the crude product was purified by silica gel column chromatography to afford 127 as a clear oil (920 mg, 66%). The other diastereoisomer was also present in the reaction mixture, but was removed by chromatography. 127: ¹H NMR (500 MHz, CDCl₃): δ 0.89 (d, J=6.71 Hz, 3H), 0.91 (d, J=6.80 Hz, 3H), 1.14 (d, J=6.33 Hz, 3H), 1.83-1.89 (m, 5H), 2.33 (m, 1H), 2.58 (m, 1H), 2.94 (m, J=6.35 Hz, 1H), 3.68 (s, 3H), 3.74 (m, 1H), 3.82 (m, 1H), 3.88 (m, 1H), 5.24 (s, 1H).

### Synthesis of Compound 128

To a solution of compound 127 (546.2 mg, 2.0 mmol) in ethanol (2 mL), NaOH (2.5 mL, 2.5 mmol, 1.0 M in H₂O) was added. The resulting mixture was stirred at room temperature overnight. Then, HCl (4 mL, 1.0 M) was added. The resulting mixture was stirred at room temperature for another 4 h. The mixture was evaporated under vacuum. The crude product was recrystallized from 2% methanol in dichloromethane to provide 128 (285 mg, 95% yield) as a white solid. This gave 58% of overall yield for above synthesis. 128: ¹H NMR (500 MHz, CDCl₃): δ 1.06 (d, J=6.92 Hz, 3H), 1.12 (d, J=6.90 Hz, 3H), 1.26 (d, J=6.12 Hz, 3H), 2.37 (m, 1H), 3.02 (m, 1H), 3.24 (d, J=12.92 Hz, 1H), 3.85 (d, 1H), 4.15 (m, 1H).

### Synthesis of Compound 133

The compound 133 (SR) isomer was synthesized following the above-mentioned route for SS-isomer starting from (R)-lactate methyl ester (129) in an over all yield of 60%. 133: ¹H NMR (500 MHz, CDCl₃): δ 1.06 (d, J=6.86 Hz, 6H), 1.12 (d, J=7.08 Hz, 3H), 2.33 (m, 1H), 3.03 (m, 1H), 3.21 (d, J=12.96 Hz, 1H), 3.68 (d, J=3.77 Hz, 1H), 4.19 (m, 1H).

### Synthesis of Compound 134

Imine 1 (1 eq) was added dropwise to a mixture of 2-pentanone (22 eq) and L-proline (0.35 eq) in dry DMSO (40 mL) at room temperature under nitrogen, and the mixture was stirred at room temperature for 2 h. The reaction mixture was diluted with phosphate buffer (pH 7.4, 150 mL), followed by extraction with ethyl acetate (3×200 mL). The organic phase was dried over MgSO₄ and concentrated under vacuum. Purification by silica gel column chromatography yielded compound 134 in 72% isolated yield.

### Synthesis of Compound 135

To a solution of compound 134 (10 mmol) in CH₃CN (6 mL) at 0° C., was added a solution of ceric ammonium nitrate (CAN, 3 eq) in water (60 mL) with stirring. The reaction mixture was stirred for 30 min at 0° C. CH₂Cl₂ (60 mL) was added to the reaction mixture and the aqueous phase was separated, and extracted twice with CH₂Cl₂: once when made acidic with 0.1 N HCl and once when made neutral (pH 7) with Na₂CO₃ (2 N). The combined organic phases were dried over MgSO₄ and concentrated under vacuum to obtain deprotected amine 135 in an isolated yield of 84%.

### Synthesis of Compound 136

To a solution of compound 135 (10 mmol) in MeOH at 0° C. was added NaBH₄ (12 mmol) and the mixture was stirred for 90 min at 0° C. After the addition of water (40 mL), the reaction mixture was extracted with CH₂Cl₂ (3×90 mL). The combined organics were dried over MgSO₄, filtered, and concentrated under vacuum to yield intermediate 136 in an isolated yield of 89%.

### Synthesis of (2S,3S,4S)-2-amino-4-hydroxy-3-methyl-hexanoic acid (compound 12b)

To a solution of compound 136 (10 mmol) in MeOH/H₂O ( 1/10, 30 mL) was added LiOH (12 mmol). The mixture was stirred at room temperature overnight. Acetic acid (12 mmol) was added and the reaction mixture was concentrated. Water was removed from the crude product by repeated addition and evaporation of absolute EtOH. The recrystallization of the crude product from EtOH gave (2S,3S,4S)-2-amino-4-hydroxy-3-methyl-hexanoic acid (compound 12b) in an isolated yield of 50%. ¹H NMR (300 MHz, D₂O): δ 0.97 (m, 6H), 1.55 (m, 1H), 2.23 (m, 2H), 3.56 (m, 1H), 3.99 (d, J=2.8 Hz, 1H). ¹³C NMR (75 MHz, D₂O): δ 9.52, 11.78, 27.48, 38.02, 56.11, 75.38, 174.77. MS (IC) m/z: 162 [M+H]⁺. Compound 13b was also isolated from silica gel column chromatography purification and ¹H NMR was in accord with the structure.

### Synthesis of Compound 142

In a 100 mL round bottom flask, tribenzyl protected (2S,3R,4S)-4-Hydroxyisoleucine (140) (420 mg, 1.0 mmol) and NaN₃ (650 mg, 10 mmol) were dissolved in 30 ml of acetonitrile. The resulting mixture was stirred at room temperature for a week and then evaporated to dryness. The crude product was eluted through a short silica gel column with ethyl acetate to obtain the organic mixture, which was further purified by silica gel column chromatography using ethyl acetate and hexane mixture to provide 433 mg of the compound 141. The azide intermediate (141) was dissolved in methanol and deprotected under hydrogenolysis conditions of H₂ (50 Psi) and Pd/C catalyst for 24 h at room temperature to afford compound 142.

### Synthesis of Compound 138

(2S,3R,4S)-4-Hydroxyisoleucine upon reaction with hydrochloric acid gave the corresponding lactone 137. 137: ¹H NMR (CDCl3, 500 MHz): δ 1.11 (d, J=7.2 Hz, 3H), 1.43 (d, J=6.4 Hz, 3H), 2.31 (m, 1H), 3.81 (1H), 4.34 (m, 1H). A reaction mixture containing lactone form of (2S,3R,4S)-4-Hydroxyisoleucine (137) (260 mg, 2.0 mmol), BnBr (10 mmol) and Cs₂CO₃ (2 g, 6 mmol) in TBME/water (1:1 mixture, 10 mL) was stirred at room temperature for 3 days. After evaporation and filtration through a short silica gel column with 5% methanol in dichloromethane, the pure dibenzyl protected lactone (122) was obtained in high purity (430 mg, 70%). This product was hydrolyzed using 0.5M LiOH (4 mL) and ethanol (2 mL). The crude product was purified by silica gel column chromatography to provide compound 138 as colorless oil (410 mg, 90%). 138: ¹H NMR (D₂O, 500 MHz): δ 7.40-7.23 (m, 5H), 3.96-3.82 (m, 5H), 3.39-3.33 (m, 1H), 2.10 (m, 1H), 1.31 (d, J=6.26 Hz, 3H), 1.00 (d, J=6.44 Hz, 3H).

### Synthesis of Compound 143

(2S,3R,4S)-4-Hydroxyisoleucine (295 mg, 2.0 mmol), NaH (80 mg, 60%, 2.0 mmol) and MeI (140 uL, 2.2 mmol) were mixed and stirred at room temperature overnight. Then the mixture was adjusted to pH 6 with 1 M HCl solution. The solvent was removed under reduced pressure to provide crude solid, which was purified by HPLC to afford compound 143 (290 mg, 90% yield) as white solid. 143: ¹H NMR (D₂O, 500 MHz): δ 1.14 (d, J=7.04 Hz, 3H), 1.46 (d, J=6.27 Hz, 3H), 2.17 (m, 1H), 3.51 (s, 3H), 4.0 (m, 1H), 4.08 (d, 1H).

### Synthesis of Compound 139

Lactone form of (2S,3R,4S)-4-Hydroxyisoleucine (137) (130 mg, 1.0 mmol) and allyl bromide (0.26 mL, 3.0 mmol) were mixed and stirred in DMF (5 mL) at room temperature overnight. The reaction mixture was extracted with CH₂Cl₂ (2×50 mL) after addition of 0.1 M NH₄Cl solution (20 mL). The organic phase was dried and evaporated to provide a colorless oil product (120 mg, 65%). A iPrOH (1.5 mL) solution of the above crude was added into LiOH solution (1 mL, 0.8M) and stirred at room temperature for 40 min. The pH of the mixture was adjusted to 3.5 at 0° C., and dried under reduced pressure. The crude product was purified by silica gel column chromatography to afford compound 139 (131.7 mg, 58%) as a white solid. 139: ¹H NMR (CD₃OD, 500 MHz): δ 0.94 (d, J=6.60 Hz, 3H), 1.21 (d, J=6.31 Hz, 3H), 2.03 (m, 1H), 3.46 (m, 2H), 3.71-3.82 (m, 4H), 5.45 (m, 4H), 5.94 (m, 2H).

### Synthesis of Compounds 146 and 147

In a round bottom flask under nitrogen atmosphere at −78° C., glycine phosphate (1.98 g, 6.0 mmol) and tBuOK (700 mg, 6.0 mmol) was mixed and stirred for 1 h, followed by the addition of the aldehyde (1.0 g, 6.0 mmol). The mixture was kept at −78° C. for another 5 h, before quenching with NH₄Cl solution. After workup, the crude product was purified by silica gel column chromatography to provide alkene intermediate 144 (2.1 g, 95%) as an oil.

A solution of compound 144 (370 mg, 1.0 mmol) in methanol (10 mL) was kept under H₂ atmosphere (50 Psi) for 4 h using catalytic amount of Pd/C. The mixture was filtered and dried, and to the obtained crude product was added NaOH (2 mL, 1 M) and CH₃OH (2 mL). The reaction mixture was stirred for 5 h at room temperature and monitored by TLC. After hydrolysis was complete, the reaction mixture was adjusted to pH 3.0 with 1 M HCl solution. The mixture was dried under reduced pressure. The crude product was purified by silica gel column chromatography to provide O-benzyl derivative (146) (200 mg, 90% yield) as a diastereoisomer mixture (d.r 3:1). 146: ¹H NMR (D₂O, 500 MHz): δ 1.32 (s, 3H), 1.98-2.17 (m, 2H), 3.85-3.99 (m, 2H), 4.54 (m, 1H), 4.69 (m, 1H), 7.46 (m, 5H). The compound 146 (223 mg, 1.0 mmol) was dissolved in 4.5% formic acid solution in methanol (15 mL) with Pd/C 10%, 100 mg). After over night reaction, the solvent was filtered off and evaporated under reduced pressure to provide compound 147 (126 mg, d.r 2: 1, 95%) as a diastereoisomer mixture. 147: ¹H NMR (D₂O, 500 MHz): δ 1.32 (m, 3H), 1.95-2.20 (m, 2H), 3.85-4.20 (m, 2H).

C) Additional Analogs of 4-hydroxyisoleucine

Analogs of 4-hydroxyisoleucine in which the 3- and 4-positions are substituted with groups other than methyl can also be prepared using standard chemistry known in the art for synthesizing α-amino acids using commercially available or known precursors. Examples of the synthetic methods that can be employed in such preparations can be found in Rolland-Fulcrand et al., Eur. J. Org. Chem., 873-773, 2004; Kassem et al., Tetrahedron:Assymetry 12:2657-61, 2001; Wang et al., Eur J. Org. Chem., 834-39, 2002; Tamura et al., J. Org. Chem. 69:1475-80, 2004; Jamieson et al., Org. Biomol. Chem. 2:808-9, 2004; Gull and Scholikopf, Synthesis 1985:1052, 1985; Inghardt et al., Tetrahedron 32:6469-82, 1991; and Dong et al., J. Org. Chem. 64:2657-66, 1999.

### Example 2

### Effect of 4-Hydroxyisoleucine on Body Weight Gain and Food Consumption of Diet Induced Obesity (DIO)-Mice

The objective of this study was to evaluate the effect of chronic administration of 4-hydroxyisoleucine (4-OH, compound 14a) on food consumption and body weight gain of DIO-mice. Both parameters were monitored for 1 week prior to the commencement of treatment, then for the 77 days of treatment and for an additional 12 days post-treatment.

C57BL/6 mice were received at 7-8 weeks of age and fed a high fat diet (60% of calories from fat) for several weeks. A total of 32 animals were used in the study. The animals were distributed into 4 groups (3 treated, 1 control group, all on high fat diet). Each group was composed of 8 animals. The mice were randomized according to body weight and basal glycemia values following a 5±0.5 hour fasting period.

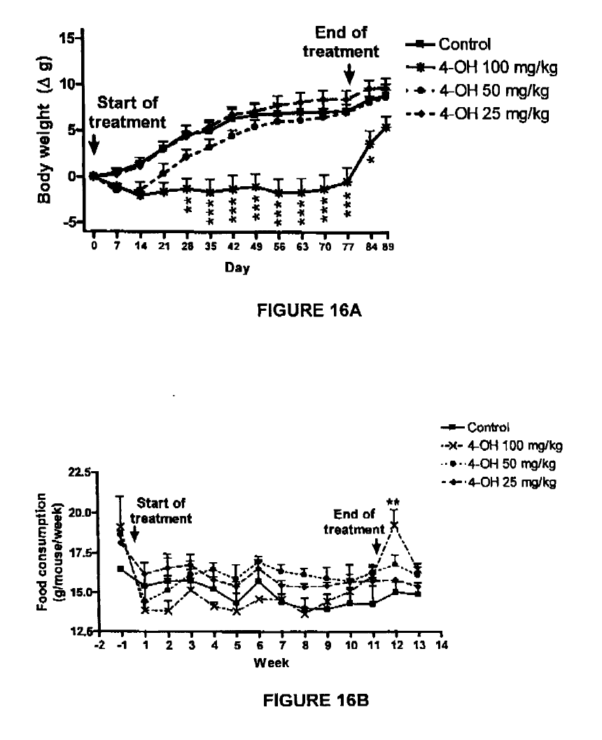

The test agent was dissolved in reverse osmosis water. 4-Hydroxyisoleucine was aliquoted and kept at 4° C. Control animals received reverse osmosis water twice daily (group 1). Mice from groups 2, 3 and 4 were treated twice daily with 4-hydroxyisoleucine (4-OH, compound 14a) at 100, 50, and 25 mg/kg, respectively. All groups were treated by oral gavage. Treatment commenced on Day 0 and ended on Day 77. Body weights were measured daily and once a week values are shown in FIG. 16A. Food consumption was measured daily and averaged on a weekly basis beginning one week before the start of treatment as shown in FIG. 16B. Similarly, food consumption was monitored during the treatment period and for 12 days after treatment was stopped as shown in FIGS. 16A and 16B.

Treatments were well-tolerated for all groups receiving 4-hydroxyisoleucine (4-OH, compound 14a). During the first three weeks of treatment, moderation of weight gain was observed for animals receiving compound 14a at 50 and 100 mg/kg (FIG. 16A). However, this effect on weight gain was sustained and highly significant from Day 28 to Day 84 of treatment for mice receiving 100 mg/kg of 4-OH twice daily. This reduction in body weight gain was paralleled with a slight decrease in food consumption during the first week of treatment (FIGS. 16A and 16B). Similarly, body weight gain and food consumption were monitored for 12 days after treatment was stopped and values from Day 84 and Day 89 are shown in FIGS. 16A and 16B. In FIGS. 16A and 16B, the body weight gain and the food consumption over time showed an increase in the first week following cessation of treatment with 100 mg/kg of 4-OH. This suggests that the continuous presence 4-OH is necessary to maintain efficacy (reduction of weight gain) in mice fed a high fat diet.

In conclusion, this study confirmed that 4-hydroxyisoleucine (4-OH) administered chronically is significantly effective at controlling body weight gain when given at a dose level of 100 mg/kg twice daily and that continuous exposure to 4-hydroxyisoleucine may be required to maintain efficacy over long periods of time, particularly if a high fat diet is maintained.

### Example 3

The objective of these studies was to determine the effect of a fixed dose of 4-hydroxyisoleucine on body weight gain of in diet induced obese (DIO) animals. C57BL/6 mice were fed a high fat diet (60% fat) for 6 weeks. Thereafter, they were treated either with vehicle or 4-hydroxyisoleucine (4-OH) for a total of 21 days. The body weight of the animals was determined on day 1 and the dose of 4-hydroxyisoleucine to be administered on that day was calculated (50, 75, 100, 125 and 150 mg/kg bid). This regime was continued for the remaining 21 day period and the dose given was not adjusted as the mice lost body weight. This method of dosing was designed to mimic the human situation when the quantity of drug given is not adjusted with progressive body weight loss.

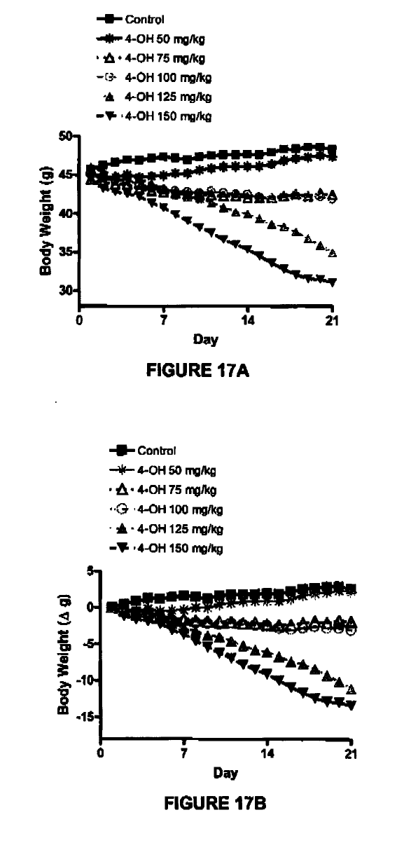

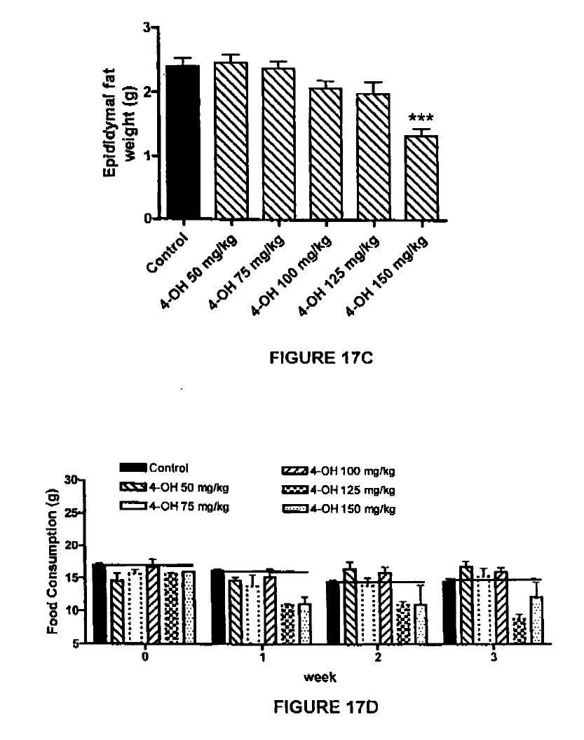

The effect of a constant fixed dose of 4-hydroxyisoleucine on body weight and body weight gain is shown in FIGS. 17A and 17B. Statistical analysis of the data was performed using a 2 way ANOVA followed by Bonferroni comparisons. Levels of significance at each time point in the 4-OH treated vs. the control group are shown in Table 4.

TABLE 4

| | | Significance |
|---|---|---|
| Dose Level | Days | vs controls |
| 75 mg/kg | 7 to 21 | P < 0.05 |
| 100 mg/kg | 7 to 21 | P < 0.05 |
| 125 mg/kg | 6 to 21 | P < 0.05 |
| 150 mg/kg | 5 to 21 | P < 0.05 |

The results in FIGS. 17A and 17B show that body weight was stable during the dosing period in the vehicle administered control animals. Oral administration of 4-OH at doses of 50, 75, 100, 125 and 150 mg/kg bid produced a dose-dependent reduction in body weight. At the highest dose administered (150 mg/kg bid), body weight plateaued at approximately 30 gm after a body weight reduction of 13 gm. On day 21 of drug administration, the animals were visually indisingishable from lean mice of the same age. The reduction of body weight was accompanied by a concomitant reduction in the weight of fat tissues, as shown in FIG. 17C.

Food intake was measured daily and then combined over a 7 day period to calculate total consumption during each week of drug administration. The effect of 4-OH on weekly food consumption is shown in FIG. 17D.

The results show that the reduction in body weight produced by 4-OH was associated with a reduction in food intake at the highest doses administered (p<0.05) during each of the 3 weeks of drug administration.

### Example 4

### Effect of Chronic Treatment with 4-Hydroxyisoleucine and Rosiglitazone, Administered Alone or in Combination

The objective of this study was to evaluate the effect of chronic administration of 4-hydroxyisoleucine (4-OH, compound 14a) and Rosiglitazone, administered alone or in combination, on food consumption and body weight gain of DIO-mice. Both parameters were monitored for 1 week prior to the commencement of treatment, then for the 28 days of treatment and for an additional 7 days post-treatment.

A total of 72 animals were used in the study. The animals were distributed into 6 groups (5 treated, 1 control group, all on high fat diet). Each group was composed of 12 animals. The mice were randomized according to body weight and basal glycemia values following a 5±0.5 hour fasting period.

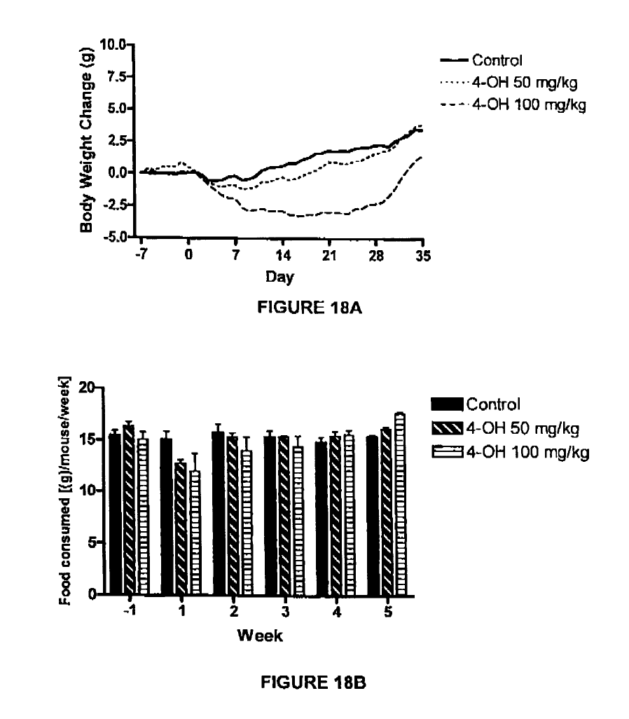

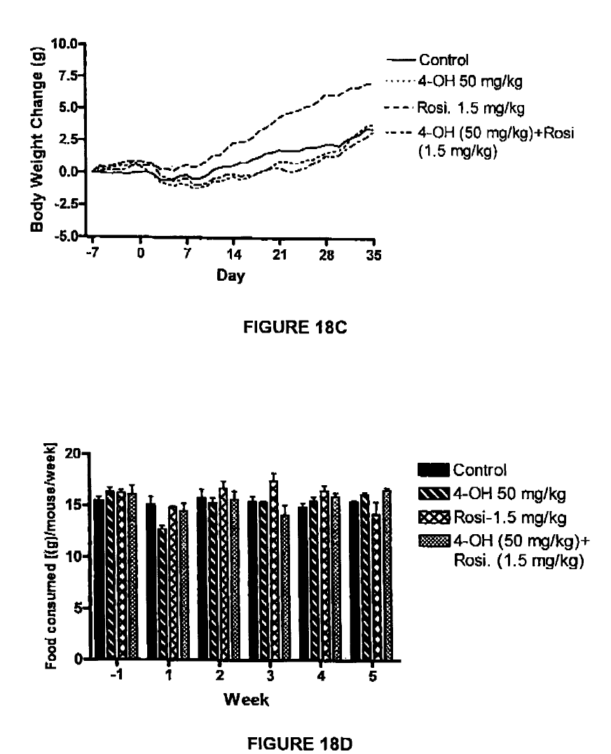

For the four weeks of treatment, the test articles were dissolved in reverse osmosis water. 4-Hydroxyisoleucine was aliquoted and kept at 4° C. (administration to groups 2, 3, and 6), while Rosiglitazone was freshly prepared daily and kept at 4° C. between the AM and PM administration to groups 4, 5, and 6. Control animals received reverse osmosis water twice daily (group 1). Mice from groups 2 and 3 were treated twice daily with 4-OH at 50 and 100 mg/kg, respectively. Animals from groups 4 and 5 received 1.5 and 5 mg/kg of Rosiglitazone, respectively. For group 6, the treatment consisted of 50 mg/kg of 4-OH plus 1.5 mg/kg of Rosiglitazone. All groups were treated by oral gavage. Treatment commenced on Day 0 and ended on Day 28 (FIGS. 18A and 18C.

Treatments were well tolerated for all groups receiving 4-hydroxyisoleucine (4-OH) or Rosiglitazone (Rosi), alone or in combination. Moderation of weight gain was observed for all animals receiving 4-OH at 100 mg/kg (FIG. 18A), or the combination of Rosiglitazone (1.5 mg/kg) with 4-OH (50 mg/kg) (FIG. 18C) relative to the group treated with Rosiglitazone alone.

Food consumption was measured and averaged on a weekly basis beginning one week before the start of treatment as shown in FIGS. 18B and 18D as week −1. Similarly, food consumption was monitored for one week after treatment was stopped and is shown as week 5 in FIGS. 18B and 18D, In FIG. 18B, the food consumption over time for various treatment groups is illustrated by the bar graph. The solid bar appearing first in each group shows the food consumption by the control group. The second and third bar in each group shows consumption by animals treated with 4-OH at 50 mg/kg or 100 mg/kg, respectively. During the first week of treatment, food consumption decreased for the 4-OH-treated groups, however consumption returned to pre-treatment levels for the remainder of the treatment phase of the study.

Rosiglitazone-treated animals had a significant increase in weight relative to the other groups that could be attributable to increased food consumption (FIG. 18D). In FIG. 18D, food consumption by the control animals is represented by the solid bar appearing first in each bar grouping. The second, third, and fourth bar in each grouping represents food consumption by animals treated with 4-OH (50 mg/kg), Rosiglitazone (1.5 mg/kg), and a combination of the drugs, respectively. Once again, 4-OH caused a reduction in food consumption during the first week, but not after, for the duration of the treatment period. Conversely, animals treated with Rosiglitazone showed an increase in food consumption; however, this effect was not observed when the two drugs were co-administered. 4-Hydroxyisoleucine was able to modulate the weight gain induced by Rosiglitazone.

Altogether, these results demonstrate that 4-hydroxyisoleucine (4-OH, compound 14a) could be used therapeutically alone to modulate weight gain. These results also suggest that the compounds according to the invention, and more particularly 4-hydroxyisoleucine, could be used in combination with Rosiglitazone to control the unwanted side effect of weight gain caused by this anti-diabetic agent.

### Example 5

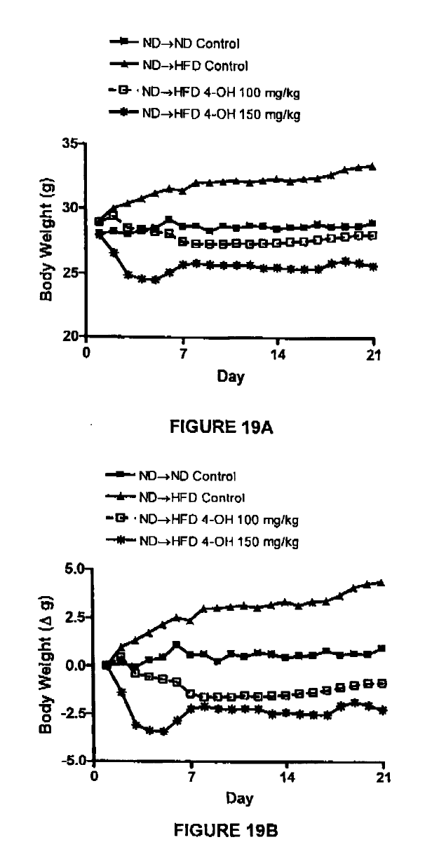

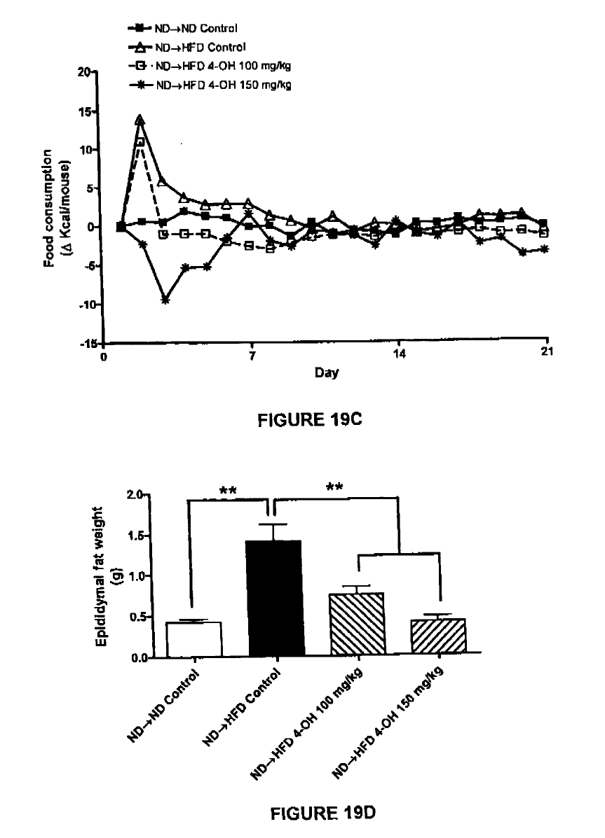

The objective of this study was to determine if oral chronic administration of 4-hydroxyisoleucine (4-OH) could prevent the development of obesity in lean mice subjected to a high fat diet upon the start of treatment. C57BL/6 mice were fed a standard chow (LabDiet #5001; 12% of calories from fat) upon arrival and during the acclimation and pre-treatment periods. At this point, mice on standard chow (ND: normal diet) were randomized according to body weight values into 4 groups of 8 mice per group. Treatment was initiated on the first day of experimentation (Day 1) and lasted 21 days. Animals from group 1 were fed the standard chow for the duration of the study and received water orally twice daily (ND: normal diet control). Mice from group 2 were given the high fat diet (HFD; 60% of calories from fat) starting on day 1 of experimentation and until day 21 of the study. These mice also received water by oral gavage and served as the high fat diet control (ND→HFD). Mice from group 3 and 4 were also fed the high fat diet upon start of treatment on day 1 and for the duration of the study but were treated orally twice daily with 100 and 150 mg/kg of 4-OH. From day 1 to day 21 of the study, body weight and food consumption were measured daily. FIGS. 19A and 19B show that mice kept on standard chow maintained stable body weight for the duration of the study. However, control mice fed with the high fat diet upon the start of treatment gained significantly more weight than the ND control throughout the study. This weight gain induced by high fat feeding was completely prevented by oral administration of 100 and 150 mg/kg of 4-OH. Furthermore, mice on the high fat diet receiving the highest dose of 4-OH had a decreased body weight compared to mice fed the standard chow (ND).

FIG. 19C shows that high fat feeding produced a transient increase of caloric intake in the HFD control compared to the ND control. This transient increase of caloric intake was also observed for mice fed the HFD receiving 100 mg/kg of 4-OH, although less pronounced. However, oral administration of 150 mg/kg of 4-OH completely abolished this increase of caloric intake in mice receiving the HFD. From day 2 to day 6 of treatment, the caloric intake of mice treated with 150 mg/kg of 4-OH was even lower than mice fed the standard chow.

After 21 days of treatment, mice from all groups were sacrificed and the weight of epididymal white tissue was determined. FIG. 19D shows that mice fed the HFD for the duration of the study had a significant increase of their epididymal fat weight compared to ND mice (p<0.01). However, administration of 100 and 150 mg/kg of 4-OH prevented significantly the increase of weight of the epididymal fat induced by the hat fat feeding (p<0.01). Therefore, these results demonstrate that 4-OH can prevent the development of obesity induced by an increased caloric intake such as high fat feeding.

### Example 6

Studies using in rodent models other than the dietary obese mouse and rat have also been conducted to assess the effect of 4-hydroxyisoleucine on weight gain. Agouti mice, which are obese, diabetic and leptin resistant, were treated orally twice daily with 50 or 100 mg/kg of 4-hydroxyisoleuciine for 21 consecutive days. Body weight and food consumption were measured daily.

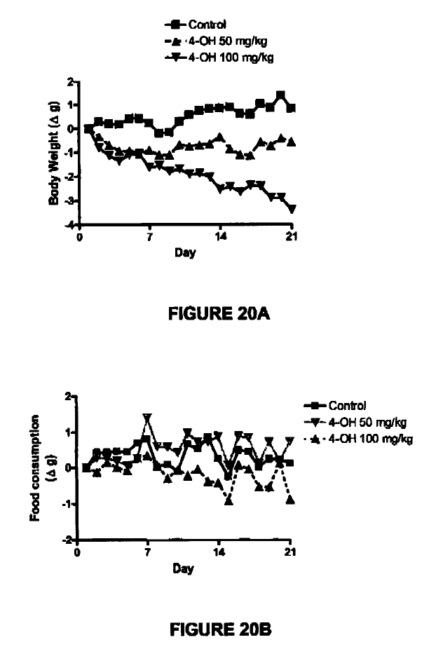

The results in FIG. 20A show that 4-hydroxyisoleucine treatment led to a significant decrease of body weight gain during the treatment period compared to vehicle treated control animals.

The results in FIG. 20B show that 4-hydroxyisoleucine has little effect on food intake despite the reduction in body weight gain in this genetic model of obesity, suggesting that 4-hydroxyisoleucine reduces body weight mainly by mechanisms that are unrelated to a reduction in food intake.

### Example 7

### Effect of 4-Hydroxyisoleucine on Body Weight Gain and Food Consumption of ob/ob Mice

The objective of this study was to evaluate the effect of chronic administration of 4-hydroxyisoleucine (4-OH, compound 14a) on food consumption and body weight gain in a genetic model of obesity, the ob/ob mouse. Body weight gain and food consumption were monitored for 1 week prior to the commencement of treatment, and then for the 56 days of treatment.

A total of 16 animals were used in the study. The animals were distributed into 2 groups (1 treated, 1 control group, all on standard chow). Each group was composed of 8 animals. The mice were randomized according to body weight values.

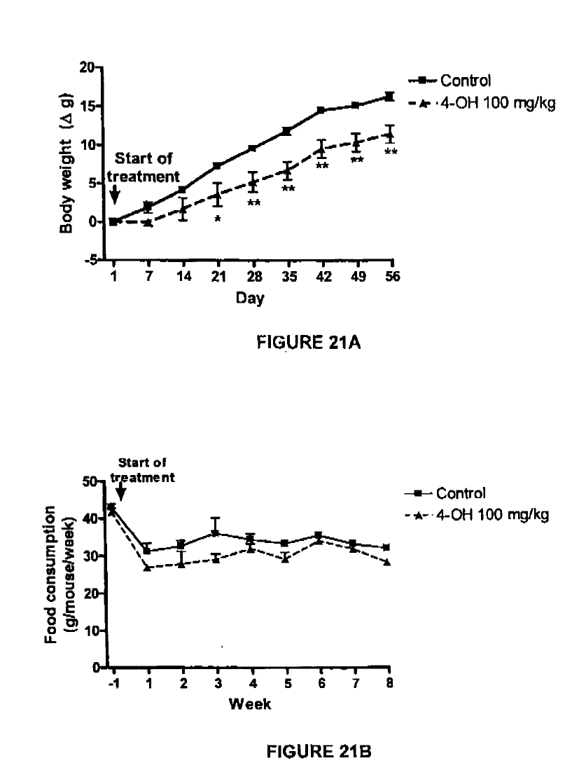

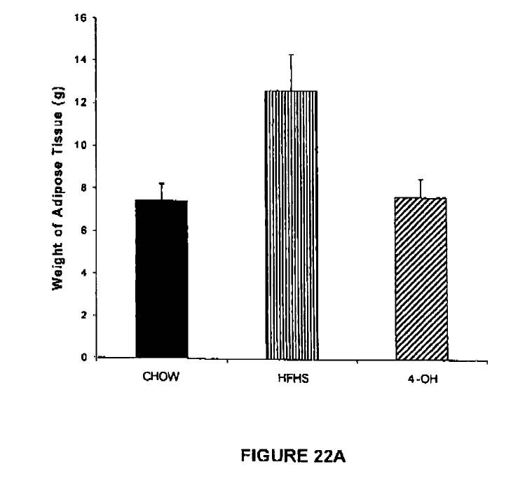

For the eight weeks of treatment, the test agent was dissolved in reverse osmosis water. 4-hydroxyisoleucine was aliquoted and kept at 4° C. Control animals received reverse osmosis water twice daily (group 1). Mice from group 2 were treated twice daily with 4-OH at 100 mg/kg. All groups were treated by oral gavage. Treatment commenced on Day 0 and ended on Day 56 (FIGS. 21A and 21B). Body weights were measured daily and once a week values are shown in FIG. 21A. Food consumption was measured daily and averaged on a weekly basis beginning one week before the start of treatment as shown in FIG. 21B. Similarly, food consumption was monitored during the treatment period as shown in FIG. 21B.

4-Hydroxyisoleucine (4-OH) treatment was well tolerated for all mice. During the course of treatment, moderation of weight gain was observed for animals receiving 100 mg/kg 4-OH (FIG. 21A). Weight gain of ob/ob mice was significantly reduced from Day 21 to Day 56 as compared to the control group. This reduction in body weight gain was paralleled with a slight decrease in food consumption during the first three weeks of treatment (FIG. 21B) but not later on.

In conclusion, chronic administration of 4-OH significantly reduced body weight gain in a severe genetic model of obesity, the ob/ob mouse model. Thus, the results of this study confirm that the compounds according to the invention, and more particularly 4-hydroxyisoleucine (4-OH, compound 14a) shows great potential for the treatment of different metabolic disorders, such as overweight, obesity, and diabetes.

### Example 8

### Prevention of Weight Gain by 4-Hydroxyisoleucine in a Rat Model of Diet-induced Obesity

The aim of this study was to evaluate the effect of chronic administration of 4-hydroxyisoleucine (4-OH, Compound 14a) on food consumption, tissue weight, and body weight gain of normal Wistar rats fed a high fat, high sucrose diet (HFHS).

The animals were acclimated for 1 week and fed standard chow prior to the commencement of treatment, then for the 28 days of the treatment the animals were fed a high fat, high sucrose diet (HFHS). A total of 30 animals were used in the study. The animals were distributed into 3 groups each composed of 10 animals: 1 group fed HFHS with treatment, 1 untreated control group fed standard chow, and 1 untreated group fed HFHS. Animals were housed separately and food consumption was monitored daily.

For the four weeks of treatment, the test compounds were dissolved in reverse osmosis water. 4-hydroxyisoleucine (4-OH) was aliquoted and kept at 4° C. Treated animals received twice daily oral administration of 4-OH at 100 mg/kg per dose. Control animals received water twice daily.

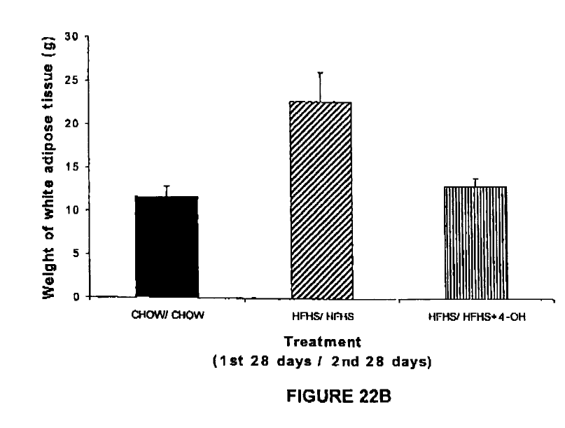

Treatment was well tolerated for the group receiving 4-OH. Moderation of weight gain was observed for all animals receiving 4-OH, and could be attributed to reduction of epididymal and peri-renal adipose tissue (FIG. 22A). Muscle, brown fat, and organ weight were not affected by the treatment (data not shown). While there was a reduction in food consumption by the treated animals, the difference in consumption relative to untreated animals could not account for the differences in weight gain (data not shown).

The results of this study support the rationale of using the compounds according to the invention, and more particularly 4-hydroxyisoleucine, for the prevention of obesity, including the prevention of weight gain and the prevention visceral fat increases.

### Example 9

### Reversal of Weight Gain by 4-Hydroxyisoleucine in a Rat Model of Diet-Induced Obesity

The aim of this study was to evaluate the effect of chronic administration of 4-hydroxyisoleucine (4-OH, Compound 14a) on food consumption, tissue weight, and body weight gain of wistar obese rats.

A total of 30 animals were used in the study. The animals were acclimated for 1 week and fed standard chow. The animals were randomized into 3 groups of 10 animals each. Two groups were fed a high fat, high sucrose diet (HFHS), and 1 untreated control group was fed standard chow over a 28 day period. Animals were housed separately and food consumption was monitored daily.

In the following period of 28 days, the feeding regimen remained the same for the 3 groups; however, 1 group fed HFHS was treated with twice daily oral administration of 4-OH at 100 mg/kg per dose. For the 28 days of treatment, 4-OH was dissolved in reverse osmosis water, aliquoted, and kept at 4° C. Untreated animals received water twice daily.

Treatment was well tolerated for the group receiving 4-OH. Moderation of weight gain was observed for all animals receiving 4-OH, and could be attributed to reduction of epididymal and peri-renal adipose tissue (FIG. 22B). Muscle, brown fat, and organ weight were not affected by the treatment. While there was a reduction in food consumption by the treated animals, the difference in consumption relative to untreated animals could not account for the differences in adiposity (data not shown).

The results of this study support the rationale of using the compounds according to the invention, and more particularly 4-hydroxyisoleucine, for the therapeutic treatment of obesity, and more particularly for reducing accumulated weight gain and visceral fat.

### Example 10

### Reduction in body Fat by Treatment with 4-hydroxyisoleucine in a Rat Model of Diet-Induced Obesity

The aim of this study was to evaluate the effect of chronic administration of 4-hydroxyisoleucine (4-OH, compound 14a) on white adipose tissue weight in the Wistar rat model of diet-induced obesity. The animals were acclimated for 1 week and fed standard chow prior to the commencement of treatment.

A total of 60 animals were used in the study. The animals were distributed into 6 groups; 1 group was fed standard chow, 1 group was fed a high fat, high sucrose (HFHS) diet with 28 days treatment, 1 untreated control group was fed a HFHS diet, and 1 untreated group was fed a HFHS diet, but with caloric intake restricted to match the caloric intake of the 4-OH treated animals (pair-fed animals). A fifth group was fed a high fat, high sucrose (HFHS) diet with 28 days treatment and received an acute oral administration of 4-OH prior to experimentation on Day 29 of the study, and a sixth group was fed a HFHS diet, but with caloric intake restricted to match the caloric intake of the 4-OH treated animals receiving an acute oral administration of 4-OH on Day 29 of the study (pair-fed animals). Each group was composed of 10 animals. Animals were housed separately and food consumption was monitored daily. Immediately prior to commencement of treatment the animals were evaluated by DEXA scanning (Dual Energy X-ray Absortiometry) to determine the baseline percentage of body weight related to fat.

For the four weeks of treatment, the test article (4-OH) was dissolved in reverse osmosis water. 4-OH was aliquoted and kept at 4° C. Treated animals received twice daily oral administration of 4-OH at 100 mg/kg per dose for 28 days (4-OH group) or twice daily oral administration of 4-OH at 100 mg/kg per dose for 28 days plus an acute oral administration of 4-OH on Day 29 (4-OH+acute group). Untreated animals received water twice daily.

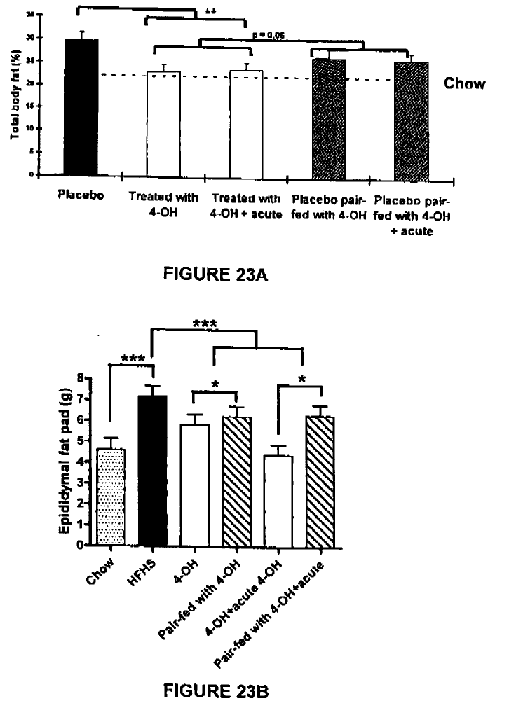

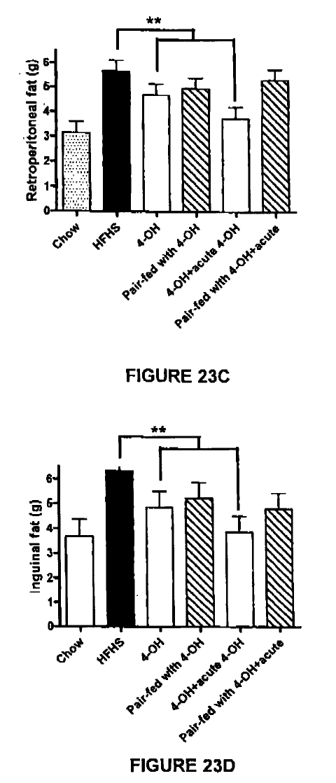

After 28 days of treatment, the animals were scanned again by DEXA and the percentage change in fat composition relative to baseline was determined (FIG. 23A). While the untreated, pair fed rats lost some fat (cross-hatched bars), relative to the untreated control animals receiving a HFHS diet (black bar), there was a significant reduction in percentage fat for the 4-OH treated groups (p<0.01) despite receiving a HFHS diet ad libitum (white bars). The treated animals had a body fat composition similar to untreated animals receiving normal chow (dashed line) throughout the study period.

The animals were sacrificed and white adipose tissue (epididymal, inguinal and retroperitoneal) was collected and weighed. FIGS. 23B, 23C, and 23D illustrate the results. The untreated pair fed animals showed some non-significant weight reduction in these tissues (cross-hatched bars) compared to the untreated HFHS controls (black bars) due to reduced caloric intake, however the 4-OH treated animals showed a significant reduction in the weight of these tissues (white bars) compared to the pair fed untreated controls (hatched bars) and the HFHS control (Black bar). The weight of these tissues in the treated animals fed a HFHS diet was the same as untreated animals receiving normal chow (Dotted bar). Those results show that the weight reduction of fat tissue cannot be explained solely by reduced caloric intake and suggest that the treated animals lose fat through increased fat metabolism.

Thus, the results of this study confirm that the compounds according to the invention, and more particularly 4-hydroxyisoleucine (4-OH, compound 14a), could be used therapeutically to reduce body fat, including visceral fat, the equivalent tissue in humans of rodent epididymal, inguinal and retroperitoneal tissues. Reduction of visceral fat is a great advantage of the invention because visceral fat is known to be a factor in development of several diseases including type 2 diabetes and cardiovascular disease. Accordingly, the current results also provide exemplary support for using the compounds according to the invention for use in the prevention or treatment type 2 diabetes and cardiovascular diseases.

### Example 11

### Increased Energy Expenditure (Oxygen Consumption) in Rats Treated with 4-hydroxyisoleucine

A study was conducted to determine whether the decrease in adiposity as described above was due to increased energy expenditure. For this study, Wistar rats were randomized into 3 groups (n=10/group) and fed HFHS diets. One group was treated with 4-hydroxyisoleucine (4-OH, compound 14a) at 100 mg/kg by oral gavage twice daily. The second group of animals served as pair fed controls, and while not treated with drug, diet in terms of caloric intake was matched to treated animals. The third group was fed a HFHS diet ad libitum without any treatment. After a 4 week period of treatment these animals were assessed by indirect calorimetry with increased oxygen consumption used as an indicator of increased energy expenditure.

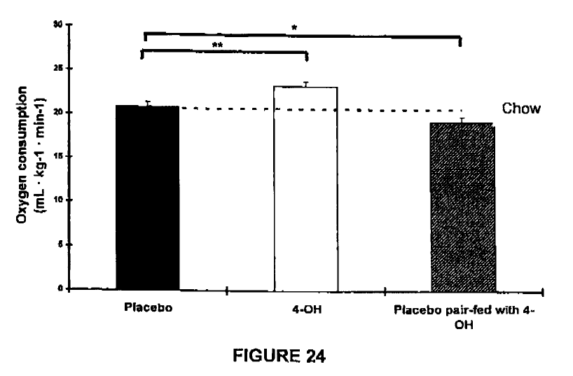

As shown in FIG. 24, energy expenditure (night phase) was increased for rats treated with 4-OH (white bar) since oxygen consumption was increased compared to the respective pair fed group (cross-hatched bar) and the HFHS control group (black bar, p<0.01). The increase in consumption was particularly evident during the night phase (19 h00 to 07 h00), the period of the day where rodents are typically the most actives.

### Example 12

### Effect of 4-OH on the Phosphorylation Level of AMPK-ACC in the Hypothalamus of HFHS Fed Rats

The AMPK pathway is known to regulate food intake in the hypothalamus. In low energy state as in a food deprivation situation, AMPK phosphorylates and in this way inactivates acetyl CoA carboxylase (ACC). Phosphorylation of ACC thus reduces its ability to catalyse the production of malonyl-CoA which is thought to be implicated in the stimulation of food consumption (.Hu, Z., Dai, Y., Prentki, M., Chohnan, S, and Lane, D. (2005) J. Biol. Chem. 280: 39681-39683).

To investigate whether 4-OH can alter activity of the AMPK/ACC in the hypothalamus, rats were treated BID with 100 mg/kg of 4-OH or water (control group) for 2 days. Each group was composed of four animals. All rats (controls and 4-OH treated) were fasted overnight before experimentation. On day 29, rats from each respective group were treated one hour before the terminal procedure with their appropriate solution (water for control and 4-OH for chronically 4-OH treated rats). After decapitation, the hypothalamus were isolated and snap frozen in liquid nitrogen. The hypothalamus was then powdered with liquid nitrogen and then solubilized in lysis buffer. Western blot was performed on the hypothalamus extract after resolution of proteins onto a 7.5% polyacrylamide SDS gel and transfer onto a nitrocellulose membrane. The membranes were incubated overnight with a polyclonal anti-pACC (phospho-acetyl Coenzyme A carboxylase) antibody from New England biolabs (Beverly, Mass.). Detection was performed with the Immobilon HRP ECL kit from Millipore (Billerica Mass.).

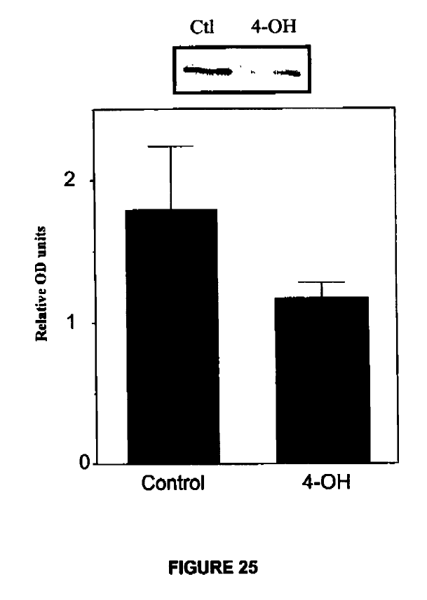

As all animal were fasted prior to experimentation, they should have an equivalent level of AMPK activity, as reflected by ACC inhibitory phosphorylation, which will reduce malonyl-CoA concentrations. This level of low phosphorylation can be observed for the control animals in FIG. 25. However, this signal is even more reduced in the 4-OH-treated group, which suggests that the food intake signal in the hypothalamus is turned down relative to control animals, despite an equivalent lack of food availability compared to the control group (overnight fasting). Therefore, these results demonstrate that the compounds according to the invention, and more particularly 4-hydroxyisoleucine (4-OH, compound 14a), can act through the AMPK/ACC pathway to alter food consumption and energy expenditure in rats fed a HFHS diet.

Those results also suggest that the compounds of the invention, including 4-OH, might have an effect on cellular signalling proteins and neurotransmitters (e.g. cAMP, leptin, adiponectin, AMP kinase, mTOR, PI3 kinase, MSH, NPY, POMC, noradrenaline, dopamine, serotonine (5-HT), MCH, orexin, POMC, CART, AgRP, etc.) in the regulation of lipolysis, adipogenesis and satiety.

### Example 13

### Effect of 4-hydroxyisoleucine on Body Weight and Lipid Profiles of SD Rats Fed Regular Chow

The objective of the study was to determine the toxicity and toxicokinetic profile of 4-Hydroxyisoleucine (4-OH, compound 14a), following oral (gavage) administration to the rats for 13 weeks.

Sprague-Dawley rats were received at 7-12 weeks of age (225-300 g). After a 2-week acclimation period, animals were randomized based on their body weight values, into control and treatment groups (n=10) and treated once daily by oral gavage with 4-OH at the doses of 100, 200, and 400 mg per kg of body weight for 13 weeks. The control group received vehicle (water) alone. Body weights were recorded once prior to group assignment, and approximately one week prior to initiation of treatment. Then, body weights were recorded for all animals up to 1 day prior to dosing and weekly thereafter during the treatment period. At the end of the treatment period, a blood sample was withdrawn to measure plasma triglyceride and cholesterol levels.

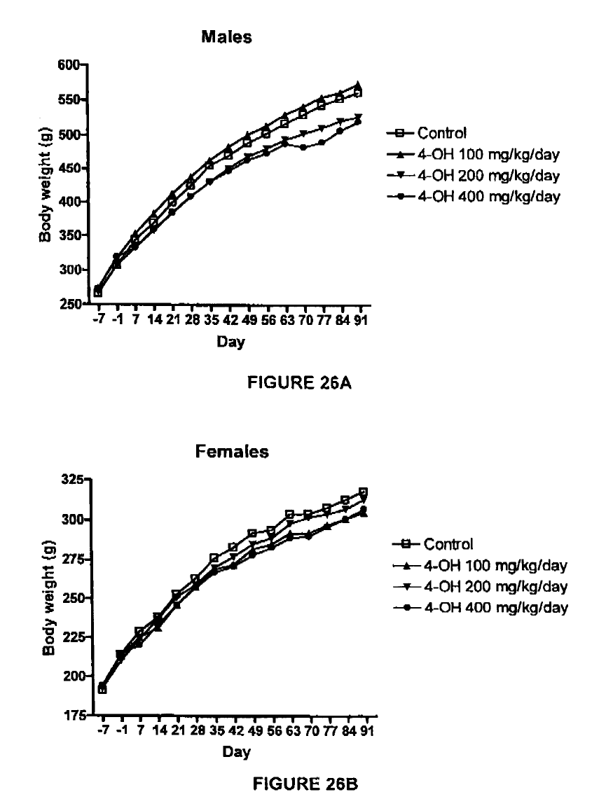

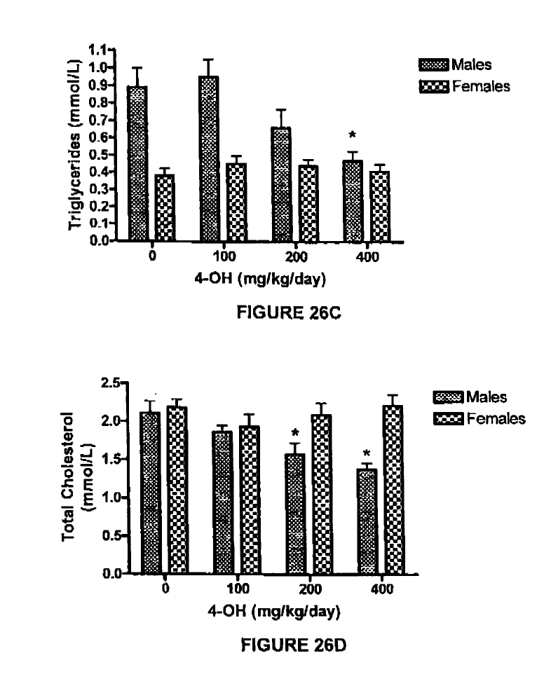

Male Sprague-Dawley rats treated with 4-OH at 200 and 400 mg/kg/day of body weight once daily showed a clear reduction of body weight compared to control rats (FIG. 26A). At the end of the treatment period, the average body weight of treated animals was 527 and 520 g for 200 and 400 mg/kg/day, respectively, versus 562 g for control animals. To a lesser extent, this effect was also observed in female Sprague-Dawley rats (FIG. 26B). Females are smaller and gained less weight than males during the course of the trial. Triglyceride and cholesterol levels are reduced in a dose-dependant manner by 4-OH in male rats (FIGS. 26C and 26D). This effect is statistically significant at 400 mg/kg/day for triglycerides and at 200 and 400 mg/kg/day for cholesterol.

These results demonstrate that 4-OH is effective at reducing body weight gain in normal rats. Interestingly, treatment with that 4-OH also reduced plasma triglyceride and total cholesterol levels in male rats. Thus, the results of this study confirm that the compounds according to the invention, and more particularly 4-OH, show great potential for the treatment of regulation lipid metabolism.

### Example 14

The effect of 4-OH on energy balance was assessed in the dietary obese rat model. In these studies, male Wistar rats were administered a high fat/high sucrose diet (65.4% fat) and treated chronically with 4-OH. Each study consisted of 4 groups of animals. The groups studied were normal chow fed rats, rats fed a high fat/high sucrose diet and rats fed a high fat high sucrose diet and orally administered 4-OH at 100 mg/kg twice per day. These three groups had access to food ad libitum throughout the study. A fourth group fed the same number of Kcal (or similar energy intake) as the 4-OH group was also included to investigate drug effects that may occur in addition to changes in food intake.

Two studies were performed: one to determine whether 4-OH could prevent the development of obesity (prevention study) and the other to determine whether 4-OH could reverse established obesity (reversal study). In the prevention study, male Wistar rats were administered the high fat/high sucrose diet for 5 weeks. During the first week of diet administration, the rats were adapted to receiving increasing doses of 4-OH. In the reversal of obesity study, the animals were first made obese by feeding the high fat/high sucrose diet for 28 days. This feeding regime continued for the duration of the study. After the period of obesity induction, the rats were also adapted to receiving increasing doses of 4-OH during a one week period. After the adaptation period, 4-OH was orally administered at a dose of 100 mg/kg twice per day for the following 4 week period in both treatment protocols.

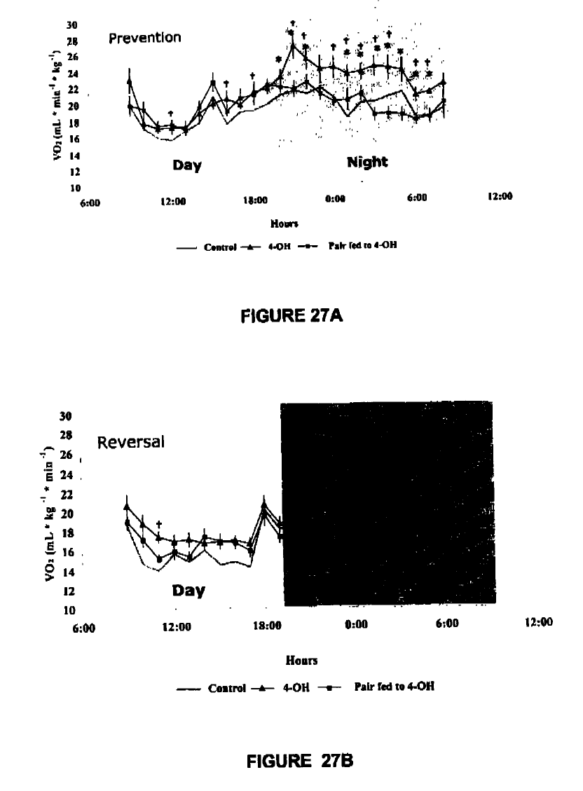

The ability of 4-OH to influence energy expenditure was determined by indirect calorimetry. On the 21^{st} day of both the prevention and reversal studies, rats were placed into metabolic cages and energy expenditure was assessed by determining oxygen consumption. The effect of 4-OH on oxygen consumption during the day/night cycle on Day 21 of both the prevention and reversal of obesity studies is shown in FIGS. 27A and 27B.

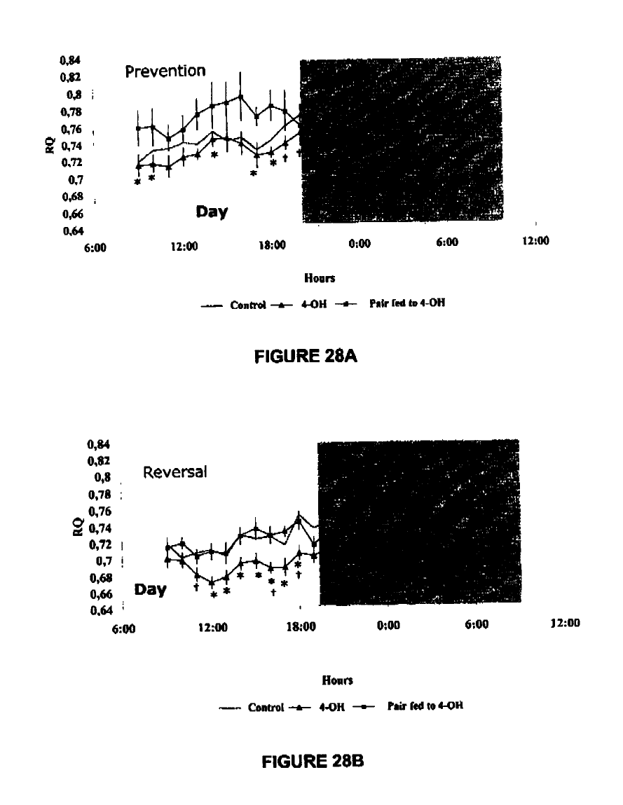

The results in FIGS. 28A and 28B show that oxygen consumption (VO₂) during the light phase of day/night cycle in both the prevention and reversal studies was essentially identical between the control, 4-OH and pair fed groups of animals. However, during the night phase, oxygen consumption expressed per kg of body weight was significantly enhanced relative to both the pair fed and control groups of animals. Even when expressed per rat, the VO₂ was greater in 4-OH treated rats as compared to its pair fed control groups. These finding indicate that 4-OH maintains or enhances energy expenditure despite reduced energy intake and body weight.

The respiratory quotient (RQ) was calculated in these studies from both the quantity of oxygen consumed and the quantity of carbon dioxide produced while the animals were present in the metabolic chambers.

A RQ of 1 is indicative that the animals are burning pure carbohydrate as the energy source. A decreasing RQ indicates that in addition to carbohydrate, progressively more fat is being burnt by the animals as a fuel source. The results in FIGS. 28A and 28B show that during the day phase, animals treated with 4-OH burn proportionally more fat than pair fed animals and in the reversal study proportionally more fat than both the pair fed and vehicle treated animals. During the night phase, when rats are actively feeding, both fat and carbohydrates are burned because insulin sensitivity for glucose uptake is also increased by 4-OH and the RQ is therefore not reduced during that period.

### Example 15

### Effect of Compound 13e on Body Weight Gain in the Diet-induced Obesity (DIO) Mouse Model

The objective of this study was to determine the effect of one analog according to the invention, namely Compound 13e, on body weight gain in the Diet-Induced Obesity (DIO) mouse model.

C57BL/6 mice were received at 7-8 weeks of age and fed a high fat diet (60% of calories from fat) for 8 weeks. Fasted glycemia and body weight values were used to randomize the mice into control and treatment groups (n=8). The average basal glycemia was between 213 and 215 mg/dL for all groups. The animals were treated twice daily by oral gavage with Compound 13e (25 or 50 mg per kg of body weight), and the control group received vehicle (200 mM bicarbonate buffer/0.1% Tween-20™, pH=9) alone. The animals were treated for 21 days. Body weight of the mice was measured on a frequent basis during the treatment. At the end of the study, the epididymal fat pads were isolated and weighed. Data are expressed as mean±SEM of body weight and mean±SEM of fat pad weight.

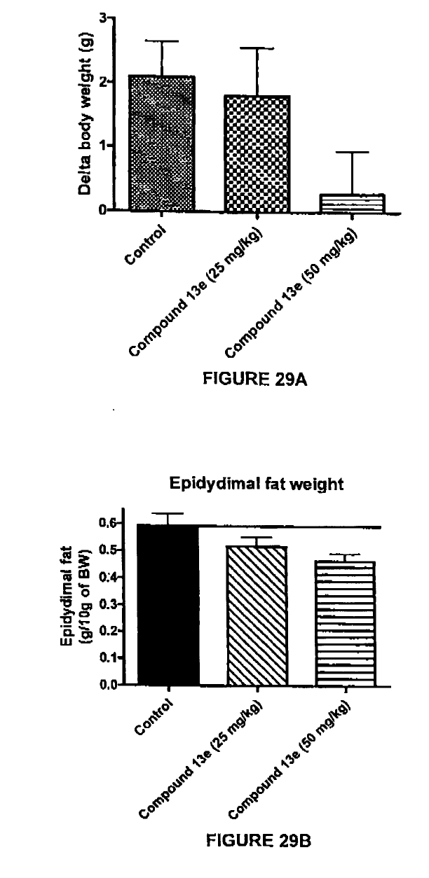

FIG. 29A shows the relative change in body weight after 21 days of treatment as expressed in delta of body weight from Day 0 of treatment. As illustrated in this figure, DIO mice treated with Compound 13e showed a reduction in body weight gain compared to vehicle treated mice and this effect was dose-dependent.

FIG. 29B shows the relative change in epididymal fat pad weight expressed in grams per 10 grams of body weight. As seen, the reduction of body weight induced by Compound 13e is correlated with a reduction of epididymal fat pad weight.

In conclusion, Compound 13e can reduce body weight gain in a well-recognized model of obesity, the DIO-mouse model. Since this effect was correlated with a reduction of the epididymal fat pad weight, this suggests that analogs according to the invention, and more particularly Compound 13e, could be beneficial for reducing visceral fat and treating obesity in humans when used as a monotherapy.

### Example 16

### Effect of Analogs and Isomers of 4-Hydroxyisoleucine on Body Weight Gain in C57BL/6 Mice Fed a High Fat Diet

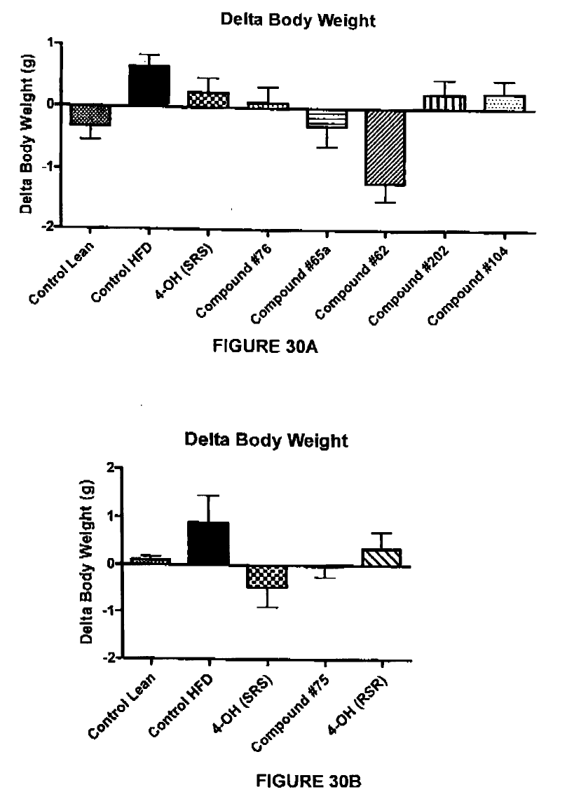

C57BL/6 mice were received at 6-7 weeks of age and fed a standard commercial chow for 1 week (acclimation period). The animals were randomized based on their body weight values, into control and treatment groups (n=6). Then, animals were shifted to a high fat diet (60% of calories from fat) and treated twice daily by oral gavage with 4-hydroxyisoleucine (4-OH, compound 14a) or different analogs and isomers of 4-OH at the dose of 100 mg per kg of body weight for 3 days. The control group (Control HFD) received vehicle (water) alone and a group was kept under standard chow (Control Lean). Body weight of the mice was recorded daily. Two different experiments were run and the effect on body weight gain of selected analogs and isomers according to the invention is presented in FIG. 30A (Experiment 1) and FIG. 30B (Experiment 2).

C57BL/6 mice under high-fat diet (Control HFD) gained weight rapidly as compared to the mice on a normal diet (Control Lean; see FIGS. 30A and 30B). Within 3 days, treatment with 4-OH at 100 mg/kg twice daily reduced body weight gain induced by the high fat diet (FIG. 30A) and in one experiment reduced body weight of the mice as compared to pre-treatment values (FIG. 30B). At the same dosage, analogs of 4-hydroxyisoleucine (compound #76, compound #65a, compound #62, compound #202, compound #104, and compound #75) and the 2R,3S,4R-isomer of 4-hydroxyisoleucine reduced body weight gain induced by the high fat diet. Two of these compounds, compound #65a and compound #62, showed a greater efficacy than the SRS isomer of 4-OH (compound #14a).

These results demonstrate that the analogs and isomers of 4-hydroxyisoleucine according to the invention, and more particularly the compounds exemplified in FIGS. 30A and 30B, are effective at reducing body weight gain of mice subjected to a high fat diet. These results also show the great potential of the compounds of the invention for the treatment of obesity.

### Example 17

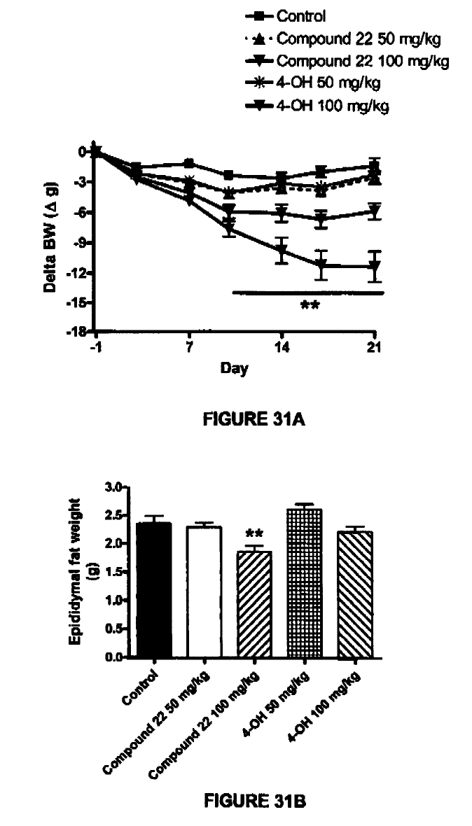

The objective of the following study was to evaluate the effect of the lactone form of 4-hydroxyisoleucine (compound 22) on the weight gain of obese mice. 40 C57BL/6 mice were fed a high fat diet (60% of calories from fat) for 6 weeks in order to induce obesity. Animals were than randomized to 5 study groups of 8 mice each according to their body weight values. Thereafter, the animals were treated orally twice daily with either the water vehicle, 50 mg/kg of compound 22, 100 mg/kg of compound 22, 50 mg/kg of 4-hydroxyisoleucine or 100 mg/kg of 4-hydroxyisoleucine. The treatment lasted 21 days and body weight of mice were determined daily from day 1 to day 21 of the study. FIG. 31A shows that both 4-hydroxyisoleucine and the lactone form of 4-hydroxyisloleucine, when given at a dose level of 100 mg/kg, induced a significant reduction of body weight compared to the water control group (p<0.01). When given at the same dose level of 100 mg/kg, the weight loss induced by the lactone form of 4-hydroxyisoleucine was significantly greater than the weight loss caused by 4-hydroxyisoleucine (p<0.05). FIG. 31B shows that the body weight loss was concomitantly associated with loss of the white fat mass (epididymal fat) for mice treated with 100 mg/kg of 4-hydroxyisoleucine or the lactone form of 4-hydroxyisoleucine (p<0.01).

### Example 18

### Effect of Compounds on Lipid Accumulation in 3T3-L1 Adipocytes

The objective of the study was to evaluate if the compounds of the present invention, and more particularly analogs of 4-Hydroxyisoleucine, can decrease the accumulation of lipids in pre-adipocytes induced to differentiate into functional adipocytes. The 3T3-L1 pre-adipocytes exhibit a fibroblast phenotype when cultured under standard conditions (DMEM plus 10% FBS). Treating the fibroblasts with a differentiation medium containing insulin, dexamethasone and 1-isobutyl-1-1-methylxanthine (IBMX) in the presence of serum induces these cells to become terminally differentiated adipocytes. These cells convert to spherical shape and accumulate lipid droplets.

3T3-L1 were cultured in presence of DMEM and 10% FBS for a week in 6-well plates. The medium was than changed to induce differentiation of cells into mature adipocytes. The medium consisted of DMEM, 10% FBS, 0.5 mM IBMX, 0.01 mg/ml insulin and 0.1 μM dexamethasone, with or without compounds of the present invention. The cells were cultured for 7 days and then stained with Oil red 0, a dye which specifically stain lipids. The fat content of treated and untreated cells was quantified by measuring the optical density (OD, 490 nm) with a spectrophotometer.

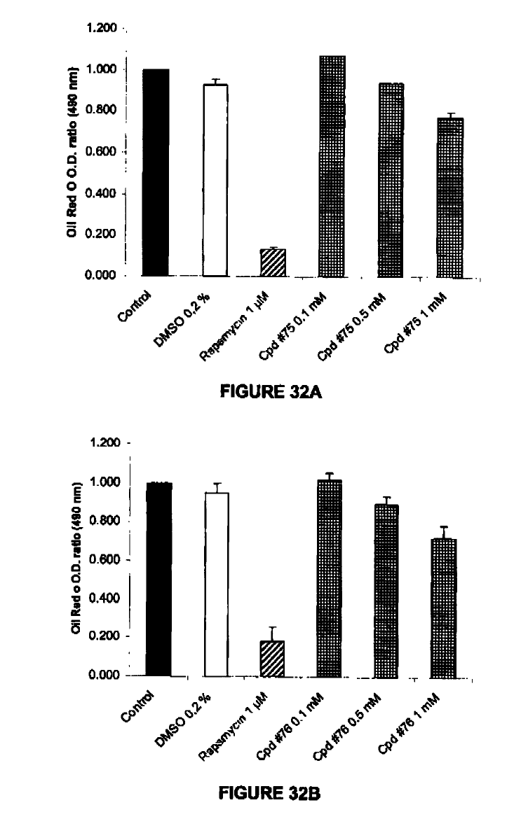

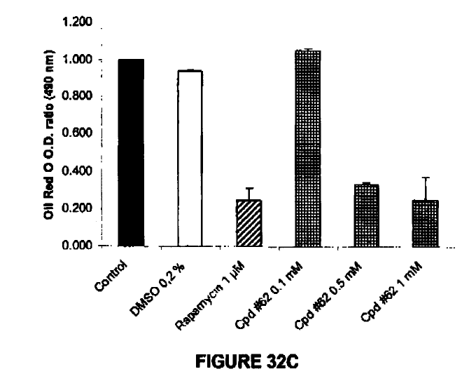

FIGS. 32A, 32B and 32C show that, compared to the controls (white and black bars), compound 75 (FIG. 32A), compound 76 (FIG. 32B), and compound 62 (FIG. 32C) (grid bars) dose-dependently decreased the lipid accumulation in 3T3-L1 cells induced to differentiate into mature adipocytes. Rapamycin (hatched bars) was used as a positive internal control for the assay.

Thus, the results of this study confirm that the compounds according to the invention show great potential for decreasing lipid accumulation into cells that are committed to become mature functional adipocytes.

### Example 19

### Effect of 4-Hydroxyisoleucine on Adipocyte Lipolysis in a Rat Model of Diet-Induced Obesity

The aim of this study was to evaluate the effect of chronic administration of 4-hydroxyisoleucine (4-OH, compound 14a) on adipocyte lipolysis in the Wistar rat model of diet-induced obesity. The animals were acclimated for 1 week and fed standard chow prior to the commencement of treatment.

A total of 60 animals were used in the study. The animals were distributed into 6 groups; 1 group was fed standard chow, 1 group was fed a high fat, high sucrose (HFHS) diet with 28 days treatment, 1 untreated control group was fed a HFHS diet, and 1 untreated group was fed a HFHS diet, but with caloric intake restricted to match the caloric intake of the 4-OH treated animals (pair-fed animals). A fifth group was fed a high fat, high sucrose (HFHS) diet with 28 days treatment and received an acute oral administration of 4-OH just prior to experimentation on Day 29 of the study, and a sixth group was fed a HFHS diet, but with caloric intake restricted to match the caloric intake of the 4-OH treated animals receiving an acute oral administration of 4-OH on Day 29 of the study (pair-fed animals). Each group was composed of 10 animals. Animals were housed separately and food consumption was monitored daily. Body weights were measured once weekly from week 1 to week 4 of the study.

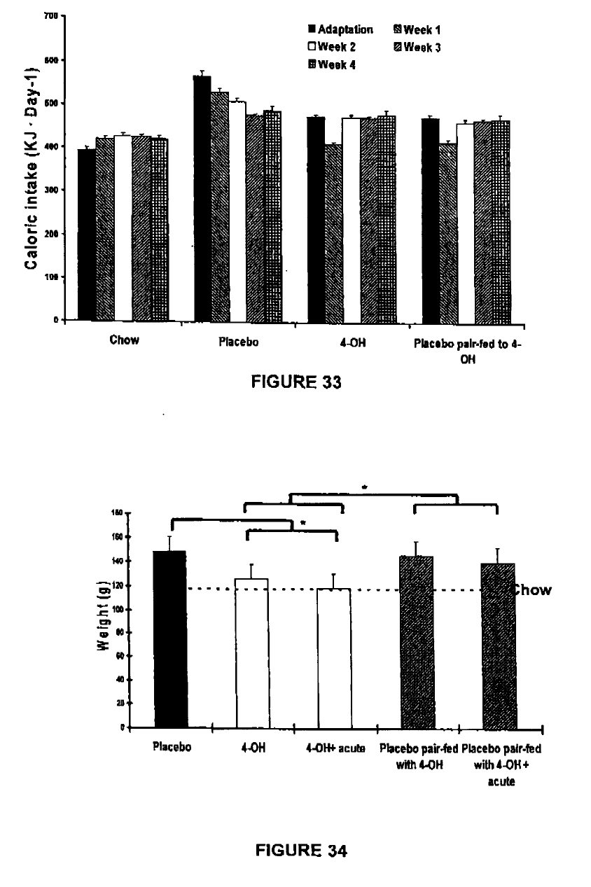

For the four weeks of treatment, the test article 4-OH was dissolved in reverse osmosis water. 4-OH aliquoted and kept at 4° C. Treated animals received twice daily oral administration of 4-OH at 100 mg/kg per dose. Untreated animals received water twice daily. As shown in FIG. 33, food consumption of 4-OH treated and pair-fed with 4-OH animals was slightly decreased during the first week of treatment compared to the HFHS control animals, suggesting that animals treated with 4-OH more easily reached satiety as compared with those of other groups. From the second to the fourth week of treatment, food consumption was similar across all groups.

Body weight gain of animals after 28 days of treatment is shown in FIG. 34. Weight gain of rats treated with 4-OH was significantly decreased compared to the pair fed group and the HFHS control group (p<0.05). Weight gain of rat receiving chronic administration of 4-OH plus an acute oral administration of 4-OH was also significantly decreased compared to the respective pair-fed group and the HFHS control group (p<0.05).

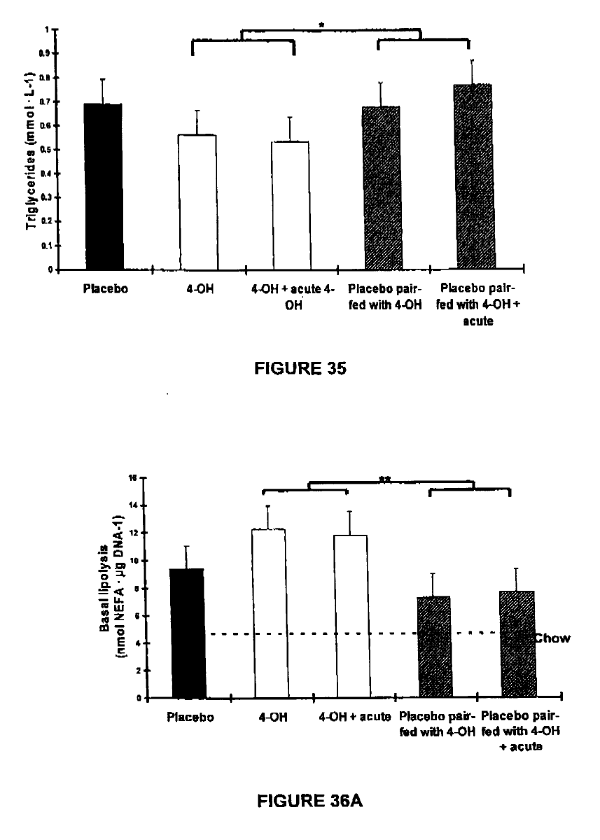

At the end of treatments, the animals were sacrificed and triglycerides plasma levels were determined. As shown in FIG. 35, triglyceride levels of treated rats (white bars) were significantly decreased compared to the respective pair fed groups (Hatched bars, p<0.05).

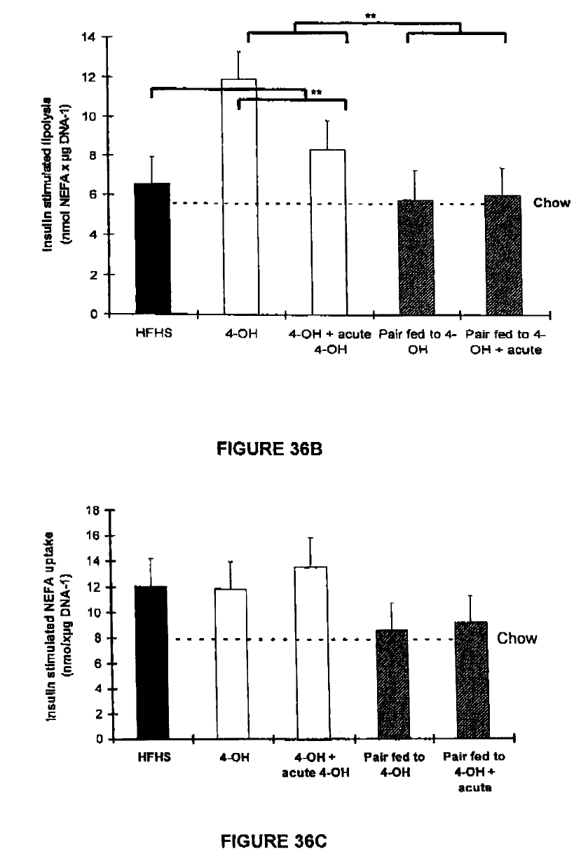

The white adipose tissues of non-treated and treated rats were collected and cultured ex vivo. After 2 hours of culture, the relative release and uptake of free fatty acids by the adipocytes was measured in absence or presence of insulin, and standardized on the basis of DNA content of the cultures. As shown in FIG. 36A, the adipocytes from untreated groups receiving a HFHS diet showed similar basal release of fatty acids (black bar and cross-hatched bars) independent of the caloric restriction in the pair-fed groups. The adipocytes from the 4-OH treated groups also receiving a HFHS diet (white bars), showed a significant increased basal fatty acid release compared to their respective pair-fed groups (hatched bars, p<0.01). FIG. 36B shows that insulin stimulated release of fatty acid was also significantly increased in groups treated with 4-OH (white bars) compared to their respective pair fed groups (hatched bars, p<0.01) and compared to the HFHS control group (Black bar, p<0.01), even if they were fed a high fat diet. Furthermore, FIG. 36C shows that fatty acid uptake in presence of insulin was not significantly different across all groups, treated or not with 4-OH. In conclusion, treatment with 4-OH enhanced lipolysis and insulin stimulated lipolysis (i.e., release of fatty acids), even in the presence of a high fat diet.

Thus, the results of this study confirm that the compounds according to the invention, and more particularly 4-hydroxyisoleucine (4-OH, compound 14a), could potentially be used therapeutically to modulate fat metabolism and reduce lipid content in adipocytes.

### Example 20

### 4-hydroxyisoleucine Modulates Expression of Genes Related to Lipid Metabolism

As the ex vivo studies on adipocytes indicated the lipolytic effect of drug was due to chronic exposure (see previous example), a role in gene regulation was assessed. RT-PCR was used to assess the expression of genes in the adipocytes from treated and control animals.

As shown in Table 5 below, there was a significant increase in expression of important genes related to lipid metabolism, suggesting that a key function of 4-hydroxyisoleucine (4-OH, compound 14a) is to regulate lipid metabolism.

TABLE 5
Fold times increase in expression of genes related to
lipid metabolism measured from adipocytes of rats fed
a high fat, high sucrose (HFHS) diet and treated or untreated
with 4-hydroxyisoleucine for 4 weeks (28 days).

| Genes | Increase (x times) |
|---|---|
| HSL | 1.3 |
| ATGL | 2.6 |
| FABP4/ap2 | 1.4 |

Of interest, three important enzymes/binding proteins were up regulated. Expression of hormone sensitive lipase (HSL), the key enzyme that hydrolyzes intracellular triacyglycerol and diacylglycerol, was increased 1.3-1.4 times, as was FABP4/ap2, a protein which can bind HSL and participates in the export of fatty acids for oxidation. The expression of ATGL, the adipose triglyceride lipase, was also increased more than two times. ATGL has been shown to be the rate limiting step in the catabolism of cellular fat depots and plays an important role in energy homeostasis. Upregulation of FatB1, the bidirectional transporter of fatty acids found in the adipocyte membrane, and CPT1, the transporter involved in shunting free fatty acids into the mitochondria for subsequent metabolism, was also observed (data not shown).

Thus, while 4-OH facilitates lypolysis, that compound also facilitates removal of lipids from systemic circulation. Accordingly, these results support using the compounds according to the invention, and more particularly 4-OH for removing lipids from systemic circulation. Furthermore it will be appreciated that since 4-OH is capable of reducing weight and/or preventing onset or progression of excessive weight gain in mammals, the above mentioned genes and genes products are thus linked to weight reduction and/or weight gain prevention. Therefore modulation of the expression of genes (and products thereof) involved in lipid metabolism can contribute to weight reduction and/or prevention of onset or progression of excessive weight gain in mammals. More particularly the modulation of the expression of the genes mentioned above, such as for example by increasing their expression, can modulate weight variations in a mammal.

Modulation of gene expression (and/or product thereof) herein can refer, for example to positive (i.e., up-regulation) or negative (i.e., down-regulation) regulation of gene transcription, and to the modulation of the gene and gene product. Methods for modulating the expression of genes and gene products are known in the art and may include without being limited to regulation of the promoter, anti-sense RNA, binding of inhibitor to the gene product or proteins involved in the gene regulation, modification of the DNA sequence of regulatory sequences, triplex-forming oligonucleotides and the like.

### Example 21

### Effect of 4-Hydroxyisoleucine and Compound 65a on Body Weight Gain and Food Consumption of Diet Induced Obesity (DIO)-Mice

The objective of this study was to evaluate the effect of chronic administration of 4-OH and compound 65a on food consumption and body weight gain. Both parameters were monitored for 1 week prior to the commencement of treatment, then for one week of treatment.

A total of 40 animals were used in the study. The animals were distributed into 5 groups (4 treated, 1 control group, all on high fat diet). Each group was composed of 8 animals. The mice were randomized according to body weight and basal glycemia values following a 5±0.5 hours fasting period.

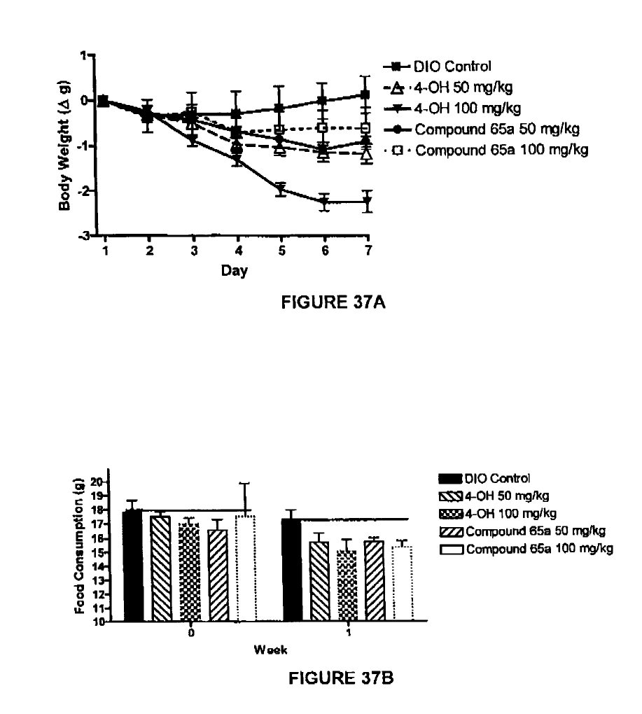

For the week of treatment, the test articles were dissolved in reverse osmosis water. 4-OH was aliquoted and kept at 4° C. Control animals received reverse osmosis water twice daily (Group 1). Mice from groups 2 and 3 were treated twice daily with 4-OH at 50 and 100 mg/kg, respectively. Mice from groups 4 and 5 were treated twice daily with 50 and 100 mg/kg of compound 65a. All groups were treated by oral gavage. Treatment commenced on Day 0 and ended on Day 7 (FIG. 37A). Body weights were measured daily. Food consumption were measured daily and averaged on a weekly basis beginning one week before the start of treatment as shown in FIG. 37B.

These results indicate that treatments were well tolerated for all groups receiving 4-OH or compounds 65a. During the first week of treatment, moderation of weight gain was observed for animals receiving 4-OH or compound 65a at 50 and 100 mg/kg (FIG. 37A). This reduction in body weight gain was paralleled with a slight decrease in food consumption during the first week of treatment (FIG. 37B). This demonstrates that both 4-OH and compound 65a can reduce weight gain and food consumption in dietary obese rodent models.

### CONCLUSIONS

Augmentation of lipolysis while adiposity is reduced and insulin sensitivity is improved suggests that the released lipids are oxidized. The data on energy expenditure in vivo fits with that hypothesis as well. Taken altogether, those results indicate that the compounds according to the invention, and more particularly 4-hydroxyisoleucine (4-OH, compound 14a), have a unique and novel mechanism of action supporting their uses to address the problem of disorders of lipid metabolism. The results presented herein also support the rationale of using the compounds according to the invention, and more particularly 4-hydroxyisoleucine, for the therapeutic treatment of obesity, and more particularly for reducing accumulated weight gain and visceral fat.

1-150. (canceled)

151. A method of regulating fat metabolism in a mammal, said method comprising administering to said mammal a compound selected from the group consisting of: isomers of 4-hydroxyisoleucine, analogs of 4-hydroxyisoleucine, and pharmaceutically acceptable lactones, salts, metabolites, solvates, and/or prodrugs of said isomers and analogs.

152. The method of claim 151, wherein said mammal is afflicted with a disease or condition selected from the group consisting of a disorder of lipid metabolism, lipodystrophy, hypercholesterolemia, atherosclerosis, and non-alcoholic fatty liver disease.

153. The method of claim 152, wherein said non-alcoholic fatty liver disease is non-alcoholic steatohepatitis.

154. The method of claim 151, wherein administration of said compound results in one or more of the following effects in said mammal: reducing caloric intake/food consumption, reducing body fat, increasing energy expenditure, increasing oxygen consumption, stimulation of lipolysis by adipocytes, enhancing insulin stimulated lipolysis, facilitating removal of lipids from systemic circulation, modulating expression of genes related to lipid metabolism, reducing intestinal lipid adsorption, modulating of AMP kinase and modulating cellular signalling proteins and neurotransmitters.

155. The method of claim 154, wherein said genes related to lipid metabolism comprises FABP4/aP2, HSL, ATGL, FatB1 or CPT-1.

156. A method selected from the group consisting of:
A) preventing or treating obesity in a mammal, said method comprising administering to said mammal a compound selected from the group consisting of: isomers of 4-hydroxyisoleucine, analogs of 4-hydroxyisoleucine, and pharmaceutically acceptable lactones, salts, metabolites, solvates, and/or prodrugs of said isomers and analogs;
B) reducing body weight and/or body fat in a mammal, said method comprising administering to said mammal a compound selected from the group consisting of: isomers of 4-hydroxyisoleucine, analogs of 4-hydroxyisoleucine, and pharmaceutically acceptable lactones, salts, metabolites, solvates, and/or prodrugs of said isomers and analogs;
C) decreasing appetite and/or decreasing food intake in a mammal, said method comprising administering to said mammal a compound selected from the group consisting of: isomers of 4-hydroxyisoleucine, analogs of 4-hydroxyisoleucine, and pharmaceutically acceptable lactones, salts, metabolites, solvates, and/or prodrugs of said isomers and analogs;
D) preventing the onset or progression of excessive weight gain in a mammal, said method comprising administering to said mammal a compound selected from the group consisting of: isomers of 4-hydroxyisoleucine, analogs of 4-hydroxyisoleucine, and pharmaceutically acceptable lactones, salts, metabolites, solvates, and/or prodrugs of said isomers and analogs; and
E) improving the bodily appearance of a mammal, said method comprising administering to said mammal a compound selected from the group consisting of: isomers of 4-hydroxyisoleucine, analogs of 4-hydroxyisoleucine, and pharmaceutically acceptable lactones, salts, metabolites, solvates, and/or prodrugs of said isomers and analogs.

157. The method of claim 151 or 156 wherein said mammal is a human.

158. The method of claim 157, wherein said human is overweight or obese.

159. The method of claim 158, wherein said human has a Body Mass Index (BMI) of at least 25.

160. The method of claim 159, wherein said human has a Body Mass Index (BMI) of at least 30.

161. The method of claim 151 or 156, wherein said compound is selected from the group consisting of:
A) an isomer of 4-hydroxyisoleucine or a pharmaceutically acceptable lactone, salt, metabolite, solvate, and/or prodrug thereof;
B) an isomer of 4-hydroxyisoleucine having the formula: 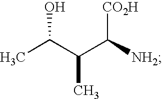
C) an isomer of 4-hydroxyisoleucine selected from the group consisting of: 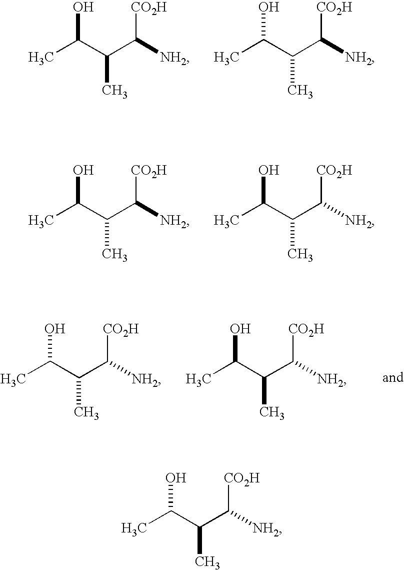
or a pharmaceutically acceptable salt, metabolite, solvate, and/or prodrug thereof; and
D) A lactone of 4-hydroxyisoleucine selected from the group consisting of: 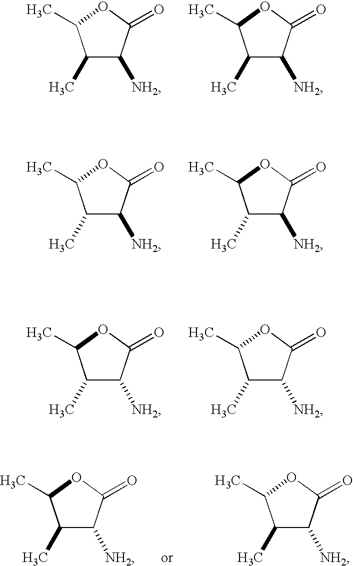
or a pharmaceutically acceptable salt, metabolite, solvate, and/or prodrug thereof.

162. The method of claim 151 or 156, wherein said compound is an analog of 4-hydroxyisoleucine or a pharmaceutically acceptable lactone, salt, metabolite, solvate, and/or prodrug thereof.

163. The method of claim 162, wherein said compound is of Formula (I): 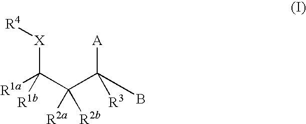
wherein
A is COR^{A1}, C(O)SR^{A1}, C(S)SR^{A1}, C(O)NR^{A2}R^{A3}, C(S)NR^{A2}R^{A3}, C(O)R^{A4}, SO₃H, S(O)₂NR^{A2}R^{A3}, C(O)R^{A5}, C(OR^{A1})R^{A9}R^{A10}, C(SR^{A1})R^{A9}R^{A10}, C(═NR^{A1})R^{A5}, P(O)(OH)₂, 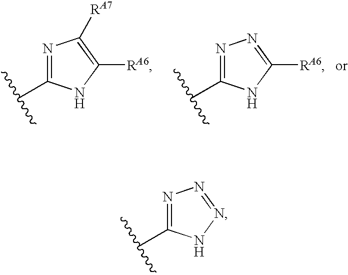
wherein
R^{A1} is hydrogen, or a substituted or unsubstituted group selected from C₁₋₆ alkyl, C₃₋₈ cycloalkyl, alkcycloalkyl, where the cycloalkyl group is of three to eight carbon atoms and the alkylene group is of one to four carbon atoms, C₂₋₆ alkenyl, C₂₋₆ alkynyl, C₆ or C₁₀ aryl, C₇₋₁₆ alkaryl, where the alkylene group is of one to four carbon atoms, C₁₋₉ heterocyclyl, or C₂₋₁₅ alkheterocyclyl, where the alkylene group is of one to four carbon atoms,
each of R^{A2} and R^{A3} is, independently, selected from the group consisting of (a) hydrogen, and a substituted or unsubstituted group selected from (b) C₁₋₆ alkyl, (c) C₃₋₈ cycloalkyl, (d) alkcycloalkyl, where the cycloalkyl group is of three to eight carbon atoms and the alkylene group is of one to four carbon atoms, (e) C₆ or C₁₀ aryl, and (f) C₇₋₁₆ alkaryl, where the alkylene group is of one to six carbon atoms, or R^{A2} taken together with R^{A3} and N forms a 5- or 6-membered ring, optionally containing O or NR^{A8}, wherein R^{A8} is hydrogen or C₁₋₆ alkyl,
R^{A4} is hydrogen, or a substituted or unsubstituted group selected from C₁₋₆ alkyl, C₃₋₈ cycloalkyl, alkcycloalkyl, where the cycloalkyl group is of three to eight carbon atoms and the alkylene group is of one to four carbon atoms, C₆ or C₁₀ aryl, C₇₋₁₆ alkaryl, where the alkylene group is of one to four carbon atoms, C₁₋₉ heterocyclyl, or C₂₋₁₅ alkheterocyclyl, where the alkylene group is of one to four carbon atoms,
R^{A5} is a peptide chain of 1-4 natural or unnatural amino acids, where the peptide is linked via its terminal amine group to C(O),
each of R^{A6} and R^{A7} is, independently, hydrogen, substituted or unsubstituted C₁₋₆ alkyl, C₁₋₄ perfluoroalkyl, substituted or unsubstituted C₁₋₆ alkoxy, amino, C₁₋₆ alkylamino, C₂₋₁₂ dialkylamino, N-protected amino, halo, or nitro, and
each of R^{A9} and R^{A10} is, independently, selected from the group consisting of (a) hydrogen, and a substituted or unsubstituted group selected from (b) C₁₋₆ alkyl, (c) C₃₋₈ cycloalkyl, (d) alkcycloalkyl, where the cycloalkyl group is of three to eight carbon atoms and the alkylene group is of one to four carbon atoms, (e) C₆ or C₁₀ aryl, and (f) C₇₋₁₆ alkaryl, where the alkylene group is of one to six carbon atoms, or R^{A9} taken together with R^{A10} and their parent carbon atom forms a 5- or 6-membered ring, optionally containing O or NR^{A8}, wherein R^{A8} is hydrogen or C₁₋₆ alkyl;
B is NR^{B1}R^{B2}, wherein
(i) each of R^{B1} and R^{B2} is, independently selected from the group consisting of (a) hydrogen, (b) an N-protecting group, and a substituted or unsubstituted group selected from (c) C₁₋₆ alkyl, (d) C₂₋₆ alkenyl, (e) C₂₋₆ alkynyl, (f) C₃₋₈ cycloalkyl, (g) alkcycloalkyl, where the cycloalkyl group is of three to eight carbon atoms, and the alkylene group is of one to ten carbon atoms, (h) C₆ or C₁₀ aryl, (i) C₇₋₁₆ alkaryl, where the alkylene group is of one to six carbon atoms, (j) C₁₋₉ heterocyclyl, (k) C₂₋₁₅ alkheterocyclyl, where the alkylene group is of one to six carbon atoms, (1) (CH₂)ₙC(O)R^{B3}, wherein n is 0, 1, 2 or 3, where R^{B3} is selected from the group consisting of hydrogen and a substituted or unsubstituted group selected from C₁₋₆alkyl, C₆ or C₁₀ aryl, C₇₋₁₆ alkaryl, where the alkylene group is of one to six carbon atoms, C₁₋₉heterocyclyl, or C₂₋₁₅ alkheterocyclyl, where the alkylene group is of one to six carbon atoms, (m) (CH₂)ₙCO₂R^{B4}, wherein n is 0, 1, 2 or 3, where R^{B4} is selected from the group consisting of hydrogen and a substituted or unsubstituted group selected from C₁₋₆ alkyl, C₆ or C₁₀ aryl, C₇₋₁₆ alkaryl, where the alkylene group is of one to six carbon atoms, C₁₋₉ heterocyclyl, or C₂₋₁₅ alkheterocyclyl, where the alkylene group is of one to six carbon atoms, (n) C(O)NR^{B5}R^{B6}, where each of R^{B5} and R^{B6} is, independently, selected from the group consisting of hydrogen and a substituted or unsubstituted group selected from C₁₋₆ alkyl, C₆ or C₁₀ aryl, C₇₋₁₆ alkaryl, where the alkylene group is of one to six carbon atoms, C₁₋₉ heterocyclyl, and C₂₋₁₅ alkheterocyclyl, where the alkylene group is of one to six carbon atoms, or R^{B5} taken together with R^{B6} and N forms a 5- or 6-membered ring, optionally containing a non-vicinal O, S, or NR′, where R′ is H or C₁₋₆ alkyl, (o) S(O)₂R^{B7}, where R^{B7} is selected from the group consisting of hydrogen and a substituted or unsubstituted group selected from C₁₋₆alkyl, C₆ or C₁₀ aryl, C₇₋₁₆ alkaryl, where the alkylene group is of one to six carbon atoms, C₁₋₉ heterocyclyl, or C₂₋₁₅ alkheterocyclyl, where the alkylene group is of one to six carbon atoms, and (p) a peptide chain of 1-4 natural or unnatural alpha-amino acid residues, where the peptide is linked via its terminal carboxy group to N, with the proviso that no two groups are bound to the nitrogen atom through a carbonyl group or a sulfonyl group, or
(ii) R^{B1} taken together with R^{B2} and N forms a substituted or unsubstituted 5- or 6-membered ring, optionally containing O or NR^{B8}, wherein R^{B8} is hydrogen or C₁₋₆ alkyl, or
(iii) a 5- to 8-membered ring is formed when R^{B1} taken together with R^{1a} is a substituted or unsubstituted C₁₋₄ alkylene, or
(iv) a [2.2.1] or [2.2.2] bicyclic ring system is formed when R^{B1} taken together with R^{1a} is a substituted or unsubstituted C₂ alkylene and R^{B1} taken together with R^{2a} is a substituted or unsubstituted C₁₋₂ alkylene, or
(v) a 4- to 8-membered ring is formed when R^{B1} taken together with R³ is a substituted or unsubstituted C₂₋₆ alkylene, or
(vi) a 6- to 8-membered ring is formed when R^{B1} taken together with R⁴ is a substituted or unsubstituted C₁₋₃ alkylene, or
(vii) R^{B1} taken together with A and the parent carbon of A and B forms the following ring: 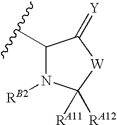
wherein each of Y and W is, independently, O, S, NR^{B8}, or CR^{A9}R^{A10}, wherein each of R^{A9} and R^{A10} is as previously defined and each of R^{A11} and R^{A12} is, independently, selected from the group consisting of (a) hydrogen and a substituted or unsubstituted group selected from (b) C₁₋₆ alkyl, (c) C₃₋₈ cycloalkyl, (d) alkcycloalkyl, where the cycloalkyl group is of three to eight carbon atoms and the alkylene group is of one to four carbon atoms, (e) C₆ or C₁₀ aryl, and (f) C₇₋₁₆ alkaryl, where the alkylene group is of one to six carbon atoms, or R^{A9} taken together with R^{A10} and their parent carbon atom forms a 5- or 6-membered ring, optionally containing O or NR^{A8}, wherein R^{A8} is hydrogen or C₁₋₆ alkyl;
X is (i) absent (ii) hydrogen, (iii) a substituted or unsubstituted group selected from a C₁₋₆ alkyl, C₃₋₈ cycloalkyl, alkcycloalkyl, where the cycloalkyl group is of three to eight carbon atoms and the alkylene group is of one to four carbon atoms C₆ or C₁₀ aryl, and C₇₋₁₆ alkaryl, where the alkylene group is of one to four carbon atoms, (iv) SO₃H; (v) O, (vi) S, or (vii) NR^{X1}, where R^{X1} is selected from the group consisting of (a) hydrogen (b) an N-protecting group, and a substituted or unsubstituted group selected from (c) C₁₋₆ alkyl, (d) C₂₋₆ alkenyl, (e) C₂₋₆ alkynyl, (f) C₃₋₈ cycloalkyl, (g) alkcycloalkyl, where the cycloalkyl group is of three to eight carbon atoms, and the alkylene group is of one to ten carbon atoms, (h) C₆ or C₁₀ aryl, (i) C₇₋₁₆ alkaryl, where the alkylene group is of one to six carbon atoms, (j) C₁₋₉ heterocyclyl, or (k) C₂₋₁₅ alkheterocyclyl, where the alkylene group is of one to six carbon atoms;
each of R^{1a} and R^{1b} is, independently, (a) hydrogen, (b) a substituted or unsubstituted group selected from C₁₋₆ alkyl, C₃₋₈ cycloalkyl, alkcycloalkyl, where the cycloalkyl group is of three to eight carbon atoms and the alkylene group is of one to four carbon atoms, C₂₋₆ alkenyl, C₂₋₆ alkynyl, C₆ or C₁₀ aryl, C₇₋₁₆ alkaryl, where the alkylene group is of one to four carbon atoms, C₁₋₉ heterocyclyl, and C₂₋₁₅ alkheterocyclyl, where the alkylene group is of one to four carbon atoms, or (c) R^{1a} together with R^{2a} and their base carbon atoms form a substituted or unsubstituted C₅₋₁₀ mono or fused ring system, or a 3- to 6-membered ring is formed when R^{1a} together with R⁴ is a substituted or unsubstituted C₁₋₄ alkylene (d) NR^{B1}R^{B2}, (e) a OR⁴ group, or (f) R^{1a} and R^{1b} together are ═O, ═N(C₁₋₆ alkyl), ═CR^{1c}CR^{1d}, where each of R^{1c} and R^{1d} is, independently, hydrogen or a substituted or unsubstituted group selected from a C₁₋₆ alkyl or a C₂₋₅ alkylene moiety forming a spiro ring;
each of R^{2a} and R^{2b} is, independently, hydrogen, F, Cl, Br, I, a substituted or unsubstituted group selected from C₁₋₆ alkyl, C₃₋₈ cycloalkyl, alkcycloalkyl, where the cycloalkyl group is of three to eight carbon atoms and the alkylene group is of one to four carbon atoms, C₂₋₆ alkenyl, C₂₋₆ alkynyl, C₆ or C₁₀ aryl, C₇₋₁₆ alkaryl, where the alkylene group is of one to four carbon atoms, C₁₋₉ heterocyclyl, or C₂₋₁₅ alkheterocyclyl, where the alkylene group is of one to four carbon atoms, or R^{2a} and R^{2b} together are ═O, ═N(C₁₋₆ alkyl), ═CR^{2c}R^{2d}, where each of R^{2c} and R^{2d} is, independently, hydrogen or substituted or unsubstituted C₁₋₆ alkyl, or a substituted or unsubstituted C₂₋₅ alkylene moiety forming a spiro ring, or R^{2a} together with R^{1a} and their base carbon atoms form a substituted or unsubstituted C₅₋₁₀ mono or fused ring system;
R³ is hydrogen, COOR^{A1}, or a substituted or unsubstituted group selected from C₁₋₆ alkyl, alkcycloalkyl, where the cycloalkyl group is of three to eight carbon atoms and the alkylene group is of one to four carbon atoms, C₂₋₆ alkenyl, C₂₋₆ alkynyl, C₇₋₁₆ alkaryl, where the alkylene group is of one to four carbon atoms, or C₂₋₁₅ alkheterocyclyl, where the alkylene group is of one to four carbon atoms; and
R⁴ is absent, hydrogen, a substituted or unsubstituted group selected from C₁₋₆ alkyl, C₃₋₈ cycloalkyl, alkcycloalkyl, where the cycloalkyl group is of three to eight carbon atoms and the alkylene group is of one to four carbon atoms, C₂₋₆ alkenyl, C₂₋₆ alkynyl, C₆ or C₁₀ aryl, C₇₋₁₆ alkaryl, where the alkylene group is of one to four carbon atoms, C₁₋₉ heterocyclyl, or C₂₋₁₅ alkheterocyclyl, where the alkylene group is of one to four carbon atoms, or a 3- to 6-membered ring is formed when R⁴ together with R^{1a} is a substituted or unsubstituted C₁₋₄ alkylene, or a 6- to 8-membered ring is formed when R⁴ taken together with RBI is a substituted or unsubstituted C₁₋₃ alkylene;
or a pharmaceutically acceptable lactone, salt, metabolite, solvate, and/or prodrug thereof.

164. The method of claim 163, wherein said compound is selected from the group consisting of or a pharmaceutically acceptable lactone, salt, metabolite, solvate, and/or prodrug thereof:
A) a compound of Formula (II): 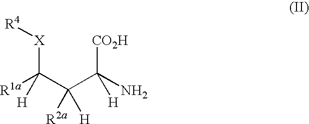
wherein each of X and R⁴ is as previously defined in reference to Formula (I) and each of R^{1a} and R^{2a} is, independently, substituted or unsubstituted C₁₋₆ alkyl or R^{1a} together with R^{2a} and their base carbon atoms form a substituted or unsubstituted 6 membered ring;
B) a compound of Formula (III): 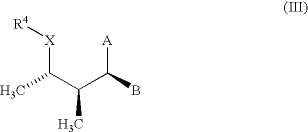
wherein A is CO₂R^{A1}, C(O)SR^{A1}, C(O)NR^{A2}R^{A3}, or C(O)R^{A5}; and each of R^{A1}, R^{A2}, R^{A3}, R^{A5}, B, X, and R⁴ is as previously defined in reference to Formula (I);
C) a compound of Formula (IV): 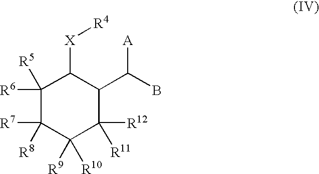
wherein A is CO₂R^{A1}, C(O)SR^{A1}, C(O)NR^{A2}R^{A3}, or C(O)R^{A5}; each of B, X, and R⁴ is as previously defined in reference to Formula (I); and each of R⁵, R⁶, R⁷, R⁸, R⁹, R¹⁰, R¹¹, and R¹² is, independently, hydrogen, or a substituted or unsubstituted group selected from C₁₋₆ alkyl, C₃₋₈ cycloalkyl, alkcycloalkyl, where the cycloalkyl group is of three to eight carbon atoms and the alkylene group is of one to four carbon atoms, C₂₋₆ alkenyl, C₂₋₆ alkynyl, C₆ or C₁₀ aryl, C₇₋₁₆ alkaryl, where the alkylene group is of one to four carbon atoms, C₁₋₉ heterocyclyl, or C₂₋₁₅ alkheterocyclyl, where the alkylene group is of one to four carbon atoms;
D) a compound of a formula selected from: 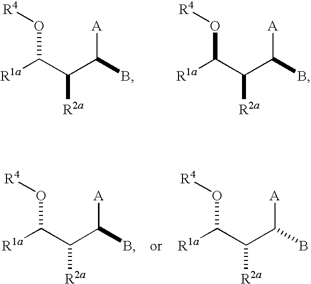
wherein each of A, B, and R⁴ is as previously defined in reference to Formula (I), and each of R^{1a} and R^{2a} is, individually, a substituted or unsubstituted group selected from C₁₋₆ alkyl, C₃₋₈ cycloalkyl, alkcycloalkyl, where the cycloalkyl group is of three to eight carbon atoms and the alkylene group is of one to four carbon atoms, C₂₋₆ alkenyl, C₂₋₆ alkynyl, C₆ or C₁₀ aryl, C₇₋₁₆ alkaryl, where the alkylene group is of one to four carbon atoms, C₁₋₉ heterocyclyl, or C₂₋₁₅ alkheterocyclyl, where the alkylene group is of one to four carbon atoms;
E) a compound of formula I as defined in claim 163, wherein A is CO₂H, B is NH-p-toluenesulfonyl, R⁴ is H, and each of R^{1a} and R^{2a} is CH₃;
F) a compound of formula I as defined in claim 163, wherein A is CO₂H, B is NH₂, R⁴ is H, and each of R^{1a} and R²¹ is a substituted or unsubstituted C₁₋₆ alkyl;
G) a compound of formula I as defined in claim 163, wherein A is CO₂H, B is NH₂, X is O, and R⁴ is H;
H) a compound of a formula selected from: 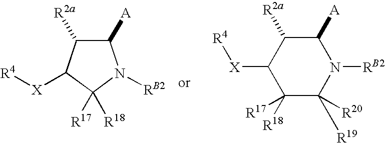
wherein each of A, X, R^{2a}, R⁴, and R^{B2} is as previously defined in reference to Formula (I), and each of R¹⁷, R¹⁸, R¹⁹, and R²⁰ is hydrogen or substituted or unsubstituted C₁₋₆ alkyl;
I) a compound of a formula selected from: 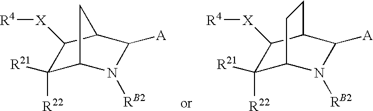
wherein each of A, X, R⁴, and R^{B2} is as previously defined in reference to Formula (I), and each of R²¹ and R²² is hydrogen or substituted or unsubstituted C₁₋₆ alkyl;
J) a compound of the formula: 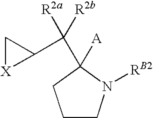
wherein each of A, X, R^{2a}, R^{2b}, and R^{B2} is as previously defined in reference to Formula (I;
K) a compound of the formula: 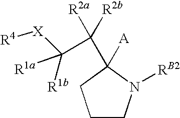
wherein each of A, X, R^{1a}, R^{1b}, R^{2a}, R^{2b}, R⁴, and R^{B2} is as previously defined in reference to Formula (I);
L) a compound of formula I as defined in claim 163, wherein R^{1a} together with R^{2a} and their base carbon atoms form a substituted or unsubstituted C₅₋₁₀ mono or fused ring system, optionally containing a non-vicinal O, S, or NR′, where R′ is H or C₁₋₆ alkyl;
M) a compound of a formula selected from: 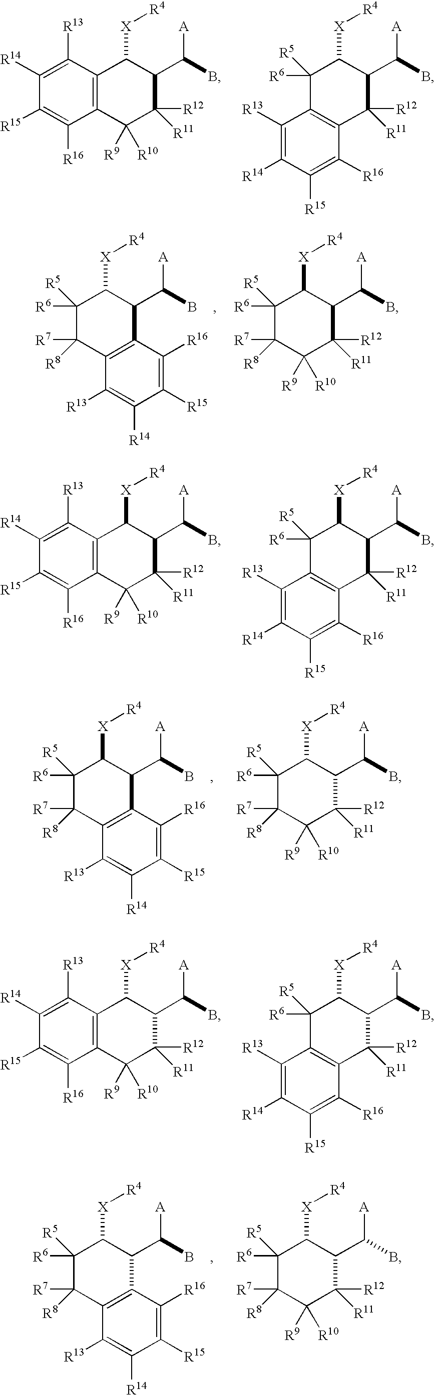 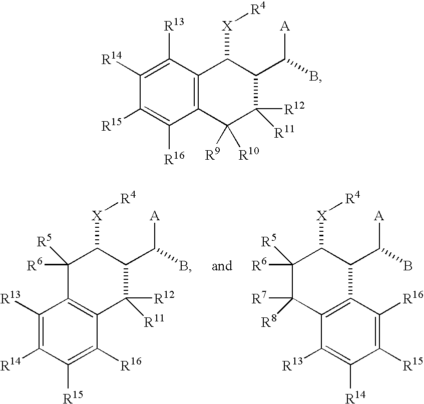
wherein each of A, B, X, and R⁴ is as defined previously in reference to Formula (I), and each of R⁵, R⁶, R⁷, R⁸, R⁹, R¹⁰, R¹¹, and R¹² is, independently, hydrogen, or a substituted or unsubstituted group selected from C₁₋₆ alkyl, C₃₋₈ cycloalkyl, alkcycloalkyl, where the cycloalkyl group is of three to eight carbon atoms and the alkylene group is of one to four carbon atoms, C₂₋₆ alkenyl, C₂₋₆ alkynyl, C₆ or C₁₀ aryl, C₇₋₁₆ alkaryl, where the alkylene group is of one to four carbon atoms, C₁₋₉ heterocyclyl, or C₂₋₁₅ alkheterocyclyl, where the alkylene group is of one to four carbon atoms; and
each of R¹³, R¹⁴, R¹⁵, and R¹⁶ is, independently, hydrogen, substituted or unsubstituted C₁₋₆ alkyl, C₁₋₄ perfluoroalkyl, substituted or unsubstituted C₁₋₆ alkoxy, amino, C₁₋₆ alkylamino, C₂₋₁₂ dialkylamino, N-protected amino, halo, or nitro;
N) a compound selected from: 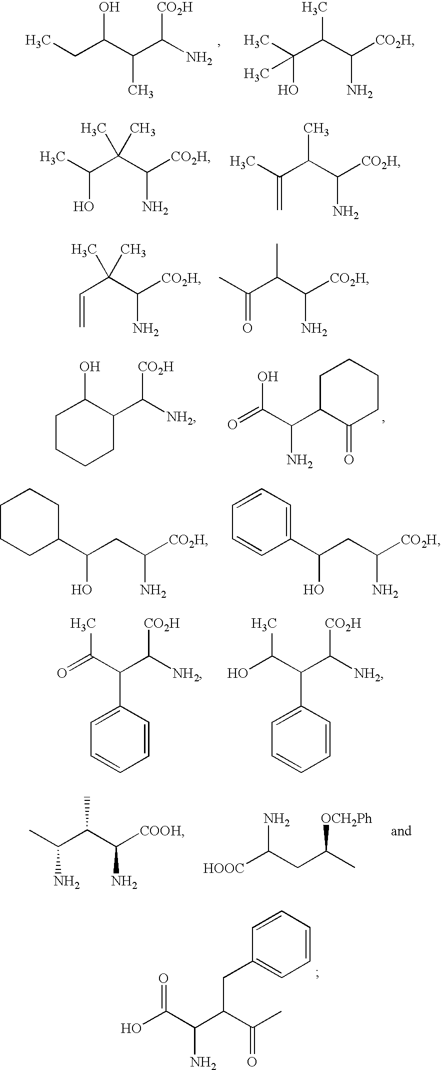
O) a compound selected from: 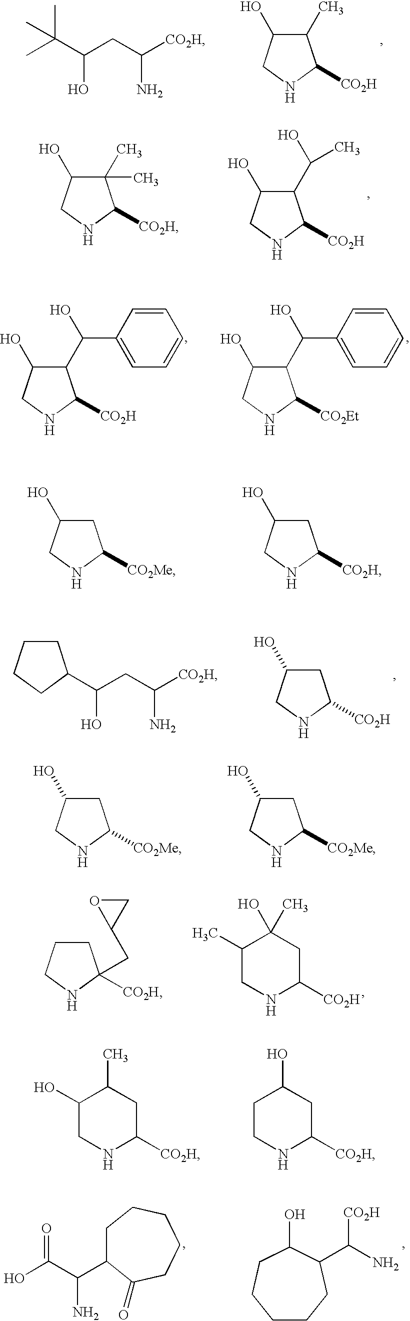 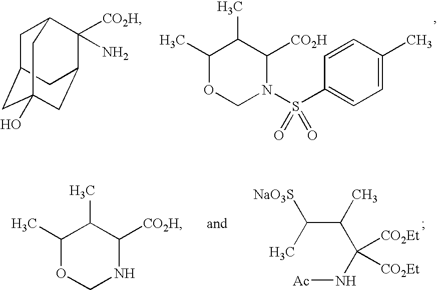
P) a compound of the formula: 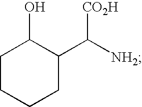
Q) a compound of the formula: 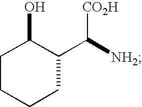
R) a compound of Formula (V): 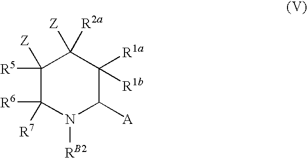
where each of A, R^{1a}, R^{1b}, R^{2a}, R⁴, and R^{B2}, are as defined previously in reference to Formula (I); R⁵, R⁶, and R⁷ are each, independently, hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms; and Z is XR⁴ or NR^{B1}R^{B2} as defined previously in reference to Formula (I);
S) a compound of Formula (V-A) 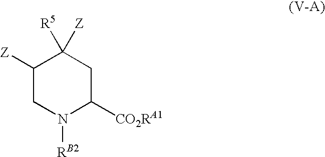
where each of R^{A1}, R^{B2}, and R⁴, are as defined previously in reference to Formula (I); R⁵ is hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms; and Z is XR⁴ or NR^{B1}R^{B2} as defined previously in reference to Formula (V);
T) a compound selected from the formula: 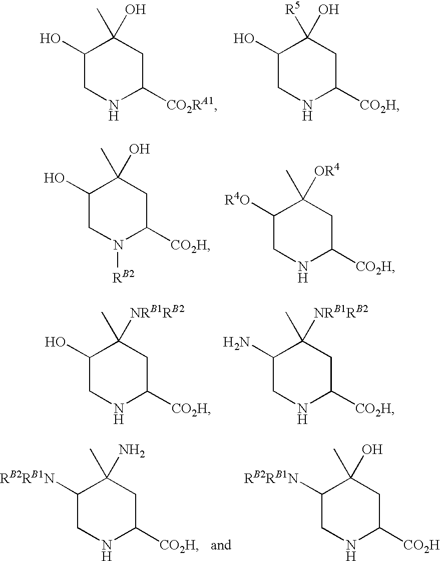
wherein R^{A1}, R^{B1}, R^{B2}, and R⁴ are as defined previously in reference to Formula (I), and where R⁵ is hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms;
U) a compound of Formula (VI): 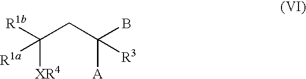
where A, B, X, R^{1a}, R^{1b}, R³, and R⁴ are as defined previously in reference to Formula (I);
V) a compound selected from: 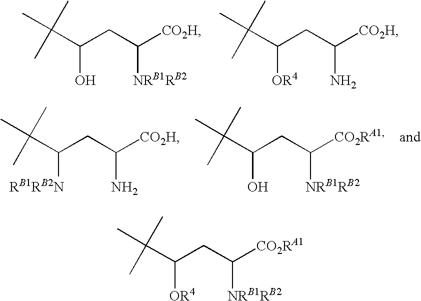
wherein R^{A1}, R^{B1}, R^{B2}, and R⁴ are as in Formula (I) as defined in claim 163;
W) a compound selected from: 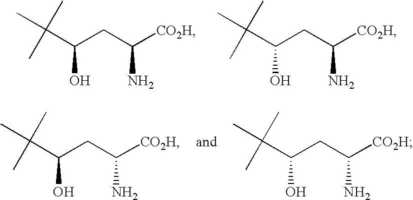
and
X) a compound selected from:

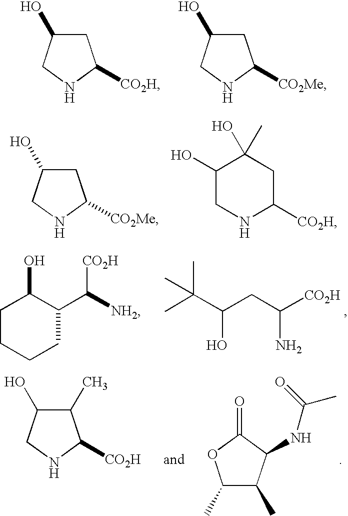

165. A pharmaceutical composition comprising: (1) a compound selected from the group consisting of: isomers of 4-hydroxyisoleucine, analogs of 4-hydroxyisoleucine and pharmaceutically acceptable lactones, salts, metabolites, solvates, and/or prodrugs of said isomers and analogs, and (2) an antiobesity agent and/or an antidiabetic agent.

166. A nutritional composition in form of a dietary supplement, medical food, complete meal, food additive or beverage comprising a compound selected from the group consisting of: isomers of 4-hydroxyisoleucine, analogs of 4-hydroxyisoleucine and pharmaceutically acceptable lactones, salts, metabolites, solvates, and/or prodrugs of said isomers and analogs.

167. A compound of Formula (I): 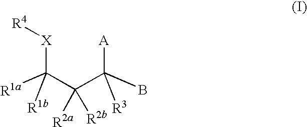
wherein
A is CO₂R^{A1}, C(O)SR^{A1}, C(S)SR^{A1}, C(O)NR^{A2}R^{A3}, C(S)NR^{A2}R^{A3}, C(O)R^{A4}, SO₃H, S(O)₂NR^{A2}R^{A3}, C(O)R^{A5}, C(OR^{A1})R^{A9}R^{A10}, C(SR^{A1})R^{A9}R^{A10}, C(═NR^{A1})R^{A5}, P(O)(OH)₂, 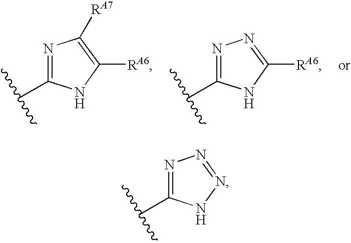
wherein
R^{A1} is hydrogen, or a substituted or unsubstituted group selected from C₁₋₆ alkyl, C₃₋₈ cycloalkyl, alkcycloalkyl, where the cycloalkyl group is of three to eight carbon atoms and the alkylene group is of one to four carbon atoms, C₂₋₆ alkenyl, C₂₋₆ alkynyl, C₆ or C₁₀ aryl, C₇₋₁₆ alkaryl, where the alkylene group is of one to four carbon atoms, C₁₋₉ heterocyclyl, or C₂₋₁₅ alkheterocyclyl, where the alkylene group is of one to four carbon atoms,
each of R^{A2} and R^{A3} is, independently, selected from the group consisting of (a) hydrogen, and a substituted or unsubstituted group selected from (b) C₁₋₆ alkyl, (c) C₃₋₈ cycloalkyl, (d) alkcycloalkyl, where the cycloalkyl group is of three to eight carbon atoms and the alkylene group is of one to four carbon atoms, (e) C₆ or C₁₀ aryl, and (f) C₇₋₁₆ alkaryl, where the alkylene group is of one to six carbon atoms, or R^{A2} taken together with R^{A3} and N forms a 5- or 6-membered ring, optionally containing O or NR^{A8}, wherein R^{A8} is hydrogen or C₁₋₆ alkyl,
R^{A4} is hydrogen, or a substituted or unsubstituted group selected from C₁₋₆ alkyl, C₃₋₈ cycloalkyl, alkcycloalkyl, where the cycloalkyl group is of three to eight carbon atoms and the alkylene group is of one to four carbon atoms, C₆ or C₁₀ aryl, C₇₋₁₆ alkaryl, where the alkylene group is of one to four carbon atoms, C₁₋₉ heterocyclyl, or C₂₋₁₅ alkheterocyclyl, where the alkylene group is of one to four carbon atoms,
R^{A5} is a peptide chain of 1-4 natural or unnatural amino acids, where the peptide is linked via its terminal amine group to C(O),
each of R^{A6} and R^{A7} is, independently, hydrogen, substituted or unsubstituted C₁₋₆ alkyl, C₁₋₄ perfluoroalkyl, substituted or unsubstituted C₁₋₆ alkoxy, amino, C₁₋₆ alkylamino, C₂₋₁₂ dialkylamino, N-protected amino, halo, or nitro, and
each of R^{A9} and R^{A10} is, independently, selected from the group consisting of (a) hydrogen, and a substituted or unsubstituted group selected from (b) C₁₋₆ alkyl, (c) C₃₋₈ cycloalkyl, (d) alkcycloalkyl, where the cycloalkyl group is of three to eight carbon atoms and the alkylene group is of one to four carbon atoms, (e) C₆ or C₁₀ aryl, and (f) C₇₋₁₆ alkaryl, where the alkylene group is of one to six carbon atoms, or R^{A9} taken together with R^{A10} and their parent carbon atom forms a 5- or 6-membered ring, optionally containing O or NR^{A8}, wherein R^{A8} is hydrogen or C₁₋₆ alkyl;
B is NR^{B1}R^{B2}, wherein
(i) each of R^{B1} and R^{B2} is, independently selected from the group consisting of (a) hydrogen, (b) an N-protecting group, and a substituted or unsubstituted group selected from (c) C₁₋₆ alkyl, (d) C₂₋₆ alkenyl, (e) C₂₋₆ alkynyl, (f) C₃₋₈ cycloalkyl, (g) alkcycloalkyl, where the cycloalkyl group is of three to eight carbon atoms, and the alkylene group is of one to ten carbon atoms, (h) C₆ or C₁₀ aryl, (i) C₇₋₁₆ alkaryl, where the alkylene group is of one to six carbon atoms, (j) C₁₋₉ heterocyclyl, (k) C₂₋₁₅ alkheterocyclyl, where the alkylene group is of one to six carbon atoms, (l) (CH₂)ₙC(O)R^{B3}, wherein n is 0, 1, 2 or 3, where R^{B3} is selected from the group consisting of hydrogen and a substituted or unsubstituted group selected from C₁₋₆ alkyl, C₆ or C₁₀ aryl, C₇₋₁₆ alkaryl, where the alkylene group is of one to six carbon atoms, C₁₋₉ heterocyclyl, or C₂₋₁₅ alkheterocyclyl, where the alkylene group is of one to six carbon atoms, (m) (CH₂)ₙCO₂R^{B4}, wherein n is 0, 1, 2 or 3, where R^{B4} is selected from the group consisting of hydrogen and a substituted or unsubstituted group selected from C₁₋₆ alkyl, C₆ or C₁₀ aryl, C₇₋₁₆ alkaryl, where the alkylene group is of one to six carbon atoms, C₁₋₉ heterocyclyl, or C₂₋₁₅ alkheterocyclyl, where the alkylene group is of one to six carbon atoms, (n) C(O)NR^{B5}R^{B6}, where each of R^{B5} and R^{B6} is, independently, selected from the group consisting of hydrogen and a substituted or unsubstituted group selected from C₁₋₆ alkyl, C₆ or C₁₀ aryl, C₇₋₁₆ alkaryl, where the alkylene group is of one to six carbon atoms, C₁₋₉ heterocyclyl, and C₂₋₁₅ alkheterocyclyl, where the alkylene group is of one to six carbon atoms, or R^{B5} taken together with R^{B6} and N forms a 5- or 6-membered ring, optionally containing a non-vicinal O, S, or NR′, where R′ is H or C₁₋₆ alkyl, (o) S(O)₂R^{B7}, where R^{B7} is selected from the group consisting of hydrogen and a substituted or unsubstituted group selected from C₁₋₆ alkyl, C₆ or C₁₀ aryl, C₇₋₆ alkaryl, where the alkylene group is of one to six carbon atoms, C₁₋₉heterocyclyl, or C₂₋₁₅ alkheterocyclyl, where the alkylene group is of one to six carbon atoms, and (p) a peptide chain of 1-4 natural or unnatural alpha-amino acid residues, where the peptide is linked via its terminal carboxy group to N, with the proviso that no two groups are bound to the nitrogen atom through a carbonyl group or a sulfonyl group, or
(ii) R^{B1} taken together with R^{B2} and N forms a substituted or unsubstituted 5- or 6-membered ring, optionally containing O or NR^{B8}, wherein R^{B8} is hydrogen or C₁₋₆ alkyl, or
(iii) a 5- to 8-membered ring is formed when R^{B1} taken together with R^{1a} is a substituted or unsubstituted C₁₋₄ alkylene, or
(iv) a [2.2.1] or [2.2.2] bicyclic ring system is formed when R taken together with R^{1a} is a substituted or unsubstituted C₂ alkylene and R^{B1} taken together with R^{2a} is a substituted or unsubstituted C₁₋₂ alkylene, or
(v) a 4- to 8-membered ring is formed when R^{B1} taken together with R³ is a substituted or unsubstituted C₂₋₆ alkylene, or
(vi) a 6- to 8-membered ring is formed when RBI taken together with R⁴ is a substituted or unsubstituted C₁₋₃ alkylene, or
(vii) R^{B1} taken together with A and the parent carbon of A and B forms the following ring: 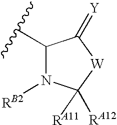
wherein each of Y and W is, independently, O, S, NR^{B8}, or CR^{A9}R^{A10}, wherein each of R^{A9} and R^{A10} is as previously defined and each of R^{A11} and R^{A12} is, independently, selected from the group consisting of (a) hydrogen and a substituted or unsubstituted group selected from (b) C₁₋₆ alkyl, (c) C₃₋₈ cycloalkyl, (d) alkcycloalkyl, where the cycloalkyl group is of three to eight carbon atoms and the alkylene group is of one to four carbon atoms, (e) C₆ or C₁₀ aryl, and (f) C₇₋₁₆ alkaryl, where the alkylene group is of one to six carbon atoms, or R^{A9} taken together with R^{A10} and their parent carbon atom forms a 5- or 6-membered ring, optionally containing O or NR^{A8}, wherein R¹⁸ is hydrogen or C₁₋₆ alkyl;
X is (i) absent (ii) hydrogen, (iii) a substituted or unsubstituted group selected from a C₁₋₆ alkyl, C₃₋₈ cycloalkyl, alkcycloalkyl, where the cycloalkyl group is of three to eight carbon atoms and the alkylene group is of one to four carbon atoms, C₆ or C₁₀ aryl, and C₇₋₁₆ alkaryl, where the alkylene group is of one to four carbon atoms, (iv) SO₃H; (v) O, (vi) S, or (vii) NR^{X1}, where R^{X1} is selected from the group consisting of (a) hydrogen (b) an N-protecting group, and a substituted or unsubstituted group selected from (c) C₁₋₆ alkyl, (d) C₂₋₆ alkenyl, (e) C₂₋₆ alkynyl, (f) C₃₋₈ cycloalkyl, (g) alkcycloalkyl, where the cycloalkyl group is of three to eight carbon atoms, and the alkylene group is of one to ten carbon atoms, (h) C₆ or C₁₀ aryl, (i) C₇₋₁₆ alkaryl, where the alkylene group is of one to six carbon atoms, (j) C₁₋₉ heterocyclyl, or (k) C₂₋₁₅ alkheterocyclyl, where the alkylene group is of one to six carbon atoms;
each of R^{1a} and R^{1b} is, independently, (a) hydrogen, (b) a substituted or unsubstituted group selected from C₁₋₆ alkyl, C₃₋₈ cycloalkyl, alkcycloalkyl, where the cycloalkyl group is of three to eight carbon atoms and the alkylene group is of one to four carbon atoms, C₂₋₆alkenyl, C₂₋₆alkynyl, C₆ or C₁₀ aryl, C₇₋₁₆ alkaryl, where the alkylene group is of one to four carbon atoms, C₁₋₉ heterocyclyl, and C₂₋₁₅ alkheterocyclyl, where the alkylene group is of one to four carbon atoms, or (c) R^{1a} together with R^{2a} and their base carbon atoms form a substituted or unsubstituted C₅₋₁₀ mono or fused ring system, or a 3- to 6-membered ring is formed when R^{1a} together with R⁴ is a substituted or unsubstituted C₁₋₄ alkylene (d) NR^{B1}R^{B2}, (e) a OR⁴ group, or (f) R^{1a} and R^{1b} together are ═O, ═N(C₁₋₆ alkyl), ═CR^{1c}R^{1d}, where each of R^{1c} and R^{1d} is, independently, hydrogen or a substituted or unsubstituted group selected from a C₁₋₆ alkyl or a C₂₋₅ alkylene moiety forming a spiro ring;
each of R^{2a} and R^{2b} is, independently, hydrogen, F, Cl, Br, I, a substituted or unsubstituted group selected from C₁₋₆ alkyl, C₃₋₈ cycloalkyl, alkcycloalkyl, where the cycloalkyl group is of three to eight carbon atoms and the alkylene group is of one to four carbon atoms, C₂₋₆ alkenyl, C₂₋₆ alkynyl, C₆ or C₁₀ aryl, C₇₋₁₆ alkaryl, where the alkylene group is of one to four carbon atoms, C₁₋₉ heterocyclyl, or C₂₋₁₅ alkheterocyclyl, where the alkylene group is of one to four carbon atoms, or R^{2a} and R^{2b} together are ═O, ═N(C₁₋₆ alkyl), ═CR^{2c}R^{2d}, where each of R^{2c} and R^{2d} is, independently, hydrogen or substituted or unsubstituted C₁₋₆ alkyl, or a substituted or unsubstituted C₂₋₅ alkylene moiety forming a spiro ring, or R^{2a} together with R^{1a} and their base carbon atoms form a substituted or unsubstituted C₅₋₁₀ mono or fused ring system;
R³ is hydrogen, COOR^{A1}, or a substituted or unsubstituted group selected from C₁₋₆ alkyl, alkcycloalkyl, where the cycloalkyl group is of three to eight carbon atoms and the alkylene group is of one to four carbon atoms, C₂₋₆ alkenyl, C₂₋₆ alkynyl, C₇₋₁₆ alkaryl, where the alkylene group is of one to four carbon atoms, or C₂₋₁₅ alkheterocyclyl, where the alkylene group is of one to four carbon atoms; and
R⁴ is absent, hydrogen, a substituted or unsubstituted group selected from C₁₋₆ alkyl, C₃₋₈ cycloalkyl, alkcycloalkyl, where the cycloalkyl group is of three to eight carbon atoms and the alkylene group is of one to four carbon atoms, C₂₋₆ alkenyl, C₂₋₆ alkynyl, C₆ or C₁₀ aryl, C₇₋₁₆ alkaryl, where the alkylene group is of one to four carbon atoms, C₁₋₉ heterocyclyl, or C₂₋₁₅ alkheterocyclyl, where the alkylene group is of one to four carbon atoms, or a 3- to 6-membered ring is formed when R⁴ together with R^{1a} is a substituted or unsubstituted C₁₋₄ alkylene, or a 6- to 8-membered ring is formed when R⁴ taken together with R^{B1} is a substituted or unsubstituted C₁₋₃ alkylene;
or a pharmaceutically acceptable lactone, salt, metabolite, solvate, and/or prodrug thereof.

168. The compound of claim 167, wherein said compound is selected from the group consisting of, or a pharmaceutically acceptable lactone, salt, metabolite, solvate, and/or prodrug thereof:
A) a compound of Formula (II): 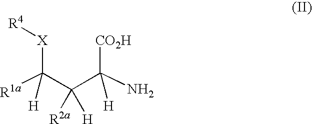
wherein each of X and R⁴ is as previously defined in reference to Formula (I) and each of R^{1a} and R^{2a} is, independently, substituted or unsubstituted C₁₋₆ alkyl or R^{1a} together with R^{2a} and their base carbon atoms form a substituted or unsubstituted 6 membered ring;
B) a compound of Formula (III): 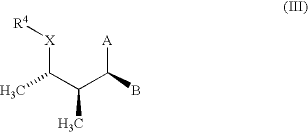
wherein A is CO₂R^{A1}, C(O)SR^{A1}, C(O)NR^{A2}R^{A3}, or C(O)R^{A5}; and each of R^{A1}, R^{A3}, R^{A3}, R^{A5}, B, X, and R⁴ is as previously defined in reference to Formula (I);
C) a compound of Formula (IV): 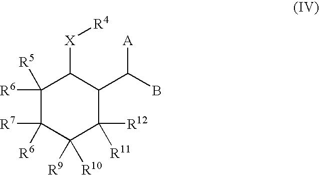
wherein A is CO₂R^{A1}, C(O)SR^{A1}, C(O)NR^{A2}R^{A3}, or C(O)R^{A5}; each of B, X, and R⁴ is as previously defined in reference to Formula (I); and each of R⁵, R⁶, R⁷, R⁸, R⁹, R¹⁰, R¹¹, and R¹² is, independently, hydrogen, or a substituted or unsubstituted group selected from C₁₋₆ alkyl, C₃₋₈ cycloalkyl, alkcycloalkyl, where the cycloalkyl group is of three to eight carbon atoms and the alkylene group is of one to four carbon atoms, C₂₋₆ alkenyl, C₂₋₆ alkynyl, C₆ or C₁₀ aryl, C₇₋₁₆ alkaryl, where the alkylene group is of one to four carbon atoms, C₁₋₉ heterocyclyl, or C₂₋₁₅ alkheterocyclyl, where the alkylene group is of one to four carbon atoms;
D) a compound of a formula selected from: 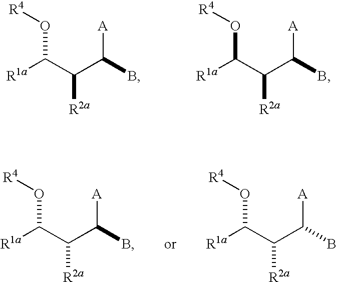
wherein each of A, B, and R⁴ is as previously defined in reference to Formula (I), and each of R^{1a} and R^{2a} is, individually, a substituted or unsubstituted group selected from C₁₋₆ alkyl, C₃₋₈ cycloalkyl, alkcycloalkyl, where the cycloalkyl group is of three to eight carbon atoms and the alkylene group is of one to four carbon atoms, C₂₋₆ alkenyl, C₂₋₆ alkynyl, C₆ or C₁₀ aryl, C₇₋₁₆ alkaryl, where the alkylene group is of one to four carbon atoms, C₁₋₉ heterocyclyl, or C₂₋₁₅ alkheterocyclyl, where the alkylene group is of one to four carbon atoms;
E) a compound of formula I as defined in claim 167, wherein A is CO₂H, B is NH-p-toluenesulfonyl, R⁴ is H, and each of R^{1a} and R^{2a} is CH₃;
F) a compound of formula I as defined in claim 167, wherein A is CO₂H, B is NH₂, R⁴ is H, and each of R^{1a} and R^{2a} is a substituted or unsubstituted C₁₋₆ alkyl;
G) a compound of formula I as defined in claim 167, wherein A is CO₂H, B is NH₂, X is O, and R⁴ is H;
H) a compound of a formula selected from: 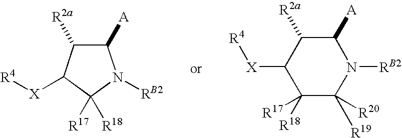
wherein each of A, X, R^{2a}, R⁴, and R^{B2} is as previously defined in reference to Formula (I), and each of R¹⁷, R¹⁸, R¹⁹, and R²⁰ is hydrogen or substituted or unsubstituted C₁₋₆ alkyl;
I) a compound of a formula selected from: 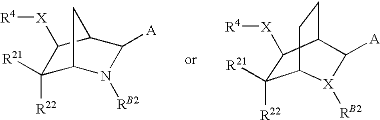
wherein each of A, X, R⁴, and R^{B2} is as previously defined in reference to Formula (I), and each of R²¹ and R²² is hydrogen or substituted or unsubstituted C₁₋₆ alkyl;
J) a compound of the formula: 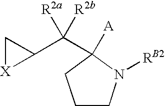
wherein each of A, X, R^{2a}, R^{2b}, and R^{B2} is as previously defined in reference to Formula (I);
K) a compound of the formula: 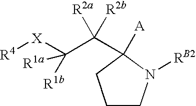
wherein each of A, X, R^{1a}, R^{1b}, R^{2a}, R^{2b}, R⁴, and R^{B2} is as previously defined in reference to Formula (I);
L) a compound of formula I as defined in claim 167, wherein R^{1a} together with R^{2a} and their base carbon atoms form a substituted or unsubstituted C₅₋₁₀ mono or fused ring system, optionally containing a non-vicinal O, S, or NR′, where R′ is H or C₁₋₆ alkyl;
M) a compound of a formula selected from: 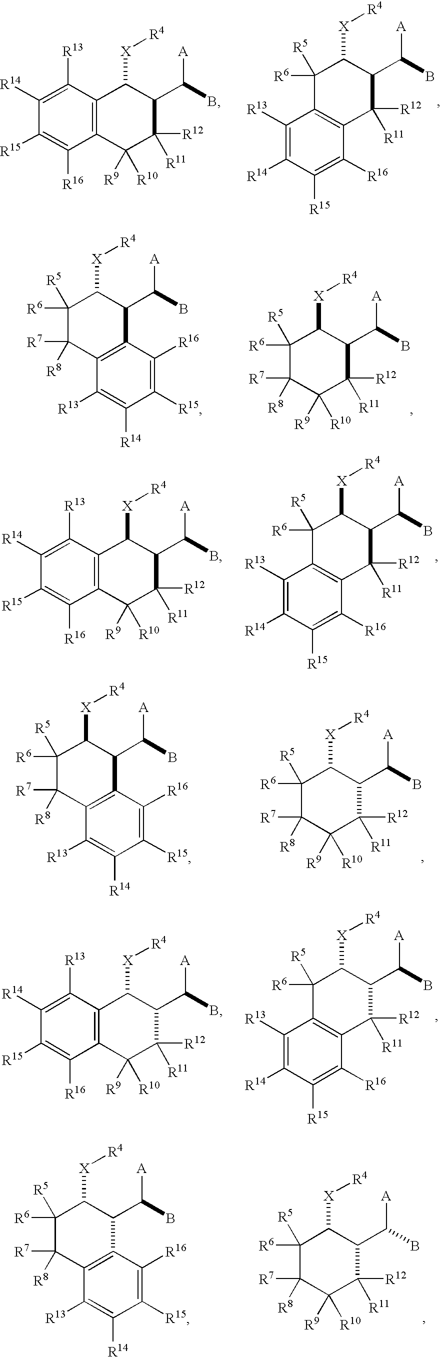 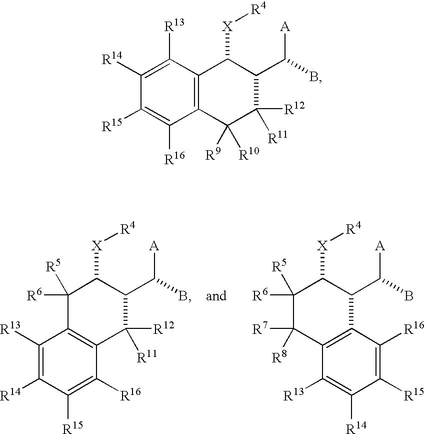
wherein each of A, B, X, and R⁴ is as defined previously in reference to Formula (I), and each of R⁵, R⁶, R⁷, R⁸, R⁹, R^{1b}, R¹¹, and R¹² is, independently, hydrogen, or a substituted or unsubstituted group selected from C₁₋₆ alkyl, C₃₋₈ cycloalkyl, alkcycloalkyl, where the cycloalkyl group is of three to eight carbon atoms and the alkylene group is of one to four carbon atoms, C₂₋₆ alkenyl, C₂₋₆ alkynyl, C₆ or C₁₀ aryl, C₇₋₁₆ alkaryl, where the alkylene group is of one to four carbon atoms, C₁₋₉ heterocyclyl, or C₂₋₁₅ alkheterocyclyl, where the alkylene group is of one to four carbon atoms; and
each of R¹³, R¹⁴, R¹⁵, and R¹⁶ is, independently, hydrogen, substituted or unsubstituted C₁₋₆ alkyl, C₁₋₄ perfluoroalkyl, substituted or unsubstituted C₁₋₆ alkoxy, amino, C₁₋₆ alkylamino, C₂₋₁₂ dialkylamino, N-protected amino, halo, or nitro;
N) a compound selected from: 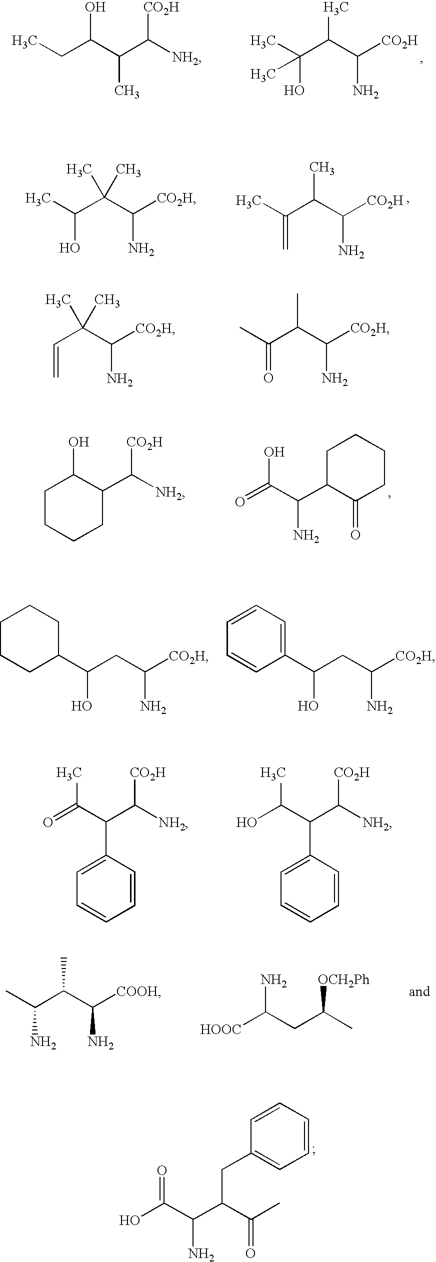
O) a compound selected from: 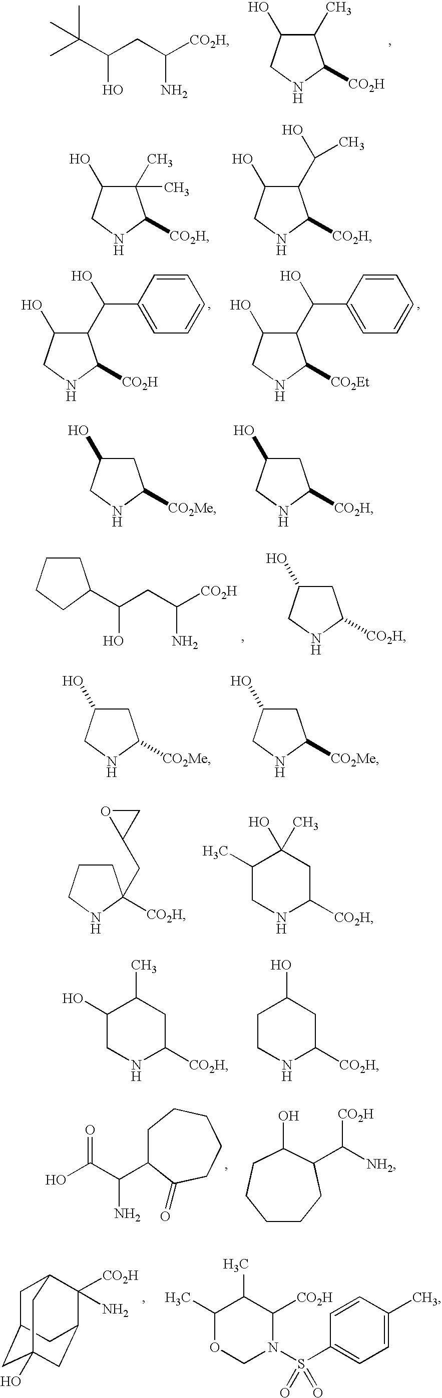 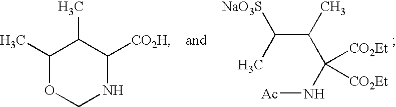
P) a compound of the formula: 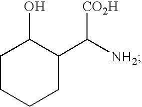
Q) a compound of a formula selected from: 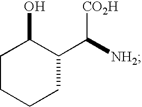
R) a compound of Formula (V): 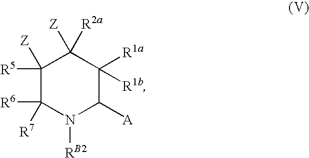
where each of A, R^{1a}, R^{1b}, R^{2a}, R⁴, and R^{B2}, are as defined previously in reference to Formula (I); R⁵, R⁶, and R⁷ are each, independently, hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms; and Z is XR⁴ or NR^{B1}R^{B2} as defined previously in reference to Formula (I);
S) a compound of Formula (V-A) 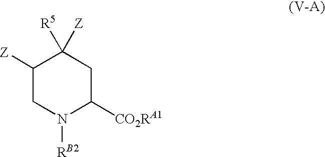
where each of R^{A1}, R^{B2}, and R⁴, are as defined previously in reference to Formula (I); R⁵ is hydrogen, substituted or unsubstituted C₁₋₆ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms; and Z is XR⁴ or NR^{B1}R^{B2} as defined previously in reference to Formula (V);
T) a compound of a formula selected from: 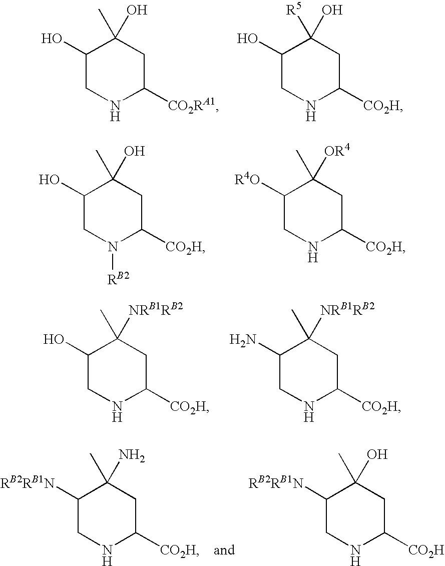
wherein R^{A1}, R^{B1}, R^{B2}, and R⁴ are as defined previously in reference to Formula (I), and where R⁵ is hydrogen, substituted or unsubstituted C₁ alkyl, substituted or unsubstituted C₃₋₈ cycloalkyl, substituted or unsubstituted alkcycloalkyl, where the cycloalkyl group is of three to eight carbon atoms and the alkylene group is of one to four carbon atoms, substituted or unsubstituted C₂₋₆ alkenyl, substituted or unsubstituted C₂₋₆ alkynyl, substituted or unsubstituted C₆ or C₁₀ aryl, substituted or unsubstituted C₇₋₁₆ alkaryl, where the alkylene group is of one to four carbon atoms, substituted or unsubstituted C₁₋₉ heterocyclyl, or substituted or unsubstituted C₂₋₁₅ alkheterocyclyl, where the alkylene group is of one to four carbon atoms;
U) a compound of Formula (VI): 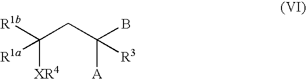
where A, B, X, R^{1a}, R^{1b}, R³, and R⁴ are as defined previously in reference to Formula (I);
V) a compound selected from: 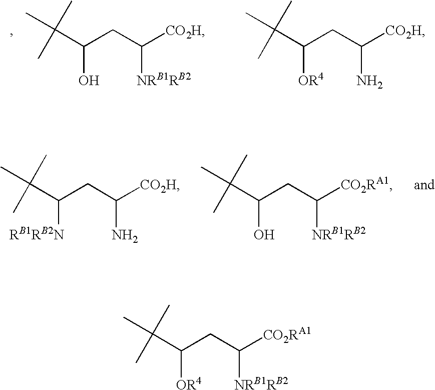
wherein R^{A1}, R^{B1}, R^{B2}, and R⁴ are as in Formula (I) as defined in claim 167;
W) a compound selected from: 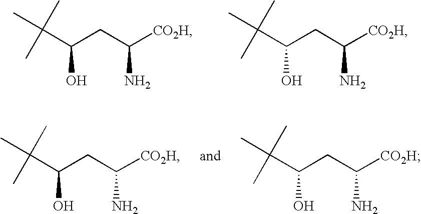
and
X) a compound selected from: [chemical structure]
